SEQUENCE LISTING (270 sequences)

SEQ ID NO: 1 - protein, 379 aa, unidentified
MKIVLVIDQFDDANNGTTISARRFAQALKNHGNEVRVIATGKPADYKYAVRQMRFFPVVEHLITSQGMRLAIPNRHVFEKAAAWADVVHFMMPSPLGIMGLKHVEKLGIPHTAAFHCQPENITFTLHMGNSRRVNDFVYNRFRDTFFNRFTHIHCPSNMIANQLRQHGYTARLHVISNGISPEYIYGKREKEPWMQGLFNVLMVGRYAGEKRQDELIDACAKSRHAREIQVILAGKGPLEKKYRRLAEKLPNPIVMEFYEPARLLEILHMADLYVHTSDAEIEAMSCMEAFACGLVPVIADSPRSATPQFALDERSLFPAGDTDALAQRIDWWIEHPEERQAMERRYAEHARQYSLEESIRQTEEMFRQAIAEQRGAKA

SEQ ID NO: 2 - protein, 379 aa, unidentified; identical to SEQ ID NO: 1

SEQ ID NO: 3 - protein, 389 aa, Helicobacter mustelae
MTIGIVIDSYNDRSNGTSMTAFRFAREFVKKGHEVRIVACNVSKSMSDEEDLKLYPVKQRYIPIVTEVSKKQHMIFGAPDLEVLQSAVVGCDIVHFYMPFALEIAGMHLCRSLRIPYISAFHVQPQHISYNMNMNFSWFNTYLFKRFYKHFYRYTHHIHCPSKFIEKELQRENYGGKKYTISNGFFGGDRVMADPYEDSFFHIASVGRFSKEKKQDIIIKAIAKNPYADKIKLHLHGVGPREKYLKNLCNKLLINKPEFGFIDNGALLEKLAKMHLYVHAAKVESEAISCLEAISLGVVPVIADSETSATVQFALDPLSLFEVNNVADLSNKITYWIEHPKELLAYKQKYAESALQYSLDKSIEETLGLYEEAIRDFRDQPALFDRINA

SEQ ID NO: 4 - protein, 367 aa, Lactobacillus sp.
MKILIVIDDYHNNSNGMSISTQRFVKEFKKLGCDVRVLAIGDVSYSLPEMKIPFFAKLIAQQGFHFALPVRKTTLKAVQWADYVHLDTPFPIGWQAGHLAKKMGKTVTGTFHIYPQNMTASVPILNQKWINNLIMFVFKKISYKNCDVIQCPTAKVKRNLQKYHFPQKLVVISNGIAQAFIDNPHKTDTQQDFTILCIGRFSNEKDQYTLFRAMQVCKYAKNINLIFAGQGPLKDKFEALAKTLPRKPIMRYFNSKQLRKLEAKSQLVVHCANVEVEGMSCMEAFASGCVPIIASSDLSSTASYSLTKNNQFKAEDYHELAKRIEYWYEHPLELGNMSKKYQTYARSLNVNKCAKMALSMIRSAKKR

SEQ ID NO: 5 - protein, 376 aa, Oscillibacter sp.
MKIVLVIDQFDDANNGTTISARRFAQALRDHGNEVRVIATGKPADYKYAVRQMRFFPIVEHLITSQGMRLAIPNRHVFEKAAAWADVVHFMMPSPLAVMGLKHVEKLGIPHTAAFHCQPENITFTLHLGNSQRANDFVYNRFRDSFFNRFTHIHCPSNMIAEQLRQHGYTAQLHVISNGISPEYFYGKQPKETWMQGFFNVLMVGRYAGEKRQDELIAACTKCRHAKEIQVILAGRGPLEKKYRRLAEKLPNPIVMNFYEPARLLEILHMADLYVHTSDAEIEAMSCMEAFACGLVPVIADSPRSATPQFALDERSLFPAGDTDVLAERIDWWLEHPEERREMELRYAEHAKQYTLEHSIQQTEEMFRMAIREQRG

SEQ ID NO: 6 - protein, 376 aa, unidentified
MKIVLVIDQFDDANNGTTISARRFAQALRDHGNEVRVIATGKPADYKYAVRQMRFFPIVEHLITSQGMRLAIPNRHVFEKAAAWADVVHFMMPSPLAVMGLKHVEKLGIPHTAAFHCQPENITFTLHLGNSQRANDFVYNRFRDSFFNRFTHIHCPSNMIAEQLRQHGYTAQLHVISNGISPEYFYGKQPKETWMQGFFNVLMVGRYAGEKRQDELIAACTKCRHAKEIQVILAGRGPLEKKYRRLAEKLPNPIVMSFYEPARLLEILHMADLYVHTSDAEIEAMSCMEAFACGLVPVIADSPRSATPQFALDERSLFPAGDTDVLAERIDWWLEHPEERREMELRYAEHAKQYTLEHSIQQTEEMFRMAIREQRG

SEQ ID NO: 7 - protein, 379 aa, Oscillibacter sp.
MKIVLVIDQFDDANNGTTISARRFAQALKNHGNEVRVIATGKPADYKYAVRQMKFFPVVEHLITSQGMRLAIPNRHVFEKAAAWADVVHFMMPSPLGIMGLKHVEKLGIPHTAAFHCQPENITFTLHLGNSRRVNDFVYNRFRDTFFNRFTHIHCPSNMIANQLRQHGYTAQLHVISNGISPEYTYGKREKEPWMQGFFNVLMVGRYAGEKRQDELIDACAKSRHAREIQVILAGKGPLEKKYRRLAEKLPNPIVMEFYEPARLLEILHMADLYVHTSDAEIEAMSCMEAFACGLVPVIADSPRSATPQFALDERSLFPAGDTDALAQRIDWWIEHPEERQAMERRYAEHARQYSLEESIRQTEEMFRQAIAEQRGAKA

SEQ ID NO: 8 - protein, 379 aa, Oscillibacter sp.
MKIVLVIDQFDDANNGTTISARRFAQALKNHGNEVRVIATGKPADYKYAVRQMKFFPVVEHLITSQGMRLAIPNRHVFEKAAAWADVVHFMMPSPLGIMGLKHVEKLGIPHTAAFHCQPENITFTLHLGNSRRVNDFVYNRFRDTFFNRFTHIHCPSNMIANQLRQHGYTAQLHVISNGISPEYTYGKREKEPWMQGFFNVLMVGRYAGEKRQDELIDACAKSRHAREIQVILAGKGPLEKKYRRLAEKLPNPIVMEFYEPARLLEILHMADLYVHTSDAEIEAMSCMEAFACGLVPVIADSPRSATPQFALDERSLFPAGDTDALAARIDWWIEHPEERQAMERRYAEHARQYSLEESIRQTEEMFRQAIAEQRGAKA

SEQ ID NO: 9 - protein, 379 aa, unidentified; identical to SEQ ID NO: 1

SEQ ID NO: 10 - protein, 379 aa, Oscillibacter sp.
MKIVLVIDQFDDANNGTTISARRFAQALKDNGNEVRVIATGKPADYKYAVRQLKLFPVVEHLLTSQGMRLAVPNKHVFEKAAAWADVVHFMMPSPLGVMGLKHVEKLGIPHTAAFHCQPENITFTLHLGNSKRVNDFVYNRFRDSFFNRFTHIHCPSNMIADQLRQHGYTAQLHVISNGISPEYTYGKRDKEPWMQGFFNVLMVGRYAGEKRQDELIDACAKSRHAQEIQVILAGKGPLEKKYRKLAEKLPNPIVMDFYEPARLLDILHMADLYVHTSDAEIEAMSCMEAFACGLVPVIADSPRSATPQFALDERSLFPAGDTDALAARIDWWIEHPEERREMELRYADHARQYSLEASIRRTEEMFRQAIAEQRGARA

SEQ ID NO: 11 - protein, 379 aa, Oscillibacter sp.
MKIVLVIDQFDDANNGTTISARRFAQALKNHGNEVRVIATGKPADYKYAVRQMRFFPVVEHLITSQGMRLAIPNRHVFEKAAAWADVVHFMMPSPLGIMGLKHVEKLGIPHTAAFHCQPENITFTLHMGNSRRVNDFVYNRFRDTFFNRFTHIHCPSNMIANQLRQHGYTARLHVISNGISPEYIYGKREKEPWMQGLFNVLMVGRYAGEKRQDELIDACAKSRHAREIQVILAGKGPLEKKYRRLAEKLPNPIVMEFYEPARLLEILHMADLYVHTSDAEIEAMSCMEAFACGLVPVIADSPRSATPQFALDERSLFPAGDTDALAQRIDWWIEHPEERQAMERRYAEHARQYSLEESIRQAEEMFRQAIAEQRGAKA

SEQ ID NO: 12 - protein, 379 aa, unidentified
MKIVLVIDQFDDANNGTTISARRFAQALKNHGNEVRVIATGKPADYKYAVRQMRFFPVVEHLITSQGMRLAIPNRHVFEKAAAWADVVHFMMPSPLGIMGLKHVEKLGIPHTAAFHCQPENITFTLHMGNSRRVNDFVYNRFRDTFFNRFTHIHCPSNMIANQLRQHGYTARLHVISNGISPEYIYGKREKEPWMQGLFNVLMVGRYAGEKRQDELIDACAKSRHAREIQVILAGKGPLEKKYRRLAEKLSNPIVMEFYEPARLLEILHMADLYVHTSDAEIEAMSCMEAFACGLVPVIADSPRSATPQFALDERSLFPAGDTDALAQRIDWWIEHPEERQAMERRYAEHARQYSLEESIRQTEEMFRQAIAEQRGAKA

SEQ ID NO: 13 - protein, 379 aa, Oscillibacter valericigenes
MKIVLVIDQFDDANNGTTISARRFAQALKASGNEVRVIATGKPADYKYAVRQMKFFPVVEHLITSQGMRLAIPNKHVFEKAAAWADVVHFMMPSPLGIMGLKHVEKLGIPHTAAFHCQPENITFTLHLGNSRRVNDFVYNRFRDTFFNRFTHIHCPSNMIADQLRSHGYTAQLHVISNGISPEYTYGKQEKEPWMQGFFNVLMVGRYAGEKRQDELIEACAKSRHAQEIQVILAGKGPLEKKYRRLAEKLPNPIVMSFYEPARLLEILHMADLYVHTSDAEIEAMSCMEAFACGLVPVIADSPRSATPQFALDGRSLFPAGDTDALAERIDWWIEHPAERQEMERRYAEHAKQYSLAESIRRTEEMFRQAIAEQRGTRA

SEQ ID NO: 14 - protein, 377 aa, unidentified
MKIVLVIDQFDDANNGTTISARRFAQALRDHGNEVRVIATGKPADYKYAVRQMRFFPVVEHLITSQGMRLAIPNRHVFEKAAAWADVVHFMMPSPLGIMGLRHVEKLGIPHTAAFHCQPENITFTLHMGNSKRANDFVYNRFRDTFFNRFTHIHCPSNMIADQLRSHGYTAQLHVISNGISPEYTYGKRPKEPWMEGFFNVLMVGRYAGEKRQDELIEAAARCRHAQQIQVILAGKGPLEKKYRRLAEKLPNPVVMGFYEPARLLEILHMADLYVHTSDAEIEAMSCMEAFACGLVPVIADSPRSATPQFALDERSLFPAGDVDALAERIDYWIDHPQERAEMERRYAVHAGQYSLRNSIFQTEEMFRQAIEEQKRA

SEQ ID NO: 15 - protein, 377 aa, unidentified
MKIVLVIDQFDDANNGTTISARRFAQALRDHGNEVRVIATGKPADYKYAVRQMRFFPVVEHLITSQGMRLAIPNRHVFEKTAAWADVVHFMMPSPLGIMGLRHVEKLGIPHTAAFHCQPENITFTLHMGNSKRANDFVYNRFRDTFFNRFTHIHCPSNMIADQLRSHGYTAQLHVISNGISPEYTYGKRPKEPWMEGFFNVLMVGRYAGEKRQDELIEAAARCRHAQQIQVILAGKGPLEKKYRRLAEKLPNPVVMGFYEPARLLEILHMADLYVHTSDAEIEAMSCMEAFACGLVPVIADSPRSATPQFALDERSLFPAGDVDALAERIDYWIDHPQERAEMERRYAVHAGQYSLRNSIFQTEEMFRQAIEEQKRA

SEQ ID NO: 16 - protein, 378 aa, Oscillibacter sp.
MKIVLVIDQFDDANNGTTISARRFASALKAHGNEVRVIATGKPADYKYAVRQMRFFPIVEHLITSQGMRLAIPNRHVFEKAAAWADVVHFMMPSPLAIMGLRHVEKIGIPHTAAFHCQPENITFTLHMGNSRRVNDFVYGKFRDTFFNRFTHIHCPSSMIANQLRSHGYTAQLHVISNGISPEYVYGKRPKEDWMEGFFNVLMVGRYAGEKRQDVLIEAAARCRHAGEIQVILPGKGPLEKKYRHLAEKLPNPIVMGFYEPARLIEILHMADLYVHTSDAEIEAMSCMEAFACGLVPVIADSPRSATPQFALDERSLFPAGDSDTLAERIDWWIEHPAERQEMERRYAEHAKQYALDRSIQQTEEMFRQAIREQGKKN

SEQ ID NO: 17 - protein, 380 aa, unidentified
MKIVLVIDQFDDANNGTTISARRFAMALKEHGNEVRVIAIGKNTDYKYAVRQMKFPPVVEHLITSQGMRLAIPNRHVFEKAAAWADVVHFMMPSPLAVMGLKHVEKLGIPHTAAFHCQPENITFTLHMGNSRKANDLLYHGFRDTFFNRFTHIHCPSNMIANQLRQHGYTAQLHVISNGISPEYFYDKRPKEDWMQGYFNVLMVGRYAGEKRQDELIEACAKSRHAREIQVILAGKGPLEKKYRKLAEKLPNPIVMQFYEPSRLIEILHMADLYVHTSDAEIEAMSCMEAFACGLVPVIANSPRSATPQFALDERSLFPAGDTDALAERIDYWIEHPQEREEMERRYAEHAKQYALERSIEQTEDMFRMAIAEQKATTKE

SEQ ID NO: 18 - protein, 378 aa, Oscillibacter sp.
MKIVLVIDQFDDANNGTTISARRFASALKAHGNEVRVIATGKPADYKYAVRQMRFFPIVEHLITSQGMRLAIPNKHVFEKAAAWADVVHFMMPSPLAVMGLRHVEKIGIPHTAAFHCQPENITFTLHMGNSRRVNDFVYGKFRDTFFNRFTHIHCPSAMIANQLRDHGYTAQLHVISNGISPEYVYGKRAKEDWMEGFFNILMVGRYAGEKRQDVLIEAAAKCRHAQEIQVILPGKGPLEKKYRHLAESLPNPVVMGFYEPARLIEILHMADLYVHTSDAEIEAMSCMEAFACGLVPVIADSPRSATPQFALDQRSLFPAGDSDMLAQRIDWWIEHPQERREMERRYAQHAGQYTLERSIEQAEEMFRQAIRERERKN

SEQ ID NO: 19 - protein, 376 aa, Oscillibacter sp.
MKIVLVIDQFDDANNGTTISARRFAAALKEHGNEVRVIATGKPTDYKYAVRQMRFLPIVEHLITSQGMRLAIPNRHVFEKAAAWADVVHFMMPSPLAIMGLKHVERLGIPHTAAFHCQPENITYTLHLGNSKRVNDFVYVKFRDTFFNRFTHIHCPSNMIAGQLRDHGYTARLHVISNGISPQYVYGRRPQEEWMQGKFNVLMVGRYAGEKRQDVLIEACARCRHREEIQVILAGKGPLEKKYRRLAEKLSNPVVMEFYEPARLLEILHMADLYVHTSDAEIEAMSCMEAFACGLVPVIADSPRSATPQFALDERSLFPAGDSAALAEKIDWWMEHPREREEMGKRYGEHARQYALERSIEQTEEMFRMAIAEQRG

SEQ ID NO: 20 - protein, 374 aa, Oscillibacter sp.
MKIVLVIDQFDDANNGTTISARRFATALKEHGNEVRVIATGKPADYKYAVRQMKFFPIVEHLITSQGMRLAIPNRHVFEKAAAWADVVHFMMPSPLAIMGLKHVERLNIPHTAAFHCQPENITFTLHLGNSRRANDFVYGKFRDTFFNRFTHIHCPSNMIANQLREHGYTAQLHVISNGISPQYQYGRQPKEDWMKDRFNVLMVGRYAGEKRQDVLIKACAQCRHGAEIQVILAGKGPLEKKYRRLARNLPNPIVMEFYEPARLLEILHMADLYVHTSDAEIEAMSCMEAFACGLVPVIADSPRSATPQFALDERSLFPAGDSGALAERIDWWFEHPEERQEMGLRYAAHAEQYALSRSIEQTEEMFRQAIAER

SEQ ID NO: 21 - protein, 379 aa, Oscillibacter sp.
MKIVLVIDQFDDANNGTTISARRFASALKEHGNEVRVIATGKPTDYKYAVRQMKFLPIVEHLITSQGMRLAIPNRHVFEKAAAWADVVHFMMPSPLAIMGLRHVERLGIPHTAAFHCQPENITFTLHMGNNRRVNDFVYNRFRDTFFNRFTHIHCPSTMIANQLREHGYTAELHVISNGISPQYSYGRAPQEDWMQGRFNVLMVGRYAGEKRQDVLIDACAKCRHKDEIQVILAGKGPLEKKYRRMAEKLSNPIVMEFYEPSRLLEILHMADLYVHTSDAEIEAMSCMEAFACGLVPVIADSTRSATPQFALDERSLFPAGDSGALAEKIDWWLEHPEEREAMGRRYGEHAKQYALERSIEQTEEMFRTAIKEQRGPRP

SEQ ID NO: 22 - protein, 376 aa, Oscillibacter sp.
MKIVLVIDQFDDANNGTTISARRFATALKEHGNEVRVIATGKPTDYKYAVRQMKFLPIVEHLITSQGMRLAIPNKHVFEKAAAWADVVHFMMPSPLAVMGLKHVERLGIPHTAAFHCQPENITFTLHLGNSKRVNDFVYTKFRDTFFNRFTHIHCPSNMIADQLRQHGYTAQLHVISNGISPQYTYGRSPQEDWMKGKFNVLMVGRYAGEKRQDVLIDACARCRHQDDIQVILAGKGPLEKKYRRLAEKLPNPIVMEFYEPGRLLEILHMADLYVHTSDAEIEAMSCMEAFACGLVPVIADSPRSATPQFALDGRSLFPAGDPGALAEKIDWWFEHPQEREEMGKRYGEHAKRYALDRSIQQTEEMFQTAIREQKR

SEQ ID NO: 23 - protein, 374 aa, Oscillibacter sp.
MKIVLVIDQFDDANNGTTISARRFATALKEHGNEVRVIATGKPADYKYAVRQMKFFPIVEHLITSQGMRLAIPNRHVFEKAAAWADVVHFMMPSPLAIMGLKHVERLNIPHTAAFHCQPENITFTLHLGNSRRVNDFVYGKFRDTFFNRFTHIHCPSNMIANQLREHGYTAQLHVISNGISPQYQYGRQPKEDWMKDRFNVLMVGRYAGEKRQDVLIKACAQCRHGAEIQVILAGKGPLEKKYRRLARNLPNPIVMEFYEPARLLEILHMADLYVHTSDAEIEAMSCMEAFACGLVPVIADSPRSATPQFALDERSLFPAGDSGALAERIDWWFEHPEERQEMGLRYAAHAEQYALSRSIEQTEEMFRQAIAER

SEQ ID NO: 24 - protein, 376 aa, Oscillibacter sp.
MKIVLVIDQFDDANNGTTISARRFATALKEHGNEVRVIATGKPADYKYAVRQMKFFPIVEHLITSQGMRLAIPNRHVFEKAAAWADVVHFMMPSPLSIMGLKHVERLNIPHTAAFHCQPENITFTLHLGNSRRANDFVYSKFRDTFFNRFTHIHCPSNMIANQLREHGYTAQLHVISNGISPQYTYGRSPKEDWMKDRFQVLIVGRYAGEKRQDVLIKACTQCRHAREIQVILAGKGPLEKKYRRLAQSLPNPAVMEFYEPARLLEILHMADLYVHTSDAEIEAMSCMEAFACGLVPVIADSPRSATPQFALDERSLFPAGDSAALAERIDWWFEHPSEREEMGKRYAEHAKQYALSRSIEQTEEMFRQAIAENEP

SEQ ID NO: 25 - protein, 378 aa, Oscillibacter sp.
MKIVLVIDQFDDANNGTTISARRFAMALKEHGNEVRVIATGKPADYKYAVRQMKFFPIVEHLITSQGMRLAIPNRHVFEKAAAWADVVHFMMPSPLGVMGLRHVEKLGIPHTAAFHCQPENITFTLHMGNNKRVNDFVYNRFRDSFFNRFTHIHCPSNMIAEQLRQHGYTAKLHVISNGISPQYIYGKRPKEDWMQGMFNVLMVGRYAGEKRQDVLIEAASKCRHAKEIQVILAGKGPLEKKYRHLAEKELPNPVVMGFYEPSRLIEILHMCDLYVHTSDAEIEAMSCMEAFACGLVPVIANSPRSATPQFALDDRSLFPAGDAAALAEQIDWWIEHPAEREKMEHRYAEHAKQYTLERSIELAEGMFQEAIDEQKQV

SEQ ID NO: 26 - protein, 374 aa, Oscillibacter sp.
MKIVLVIDQFDDANNGTTISARRFATALKEHGNEVRVIATGKPADYKYAVRQMKFFPIVEHLITSQGMRLAIPNRHVFEKAAAWADVVHFMMPSPLAIMGLKHVERLNIPHTAAFHCQPENITFTLHLGNSRRANDFVYGKFRDTFFNRFTHIHCPSNMIANQLREHGYTAQLHVISNGISPQYQYGRQPKEDWMKDRFNVLMVGRYAGEKRQDVLIKACAQCRHGAEIQVILAGKGPLEKKYRRLARNLPNPIVMEFYEPARLLEILHMADLYVHTSDAEIEAMSCMEAFACGLVPVIADSPRSATPQFALDERSLFPAGDSGALAERIDWWFERPEERQEMGLRYAAHAEQYALSRSIKQTEEMFRQAIAER

SEQ ID NO: 27 - protein, 378 aa, Oscillibacter sp.
MKIVLVIDQFDDANNGTTISARRFAAALKAHGNEVRVIATGKPADYKYAVRQMRFFPIVEHLITSQGMRLAIPNKHVFEKAAAWADVVHFMMPSPLAIMGLKHVEKIGIPHTAAFHCQPENITFTLHMGNSRRMNDFVYGKFRDTFFNRFTHIHCPSNMIANQLREHGYTAQLHVISNGISSEYVYGKRPKENWMEGFFNILMVGRYAGEKRQDVLIEAAAKCRHAGEIQVILPGKGPLEKKYRHLAEKLPNPVVMGFYEPARLIEILHMADLYVHTSDAEIEAMSCMEAFACGLVPVIADSPRSATPQFALDERSLFPAGDSDVLAERIDWWIEHPEERREMERRYAEHAEQYTLERSIEQTEEMFRQAIGEKSRTV

SEQ ID NO: 28 - protein, 377 aa, Oscillibacter sp.
MKIVLVIDQFDDANNGTTISARRFARALKAHGNEVRVIATGKPADYKYAVRQMRFFPIVEHLITSQGMRLAIPNRHVFEKAAAWADVVHFLMPSPLAVMGLKHVEKLGIPHTAAFHCQPENITFSLHMGNSRRVNDFVYEKFRDTFFNRFTHIHCPSHMIAGQLEAHGYTAQLHVISNGISPEYIYGRQPKEPWMEGKFNILMVGRYAGEKRQDVLIDAAAKCRRAGEIQVILAGKGPLEKKYRHLAENLPNPIVMQFFEPSRLLEILHMADLYVHTSDAEIEAMSCMEAFACGLVPVIADSPRSATPQFALDGRSLFPAGDSSALAEKIDWWMEHPEERREMGLRYGEHARQYALERSIAQMEEMFRLAIQEQRDR

SEQ ID NO: 29 - protein, 377 aa, Oscillibacter sp.
MKIVLVIDQFDDANNGTTISARRFAMALKEHGNEVRVIAIGKPADYKYAVRQMRFFPIVEHLITSQGMRLAIPNKHVFEKAAAWADVVHFMMPSPMAIMGLKHVERLGIPHTAAFHCQPENITFTLHLGNSTRVNDFVYNRFRDTFFNRFTHIHCPSNMIAEQLRRHGYTAQLHVISNGISPQYVYGKRPKEEWMQGYFNVLSVGRYAGEKRQDVLIEAAAKCRHAQEIQVILAGKGPLEKKYRKLAEKLPNPAVMGFYEPERLLDILHMADLYVHTSDAEIEGMSCTEAFACGLVPVIAVAPRSATSQFALDMRSLFPGGDTDALAEKIDWWIEHPAEREEMEHRYAEHAKQYTLERSIELTEEMFHQAIAEQTHG

SEQ ID NO: 30 - protein, 376 aa, Oscillibacter sp.
MKIVLVIDQFDDANNGTTISARRFAMALKEHGNEVRVIAIGKPADYKYAVRQMRFFPIVEHLITSQGMRLAIPNKHVFEKAAAWADVVHFMMPSPMAIMGLKHVERLGIPHTAAFHCQPENITFTLHLGNSTRVNDFVYNRFRDTFFNRFTHIHCPSNMIAEQLRRHGYTAQLHVISNGISPQYVYGKRPKEEWMQGYFNVLSVGRYAGEKRQDVLIEAAAKCHHAREIQVILAGKGPLEKKYRKLAEKLPNPAVMGFYEPERLLDILHMADLYVHTSDAEIEGMSCTEAFACGLVPVIAVAPRSATSQFALDMRSLFPGGDTDALAEKIDWWIEHPAEREEMEHRYAEHARQYTLERSIELTEEMFRQAIAEQTH

SEQ ID NO: 31 - protein, 376 aa, Oscillibacter sp.
MKIVLVIDQFDDANNGTTISARRFATALKEHGNEVRVIATGKPTDYKYAVRQMRFLPIVEHLITSQGMRLAIPNRHVFEKAAAWADVVHFMMPSPLAVMGLKHVERLGIPHTAAFHCQPENITFTLHLGNSKRVNDFVYDKFRDTFFNRFTHIHCPSNMIAGQLRDHGYTAQLHVISNGISPRYTYGRSPQEDWMQGKFNVLMVGRYAGEKRQDVLIDACARCRHKDAVQVILAGKGPLEKKYRRLAEALSNPIVMEFYEPDRLLEILHMADLYVHTSDAEIEAMSCMEAFACGLVPVIADSPRSATPQFALDGRSLFPAGDAAALAEKIDWWFEHPEEREEMGKRYGEHAKRYALDRSIRQTEEMFRTAIREQRT

SEQ ID NO: 32 - protein, 376 aa, Oscillibacter sp.
MKIVLVIDQFDDANNGTTISARRFATALKEHGNEVRVIATGKPTDYKYAVRQMKFLPIVEHLITSQGMRLAIPNRHVFEKAAAWADVVHFMMPSPLAVMGLKHVERLGIPHTAAFHCQPENITFTLHLGNSKRVNDFVYDKFRDTFFNRFTHIHCPSNMIAGQLRDHGYTAQLHVISNGISPRYAYGRSPQEDWMRGKFNVLMVGRYAGEKRQDVLIDACARCRHKDAVQVILAGKGPLEKKYRRLAETLSNPIVMEFYEPDRLLEILHMADLYVHTSDAEIEAMSCMEAFACGLVPVIADSPRSATPQFALDERSLFPAGDAAALAEKIDWWFEHPEEREEMGKRYGEHAKRYALDRSIRQTEEMFRTAIREQRT

SEQ ID NO: 33 - protein, 377 aa, Oscillibacter sp.
MKIVLVIDQFDDANNGTTISARRFATALKEHGNEVRVIATGKPADYKYAVRQMRFFPIVEHLISSQGMRLAIPNRHVFEKAAAWADVVHFMMPSPLAIMGLKHVERLGIPHTAAFHCQPENITFTLHMGNSRRVNDFVYVKFRDTFFNRFTHIHCPSNMIANQLRDHGYTARLHVISNGISPQYTYGRTPQEDWMRGRFNVLMVGRYAGEKRQDVLIDACAQCRHKDEIQVILAGKGPLERKYRRQAEKLPNPIVMEFYEPSRLLEILHMADLYVHTSDAEIEAMSCMEAFACGLVPVIADSPRSATPQFALDERSLFPAGDAAALAEKIDWWFEHPEAREEMGRRYAQHAEQYALDRSIVQTEEMFRLAIQEQKRG

SEQ ID NO: 34 - protein, 376 aa, Oscillibacter sp.
MKIVLVIDQFDDANNGTTISARRFATALKEHGNEVRVIATGKPTDYKYAVRQMKFLPIVEHLITSQGMRLAIPNKHVFEKAAAWADVVHFMMPSPLSIMGLKHVERLGIPHTAAFHCQPENITFTLHLGNSKRVNDFVYVKFRDTFFNRFTHIHCPSNMIANQLRQHGYTAQLHVISNGISPQYTYGRSPQEDWMRGKFNVLMVGRYAGEKRQDVLIDACLQCRHKDAIQVILAGKGPLEKKYRHLAEKLPNPIVMEFYEPSRLLEILHMADLYVHTSDAEIEAMSCMEAFACGLVPVIADSPRSATPQFALDERSLFPAGDAGALAEKIDWWFEHPKEREDMGRRYAKHAGRYALDRSIGQTEEMFQMAIREQKA

SEQ ID NO: 35 - protein, 381 aa, Oscillibacter sp.
MKIVLVIDQFDDANNGTTISGRRFAAALKEHGNEVRVIAIGKPADYKYAVRQMKFPPIVEHLVTSQGMRLAIPNKHVFEKAAAWADVVHFMMPSPMAVMGLKHVERLGIPHTAAFHCQPENITFTFHMGNNKRVNDFVYDKFRDTFYNRFNHIHCPSEMIANQLRQHGYTAQLHVISNGISPQYTYGKLPKEDWMQGYFNVLMVGRYAGEKRQDVLIEAAAKCRHAKEIQVILAGKGPLEKKYRQLAEKLPNPVVMGFYEPERLLDILHMADLYVHTSDAEIEGMSCTEAFACGLVPVIAVAPRSATSQFALDERSLFPGGDSDALAERIDWWIEHPQERQEMELKYAEHAKQYTLERSIELTEEMFRQAIEEQEKLNLTK

SEQ ID NO: 36 - protein, 376 aa, Oscillibacter sp.
MKIVLVIDQFDDANNGTTISARRFATALKEHGNEVRVIATGKPTDYKYAVRQMKFLPIVEHLITSQGMRLAIPNRHVFEKAAAWADVVHFMMPSPLSIMGLKHVERLGIPHTAAFHCQPENITYTLHLGNSKRVNDFVYTKFRDTFFNRFTHIHCPSNMIANQLRDHGYTAQLHVISNGISPQYTYGRAPQEDWMRGKFNVLMVGRYAGEKRQDVLINACAQCRHKEDIQVILAGKGPLEGKYRRLAEKLPNPIVMEFYEPGRLLEILHMADLYVHTSDAEIEAMSCMEAFACGLVPLIADSPRSATPQFALDGRSLFPAGDAAALAEKIDWWFEHPQELEEMGRRYGGHAKQYALDRSIQQTEEMFRTAIREQRK

SEQ ID NO: 37 - protein, 379 aa, Oscillibacter sp.
  VARIANT 260: note = Any amino acid
  VARIANT 266: note = Any amino acid
  VARIANT 267: note = Any amino acid
  VARIANT 272: note = Any amino acid
  VARIANT 352: note = Any amino acid
MKIVLVIDQFDDANNGTTISARRFATALKQHGNEVRVIATGKPTDYKYAVRQMKFLPIVEHLITSQGMRLAIPNRHVFEKAAAWADVVHFLMPSPLAIMGLKHVEKLNIPHTAAFHCQPENITFTLHLGNSRRVNDFVYSKFRDTFFNRFTHIHCPSQMIANQLTEHGYTAQLHVISNGISPEYVYGKQPKEDWMAGKFNILMVGRYAGEKRQDELINAAAKCRHAQEIQVILPGKGPLEHKYRKLAEKLPNPIVMQFYXPARLLXXLHMAXLYVHTSDAEIEAMSCMEAFACGLVPVIADSPRSATPQFALDERSLFPAGDVDALAERIDYWIDHPEERKAMELRYGEHAXQYALDRSIQLTEEMFRQAIAEQPAKRR

SEQ ID NO: 38 - protein, 382 aa, Oscillibacter valericigenes
MKIVLVIDQFDDANNGTTISGRRFAAALKEHGNEVRVIATGKPADYKYAVRQMKFPPVVEHLVTSQGMRLAIPNKHVFEKAAAWADVVHFMMPSPLAVMGLKHVERLGIPHTAAFHCQPENITFTFHMGNSKRANDFVYEKFRDTFFNRFGHIHCPSKMIADQLRQHGYTSQLHVISNGISPQYTYGKLPKEDWMQGYFNVLMVGRYAGEKRQDVLIEAAARCRHAKEIQVILAGKGPLEKKYRQMAEKLPNPVVMGFYEPERLLDILHMADLYVHTSDAEIEGMSCTEAFACGLVPVIAVAPRSATSQFALDERSLFPGGDSGALAERIDWWIEHPRERQEMELKYAEHAKQYTLERSIELTEEMFRQVIEEQKELNQTKP

SEQ ID NO: 39 - protein, 376 aa, Oscillibacter sp.
MKIVLVIDQFDDANNGTTISARRFATALKEHGNEVRVIATGKPTDYKYAVRQMRFLPIVEHLITSQGMRLAIPNKHVFEKAAAWADVVHFMMPSPLAIMGLKHVERLGIPHTAAFHCQPENITFTLHMGNSKRVNDFVYTKFRDTFFNRFTHIHCPSNMIADQLRQHGYTARLHVISNGISPRYTYGRAPQEDWMRGKFNVLMVGRYAGEKRQDVLIDACAQCRHKDGIQVILAGKGPLEKKYRRLAEKLPNPIVMEFYEPDRLLEILHMADLYVHTSDAEIEAMSCMEAFACGLVPVIADSPRSATPQFALDGRSLFPAGDAAALAEKIDWWFEHPEEREEMGKRYGEHAGRYALDRSIEQAEEMFRTAIQEQRK

SEQ ID NO: 40 - protein, 376 aa, Oscillibacter sp.
MKIVLVIDQFDDANNGTTISARRFATALKEHGNEVRVIATGKPTDYKYAVRQMKFLPIVEHLITSQGMRLAIPNRHVFEKAAAWADVVHFMMPSPLAIMGLRHVERLGIPHTAAFHCQPENISFTLHLGNSKRVNDFIYTKFRDTFFNRFTHIHCPSSMIAGQLREHGYTARLHVISNGISPQYAYGRSPQEDWMRGKFNVLMVGRYAGEKRQDVLIDACAQCRHKDEIQVILAGKGPLEKRYRRLAEKLPNPIVMEFYEPSRLLEILHMADLYVHTSDAEIEAMSCMEAFACGLVPIIADSPRSATPQFALDGRSLFPAGDPAALAEKIDWWFDHPREREEMGRRYGEHAKQYALDRSIEQTEEMFRTAIREQGK

SEQ ID NO: 41 - protein, 377 aa, Oscillibacter sp.
MKIVLVIDQFDDANNGTTISARRFASALKAHGNEVRVIATGKPTDYKYAVRQMRFLPIVEHLITSQGMRLAIPNKHVFEKAAAWADVVHFMMPSPLAIMGLKHVERLGIPHTAAFHCQPENITFTLHMGNSKRANDFVYNRFRDTFFNRFTHIHCPSHMIANQLKEHGYTAQLHVISNGISPQYVYGRSPKEDWMSGMFNVLMVGRYAGEKRQDVLIDACAECRHKDEIQVILAGKGPLERKYRKLCQKLPNPAVMEFFEPARLLEILHMADLYVHTSDAEIEAMSCMEAFACGLVPVIADSPRSATPQFALDDRSLFPAGDSAALAEKIDWWIEHPREREEMGRRYGESARQYALERSIEQTEDMFRQAILETDKR

SEQ ID NO: 42 - protein, 376 aa, Oscillibacter sp.
MKIVLVIDQFDDANNGTTISARRFATALKEHGNEVRVIATGKPTDYKYAVRQMRFLPIVEHLITSQGMRLAIPNKHVFEKAAAWADVVHFMMPSPLAIMGLKHVERLGIPHTAAFHCQPENITFTLHMGNSKRVNDFVYTKFRDTFFNRFTHIHCPSNMIADQLRQHGYTARLHVISNGISPRYTYGRAPQEDWMRGKFNVLMVGRYAGEKRQDVLIDACAQCRHRDGIQVILAGKGPLEKKYRRLAEKLPNPIVMEFYEPDRLLEILHMADLYVHTSDAEIEAMSCMEAFACGLVPVIADSPRSATPQFALDGRSLFPAGDAAALAEKIDWWFEHPEEREEMGKRYGEHAGRYALDRSIEQAEEMFRTAIQEQGK

SEQ ID NO: 43 - protein, 376 aa, Oscillibacter sp.
MKIVLVIDQFDDANNGTTISARRFATALKEHGNEVRVIATGKPTDYKYAVRQMRFLPIVEHLITSQGMRLAIPNKHVFEKAAAWADVVHFMMPSPLAIMGLKHVERLGIPHTAAFHCQPENITFTLHLGNSRRVNDFVYVKFRDTFFNRFTHIHCPSNMIANQLREHGYTAQLHVISNGISPQYTYGRSPQEDWMRGKFNVLMVGRYAGEKRQDVLIDACAQCRCREEIQVILAGKGPLEGKYRRRAEKLLNPIVMEFYEPSRLLEILHMADLYVHTSDAEIEAMSCMEAFACGLVPVIADSPRSATPQFALDERSLFPAGDAAALAEKIDWWHDHPQEREEMGKCYAEHAKQYALDRSIEQTEEMFRLAIREQRV

SEQ ID NO: 44 - protein, 383 aa, Oscillibacter valericigenes
MKIVLVIDQFDDANNGTTISARRFAKALKDHGNEVRVIAIGKPADYKYAVKQMKFLPVMEHLVTSQGMRLAIPNKHVFEKAAAWADVVHFMMPSPLAVTGLRHVEKLGIPHTAAFHCQPENITYTFHLGRSKRANDLLYSGFRDTFYNRFTHIHCPSNMITNQLRQHGYTAQLHVISNGISPQYFYGKLPKEDWMKGYFNVLMVGRYAGEKRQDVLIEACARSHHAKEIQVILAGKGPLEKKYRKLAEKLQNPIVMQFYEPERLIDILHMADLYVHTSDAEIEAMSCMEAFACGLVPIIAESSRSATPQFALDGRSLFPAGDSAALAAQIDWWIEHPEERKTMELRYAEHAKQYAMEESIKKTEEMFRMAIAEQGAARKAVHP

SEQ ID NO: 45 - protein, 375 aa, Oscillibacter sp.
MKIVLVIDQFDDANNGTTISARRFAQALKEHGNEVRVIATGKPADYKYAVRQMRFFPIVEHLITSQGMRLAIPNRHVFEKAAAWADVVHFMMPSPLAIMGLKHVEKLGIPHTAAFHCQPENITFTLHLGNSQRVNDFVYTRFRNTFFNRFTHIHCPSEMIADQLRRHDYTAQLHVISNGISPQYFYEKRPKEDWMQGKFNVLMVGRYAGEKRQDVLIQAAAKSRHSGELQVILAGKGPLEKKYRRMAEVLPNPIVMDFYQPERLIEILHMADLYVHTADAEIEAMSCMEAFACGLVPVIANSARSATPQFALDGRSLFPAGDSSALAAQMDWWIEHPEQRAAMERRYAEHAEQYALARSIEQTEEMFRQAIAERN

SEQ ID NO: 46 - protein, 382 aa, Oscillibacter sp.
MKRMKIVLVIDQFDDANNGTTISARRFAMALKEHGNEVRVIAIGKPGPNKYAVRQMRFLPIVEHLITSQGMRLAVPNRHVFEKAAAWADVVHFMMPSPLAVAGLKHVEKLGIPHTAAFHCQPENITFTLHLGNSKRANDFLYNRFRDSFYNRFTHIHCPSNMIAEQLRQHGYTAQLHVISNGISPQYHYRKLPKEDWMLGKFNILMVGRYAGEKRQDVLIDACARSRYAQQLQVILAGKGPLERKYRRLAEKLPNPIVMEFYEPERLLDIFAMCDLYVHTSDAEIEAMSCMEAFACGLVPVIADSPRSATPQFALDERSLFPAGDSLALARRIDWWIDHPAQRQEMEVRYAEHAKQYALERSIQLTEEMFRQAIEEQRPASR

SEQ ID NO: 47 - protein, 378 aa, Oscillibacter sp.
MKIVLVIDQFDDANNGTTISARRFAMALKAHGNEVRVIATGKPADYKYAVRQMRFFPIVEHLMTSQGMRLAIPNRHVFEKAAAWADVVHFMMPSPLAIMGLRHVEKIGIPHTAAFHCQPENITFTLHMGNSRRMNDFVYGKFRDTFFNRFTHIHCPSGMIANQLRDHGYTAQLHVISNGISSEYVYGKRPKEDWMEGFFNILMVGRYAGEKRQDVLIEAAARCRHASEIQVILPGKGPLEKKYRHLAKKLPNPVVMGFYEPARLIELLHMADLYVHTSDAEIEAMSCMEAFACGLVPVIADSPRSATPQFALDGRSLFPAGDSATLAERIDWWIEHPEERREMERRYAEHAKQYTLERSIEQTEEMFLQAIQEQGKNN

SEQ ID NO: 48 - protein, 376 aa, Oscillibacter sp.
MKIVLVIDQFDDANNGTTISARRFAAALKEHGNEVRVIATGKPTDYKYAVRQMKFLPIVEHLITSQGMRLAIPNRHVFEKAAAWADVVHFMMPSPLAIMGLKHVERLGIPHTAAFHCQPENITFTLHLGNSKRVNDFVYVKFRDTFFNRFTHIHCPSRMIAEQLRQHGYTARLHVISNGISPQYAYGRSPQESWMKGRFNVLMVGRYAGEKRQDLLIEACARCRCREQVQVILAGKGPLEKKYRRLARKLANPIVMEFYPPERLLELLHQADLYVHTSDAEIEAMSCMEAFACGLVPVIADSPRSATPQFALDERSLFPAGDAGALAEKIDWWFEHPREREEMGRRYGEAAKRYALDRSIDQTEEMFRTAIREQRG

SEQ ID NO: 49 - protein, 376 aa, Oscillibacter sp.
  VARIANT 223: note = Any amino acid
  VARIANT 226: note = Any amino acid
  VARIANT 312: note = Any amino acid
MKIVLVIDQFDDANNGTTISARRFATALKEHGNEVRVIATGKPTDYKYAVRQMRFLPIVEHLITSQGMRLAIPNKHVFEKAAAWADVVHFMMPSPLAIMGLKHVERLGIPHTAAFHCQPENITFTLHMGNSKRVNDFVYTKFRDTFFNRFTHIHCPSNMIADQLRQHGYTARLHVISNGISPRYTYGRAPQEDWMRGKFNVLMVGRYAGEKRQDVLIDACAQXRHXDGIQVILAGKGPLEKKYRRLAEKLPNPIVMEFYEPDRLLEILHMADLYVHTSDAEIEAMSCMEAFACGLVPVIADSPRSATPQFAXDGRSLFPAGDAAALAEKIDWWFEHPEEREEMGKRYGEHAGRYALDRSIEQAEEMFRTAIQEQGK

SEQ ID NO: 50 - protein, 377 aa, Oscillibacter sp.
MKIVLVIDQFDDANNGTTISAQRFARALIQHGNQVRVIACGKPADYKYAVRQMKFLPVVEHLITSQGMRLAIPNRHVFEKAAAWADVVHFMMPSPLGIMGLRHVEKLGIPHTAAFHCQPENITFSVHLGNSRRVNDFVYNRFRDTFFNRFTHIHCPSNMIADQLRQHGYTAQLHVISNGISPAYFYHKREKEAFLQEKFTILMVGRYSGEKRQDVLIDACAKSRHAAHIQLILAGKGPMEKKLRRQCEAQLANPAILEFYSPQRLIEIISQCDLYVHTSDAEIEAMSCMEAFACGLVPVIAQSPRSATPQFALDERSLFPAGDSRLLAKRIDYWYEHPQERQEMEKRYAESAKDYSLEASICQTEEMFRAAIREMRH

SEQ ID NO: 51 - protein, 380 aa, Oscillibacter sp.
MKIVLVVDQFDDANNGTTISGQRFARALVAHGNQVRVIACGKPAEYKYAVHQLHFPFFVEHLITSQGMRLAVPNRHVFEKAAAWADVVHFMVPTPLGVMGLRHVEKLGIPHTAAFHCQPENITFSVHLGNNKKVNDWIYNRFRDSFYNRFTHIHCPSNMIANQLRDHGYTAQLHVISNGIGPAYHYCKNPKEDWMKEKFAIIMVGRYSGEKRQDELIEACKRSRHARRIQLILAGKGPLEKKYRKLCESLPNPVVMDFYSPERLIEILSQCDLYVHTSDAEIEAMSCMEAFACGRVPVIADSPRSATPQFAIDERSLFPAGDVDALAKRIDYWIEHPDEREEMEHRYAEHAKQWSLSHSIEMTEEMFRTAIREQGKGTAE

SEQ ID NO: 52 - protein, 377 aa, Oscillibacter valericigenes
MKIVLVIDQFDDANNGTTISAQRFARALMAHGNQVRVIACGKPADYKYAVRQMRFLPVVEHLITSQGMRLAIPNRHVFEKAAAWADVVHFMMPSPLGIVGLRHVEKVGIPHTAAFHCQPENITFSVHLGNSRRANDFVYNRFRDTFFNRFTHIHCPSRMIADQLRQHGYTAQLHVISNGISPAFFYQKREKEDFLKGKFAVLMVGRYSGEKRQDVLIDACSKSRHASQLQLILAGKGPTEKKLRRRCEERLQNPVVMEFYSPQRLIEIIDQCDLYVHTSDAEIEAMSCMEAFASGLVPVIAQSPRSATPQFALDERSLFPAGDSTVLAKRIDYWFEHPEERREMERRYAESAKNYSLENSILQTEEMFGMAIREIWP

SEQ ID NO: 53 - protein, 376 aa, unidentified
MRLLIVVDQFDSGNNGTTISAQRFARALRERGHEVRVAAAGKPAEGKYALPRIHFLPLVDNIIKSQGMCFARPRRATLRAAMEWADVVHFMMPLPLEQVGRKMACQMNKPHTAAFHVQPENITSTLGLRRARQVNEALYAWFRDSFYNHFTHVHCPSEFIAGQLRAHGYTARLHVISNGVDDCFYPRREEKQGPLAGKYAVLMIGRFSEEKRQEVLLQAVRESRHAKQIQLVLAGQGPREKHLRKLGERLPNPPIMGFYSTRQLCRLMAMTDLYVHTAVAEIEAIACLEAVASGLVPVIADSPLSATPQFALDGRSLFPADDAAALARKIDYWLENGAEREAMGRRYAESAARYRLASSAQKAEEMFHEAIAEARG

SEQ ID NO: 54 - protein, 376 aa, unidentified
MRLLIVVDQFDSGNNGTTISAQRFARALRERGHEVRVAAAGKPAEGKYALPRIHFLPLVDNIIKSQGMCFARPRRATLRAAMEWADVVHFMMPLPLEQVGRKMACQMNKPHTAAFHVQPENITSTLGLRRAKPVNEALYAWFRDSFYNHFTHVHCPSEFIAGQLRAHGYTARLHVISNGVDDCFYPRREEKQGPLAGKYVVLMIGRFSEEKRQEVLLQAVRESRHAKQIQLVLAGQGPREKHLRKLGERLPNPPIMGFYSTRQLCRLMAMTDLYVHTAVAEIEAIACLEAVASGLVPVIADSPLSATPQFALDGRSLFPADDAAALACKIDYWLENGGEREAMGRRYAESAARYRLAGSAQKAEEMFHEAIAEARG

SEQ ID NO: 55 - protein, 388 aa, unidentified
MVDIPGEKGKIMRILLVMDQLDDKNNGTTISAQRLAVTLREHGNEVRTVSVGEASHDRYSLKELKLLPGVRGIVHNQGMLFAYPDRVVLEKAIRWADVVHFLMPFWVSSKGRRIAQRLGVPHTAAFHVQPENVSYNIGLGKYEAVNSTIYAYFRQFYNQFTHVHCPSKFIARELKRHGYTAQLHVISNGVDRDFVYRKSPKPKQLEGKFVILMVGRLSNEKRQDVLIEAVRKSKYENRIQLLLAGRGPKQKKYESLAQGLTNRPIIGFYTKQELLDLLSICDLYAHAADIEIEAISCIEAFSSGLVPVIADSPKSATPQFALDGRSLFRAGDSGDLAAKIDYWIEHEEERKRMEIVYSEHGKQYNIDACVGQMEEMFTAAIEENKCAR

SEQ ID NO: 56 - protein, 385 aa, unidentified
MKILLVLDQYDDCNNGTTVSAQRFAEGLTKRGHEVFIASTGKPAQNKFIVKPLPLPPGISWLIKSQGMVFALPTESVLKEAISKVDIVHFYLPFFLSRGGVKIAEELHIPHTAAFHSQPENITYTLGLGRSQKANDIIFNYYRSFYNKFSHVHCPSQFIADELKSHNYKSQLHVISNGIQPEFKYIKSNKTDDLKDKFVITMVGRYSNEKRQDLLFEAVNKSKYRDKIQVVLAGKGPNTKKYAKLAKTLTNEPVMKFFSKDDLVKMLSMTDLYVHASDAEIEGISCIEAIACGIVPIIAKGKKAATPQFALDERSLFEAGNASDLADKIDYWLENPDERKKMEFEYAKSADKYSMNKSMEQIEEMFYEAIKECKEEQSDITQPAF

SEQ ID NO: 57 - protein, 382 aa, unidentified
MKILLVSDQYYAANNGMTISARRFAGVLRQHGHEVRIMSYGTPDMVEDTDSAYLLDKYYVPIFNRLVTSQGMVFAKRTRKVVEAAVDWADLIHILSPFFLSHKTIRIAQKKNKPYTAAFHVQPQNITSSIYLGKVNWINDLLYHFFHRYIYRYCTHIHCPSRFIAQQLVRTGYTEQLHVISNGIDPDFHYFKRHKPRELRDKFVILQTGRLSIEKRPDVLIRAVAMSRHADQIQLVLAGKGPRKKKLQKLADKLLKNPVIIQFYSKPELIQLLGYCDLYVHAADVEIEAMSCMEAFASGLVPVIANSPTSATPQFALDERSLFEVGNSKELAEKIDWWLEHPQEREQMEFRYAELGKKYALEDCVRQAEAMFEQAVRENHGG

SEQ ID NO: 58 - protein, 376 aa, Subdoligranulum sp.
MNYLFVLDQYGAENNGVSVSARRYAAVLRARGHGVRILSTGDCGPDGYSVPELRIPVFDKLIKSHGMVFGKPEKRVLEQALAWADHVHFLMPFTLSIVTAQRARKMGKPATAAFHVQPQNISYSIGMGRWTPTNAFIYWLFRTDFYGRFRYIHCPSAFIAKQLKAHGYGAECRVISNGITPEFTFRRDGKEAKWRGRFLILMIGRLSGEKRQDVLIEAVKRSRHAQDIQLIFAGQGPLHDAYAKQARGLAHPLQMRFLSQEELRSVIAQCDLYVHTADAEIEAMSCMEAFACGRVPVIANSPFSATPQFALDERSLFCPGDASDLAEKIDYWIEHPDERLAMERAYSESAKKYAIENCVTQFEQLAAEAKEKGSWA

SEQ ID NO: 59 - protein, 376 aa, Subdoligranulum sp.
  VARIANT 189: note = Any amino acid
  VARIANT 273: note = Any amino acid
MNYLFVLDQYGAENNGVSVSARRYAAVLRARGHGVRILSTGDCGPDGYSVPELRIPVFDKLIKSHGMVFGKPEKRVLEQALAWADHVHFLMPFTLSIVTAQRARKMGKPATAAFHVQPQNISYSIGMGRWTPTNAFIYWLFRTDFYGRFRYIHCPSAFIAKQLKAHGYGAECRVISNGITPEFTFRRDXKEAKWRGRFLILMIGRLSGEKRQDVLIEAVKRSRHAQDIQLIFAGQGPLHDAYAKQARGLAHPLQMRFLSQEELRSVIAQCDLXVHTADAEIEAMSCMEAFACGRVPVIANSPFSATPQFALDERSLFCPGDASDLAEKIDYWIEHPDERLAMERAYSESAKKYAIENCVTQFEQLAAEAKEKGSWA

SEQ ID NO: 60 - protein, 489 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLAHPLLRRRAAAG

SEQ ID NO: 61 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLERHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPNCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFTTAVRAGCPVVPMMLCRRPARGLWAWRKKPCFTLRIGAPLYADASLPKKQAARDLQLRAEAAMLALERGETPPLAVGEPALEAE

SEQ ID NO: 62 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFATAVRAGCPVVPMMLCRRPARGLWAWRKKPCFTLRIGAPLYADASLPKKQAARDLQLRAEAAMLALERGETPPLAVGEPALDAE

SEQ ID NO: 63 - protein, 629 aa, Subdoligranulum sp.
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPEDIHGMAALERGMEARIRGGAFVHFYPEGMLVPYHEGLRAFHPGAFATAVRAGCPVVPMMLCRRPARGLWAWRKKPCFTLRIGAPLYADASLPKKQAARDLQLRAEAAMLALERGETPPLAVGEPALDAE

SEQ ID NO: 64 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFTTAVRAGCPVVPMMLCRRPARGLWAWRKKPCFTLRIGAPLYADASLPKKQAARDLQLRAEAAMLALERGETPPLAVGEPALDAE

SEQ ID NO: 65 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFTTAVRAGCPVVPMMLCRRPARGLWAWRKKPCFTLRIGAPLYADASLPKKQAARDLQLRAEAAMLALERGETPPLAVGEPALEAE

SEQ ID NO: 66 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPEDIHGMAALERGMEARIRGGAFVHFYPEGMLVPYHEGLRAFHPGAFTTAVRAGCPVVPMMLCRRPARGLWAWRKKPCFTLRIGAPLYADASLPKKQAARDLQLRAEAAMLALERGETPPLAVGEPALDAE

SEQ ID NO: 67 - protein, 629 aa, Subdoligranulum sp.
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPEDIHGMAALERGMEARIRGGAFVHFYPEGMLVPYHEGLRAFHPGAFATAVRAGCPVVPMMLCRRPARGLWAWRKKPCFTLRIGAPLYADAALPKKQAARDLQLRAEAAMLALERGETPPLAVGEPALDAE

SEQ ID NO: 68 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYVSAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFMHFYPEGMLVPYHEGLRAFHPGAFATAVRAGCPVVPMMLCRRPARGLWAWRKKPCFTLRIGAPLYADASLPKKQAARDLQLRAEAAMLALERGETPPLAVGEPALDAE

SEQ ID NO: 69 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGISTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFATAVRAGCPVVPMMLCRRPARGLWAWRKKPCFTLRIGAPLYADASLPKKQAARDLQLRAEAAMLALERGETPPLAVGEPALDAE

SEQ ID NO: 70 - protein, 629 aa, unidentified
MKIVLVIDQFDDSNNGTTVTARRFAGQLRRRGHEVVILAGGAPCEGKICAPVHRIPVFQKLIESQGMCFAKPDEEAYYTAFKDADIVHFYMPFRFCRRGEELARQMGISTVAAFHVQPENITSSIFLGKNRRVNDFLYWWFYKVFYNRFDHIHCPSAFIARQLESHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPNCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFATAVRAGCPVVPMMLCRRPARGLWAWRKKPCFTLRIGAPLYADASLPKKQAARDLQLRAEAAMLALERGETPPLAVGEPALDAE

SEQ ID NO: 71 - protein, 634 aa, unidentified
MNIVLVIDQFDNGNNGTTITARRYAEQLRRLGHRVTILAGGEAAEGKICAPVHKIPVFQPLVEKQGFGFAKPDEEAYYQAFRDADVVHFYLPFRFCRRGEEIARQMRVPTVAAFHMQPENVTYSIGMGKSKRLNDFLYHWCYRKFYNRFRYIHCPSEFIAGQLKAHGYDAELRVISNGVDPLFRPVDTPRPPEFEGKFVILMVGRLSGEKRQDLIIEAAKKSRYADKIQLVFAGKGPKEKEYRRLSAGLAHPPIMGFYGQEELLRLLNSCDLYVHASDAEIEGISCMEALACGLVPVIANSPLSATPQFALDDRSLFQAGNAGDLADKIDYWIEHPEERAAQGRAYAARGDEMRVDACVARAEDMYREAIHDCRFHGYKPPRQGRLRRLTHPDPDKANQQFYHASALKKTVFGAVTNFLTPLLLFIDGLFFGLRVEGRQHLRGIRGGAVTVMNHVHPMDCTMAKIAAFPHRQYFVSLRRNLELPFTGWLVKLCGGLPLPPTASTMVPYQRHLEQAIRQGDFVHFYPEGLLVRYHKGLRPFHGGAFLTAARAGCPIVPMAVVFRKPTGLRALFRRGDDMTLRIGEPLYPNPALGPKAAAQALQLRTRYAMEMLLGDSAGGYPLPATDWEEAEDNI

SEQ ID NO: 72 - protein, 376 aa, unidentified
MTIVIVIDTLALNNGTTMTAYRFANMLRAHGHTVRFVSTGPQEEGKFVVRERYFPLATPIARKQGIIFARSDRRVFRQAFEGADVVHLMMPFPFEHNAMRVAREMGIPCSTAFHVQPENITYITHTDALPRMNESVYRLLYRMFYKDFHHIHCPSKFIAGQLEKNGYDAKLYVISNGVDAAFRPAEKKDTMHDDLFRILMIGRLSPEKRQNILIKAVSLSRHRDRIQLYLAGKGPRAEALRNMGKTLPHPPVIGFYSQEELIRLIHSCDLYVHASVVEIEAISCMESFACGLVPVICDAPQSATRQFALDERSLFKPDDPRDLAAKIDYWIEHPEERQKMSVQYAREGDEYRVERSVEKAEQMFREVIEDYRNGIL

SEQ ID NO: 73 - protein, 636 aa, unidentified
MNIVLVIDQFDDGNNGTTVTARRYAHELRRLGHRVTILAGGEPAAHKICAPAHKIPFFEKLIESQGMQFAKPDDRAYYEAFKDADVVHFYMPFRFCRRGEELARQLHIPTVAAFHVQPENITSSIGLGKRKNVNRFLYWWFYHVFYNRFRLIHCPSRFIAGQLAAHNYDAELCVISNGVDDAFIPHPEWKAERKPEDVLNVLMIGRLSGEKRQDLIIEAAKRSRYRDRIQLHFAGKGPKEAEYRRLSKGLPRPPVFGFYSTEELVGLINRCDLYIHSSDAEIEGISCMEALACGLVPVISDSVLSATRQFALDERSLFKAGDPSDLARRMDYWFEHPEERLKMEAAYAQKGEEMRVSACVRQADAMYCKAVEEQRKNGYVRPKELKIRRITHPDADRINKRYAKHSPVRRGLTRAFTNLLAVILSVFDSLFLGFSIKGKEHLSSVEGGAVTVCNHVHPMDCTMVKIALFPRLVRYVSLRRNLELPLIGWILKACGVLPLPEHPIRIARFQKELEKGIAAGEWVHYYPEGMLVKYYEGLRPFQPGAFLTAVRANCPVIPLRINYKQPHGPCALWRKRPFLELVVKAPLYADQTLPQKQAALDLMQRTLRAMGGVEEKDPAPGYLSEPEDAPIAGTVT

SEQ ID NO: 74 - protein, 597 aa, unidentified
MIITLVNDTFNINNNGTTISAMRFAEALSQRGHQIRIITCGDPLKSGKDPDTGFEMFYLPELKIPIASRLAHKQNTLFAKPVRSILKKAISGSDVVHIYQPWPLGSAAQRVARQMNIPAIAAFHIQPENITFNIGLKRFSPAAHLTYFLFYLFFYRRFSHIHCPSKFIAAQLRSHGYKARLHVISNGVHPAFCAPAKPREHTFKPIKILMIGRLSPEKRQDVLIRAVMKSRYADRIQLYFAGSGPWEKKLRRLGNKLPNPPVFGYYNRDELIKLIHECDLYVHASDAEIEGISLIEAFACGLVPIISDSKQSAAAQFALGPQNLFKAGSPESLAKKIDYWLDHPEQLKEAEKKYAQLGKQYALEHSIRKIEKVYSSMTKNHKNEYHRSIFFRLSTRLFQIVIACPILLLWTRFVLGAKVYGRENIRGLKSGVTVCNHVHLLDSALIGVTFFPRRVVFPTLTQNVKTLWPGKLVRILGGFAIPDNIMELKAFFDEMEFLLMKNCIVHFFPEGELRPYDTGLQNFKKGAFYLAAQAQVPIVPMLITFEPPKGLIKIIRKKPVMRLHIGKPIHPMSKDIEIDSELRMKAVCKKIEAITSV

SEQ ID NO: 75 - protein, 394 aa, unidentified
MHIVFVVDMYDVQKNGTTMTARRMAEALARRGHEVRVIYAASSDQDCIRPQAGDPVRLFSVRQLKVPVLYQFMKAQHVTMGRPDEAVIRQALSGADVVHLFLPFPLEKQALRVAKELKIPVTAAFHLQPENVSYNIGLGRSKSFNRAIYNYFNRYLYQYVSRVHCPSEFIARQLVAHQYKARLYVVSNGVPDQFKPAILPHPEPEAGAEIPVIMVGRLSPEKNQALLLKAVLKSRFAKQIRVTLAGQGPDEAKLRRLGSRLPLAPEIGYYTQLELLQKLQTSILYIHTASAEIEAIACLEAVACGLVPLISNSDQSATPQFALDERSLFMPDSVDDLAQKLDYWLEHPGERQRMEQQYAKSAQAYRLDKVTAKLEQMLTEAVEYQHEPEAAGYL

SEQ ID NO: 76 - protein, 381 aa, unidentified
MIICVVCDVLGKANNGTTMAALDLIQALKERGHLVRVVCPDLDRMGQENFYVVPVRNLGPLNNYLQKNGVELAKPERRILEQAMHGADHVHIMLPFPLGIKALKVAKEQGLPVTAGFHCQAENFTSHIFMKDFKPANTITYKTFYRLFYRHVDAVHYPTEFIRETFENATGKTNAYVISNGVRSAFQPAPSLRPKELEGRFVILFIGRYSKEKAHKVLIDAAALSRHAKELQLIFAGSGPLREKLEKRSRKLANPPIFRFFSRDELVRAINYADLYVHPSEVEIEAISCLEAISCGLVPVISDSYRSATRFFALDRKNLFTCNDPQALADRIDYWLEHPAERQERSRAYLGYTVQFNYENCMDRMEQMIVETHDRVRSGKR

SEQ ID NO: 77 - protein, 401 aa, unidentified
MDTFDRESGRDLPSLPGKAAQPIPQRRTAAMKVAMICDVMGQPNNGTTLAALNLIRYLRDAGHTVTVVAPGGEASDGYLPVRVWHAGPLIDRILRMNGVELAVPDKQLLEAVIREADVVHLLIPLPLARAALKIARRLGKPVTASFHCQAENITAHLGMMNAGWLNRLIYRNFYRKVYRWCTAVHYPTEFIREVFETATHPTPAHVISNGVNDMFRLPDSRPENGKFTVVCSGRYSREKAQQQLLRAAALCRHRDDIRLILAGDGPRRKHYLRLAKQYGLDCQFAFFPRQELLHILQTADLYVHTAIIEIEAIACTEAICCGLVPVICNSDRSATRFFACGDHTLFEPGDVRALADLLDYWYEHPAARVERAAEYADLRHSFDQTACMQQMEQMLKEAAGL

SEQ ID NO: 78 - protein, 379 aa, unidentified
MTICVVCDVLGRENNGTTIAAMNLIRSLHAKGHTVRIVCPDKERKGEPDYFVVPTLKLGPFDPYVRKNGVVIAKPRHKVLEAALDGVDHLHLMVPFMVSRFVLKMARKKGISVTAGFHCQAENLTSHLFLKNNRLANRLVYRNFYRRFYRYVDGVHFPSEFIHGVFERYGGKTNAYVISNGVNKQFKPMEVERPKELEGKRVILFTGRFSKEKSHTVLIDAAKMSRHVDELQLVFAGDGPLKKKLEKRGRGLRNPPIFRFFSREELLRVINSADLYVHPAEIEIEAISCLEAISCGLVPVISNSERSATRFFALSERNLFPCNDSAALAERIDYWLEHPEEKEACSREYLGYTKQFDFDVCMDRMEQMILETYENKHKA

SEQ ID NO: 79 - protein, 380 aa, unidentified
MTICIVCDVLGRENNGTTIAAMNLIRSLRAKGHSVRIVCPDRERSGEPDCYIVPTLPLGPLNGYVRKNGVVIARPSRKVLEAALSGVDHVHLMLPFPVSCAMLRLARERGISVTAGFHCQAENFTSHIFMKDSRLVNRLVYKAFYRHCYRYVDAVHYPTEFIREVFERYGGKTNAWVISNGVNRQFRPIPVSRPEELEGKYVILFIGRLSKEKSHRVLIDAAKQSRHAEELQLVFAGDGPLKEKLMKRSKALKNPPIFRFFSREELVKTINSADLYVHPAEIEIEAISCLEAISCGLVPVISDSPRSATRFFALGEQNLFHSNDSKALAERIDWWLEHPAEQEACSKAYLGYTKQFDFDACMDRMEQMILETHAAKHKEG

SEQ ID NO: 80 - protein, 380 aa, unidentified
MTICIVCDVLGRENNGTTIAAMNLIRSLRAKGHSVRIVCPDRERSGEPDCYIVPTLPLGPLNGYVRKNGVVIARPSRKVLEAALSGVDHVHLMLPFPVSCAMLRLARERGISVTAGFHCQAENFTSHIFMKDSRLVNRLVYKAFYRHCYRYVDAVHYPTEFIREVFERYGGKTNAWVISNGVNRQFRPIPVSRPEELEGKYVILFIGRLSKEKSHRVLIDAAKQSRHAEELQLVFAGDGPLKEKLMKRSKALKNPPIFRFFSREELVKTINSADLYVHPAEIEIEAISCLEAISCGLVPVISDSPRSATRFFALGEQNLFRCNDSKALAERIDWWLEHPAEQETCSKAYLGYTKQFDFDACMDRMEQMILETHAAKHKEG

SEQ ID NO: 81 - protein, 375 aa, Ruminococcus sp.
MKITVVCDVLGAENNGTTIAAMNLIRAMNKRGHEVTVVCSDEDKKGKEGYVVMPKLNLGPLNNYVSKNGVSLSRANKKVLEPVIKNSDVVHIMIPFMLGAAAVKLCRKYNIPVTAGFHCQAENFTSHIFMKDNSLANQITYNSIYRSVYRYVDAVHYPTEFIRNVFEQAVHKRTNGYVISNGVSERFKPIKTEKPPEFSNRFVILFSGRYSKEKSHSVLIDAAALSKHSREIQLVFAGDGPLKQKLKEQAKKLPNRPLFNFFPHAQMLNVLNYADLYVHPAEIEIEAIACMEALACGQVPIISDSPRSATVKFALDERNLFKNRDPEDLAKKIDFFIDNPDVLEEYRERYKGIVAEFSQKVCMDKMEKMFYEALR

SEQ ID NO: 82 - protein, 379 aa, unidentified
MTICVVCDVLGRENNGTTIAAMNLIRSLRAKGHAVRVVCPDAERKGEPDCFVVPTLNLGPINAYVRKNGVVIAKPERKVLEAALDGVDHVHLMVPFAVSRAALKLARERGLSVTAGFHCQAENFTSHIFMKNNRLANLLTYRNFYQHCYRWVDGVHYPSEFIRGVFERYGGKTNAYVISNGVNRQFRPMQVERPKELEGKRVILFTGRYSKEKSHTVLIDAAKLSRHADELQLVFAGDGPLKKKLEKRSRGLKHPPIFRFFSREELLQVINSADLYVHPAEIEIEAISCLEAISCGLVPVISDSERSATRFFALREENLFPCNDSRTLATRIDYWLEHPQEKEKCSRAYLGYTKQFDFDVCMDRMEQMILETHENKHRA

SEQ ID NO: 83 - protein, 380 aa, unidentified
MTIAVICDVLGKENNGTTIAAMNLIRSLREKGYDVRVVCSDPERAGQKDFYVVPTLNLGPLNAYVAKNGVALANPDRNILTAALNGVDHVHVMMPFALGSAAARLAHELGLPLTAGFHCQAENFTGHIFMKNFSPANRIAYHVFYRNLYRYCDCIHYPSQFICDTFEAIVGPTQHRIISNGVNQIFQPKPAEKPKELKGRFVVLFTGRYSPEKSHKVLIEAVARSRYKDEIQLIFAGDGPLRQSLARQAKRRGVHDPIMRFYSREELVNVINYADLYVHPAEIEIEAIACLEAIACGKVPLIADSPRSATRYFALTDRNLFRYNDPQDLADRMDWWLEHPEERLSCSKSYLGYAAQFDFTSCMDQMEKMILDVSEARHES

SEQ ID NO: 84 - protein, 380 aa, unidentified
MTIAVICDVLGKENNGTTIAAMNLIRSLREKGYDVRVVCSDPERAGQKDFYVVPTLNLGPLNAYVAKNGVALANPDRNILTAALNGVDHVHVMMPFALGSAAARLAHELGLPLTAGFHCQAENFTGHIFMKNFSPANRIAYHVFYRNLYRYCDCIHYPSQFICDTFEAIVGPTQHRIISNGVNQIFQPKPAEKPKELKGRFVVLFTGRYSPEKSHKVLIEAVARSRYKDEIQLIFAGDGPLRQSLARQAKRRGVHDPILRFYSREELVKVINYADLYVHPAEIEIEAIACLEAIACGKVPLIADSPRSATRYFALTDRNLFRYNDPQDLADRMDWWLEHPEERLSCSKSYLGYAAQFDFTSCMDQMEKMILDVSEARHES

SEQ ID NO: 85 - protein, 384 aa, unidentified
MNILFVINNLYSDGNGLCASARRTIGLLRERGPEVRVLSACGPDGQTPDYPLPDWHMPLFGPLVARQGYRFAARDKAVIRQALEWADLVHLEEPFFLQMTVCRMARSMGVPCVATYHLHPENFFASVGLQRSRYFNAATLWVWRKYVYDHCAIVQCPTERVRERLARRGFRAELRVISNGLLPEERPGAGPHIHTPGEPYNILCVGRYSEEKDQMTLLRAMEYTKHAHEIRLILAGRGPKEEKLRAAAEKLCRDGVLTIPPVFGFYSAAGLEELFAQADLYIHCATVEVEGLSCMEAVRTGIVPLIADGPLTATAQFALSRESRFPVGDAKALAAGIDYWLSDDEARRREAARYTALGEQYAIARSIDALVQMYRDALTAPGVR

SEQ ID NO: 86 - protein, 384 aa, unidentified
MRIAVICDVLGDKNNGTSIVAYNLIDFLRQKGHEVRIVCPDEEHTGEEGYWVVPKMHFGIFQSYVDRNGVAPAKLDKRILYDAIHDVDLIHVMIPFALGKAAAKYANQHGIPLTAGFHCQAENITSHIFLKDSRFANRVTYKILNERLYKYCDGIHYPTEFIRDTFERIVGTTPHFVISNGVGDEFRPMKAERPEALRDKYILLFTGRLSREKSHNVLIDAVAKSKHRDKIQLFFAGEGPLERELLAHAERVGISAPVIKFYSRAKLLNIINMADLYVHPAEIEIEAISCLEAIACGKVPLIADSPRSATRFFALSEKNLFKSGDAVDLSHKLDFWLDHPDERKKCSEEYLGYAERFRLKDCMEAMEDMLKKVANEKKKDCLVR

SEQ ID NO: 87 - protein, 381 aa, unidentified
MKIAVICDVLGKENNGTTIAAMNLIRSLRAKGHNVRVVCADPERAGQQDYYIVPKMNFGPINGYVAKNGVAPARVDRAILEAAIADVDLIHVMMPFAVGCAAAAYAHEHHIPLTAGFHCQAENVTGHIFMMDFPLANTIAYKTFYHKLYRYCDCIHYPSQFICDTFEGIVGPTPHRIISNGVSSTFQPIPVEKPEALRGRFVVLFTGRYSPEKSHKVLIDAVALSQHKDEIQLIFAGTGPLKLTLQRQARRRGIPQPIMEFYDRNTLIDMINSADLYVHPAEIEIEAISCLEAIACGRVPLIADSPRSATRYFALSDDNLFRYDDPADLAQKMDWWLEHPEERERCSQAYLGYAKQFDFQTCMDAMENMMLDTVEKKRHEK

SEQ ID NO: 88 - protein, 380 aa, unidentified
MILTVVCDVLGEENNGTTIAAMNLIRSMRQKGYTVRVVCPDESRQGQPGFFIVPTYNLRVFNGYVQKNGVTLAKPVQSILEAAIVGADVVHIIVPFALGRAALSLAKAQGIPVTAGFHCQAENITNHIFLMNANLANRLIYKVFYRSFYQYCDCIHYPTQFICDLFEKETKPTNHYVISNGVNRSFVRRDCPKPEELQDKFVILSTGRYSKEKSQQILLRAVALSKFKDKIQLILAGKGPRQAFLEQEAEKLGLLPPIFRFFSREALIDVINYADLYVHPAEIEIESIACLEAIACGKVPIIADSPRSAARNFALTPENLFACNDPADLAKRIDGWLSDPQARAACSEKYLGYAQAFAFDRCMEQMEQMLLDAVEGKRHG

SEQ ID NO: 89 - protein, 375 aa, Ruminococcus sp.
MKITVVCDVLGVENNGTTISAMNLIRAMAKRGHEVTVVCSDEDKRGKDGYVVMPTLSLGPLNNYVSKNGVSLSRANKRVLKPVIQNSDVVHVMMPFMLGTAAVKLCKKYDIPVTAGFHMQAENFTSHVFMKDNPLANQITYHTIYRNVYRHVDAVHYPTEFIRNVFEQAVHHRTNGYVISNGVGERFKPIKIEKPAEFANRFVIIFSGRYSKEKCHSVLIDAAALSKHSRDIQLIFAGEGPLKQKLRERAKKLYNKPLFNFFPHDQMLNVLNYADLYVHPAEIEIEAIACMEALACGQVPIIADSPRSATVKFALDERNLFKNRDAQDLADKIDFFIEHPDVLEEYRERYKGIVADFSQEACMDKMEKMFYEAMK

SEQ ID NO: 90 - protein, 380 aa, unidentified
MTIAVICDVLGKENNGTTIAAMNLIRSLREKGYDVRVVCSDPERAGQKDFYVVPTLNLGPLNAYVAKNGVALANPDRNILTAALNGVDHVHVMMPFALGSAAARLAHELGLPLTAGFHCQAENFTGHIFMKDFPLANRIAYHVFYRNLYRYCDCIHYPSQFICDTFEAIVGPTPHRIISNGVNHIFQPKPAEKPKELKDRFVVLFTGRYSPEKSHKVLIEAVARSRYKDEIQLIFAGDGPLRQRLARQAKRQGVHDPIMRFYSREELVNVINYADLYVHPAEIEIEAIACLEAIACGKVPLIADSPRSATRHFALTDHNLFHYNDPQDLADRMDWWLEHPEERLSCSKSYLGYAAQFNFTSCMDQMEKMILDVSEGRHES

SEQ ID NO: 91 - protein, 375 aa, Ruminococcus sp.
MKITVVCDVLGVENNGTTISAMNLIRAMAKRGHEVTVVCSDEDKRGKDGYVVMPTLSLGPLNNYVSKNGVSLSRANKRVLKPVIQNSDVVHVMMPFMLGTAAVKLCKKYDIPVTAGFHMQAENFTSHVFMKDNPLANQITYHTIYRNVYRHVDAVHYPTEFIRNVFEQAVHHRTNGYVISNGVGERFKPIKIEKPAEFANRFVIIFSGRYSKEKCHSVLIDAAALSKHSRDIQLIFAGEGPLKQKLRERAKKLYNKPLFNFFPHDQMLNVLNYADLYVHPAEIEIEAIACMEALACGQVPIIADSPRSATVKFALDERNLFKNRDAQDLADKIDFFIKHPDVLEEYRERYKGIVADFSQEACMDKMEKMFYEAMK

SEQ ID NO: 92 - protein, 380 aa, unidentified
MTIAVICDVLGKENNGTTIAAMNLIRSLREKGYDVRVVCSDPERAGQKDFYVVPTLNLGPLNAYVAKNGVALANPDRNILTAALNGVDHVHVMMPFALGSAAARLAHELGLPLTAGFHCQAENFTGHIFMKDFPLANWIAYHVFYRNLYRYCDCIHYPSQFICDTFEAIVGPTPHQIISNGVNHIFQPKPAEKPKELKDRFVVLFTGRYSPEKSHKVLIEAVARSRYKDEIQLIFAGDGPLRQSLARQAKRRGVHDPIMRFYSREELVKVINYADLYVHPAEIEIEAIACLEAIACGKVPLIADSPRSATRHFALTDHNLFHYNDPQDLADKMDWWLEHPEERLSCSKSYLGYAAQFNFTSCMDQMEKMILDVSEGRHES

SEQ ID NO: 93 - protein, 381 aa, unidentified
MIVAVICDVLGEENNGTTIAAMNLIRSLQAKGHEVRVVCADEARAGQAGFFLVPKFNFGIFNAYVQKNGVAPARVDHKVLEQAIHDADIIHVMLPFGLGKAAAQYASEHGIPLTAGFHCQAENITSHIFLKDFAWGNRAVYRILYRRLYRYCDCIHYPTQFICDTFEQVVGPTPHRIISNGVDRAFQPMTVPRPKTLEGKFVVLFTGRFSREKAHSVLIRAAAKSRHRDKLQLIFAGSGPLEAELRQEAEQAGILRPIMRFYSRAELIELINQANLYVHPAEIEIEAISCLEAISCGKVPLIADSPRSATRYFALSDRNLFRNRDPQDLADKLDWWIEHPEEIEQAQKAYLGYARQFDFDLCMDNMERMLLETWEAKHAAG

SEQ ID NO: 94 - protein, 375 aa, Ruminococcus sp.
MKITVVCDVLGAENNGTTIAAMNLIRALKAKGHDVIVVCSDEDKKGKNGYVVMPQLNLGPLNGYVKKNGVALSRADNKVLEPVIWDSDVVHIMLPFALGRAALKLCLKHNIPVTAGFHMQAENFTSHVFMKDNPVANNLTYRYIYNNLYKYVNAVHYPTDFIREVFENAVGHKTNAYVISNGVSDRFKPNGAKKPPEFRNKFVILFSGRYSKEKSHSVLIDAAALSKHSSEIQLIFAGDGPLKDMLKERSKKLPNKPVFSFFPHHQMLNVLNYADLYVHPAEIEIEAIACMEALSCGQVPIIANSPRCATKKFALDERNLFENRNPQDLADKIDYFIEHPEAIEEYRERYKGIVAELSQEACMNRMEEMLYEARG

SEQ ID NO: 95 - protein, 626 aa, Acetivibrio sp.
MLFLLTVFFFVFEKLKVNAARKERGVKFLMIITLVNDTFNINNNGTTISAMRFAEALSQRGHQIRIITCGDPLKSGKDPDTGFEMFYLPELKIPIASRLAHKQNTLFAKPVRSILKKAISGSDVVHIYQPWPLGSAAQRVARQMNIPAIAAFHIQPENITFNIGLKRFSPAAHLTYFLFYLFFYRRFSHIHCPSKFIAAQLRSHGYKARLHVISNGVHPAFCAPAKPREHTFKPIKILMIGRLSPEKRQDVLIRAVMKSRYADRIQLYFAGSGPWEKKLRRLGNKLPNPPVFGYYNRDELIKLIHECDLYVHASDAEIEGISLIEAFACGLVPIISDSKQSAAAQFALGPQNLFKAGSPESLAEKIDYWLDHPEQLKEAEKKYAQLGKQYALEHSIRKIEKVYSSMTKNHKNEYHRSIFFRLSTRLFQIVIACPILLLWTRFVLGAKVYGRENIRGLKSGVTVCNHVHLLDSALIGVTFFPRRVVFPTLTQNVKTLWPGKLVRILGGFAIPDNIMELKAFFDEMEFLLMKNCIVHFFPEGELRPYDTGLQNFKKGAFYLAAQAQVPIVPMLITFEPPKGLIKIIRKKPVMRLHIGKPIHPMSKDIEIDSELRMKAVCKKIEAITSV

SEQ ID NO: 96 - protein, 389 aa, Helicobacter pylori
MVIVLVVDSFKDTSNGTSMTAFRFFEALKKRGHAMRVVAPYVDNLGSEEEGYYNLKERYIPLVTEISHKQHILFAKPDEKILRKAFKGADMIHTYLPFLLEKTAVKIAREMQVPYIGSFHLQPEHISYNMKLGQFSWFNMMLFSWFKSSHYRYIHHIHCPSKFIVEELEKYNYGGKKYAISNGFDPMFKCEHPQKSLFDTIPFKIAMVGRYSNEKNQSVLIKAVALSRYKQDIVLLLKGKGPDEKKIKLLAQKLGVKTEFGFVNSNELLEILKTCTLYVHAANVESEAIACLEAISVGIVPVIANSPLSATRQFALDERSLFEPNNAKDLSTKIDWWLENKLERERMQNKYAKSTLNYTLENSVIQIEKVYEEAIRDFKNNPHLFKTLS

SEQ ID NO: 97 - protein, 597 aa, Acetivibrio sp.
MVITLVNDTFNINNNGTTISAMRFAEALSQRGHQIRIITCGDPLKSGKDPDTGFEMFYLPELKIPIASRLAHKQNTLFAKPVRSTLKKAISGSDVVHIYQPWPLGSAAQRVARQMNIPAIAAFHIQPENITFNIGLKRFSPAAHLTYFLFYLFFYRRFSHIHCPSKFIAAQLRSHGYKARLHVISNGVHPAFCAPAKPREHTFKPIKILMIGRLSPEKRQDVLIRAVMKSRYADRIQLYFAGSGPWEKKLCHLGNKLPNPPVFGYYNRDELIKLIHECDLYVHASDAEIEGISLIEAFACGLVPIISDSKQSAAAQFALGPQNLFKAGSPESLAEKIDYWLDHPEQLKEAEKKYAQLGKQYALEHSIRKIEKVYSSMTKNHKNEYHRSIFFRLSTRLFQIVIACPILLLWTRFVLGAKVYGRENIRGLKSGVTVCNHVHLLDSALIGVTFFPRRVVFPTLTQNVKTLWPGKLVRILGGFAIPDNIMELKAFFDEMEFLLMKNCIVHFFPEGELRPYDTGLQNFKKGAFYLAAQAQVPIVPMLITFEPPKGLIKIIRKKPVMRLHIGKPIHPMSKDIEIDSELRMKAVCKKIEAITSV

SEQ ID NO: 98 - protein, 396 aa, Bifidobacterium bifidum
MRDNKNEEPRPQDRSVLDRPMTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAAGQFALLDESLFPARDVAMLARRIDWWIDHPHELAVWGGRYAEHAKADYSVEASVAKFVAMEREAIADAKA

SEQ ID NO: 99 - protein, 396 aa, Bifidobacterium bifidum
MRDNKNEEPRPQDRSVLDRPMTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 100 - protein, 396 aa, Bifidobacterium bifidum
MRDNKNEEPRPQDRSVLDRPLTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 101 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVARQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVIASGRLASEKDQITLIKAVSMSRHAGDIQLVIAGTGPLKQYLKFRAGRLLARKADIGFHKHADMPDLLRSGDLFVHCSIADIESVSVIEAMACGLVPVIAASELSAASQFALIDESLFPVRDAALLARRIDWWIAHQAERAEWGEKYAEYTKAHYSVEASVHKFVDMERKAIGE

SEQ ID NO: 102 - protein, 396 aa, Bifidobacterium sp.
MRDNKNEEPHPQDRSVLDRPMTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 103 - protein, 396 aa, Bifidobacterium bifidum
MRDNKNEEPRPQDRSALDRPMTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 104 - protein, 648 aa, unidentified
MGMRIVFVIDSFNLNNGTAATARRYAEELRKRGHEVTILAAGSADGNKTGIRKLRIPFFQPLIEKQGFCFARPNDEAYYRAFRNADIIHFFLPTWFCRRGEFIARQMRIPTVSAFHLQPENVTYSIGLGKSKRANDVIYRYFYKCFYNRFRFIHCPSEMIAEQLMQHGYDAECRVISNGVNDLFRPAEVRRPEYLKGKIVLLMVGRLSGEKRQDLLIEAVQYSKYRDKIQLVFAGMGPKEKKYRKLSRNLKNKPIFRFFTQEELLQMYNICDLYIHTSDAEIEGISCIEAMACGAVPVISDSHLSATKSFALHPNCLFKAGDARSLAEKIDYWIEHPEERSKMSRIYAEKAETIRVSKCVAEMERLYKDVISDYHKNGYKQPEKSRLRKLLHPDTDAVNVAYSKRTPARQALFYVFTNLIAIIIYFIDTVFFGLVIEGKDKLKKVRGGAVTVMNHIHPMDCTMVKLAVFPRPIYFTSLVNNLELPLVGWLIRFCGALPVPNGKGKLVGFMKHIKHGIQNGDLVHFYPEGMLIRNYEGLREFQPGAFYTAVHTGCPVIPMVLVNIHPQGIWRIAGGRRMHLFIGEPQYPNSELSPKESVTELKNRTWQIMNEMMNEPVEAERISFSVAVRIACILYIASQVVRIIAIRL

SEQ ID NO: 105 - protein, 396 aa, Bifidobacterium bifidum
MRDNKNEEPHPQDRSVLDRPMTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIVCGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 106 - protein, 597 aa, Acetivibrio sp.
MIITLVNDTFNINNNGTTISAMRFAEALSQRGHQIRIITCGDPLKSGKDPDTGFEMFYLPELKIPIASRLAHKQNTLFAKPVRSILKKAISGSDVVHIYQPWPLGSAAQRVARQMNIPAIAAFHIQPENITFNIGLKRFSPAAHLTYFLFYLFFYRRFSHIHCPSKFIAAQLRSHGYKARLHVISNGVHPAFCAPAKPREHTFKPIKILMIGRLSPEKRQDVLIRAVMKSRYADRIQLYFAGSGPWEKKLRRLGNKLPNPPVFGYYNRDELIKLIHECDLYVHASDAEIEGISLIEAFACGLVPIISDSKQSAAAQFALGPQNLFKAGSPESLAEKIDYWLDHPEQLKEAEKKYAQLGKQYALEHSIRKIEKVYSSMTKNHKNEYHRSIFFRLSTRLFQIVIACPILLLWTRFVLGAKVYGRENIRGLKSGVTVCNHVHLLDSALIGVTFFPRRVVFPTLTQNVKTLWPGKLVRILGGFAIPDNIMELKAFFDEMEFLLMKNCIVHFFPEGELRPYDTGLQNFKKGAFYLAAQAQVPIVPMLITFEPPKGLIKIIRKKPVMRLHIGKPIHPMSKDIEIDSELRMKAVCKKIEAITSV

SEQ ID NO: 107 - protein, 398 aa, Bifidobacterium tsurumiense
MNRDREVPSGQRENGDIDVRPLTIALVVDTIGNHGNGTSNSALQYAQELERQGHHVRLVGIGSQEYPAKVNRIPLVSHIAARQQMQFAKPSDTLFRTAFAGVDVVHIYMPFSFGRRARIIAQEMGIPVTAGFHVQPENVLYSAGPLRFVPGASRFIYWLFKRWLYRYVDHIHTPTQMIAEELRKHHYDAKLHAISNGYSPRFVPRQPRAAADIRPPYRVAASGRLSLEKSQITLIKAISLCRHGSDIELSICGTGPMRRYLQMRAKRLLNSPVRIGFQPNDRMPGLLRSQDLLVHASVVDIESLSVMEGIGSGVVPIIARSGLSAASQFALTDQSLFEARNAQQLADRIDWWLDHPMELVSWERKYARFAAQRYSVESSVRQFIEMERESIAEYESSH

SEQ ID NO: 108 - protein, 403 aa, Bifidobacterium thermacidophilum
MKNQEFSQAAGSTEDAGRPLVIALVVDTAGNEGNGTSNSALQYAKELRRQGHTVRLVGIGSPEYPIRVRHIPLVSWIAAKQQMQFAQPDRAMFRKAFEGADVVHVYLPFKYGRMAVRTAHEMGIPATAGFHLQPENVLYSAGPLRYIPGASAFIYWLFDRILYRHIRHIHTPSHMIAAQLRAHGYRQRLHVISNGYSPRFTAKQQREPGSPSPRPFRIVASGRLASEKDHVTLIRAVALSRHAEDIELSICGTGPLQHELRRMAAQLLPGRWSIGFHDNAQMPVFLRSCDLLVHPSIVDIESLSVLEGMASGLVPVIARSTQSAAGQFALCDHSLFEAQDPAMLAQRIDWWIDHPDELSRWGAIYAEHTREEYSIESCVRKFVAMEREAIADFDSGSRMTGAI

SEQ ID NO: 109 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVAHQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVIASGRLASEKDQITLIKAVSMSRHAGDIQLVIAGTGPLKQYLKFRAGRLLARKADIGFHKHADMPDLLRSCDLFVHCSIADIESVSVIEAMACGLVPVIAASELSAAGQFALIDESLFPVRDAALLARRIDWWIAHQAERAEWGEKYAEYTKVHYSVEASVHKFVDMERKAIGE

SEQ ID NO: 110 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVAHQMGIPVTAGFHLQPENVLYSAGPLRRIPGISSFLYWLFKHWLYKHIDHIHVPTEMTASLLRAHGYKAKLHVISNGYSPVYSPKKPADPDAPAPVPFRVVASGRLASEKDQITLIKAVSMSRHAGDIQLVIAGTGPLKQYLKFRAGRLLARKADIGFHKHADMPDLLRSCDLFVHCSIADIESVSVIEAMACGLVPVIAASELSAAGQFALIDESLFPVRDAALLARRIDWWIAHQAERAEWGEKYAEYTKVHYSVEASVHKFVDMERKAIGE

SEQ ID NO: 111 - protein, 398 aa, Bifidobacterium boum
MSQSSKFTRATGVNGDADRPLTIALVVDTAGNEGNGTSNSALQYARELRRQGHTVRLVGIGSPEYPIRVHHIPLVSWIAAKQQMQFAQPDRAVFRKAFVGVDVVHVYLPFKYGRVAVQTAHEMGIPATAGFHLQPENVLYSAGPLRYIPGASAFIYWLFDRMLYRHIRHIHTPSHMIAAQLRAHGYRQRLHVISNGYSPRFTPKRQREPGSPSPRPFRIVASGRLASEKDHVTLIRAVALSRHAEDIELVICGTGPLQHELRRMAGKLLPGRWSIGFHDNAQMPELLRSCDLLVHPSIVDIESLSVLEGMASGLVPVIARSTQSAAGQFALNDHSLFEAQDSAMLAQRIDWWIDHPDELSRWGAVYAEHTREEYSIASCVRKFVAMEREAIADFDGRL

SEQ ID NO: 112 - protein, 386 aa, Bifidobacterium saguini
MRENGSEESIDRPLTIALVVDTVGNQGNGTSNSALQWAAELERQGHHVRLVGVGAPEYPARVNKVPLVSWVASKQLMQFAQPSDTLFRTAFQGVDVVHIYMPFKFGRHAAKVARQMGIPVTAGFHLQPENVLYSAGPLRHIPGVSSFLYWLFKHWLYRRIDHIHVPTEMTASLLRAHGYKAKLHVISNGYSPAYKPLKSVDPAAPTPVPFRIIASGRLAREKDQITLIKAISMSRHAADIQLVIAGTGPLRRYLSFRAGRRLARKADIGFHKHAEMPELLRSGDLFVHCSIADIESVSVIEAMACGLVPVIAASELSAASQFALLDESLFPVRDAALLARRIDWWISHQQERAEWSAKYAENTKAHYSVEASVHKFVDMEREAIRE

SEQ ID NO: 113 - protein, 399 aa, Bifidobacterium stellenboschense
MREQENAEEFGKTSIVEEDIDRPLTIALVVDTVGNQGNGTSNSALQWAAELERQGHHVRLVGVGAPDYPARVNRVPLVSWVAAKQQMQFAEPSDTLFRTAFQGVDVVHIYMPFKFGRRAAKIARQMHIPVTAGFHLQPENVLYSAGPLRYIPGMEKFLYWLFRQWLYKRVDHIHTPTEMTASLLREHGYKGTMHVISNGYSPRFTARKPLDSDAPAHVPFRVVASGRLAHEKDQITLIKAISMSRHASDIQLVIAGTGPLKRYLTFRANRRLKRKADIGFHRNADMPALLRSCDLFVHCSIADIESVSVIEAMACGLVPVIAASELSAASQFALTDASLFPVRDAAMLARRIDWWIDHPDELARWGEIYAEHTREHYSVEASVHRFVDMEREAIATAGR

SEQ ID NO: 114 - protein, 391 aa, Bifidobacterium sp.
MHENGTGEESIDRPLTIALVVDTVGNQGNGTSNSALQWAAELERQGHHVRLVGIGAPEYPARVNKVPLVSWVASKQLMQFAQPSDTLFRTAFRGVDVVHVYMPFKFGRHAAKVARQMGIPVTAGFHLQPENVLYSAGPLRHIPGISGFLYWLFKHWLYKRIDHIHVPTEMTASLLRSHGYQAKLHVISNGYSPVYTPKPPAESSEDPDAASPVPFRIIASGRLAREKDQITLIKAVSMSRHAADIQLVIAGTGPLKHYLKFRAGRLLARKADIGFHKHADMPELLRSGDLFVHCSIADIESVSVIEAMACGLVPVIAASELSAASQFALLDESLFPVRDAALLARRIDWWIAHQRERVAWGARYAQNTKEHYSVEASVHKFVDMEREAIEE

SEQ ID NO: 115 - protein, 403 aa, Bifidobacterium sp.
MKNQEFSVTAGSTEDAGRPLVIALVVDTAGNEGNGTSNSALQYAGELRRQGHTVRLVGIGSPEYPIRVRHIPLVSWIAAKQQMQFAQPDRAMFRKAFEGADVVHVYLPFKYGRMAVRTAHEMGIPATAGFHLQPENVLYSAGPLRYIPGASAFIYRLFDRMLYRHIRHIHTPSHMIATQLRAHGYRQRLHVISNGYSPRFTAKQQREPGSPSPRPFRIVASGRLASEKDHVTLIRAVALSRHAEDIELSICGTGPLQHELRRMAAQLLPGRWSIGFHDNAQMPVFLRSCDLLVHPSIVDIESLSVLEGMASGLVPVIARSTQSAAGQFALCDHSLFEAQDPAMLAQRIDWWIDHPDELSRWGAVYAEHTREEYSIESCVRKFVAMEREAIADFDGGSRMTGAI

SEQ ID NO: 116 - protein, 397 aa, Bifidobacterium thermophilum
MKNQEFSVAAGSTEDAGRPLVIALVVDTAGNEGNGTSNSALQYAKELRRQGHTVRLVGIGSPEYPIRVRHIPLVSWIAAKQQMQFAQPDRAMFRKAFEGADVVHVYLPFKYGRMAVRTAHEMGIPATAGFHLQPENVLYSAGPLRYIPGASAFIYWLFDRMLYRHIRHIHTPSHMIAAQLRAHGYRQRLHVISNGYSPRFTAKQQREPGSPSPRPFRIVTSGRLASEKDHVTLIRAVALSRHAEDIELSICGTGPLQHELRRMAAQLLPGRWSIGFHDNAQMPAFLRSCDLLVHPSIVDIESLSVLEGMASGLVPVIARSTQSAAGQFALCDHSLFEAQDPAMLAQRIDWWIDHPDELSRWGAIYAEHTREEYSIESCVRKFVAMEREAIADFDGRL

SEQ ID NO: 117 - protein, 396 aa, Bifidobacterium bifidum
MRDNKNEEPRPQDRSVLDRPMTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 118 - protein, 397 aa, Bifidobacterium scardovii
MLENGNEGNGGLPEIDRPLTIVLVMDTIGNQGNGTSNSALQYAHELERQGHHVRLVGIGAPEYPVRVNKVPLVSWVAAKQQMQFAEPSETLFRTAFAGADVVHIYLPFKFGRCAAKVARRMGIAVTAGFHLQPENVTYSAGPIKYIPGISNFLYALFRHWLYRNIGHIHVPTEMIAGQLRAHGYTAKLHVISNGYVPRFTPKRQRRADAPAPVPFRIVASGRLSHEKDHITLIKAIARCRHAKDIELTICGTGPLRRYLRFRAKMLLSRPASIGFHKNAEMPALLRSCDLFAHPSIVDIESLSVIEGMASGLVPVIASAELSAAGQFALLDESLFPAHDTVTLARRIDWWIDHPAELAEWGARYAAHTKAHYSVEASVAQFVAMEREAIADNARDAR

SEQ ID NO: 119 - protein, 406 aa, Bifidobacterium minimum
MRDDGRGTPHDGRPLTIVMVVDTIGRNGNGTSNSAMQYAGRLRELGHRVRLVGIGSEDYPAASHHVPIASWASAKQQIQFARPDADLMRRAFAGADVVHIYLPFAFGRCARAVAREMDVPVTAGFHLQPENVTYSAGPLRYVPGVSAFLYRLFRRWLYAGIGHVHAPTRMIADQLRSHGYDNEIHVISNGFSPMFSPSHGGEPDGRDGRDRGRRFRIVASGRLSHEKDHITLIRAVARCRHAADIDLAICGTGPLDSYLRAQARRLLPRTRWSIGFHSHDEMPRLLRACDLIVHPSIVDIESLSVLEGIACGLVPVIAESDLSAARQFALTDESLFPARDVQALADGIDWWVEHAEDRRYWAARYADEARSRYSLERCVDRFVDMERAAIADHQALAISRPVEAAS

SEQ ID NO: 120 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPASVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 121 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 122 - protein, 386 aa, Bifidobacterium breve
MRENGIEKSIDRPLTIALVVDTVGNQGNGTSNSALQWAAELERQGHHVRLVGVGAPEYPARVNKVPLVSWVAAKQLMQFAEPSDTLFRTAFQGVDVVHVYMPFKFGRRAAKVAHQMGIPVTAGFHLQPENVLYSAGPLRHIPGISSFLYWLFKHWLYKRIDHIHVPTEMTASLLRAHGYKAKLHVISNGYSPVYSPKKPVDPEASAPVPFRVIASGRLASEKDQITLIKAVSMSKHAGGIQLIIAGTGPLEQYLRFRAGRLLARKADIGFHKHADMPDLLRSGDLFVHCSIADIESVSVIEAMACGLVPVIAASELSAASQFALLDESLFPVQNAAMLARRIDWWIEHQVERAEWGEKYAEYTKANYSVEASVHKFVDMEREAIGE

SEQ ID NO: 123 - protein, 388 aa, Bifidobacterium sp.
MLKNASEHVEQPLTIALVVDSIGNQGNGTSNSALQWASELERQGHHVRLVGVGAPEYPARVNKVPLVSWVAAKQQMQFAEPSDTLFRTAFAGADVVHIYMPFKFGRRAAKIARDMGIPVTAGFHLQPENVLYSAGPLRHIPGMQNLLYRLFHWWLYRRIDHIHTPTEMTASLLRSHGYRGTLHVISNGYSPRFEAREPLSAQEAVRTPLRIVASGRLAHEKDQITLIKAISMSRYASDIQLIIAGTGPLRRYLQFRADLLLARKAQIGFHKNADMPALLRSCNLFVHCSIADIESVSVIEAMACGLVPVIAASELSAASQFALLDESLFPVRNAALLARRIDWWFDHPEELAAWGARYAEHTESHYSVESSVRRFVAMEREAIADAAK

SEQ ID NO: 124 - protein, 412 aa, Bifidobacterium sp.
MREQEVEEDRIVSGRDARTVKSGDDAGVTEEGIDRPLTIALVVDTIGNQGNGTSNSALQWAAELRRQGHHVRLVGIGAPEYPARVNHVPLVSWVAAKQQMQFAEPSDTLFRTAFRGVDVVHIYMPFKFGRRAAKIARQMGIPVTAGFHLQPENVLYSAGPLRYIPGMERFLYWLFRQWLYKRVDHIHTPTEMTASLLREHGYKGVMHVISNGYAPRFTARAPLAPDAAAHVPFRVIASGRLAHEKDQITLIKAISMSRHASDIQLVIAGTGPLRRYLTFRANRRLKRKADIGFHRNADMPALLRSCDLFVHCSIADIESVSVIEAMACGLVPVIAASELSAASQFALLDESLFPVRDATMLARRIDWWIDHPDELARWGATYARHTREHYSVEASVHRFVDMEREAMAQGAR

SEQ ID NO: 125 - protein, 388 aa, Bifidobacterium sp.
MLKNASEHVEQPLTIALVVDSIGNQGNGTSNSALQWASELERQGHHVRLVGVGAPEYPARVNKVPLVSWVAAKQQMQFAEPSDTLFRTAFAGADVVHIYMPFKFGRRAAKIARDMGIPVTAGFHLQPENVLYSAGPLRHIPGMQNLLYRLFHLWLYRRVDHIHTPTEMTASLLRSHGYRGTLHVISNGYSPRFEAREPLSAQEAVRMPLRIVASGRLAHEKDQITLIKAVSMSRYASDIQLIIAGTGPLRRYLQFRADLMLARKAQIGFHRNSDMPALLRSCNLFVHCSIADIESVSVIEAMACGLVPVIAASELSAASQFALLDESLFPVRNAALLARRIDWWFDHPEELAAWGARYAEHTESHYSVESSVRRFVAMEREAIADAAK

SEQ ID NO: 126 - protein, 386 aa, Bifidobacterium sp.
MRENGSEESLDRPLTIALVVDTVGNQGNGTSNSALQWATELERQGHHVRLVGIGAPEYPARVNKVPLVSWVAAKQLMQFAQPSDTLFRTAFQGVDVVHIYMPFKFGRHAAKVARQMGIPVTAGFHLQPENVLYSAGPLRHIPGISTFLYWLFKHWLYKRVDHIHVPTEMTASLLRSHGYQATLHVISNGYSPEYQPGELPDPKAASPVPFRIIASGRLAHEKDQITLIKAIAMSRHAEDIQLVIAGTGPLRHYLRFRAGRLLARKAEIGFHRHAEMPSLLRSGDLFVHCSIADIESVSVIEAMACGLVPVIAASELSAASQFALLDESLFPVRDVALLARRIDWWIAHPRERAEWSAKYAESTKEHYSVEASVHKFVDMEREAIEG

SEQ ID NO: 127 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVARQMGIPVTAGFHLQPENVLYSAGPLRRIPGISSFLYWLFKHWLYKHIDHIHVPTEMTASLLRAHGYKAKLHVISNGYSPVYSPKKPADPDASASVPFRVIASGRLASEKDQITLIKAVSMSRHAGDIQLVIAGTGPLKQYLKFRAGRLLARKADIGFHKHADMPDLLRSGDLFVHCSIADIESVSVIEAMACGLVPVIAASELSAASQFALIDESLFPVRDAALLARRIDWWIAHQAERAEWGEKYAEYTKAHYSVEASVHKFVDMERKAIGE

SEQ ID NO: 128 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVAHQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVVASGRLASEKDQITLIKAVSMSRHAGDIQLVIAGTGPLKQYLKFRAGRLLARKADIGFHKHADMPDLLRSCDLFVHCSIADIESVSVIEAMACGLVPVIAASELSAAGQFALIDESLFPVRDAALLARRIDWWIAHQAERAEWGEKYAEYTKAHYSVEASVHKFVDMERKAIGE

SEQ ID NO: 129 - protein, 397 aa, Bifidobacterium thermophilum
MKNQEFSVAAGSTEDAGRPLVIALVVDTAGNEGNGTSNSALQYAKELRRQGHTVRLVGIGSPEYPIRVRHIPLVSWIAAKQQMQFAQPDRAMFRKAFEGADVVHVYLPFKYGRMAVRTAHEMGIPATAGFHLQPENVLYSAGPLRYIPGASAFIYWLFNRMLYRHIWHIHTPSHMIAAQLRAHGYRQRLHVISNGYSPRFTAKQQREPGSPSPRPFRIVASGRLASEKDHVTLIRAVALSRHAEDIELTICGTGPLQHELRRMAGKLLPGRWSIGFHDNAQMPELLRSCDLLVHPSIVDIESLSVLEGMASGLVPVIARSTQSAAGQFALNDHSLFEAQDPAMLAQRIDWWIDHPDELSRWGAIYAEHTREEYSIASCVRKFVAMEREAIADFDGRL

SEQ ID NO: 130 - protein, 397 aa, Bifidobacterium thermophilum
MKNQEFSVAAGSTEDAGRPLVIALVVDTAGNEGNGTSNSALQYAKELRRQGHTVRLVGIGSPEYPIRVRHIPLVSWIAAKQQMQFAQPDRAMFRKAFEGADVVHVYLPFKYGRMAVRTAHEMGIPATAGFHLQPENVLYSAGPLRYIPGASAFIYWLFNRMLYRHIWHIHTPSHMIAAQLRAHGYRQRLHVISNGYSPRFTAKQQREPGSPSPRPFRIVASGRLASEKDHVTLIRAVALSRHTEDIELTICGTGPLQHELRRMAAQLLPGRWSIGFHDNAQMPAFLRSCDLLVHPSIVDIESLSVLEGMASGLVPVIARSTQSAAGQFALCDHSLFEAQDPAILAQRIDWWIDHPDELSRWGAIYAEHTREEYSIASCVRKFVAMEREAIADFDGRL

SEQ ID NO: 131 - protein, 386 aa, Bifidobacterium sp.
MRENGSEESIDRPLTIALVVDTVGNQGNGTSNSALQWAAELERQGHHVRLVGVGAPEYPARVNKVPLVSWVASKQLMQFAQPSDTLFRTAFQGVDVVHIYMPFKFGRHAAKVARQMGIPVTAGFHLQPENVLYSAGPLRHIPGISSFLYWLFKHWLYKRIDHIHVPTEMTASLLRAHGYKAKLHVISNGYSPAYKPLKSVDPAAPTPVPFRIIASGRLAREKDQITLIKAISMSRHAADIQLVIAGTGPLRRYLSFRAGRRLARKADIGFHKHAEMPELLRSGDLFVHCSIADIESVSVIEAMACGLVPVIAASELSAASQFALLDESLFPVRDAALLARRIDWWISHQQERAEWSAKYAENTKAHYSVEASVHKFVDMEREAIRE

SEQ ID NO: 132 - protein, 386 aa, Bifidobacterium breve
MRENGIEKSIDRPLTIALVVDTVGNQGNGTSNSALQWAAELERQGHHVRLVGVGAPEYPARVNKVPLVSWVAAKQLMQFAEPSDTLFRTAFQGVDVVHVYMPFKFGRRAAQVARQMGIPVTAGFHLQPENVLYSAGPLRHIPGISSFLYWLFKHWLYKRIDHIHVPTEMTASLLRAHGYKAKLHVISNGYSPVYSPKKSVDPEASAPVPFRVIASGRLASEKDQITLIKAVSMSKHAGDIQLIIAGTGPLKQYLRFRAGRLLARKADIGFHKHADMPDLLRSGDLFVHCSIADIESVSVIEAMACGLVPVIAASELSAASQFALLDESLFPVQNAAMLARRIDWWIEHQVERAEWGEKYAEYTKSHYSVEASVHKFVDMEREAIGE

SEQ ID NO: 133 - protein, 399 aa, Bifidobacterium callitrichos
MRERAIEAIEEDHEERIDRPLTIALVVDTVGNQGNGTSNSALQWAAELKRQGHHVRLVGIGAPDYPARVNKVPLVSWVAAKQQMQFAEPSDTLFRTAFRGVDVVHIYMPFKFGRRAAKVARQMGIPVTAGFHLQPENVLYSAGPLRYIPGMEKFLYWLFRQWLYKRVDHIHTPTEMTASLLREHGYRGVMHVISNGYSPRFTAREPIDPQAPARVPFRVVASGRLAHEKDQITLIKAISMSRHASDIQLIIAGTGPLKRYLTFRANRRLKRRADIGFHNNADMPALLRSCDLFVHCSIADIESVSVIEAMASGLVPVIAASELSAASRFALLDESLFPVRDAAMLARRIDWWIDHPAELARWGARYATHTREHYSVEASVHKFVDMEREAIDAKATAAR

SEQ ID NO: 134 - protein, 401 aa, unidentified
MDTFDRESGRDLPSLPGRAAQPIPQRRTAAMKVATICDVMGQPNNGTTLAALNLIRYLRDAGHTVTVVAPGGEASDGYLPVRVWHAGPLIDRILRMNGVELAVPDKQLLEAVIREADVVHLLIPLPLARAALKIARRLGKPVTASFHCQAENITAHLGMMNAGWLNRLIYRNFYRKVYRWCTAVHYPTEFIREVFETATHPTPAHVISNGVNDMFRLPDSRPENGKFTVVCSGRYSREKAQQQLLRAAALCRHRDDIRLILAGDGPRRKHYLRLAKQYGLDCQFAFFPRQELLHILQTADLYVHTAIIEIEAIACTEAICCGLVPVICNSDRSATRFFACGDHTLFEPGDVRALADLLDYWYEHPAARVERAAEYADLRHSFDQTACMQQMEQMLKEAAGL

SEQ ID NO: 135 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 136 - protein, 396 aa, Bifidobacterium bifidum
MRDNKNEEPRLQDRSALDRPLTIVLVMDTIGNKGNGTSNSALQYAHELERQGHRVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQCSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 137 - protein, 396 aa, Bifidobacterium bifidum
MRDNKNEEPRPQDRSVLDRPLTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 138 - protein, 396 aa, Bifidobacterium bifidum
MRDNKNEEPHPQDRSVLDRPMTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 139 - protein, 394 aa, Bifidobacterium sp.
MAENESREEDARPPQIERPLTIVLVMDTIGNKGNGTSNSALQYAHELERQGHHVRLVGIGAPEYPARINKVPLVSWVAAKQQMQFAQPSETLFRTAFAGADIVHIYLPFKFGRQAYRIARSMGIPVTAGFHLQPENVTYSAGPLKYVPGISSFLYWLFNMWLYRRVDHIHAPTEMIAEQLRSHGYKAKLHVISNGYTARFTPKQQRSANAPTPVPFRVVASGRLTHEKDHITLIKAIARCRHARDIELTICGTGPLAHYLRFRAKRLLTRPASIGFHDNADMPALLRSCDLFVHPSIVDIESLSVIEGMASGLVPVIAASELSAASQFALLDESLFPARDATLLARRIDWWIDHPDELAKWGERYARHTKEHYSVEASVTQFVDMERQAIADNH

SEQ ID NO: 140 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGLYAEHAKADYSVEASVAKFVAMEREAIADAKA

SEQ ID NO: 141 - protein, 386 aa, Bifidobacterium breve
MRENGIEKSIDRPLTIALVVDTVGNQGNGTSNSALQWAAELERQGHHVRLVGVGAPEYPARVNKVPLVSWVAAKQLMQFAEPSDTLFRTAFQGVDVVHVYMPFKFGRRAAKVAHQMGIPVTAGFHLQPENVLYSAGPLRHIPGISSFLYWLFKHWLYKRIDHIHVPTEMTASLLRAHGYKAKLHVISNGYSPVYSPKKPVDPEASAPVPFRVIASGRLASEKDQITLIKAVSMSKHAGDIQLIIAGTGPLKQYLRFRAGRLLARKADIGFHKHADMPDLLRSGDLFVHCSIADIESVSVIEAMACGLVPVIAASELSAASQFALLDESLFPVQNAAMLARRIDWWIEHQVERAEWGEKYAEYTKAHYSVEASVHKFVDMEREAIGE

SEQ ID NO: 142 - protein, 399 aa, Bifidobacterium reuteri
MRENGTGEESIDRPLTIALVVDTVGNQGNGTSNSALQWAAELERQGHHVRLVGIGAPEYPARVNKVPLVSWVASKQLMQFAQPSDTLFRTAFRGVDVVHVYMPFKFGRHAAKVARQMGIPVTAGFHLQPENVLYSAGPLRHIPGISGFLYWLFKHWLYKRIDHIHVPTEMTASLLRSHGYQAKLHVISNGYSPVYTPKPPAESSEDPDAASPVPFRIIASGRLAREKDQITLIKAVSMSRHAADIQLVIAGTGPLKHYLKFRAGRLLARKADIGFHKHADMPELLRSGDLFVHCSIADIESVSVIEAMACGLVPVIAASELSAASQFALLDESLFPVRDAALLARRIDWWIAHQRERVAWGAKYAQNTKEHYSVEASVHKFVDMEREAITDNAYSSAMS

SEQ ID NO: 143 - protein, 399 aa, Bifidobacterium sp.
MRERAIEAIEEDHEERIDRPLTIALVVDTVGNQGNGTSNSALQWAAELKRQGHHVRLVGIGAPDYPARVNKVPLVSWVAAKQQMQFAEPSDTLFRTAFRGVDVVHIYMPFKFGRRAAKVARQMGIPVTAGFHLQPENVLYSAGPLRYIPGMEKFLYWLFRQWLYKRVDHIHTPTEMTASLLREHGYRGVMHVISNGYSPRFTAREPVDPQAPARVPFRVVASGRLAHEKDQITLIKAISMSRHASDIQLIIAGTGPLKHYLTFRANRRLKRRADIGFHNNADMPALLRSCDLFVHCSIADIESVSVIEAMASGLVPVIAASELSAASQFALLDESLFPVRDAAMLARRIDWWIDHPAELARWGARYATHTREHYSVEASVHKFVDMEREAIDAKATAAR

SEQ ID NO: 144 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPRELAVWGGRYAEHAKADYSVEASVAKFVAMEREAIADAKA

SEQ ID NO: 145 - protein, 396 aa, Bifidobacterium bifidum
MRDNKNEEPHPQDRSVLDRPMTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASGAKFVAMEREAIADAKA

SEQ ID NO: 146 - protein, 396 aa, Bifidobacterium sp.
MLANRGKENDGRTQAPQIEAPLTIALVVDTIGNKGNGTSNSALQYARELERQGHHVRLVGVGAPEYPAKVNKVPLVSWVAAKQQMQFARPSDTLFRTAFAGADVVHVYLPFKFGRRACKVAHDMGIPVTAGFHLQPENVMYSAGPLKYLPGMSAFLYALFRFWLYRHVRHIHAPTEMIAGQLRAHGYKAKLHVISNGYVPRFTPKRQPAADAPVPVPFRIVASGRLTHEKDHITLIKAIARCRHAKDIELVICGTGPLQRYLRFRAKRLLEREAQIGFHRNADMPALLRSCDLLVHPSIVDIESLSVIEGMASGLVPVIAASDLSAASQFALLDESLFPARDVTMLARRIDWWIDHPDELRRWGAQYARHAKEHYSVEASVTKFVAMEREAVAEGV

SEQ ID NO: 147 - protein, 393 aa, Bifidobacterium sp.
MPEIEDEARETHEAGDRPLTIALVMDTIGTQGNGTSNSALQYARELERQGHHVRLVGLGSEEWPARENHVPLVSWVAKKQHMRFAKPSDTLFRTAFRGVDVVHIYLPFSFGRRAWKIAREMGIPVTAGFHLQPENVSYNAGPIKWIPGVNDFMYALFRFWLYRRVGHIHTPTEMIATQLREHDYKAKLHVISNGYSPRFTPKTQRKAGAREPWRYRVVASGRLAREKDHATLIRAVAMCRHAERIELHIAGTGPRERYLKRRAKRMLPNPAYIGFHRNADMPAFLKTCDLFVHPSIVDIESLSVIEGMASGLVPVIAESELSAAGQFALLDESLFPAGDAAALAERIDWWLDHPRELATWGAKYAGHTREHYSIESCVRRFVAMEREAIADAA

SEQ ID NO: 148 - protein, 400 aa, Bifidobacterium sp.
MKNQGSNQGSVAAGNIADAGRPLVIALVVDTAGNEGNGTSNSALQYAKELRRQGHTVRLVGIGSPEYPIRVHHVPLVSWIAAKQQMQFAQPDRSMFRKAFAGADVVHVYLPFKYGRVAVRTAHEMGIPATAGFHLQPENVLYSAGPLRYIPGASAFIYWLFDRMLYRHIRHIHTPSHMIAAQLRAHGYRQRLHVISNGYAPRFTAKRQREPGSPVPRPFRIVASGRLASEKDHVTLIRAVALSRHAEDIELVICGTGPLQHELRRMAGKLLPGRWSIGFHDNAQMPQLLRSCDLLVHPSIVDIESLSVLEGMASGLVPVIARSAQSAAGQFALNDHSLFEAQDPAMLAQRIDWWIDHPDELSRWGAIYAEHTREEYSIASCVRKFVAMEREAIADFDGRL

SEQ ID NO: 149 - protein, 398 aa, Bifidobacterium sp.
MREQQEDGVAADAAASQPQLDRPLTIVLVMDTIGNQGNGTSNSALQYAHELERQGHHVRLVGIGAPEYPARVSHVPLVSWVAAKQQMQFAEPSETLFRTAFRGADVVHIYLPFRFGRHAAKVARAMGIPVTAGFHLQPENVTYSAGPLRHVPGMSDFLYWLFRMWLYRGVGHIHAPTEMIAGQLRAHGYRAKLHVISNGYTARFTPKRQREPGTPSPVPFRVVASGRLTHEKDHITLIKAIARCRHAKDIELTICGTGPLQRYLRFRAKMLLGRPASIGFHQNADMPALLRSCDLFVHPSIVDIESLSVIEGMASGLVPVIASAELSAAGQFALLDKSLFPVRDAAALAQRIDWWIDHPDELAEWGARYAEHTKEHYSVEASVAKFVDMEREAIADVG

SEQ ID NO: 150 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFHFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLQHYLRFRADRLLERKAQIGFHKNAEMPTLLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 151 - protein, 398 aa, Bifidobacterium tsurumiense
MNRDREVPSGQRENGDIDVRPLTIALVVDTIGNHGNGTSNSALQYAQELERQGHHVRLVGIGSQEYPAKVNRIPLVSHIAARQQMQFAKPSDTLFRTAFAGVDVVHIYMPFSFGRRARIIAQEMGIPVTAGFHVQPENVLYSAGPLRFVPGASRFIYWLFKRWLYRYVDHIHTPTQMIAEELRKHHYDAKLHAISNGYSPRFVPRQPRAAADIRPPYRVAASGRLSLEKSQITLIKAISLCRHGADIELSICGTGPMRRYLQMRAKRLLNSPVRIGFQPNDRMPGLLRSQDLLVHASVVDIESLSVMEGIGSGVVPIIARSGLSAASQFALTDQSLFEARNAQQLADRIDWWLDHPMELVSWEQKYARFAAQRYSVESSVRQFIEMERESIAEYESSH

SEQ ID NO: 152 - protein, 395 aa, unidentified
MAGKIPGGRNQDLELGGSMVITFVIDMYDVKKNGTTMTAQRFAHYLQQLGHEIRVVSTGDPGPGKYPVPQWKIPIVQHFSSKQSFIIAKPDDEQIRRAIEGSDLVHLFLPFPLEVRAYHIAREMGIPCSAAFHLQPENITYNIHMGRWRGLSDFVYYLFREYFYKNFEHIHCPSYFMAEQLKAHGYKAKLHVISNGIIPEFCPGQATRQDELIHILMVGRLSPEKRQDLLIKAAGMSRHAEKIQLHFAGSGPWKQMLAHRGKKLKHPPVFGFYSKPELIELIRSCDLYVHASDAESEAIACLEAIACGLVPVISDSKLSATGQFALDERSLFHAGDAASLAEQIDYWIDHPEEKARMSSEYAKSTEKYRIEHCVREAETMFEEAVRDARGTGRPA

SEQ ID NO: 153 - protein, 409 aa, Bifidobacterium sp.
MREHESERNGRRPAGIEGFGADGVGFEEDIDRPLTIALVVDTVGNQGNGTSNSALQWAAELERQGHHVRLVGIGAPEYPARVNKVPLVSWVAAKQQMQFAQPSDTLFRTAFQGVDVVHVYMPFKFGRRAAKVARQMGIPVTAGFHLQPENVLYSAGPLRYVPGMERFLYWLFRQWLYKRVDHIHTPTEMTAGLLREHGYRGVTHVISNGYSPRFTAREPLDPDAPAHVPFRVVASGRLAHEKDQITLIKAVSMSRHAGDIHLTIAGTGPLKRYLTFRAHRRLRRNADIGFHRNADMPALLRSCDLFVHCSIADIESVSVIEAMACGLVPVIAASELSAASQFALTDESLFPVRDAAMLARRIDWWIDHPDELARWGAKYAEHTREHYSVEASVHRFVDMEREAIATAGR

SEQ ID NO: 154 - protein, 398 aa, Bifidobacterium boum
MSQSSKFTRATGVNGDADRPLTIALVVDTAGNEGNGTSNSALQYARELRRQGHTVRLVGIGSPEYPIRVHHIPLVSWIAAKQQMQFAQPDQAVFHKAFAGVDVVHVYLPFKYGRVAVQTAHEMGIPATAGFHLQPENVLYSAGPLRYIPGASAFIYWLFDRMLYRHIRHIHTPSHMIAAQLRAHGYRQRLHVISNGYSPRFTPKRQREPGSPSPRPFRIVASGRLASEKDHVTLIRAVALSRHAEDIELVICGTGPLQHELRRMAGKLLPGRWSIGFHDNAQMPQLLRSCDLLVHPSIVDIESLSVLEGMASGLVPVIARSTQSAAGQFALNDHSLFEAQDPAMLAQRIDWWIDHPDELSHWGAIYAEHTHEEYSIASCVRKFVAMEREAIADFDGRL

SEQ ID NO: 155 - protein, 397 aa, Bifidobacterium thermophilum
MKNQGFSVAAGSTEDAGRPLVIALVVDTAGNEGNGTSNSALQYAKELRRQGHTVRLVGIGSPEYPIRVRHIPLVSWIAAKQQMQFAQPDRAMFRKAFEGADVVHVYLPFKYGRVAVRTAHEMGIPATAGFHLQPENVLYSAGPLRYIPGASAFIYWLFDRILYRHIRHIHTPSHMIAAQLRAHGYRQRLHVISNGYSPRFTAKQQREPGSPSPRPFRIVASGRLASEKDHVTLIRAVALSRHAEDIELSICGTGPLQHELRRMAAQLLPGRWSIGFHDNAQMPAFLRSCDLLVHPSIVDIESLSVLEGMASGLVPVIARSTQSAAGQFALCDHSLFEAQDPAMLAQRIDWWIDHPDELSRWGAIYAEHTREEYSIASCVRKFVAMEREAIADFDGRL

SEQ ID NO: 156 - protein, 380 aa, Bifidobacterium sp.
MVEQPLTIALVIDTLANRGNGTSNSALQFADELQRQGHHVRLVGVGSPDYPARINKVPLVSWVASKQLMQFAEPSDTLFRTAFAGVDVVHIYMPFKFGRRAAKIARDMGIPVTAGFHLQPENVLYSAGPLRHIPGLSDFLYWLFRQWLYRRIDHIHVPTQMTASLLRAHGYKAKLHVISNGYSPDYTPGAPRDPNAPNPVPFRIIASGRLAREKDQITLIKAVALSKHAADIQLVIAGTGPLKRYLKFRSGRLLARKADIGFYKHADMPELLRSGDLFVHCSIADIESVSVIEAMACGLVPVIAASELSAASQFAMLDESLFPVHDAALLARRIDWWIDHPAERAQWSAEYAAHTKAHYSVEASVHKFADMEREAVTAAR

SEQ ID NO: 157 - protein, 393 aa, Bifidobacterium sp.
MAKNRENGAVAADGGRALTIVLVMDTIGTQGNGTSNSALQYARELERQGHRVRLVGLGSEEYPARENHVPLVSWVAAKQHMQFAKPSDTLFRTAFQGADVVHLYLPFAFGRRAWKVAREMGIPVTAGFHLQPENVSYNAGPIKAIPGVNDFMYLLFRHWLYRHVGHIHAPTEMIAEQLRAHGYKAKLHVISNGYSPRFTPKSQRKPTDREPLPLRVVASGRLAREKDHITLIRAVAMCRHAERIELHIAGTGPRQRYLRRRAKRLLPRPASIGFHRNADMPAFLKSCDLFVHPSIVDIESLSVIEGMASGLVPVIAQSELSAASQFALLDESLFPARDAAALAERIDWWLDHPRELAEWGARYAEHTREHYSIESCVRAFVAMEREAIADAAR

SEQ ID NO: 158 - protein, 389 aa, Bifidobacterium sp.
MREQSREKTTDRPLVIALVVDTVGNQGNGTSNSALQWAAELRRQGHTVRLVGIGSAEYPARVRRVPLVSWVAAKQQMQFAEPSDTLFRKAFEGADVVHIYTPFAFGRRAAHVARTMGVPVTAGYHVQPENVLYSAGPLRLIPGAQRFIYWLFNRWLYHDIAHIHTPTEMTASLLREHGYRAKLHVISNGYAPRFTARETSEPQADVQAEPHVPFRIIASGRLAHEKDHITLIKAVAMSRHAGDIQLIIAGTGPLKRYLTFRANRLLARKADIGFHPNAEMPELLRSCDLFVHCSIADIESISVIEAMACGLVPVIATSELSAASQFALLDESLFGVRDVAMLARRIDWWIDHPCELAEWGARYAAHTREHYSVASSVRQFVQMEREAAS

SEQ ID NO: 159 - protein, 396 aa, Bifidobacterium bifidum
MRDNKNEEPRPQDRSVLDRPMTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPASVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 160 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVARQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVIASGRLASEKDQITLIKAVSMSRHAGDIQLVIAGTGPLKQYLKFRAGRLLARKADIGFHKHADMPDLLRSGDLFVHCSIADIESVSVIEAMACGLVPVIAASELSAASQFALIDESLFPVRDAALLARRIDWWIAHQAERAEWGEKYAEYTKAHYSVEASVLKFVDMERKAIGE

SEQ ID NO: 161 - protein, 396 aa, Bifidobacterium bifidum
MRDNKNEEPRPQDRSALDRPMTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPASVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 162 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVAHQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVVASGRLASEKDQITLIKAVSMSRHAGDIQLVIAGTGPLEQYLKFRAGRLLARKADIGFHKHADMPDLLRSGDLFVHCSIADIESVSVIEAMACGLVPVIAASELSAAGQFALIDESLFPVRDAALLARRIDWWIAHQAERAEWGEKYAEYTKVHYSVEASVHKFVDMERKAIGE

SEQ ID NO: 163 - protein, 386 aa, Bifidobacterium longum
MRENGIEESIDRPLTIALVVDTVGNQGNGTSNSALQWAAELERQGHHVRLVGIGAPEYPARVNRVPLVSWVAAKQLMQFAEPSDTLFRTAFQGVDVVHIYMPFKFGRRAAKVAHQMGIPVTAGFHLQPENVLYSAGPLRRIPGISSFLYWLFKHWLYKHIDHIHVPTEMTASLLRAHGYKAKLHVISNGYSPMYSPKKPADPDASAPVPFRVIASGRLASEKDQITLIKAVSMSRHAGDIQLVIAGTGPLKQYLKFRAGRLLARKADIGFHKHADMPDLLRSGDLFVHCSIADIESVSVIEAMACGLVPVIAASELSAASQFALIDESLFPVRDAALLARRIDWWIAHQAERAEWGEKYAEYTKAHYSVEASVHKFVDMERKAIGE

SEQ ID NO: 164 - protein, 386 aa, Bifidobacterium longum
MRENGIEESIDRPLTIALVVDTVGNQGNGTSNSALQWAAELERQGHHVRLVGIGAPEYPARVNKVPLVSWVAAKQLMQFAEPSDTLFRTAFQGVDVVHIYMPFKFGRRAAKVAHQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVVASGRLASEKDQITLIKAVSMSRHAGDIQLVIAGTGPLKQYLKFRAGRLLARKADIGFHKHADMPDLLRSGDLFVHCSIADIESVSVIEAMACGLVPVIAASELSAAGQFALIDESLFPVRNAALLARRIDWWIAHQAERAEWGEKYAEYTKVHYSVEASVHKFVDMERKAIGE

SEQ ID NO: 165 - protein, 387 aa, Bifidobacterium pullorum
MRENENERRIDGPLTIALVVDTVGNQGNGTSNSALQWAQELERQGHHVRLVGVGAPEYPARVNKVPLVSWVAAKQQMQFAEPSDTLFRAAFAGCDVVHIYMPFKFGRHAAKVARRMGIPVTAGFHLQPENVLYSAGPLRFIPGMQSLLYALFRHWLYRKVDHIHVPTEMTASLLRGHGYQARLHVISNGYSTRFTAREPRDPEAAAPVPFRIVASGRLAREKDQVTLVKAVAMSRHASNIQLVIAGTGPLKHYLKFRAGWLPRHAEIGFHRNADMPALLRSCDLFVHCSIADIESVSVIEAMACGLVPVIAASELSAASQFALTDRSLFPVRDAAILANRIDWWIDHPGELARWGAVYGEHTKEHYSVEASVHRFVDMEREAIADAG

SEQ ID NO: 166 - protein, 391 aa, Bifidobacterium sp.
MREHGDEAEHEESIDRPLTIALVVDTVGNQGNGTSNSALQWAAELERQGHHVRLVGIGAPEFPARVNRVPLVSWVAAKQQMQFAEPSDTLFRAAFDGVDVVHIYMPFKFGRRAAKVARQMGIPVTAGFHLQPENVLYSAGPLRYIPGMERFLYWLFRQWLYKRVGHVHTPTEMTASLLREHGYKGVMHVISNGYSPRFTARRPLDPSAPAHVPFRVVASGRLAHEKDQITLIKAISMSRHASDIQLVIAGTGPLKRYLTFRANRRLKRRADIGFHRNADMPALLRSCDLFVHCSIADIESVSVIEAMACGLVPVIAASELSAASQFALLDESLFPVRDAAILARRIDWWIDHPDELARWGARYAEHTCEHYSVEASVHRFVDMEREAIADS

SEQ ID NO: 167 - protein, 386 aa, Bifidobacterium sp.
MREKGNEESIDRPLTIALVVDTVGNQGNGTSNSALQWAAELERQGHRVRLVGIGAPEYPARVNKVPLVSWVASKQLMQFAEPSDTLFRTAFQGVDVVHIYMPFKFGRHAAKVARQMGIPVTAGFHLQPENVLYSAGPLRHIPGVSGFLYWLFKHWLYKRVDHIHVPTEMTASLLRSHGYKARLHVISNGYSPAYSPKEPVDPQAPTPVPFRIIASGRLAHEKDQITLIKAISMSRHAGDIQLVIAGTGPLRRYLTFRAGRLLARKAEIGFHKHSEMPELLRSGDLFVHCSIADIESVSVIEAMACGLVPIIAASELSAASQFALLDESLFPVRDAALLARRIDWWIAHQTERAEWGAKYAEHTKEHYSVEASVHKFVDMEREAIAE

SEQ ID NO: 168 - protein, 398 aa, Bifidobacterium sp.
MREHGVEEAHGEYGQEEGIDRPLTIALVVDTVGNQGNGTSNSALQWAAELERQGHHVRLVGIGAPEYPARVNRVPLVSWVAAKQQMQFAEPSDTLFRTAFRGVDVVHVYMPFKFGRRAAKVARQMGIPVTAGFHLQPENVLYSAGPLRYVPGAERFLYWLFRQWLYKRIDHIHTPTEMTASLLREHGYRGTLHVISNGYSPRFTAREPADPSAPAHVPFRVVASGRLAHEKDQITLIKAISMSRHAGDIQLVIAGTGPLKRYLTFRAHRRLKRRADIGFHRNADMPALLRSCDLFVHCSIADIESVSVIEAMASGLVPVIAASELSAASQFALLDESLFPVRDAALLSRRIDWWIDHPSELARWGAEYATHTREHYSVEASVHRFVDMEREAIATAAR

SEQ ID NO: 169 - protein, 386 aa, Bifidobacterium sp.
MRENGKEESIDRPLTIALVVDTVGNQGNGTSNSALQWAAELERQGHHVRLVGVGAPEYPARVNKVPLVSWVASKQLMQFAQPSDTLFRTAFQGVDVVHIYMPFKFGRHAAKVARQMGIPVTAGFHLQPENVLYSAGPLRHIPGVSAFLYWLFKHWLYKRIDHIHVPTEMTASLLRSHGYKAKLHVISNGYSPVYKPVKPVDPDAATPVPFRIVASGRLAREKDQITLIKAISMSRHAADIQLVIAGTGPLRRYLTFRAGRLLARKADIGFHKHVEMPQLLRSGDLFVHCSIADIESVSVIEAMACGLVPVIAASELSAASQFALLDESLFPVRDAALLARRIDWWITHQRERAEWSAKYAQNTKEHYSVEASVHKFVDMEREAIGE

SEQ ID NO: 170 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRAAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRLDGAPAPVPFRIVASGRLSHEKDQITLIKAISRCRHAKDIELIICGTGPLQHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGRYAEHAKADYSVEASVAKFVAMEREAIADAKA

SEQ ID NO: 171 - protein, 386 aa, Bifidobacterium breve
MRENGIEKSIDRPLTIALVVDTVGNQGNGTSNSALQWAAELERQGHHVRLVGVGAPEYPARVNKVPLVSWVAAKQLMQFAEPSDTLFRTAFQGVDVVHVYMPFKFGRRAAKVAHQMGIPITAGFHLQPENVLYSAGLLRHIPGISSFLYWLFKHWLYKRIDHIHVPTEMTASLLRAHGYKAKLHVISNGYSPVYSPKKPVDPEASAPVPFRVIASGRLASEKDQITLIKAVSMSKHAGGIQLIIAGTGPLEQYLRFRAGRLLARKADIGFHKHADMPDLLRSGDLFVHCSIADIESVSVIEAMACGLVPVIAASELSAASQFALLDESLFPVQNAAMLARRIDWWIEHQVERAEWGEKYAEYTKANYSVEASVHKFVDMEREAIGE

SEQ ID NO: 172 - protein, 386 aa, Bifidobacterium longum
MRENGIEESIDRPLTIALVVDTVGNQGNGTSNSALQWAAELERQGHHVRLVGIGAPEYPARVNRVPLVSWVAAKQLMQFAEPSDTLFRTAFQGVDVVHIYMPFKFGRRAAKVARQMGIPVTAGFHLQPENVLYSAGPLRRIPGISSFLYWLFKHWLYKHIDHIHVPTEMTASLLRAHGYKAKLHVISNGYSPVYSPKKPADPDASAPVPFRVIASGRLASEKDQITLIKAVSMSRHASDIQLVIAGTGPLKQYLKFRAGRLLARKADIGFHKHADMPDLLRSGDLFVHCSIADIESVSVIEAMACGLVPVIAASELSAASQFALIDESLFPVRDAALLARRIDWWIAHQAERAEWGEKYAEYTKAHYSVEASVHKFVDMERKAIGE

SEQ ID NO: 173 - protein, 393 aa, Bifidobacterium sp.
MQENKVKVEDPLTIAMVVDTSGNRGNGTSNSALQWARELERQGHRIRLVGIGAPDFPAKVNHVPLVSWVARKQQMQFAEPSDTLFRAAFLGADVVHIYTPFRFGQHACRVARQMGVPVTAGYHVQPENITYSAGPLKYVPGIDSFIYWLFRHWLYRNVGHVHVPTELGAELLRSHGYTSKLHVISNGYEPRFTAKRQRPGDEPPDWPIRIIASGRLSNEKDQITLIRAVALSRHADDIQLTIAGTGPLKRRLQREAARLLPRPASIGFHRNAEMPALLRSADLFVHPSIADLESVSVLEGMASGLVPIIASSPLSAAGHFALREESLFPVGDAQALADRIDWWLDHPGELNAWGARYAEHTREHYSVEESVRRFVAMEREAIADARGHELTAA

SEQ ID NO: 174 - protein, 391 aa, Bifidobacterium sp.
MREHGDEAEHEESIDRPLTIALVVDTVGNQGNGTSNSALQWAAELERQGHHVRLVGIGAPEFPARVNRVPLVSWVAAKQQMQFAEPSDTLFRAAFDGVDVVHIYMPFKFGRRAAKVARQMGIPVTAGFHLQPENVLYSAGPLRYIPGMERFLYWLFRQWLYKRVGHVHTPTEMTASLLREHGYKGVMHVISNGYSPRFTARRPLDPSAPAHVPFRVVASGRLAHEKDQITLIKAISMSRHASDIQLVIAGTGPLKRYLTFRANRRLKRRADIGFHRNADMPALLRSCDLFVHCSIADIESVSVIEAMACGLVPVIAASELSAASQFALLDESLFPVRDAAILARRIDWWIDHPDELARWGARYAEHTREHYSVEASVHRFVDMEREAIADS

SEQ ID NO: 175 - protein, 398 aa, Bifidobacterium boum
MSQSSKFTRATGVNGDADRPLTIALVVDTAGNEGNGTSNSALQYARELRRQGHTVRLVGIGSPDYPVPVHWIPLVSWIAAKQQMQFAQPDRAVFHKAFVGVDVVHVYLPFKYGRVAVQTAHEMGIPATAGFHLQPENVLYSAGPLRYIPGASAFIYWLFDRMLYRHIRHIHTPSHMIAAQLRAHGYRQRLHVISNGYAPRFTAKQQRAPGSPVPKPFRVVASGRLASEKDHVTLIRAVALSRHAEDIELVICGTGPLQHELRRMAGKLLPGRWSIGFHDNAQMPQLLRSCDLLVHPSIVDIESLSVLEGMASGLVPVIARSTQSAAGQFALNDHSLFEAQDSAMLAQRIDWWIDHPDELSRWGAVYAEHTREEYSIASCVRKFVAMEREAIVDFDGRL

SEQ ID NO: 176 - protein, 626 aa, Acetivibrio sp.
MLFLLTVFFFVFEKLKVNAARKERGVKFLMVITLVNDTFNINNNGTTISAMRFAEALSQRGHQIRIITCGDPLKSGKDPDTGFEMFYLPELKIPIASRLAHKQNTLFAKPVRSTLKKAISGSDVVHIYQPWPLGSAAQRVARQMNIPAIAAFHIQPENITFNIGLKRFSPAAHLTYFLFYLFFYRRFSHIHCPSKFIAAQLRSHGYKARLHVISNGVHPAFCAPAKPREHTFKPIKILMIGRLSPEKRQDVLIRAVMKSRYADRIQLYFAGSGPWEKKLCHLGNKLPNPPVFGYYNRDELIKLIHECDLYVHASDAEIEGISLIEAFACGLVPIISDSKQSAAAQFALGPQNLFKAGSPESLAEKIDYWLDHPEQLKEAEKKYAQLGKQYALEHSIRKIEKVYSSMTKNHKNEYHRSIFFRLSTRLFQIVIACPILLLWTRFVLGAKVYGRENIRGLKSGVTVCNHVHLLDSALIGVTFFPRRVVFPTLTQNVKTLWPGKLVRILGGFAIPDNIMELKAFFDEMEFLLMKNCIVHFFPEGELRPYDTGLQNFKKGAFYLAAQAQVPIVPMLITFEPPKGLIKIIRKKPVMRLHIGKPIHPMSKDIEIDSELRMKAVCKKIEAITSV

SEQ ID NO: 177 - protein, 381 aa, unidentified
MTIVLVVDVFDNLTNGTTMTAYRFAQSLRENGHEVRAVAIGDKEENPYALNEAQLPVATKIAHKQGFAFAKADVTTFERAFQGADVVHFLVPLFMDHKALKVAKKMDIPICGAFHLQPENVTFILHVSKAQWLARSIYRFFNRVFYRNFDHIHCPSYFIANQLAKNGYTAKLHVISNGVDDDFCPGEVRERFDDGLIHILMVGRLSPEKRQRILIDAVAKSQYADRIQLHFAGKGPCREKLIEQGKILPHPPTFAFYSKRELISLIRRCDLYVHASVVEIEAISCLEAFSCGLVPVICDSDMSATVQFALDERSLFRPDDAKDLAAKIDYWLSHPEEKARMEQEYAKQGDSYRISASVKKAEEMFLDVIRDYREKHRVSRY

SEQ ID NO: 178 - protein, 542 aa, unidentified
MRIPTVSAFHLQPENVTYSIGLGKSKRANDVIYRYFYKCFYNRFRFIHCPSEMIAEQLMQHGYDAECRVISNGVNDLFRPAEVRRPEYLKGKIVLLMVGRLSGEKRQDLLIEAVQYSKYRDKIQLVFAGMGPKEKKYRKLSRNLKNKPIFRFFTQEELLQMYNICDLYIHTSDAEIEGISCIEAMACGAVPVISDSHLSATKSFALHPNCLFKAGDARSLAEKIDYWIEHPEERSKMSRIYAEKAETIRVSKCVAEMERLYKDVISDYHKNGYKQPEKSRLRKLLHPDTDAVNVAYSKRTPARQALFYVFTNLIAIIIYFIDTVFFGLVIEGKDKLKKVRGGAVTVMNHIHPMDCTMVKLAVFPRPIYFTSLVNNLELPLVGWLIRFCGALPVPNGKGKLVGFMKHIKHGIQNGDLVHFYPEGMLIRNYEGLREFQPGAFYTAVHTGCPVIPMVLVNIHPQGIWRIAGGRRMHLFIGEPQYPNSELSPKESVTELKNRTWQIMNEMMNEPVEAERISFSVAVRIACILYIASQVVRIIAIRL

SEQ ID NO: 179 - protein, 542 aa, unidentified
MRIPTVSAFHLQPENVTYSIGLGKSKRANDVIYRYFYKCFYNRFRFIHCPSEMIAEQLMQHGYDAECRVISNGVNDLFRPAEVRRPEYLKGKIVLLMVGRLSGEKRQDLLIEAVQYSKYRDKIQLVFAGMGPKEKKYRKLSRNLKNKPIFRFFTQEELLQMYNICDLYIHTSDAEIEGISCIEAMACGAVPVISDSHLSATKSFALHPNCLFKAGDARSLAEKIDYWIEHPEERSKMSRIYAEKAETIRVSKCVAEMERLYKDVISDHHKNGYKQPEKSRLRKLLHPDTDAVNVAYSKRTPARQALFYVFTNLIAIIIYFIDTVFFGLVIEGKDKLKKVRGGAVTVMNHIHPMDCTMVKLAVFPRPIYFTSLVNNLELPLVGWLIRFCGALPVPNGKGKLAGFMKHIKHGIQNGDLVHFYPEGMLIRNYEGLREFQPGAFYTAVHTGCPVIPMVLVNIHPQGIWRIAGGRRMHLFIGEPQYPNSELSPKESVTELKNRTWQIMNEMMNEPVEAERISFSVAVRIACILYIASQVVRIIAIRL

SEQ ID NO: 180 - protein, 396 aa, Bifidobacterium bifidum
MRDNKNEEPRPQDRSALDRPLTIVLVMDTIGNKGNGTSNSALQYAHELERQGHHVRLVGIGAPEYPARVNKVPLVSWVAAKQQMQFAKPSDTLFRTAFAGADVVHLYLPFKFGRCAYKVARSMGIPVTAGFHLQPENVMYSAGPLKYLPGMSGFLYALFRFWLYRRIGHIHAPTEMIAGQLRAHGYKAKLHVISNGYVPRFTPKRQRSDGAPAPVPFRIVASGRLSHEKDQITLIKAISRCRHAKDIELIICGTGPLRHYLRFRADRLLERKAQIGFHKNAEMPALLRSCDLFVHPSIVDIESLSVIEGMASGLVPVIASAELSAASQFALLDESLFPARDVAMLARRIDWWIDHPHELAVWGGLYAEHAKADYSVEASVAKFVAMEREAIADAKA

SEQ ID NO: 181 - protein, 386 aa, Bifidobacterium breve
MRENGIEKSIDRPLTIALVVDTVGNQGNGTSNSALQWAAELERQGHHVRLVGVGAPEYPARVNKVPLVSWVAAKQLMQFAEPSDTLFRTAFQGVDVVHVYMPFKFGRRAAKVAHQMGVPVTAGFHLQPENVLYSAGPLRHIPGISSFLYWLFKHWLYKRIDHIHVPTEMTASLLRAHGYKAKLHVISNGYSPVYSPKKPVDPEASAPVPFRVIASGRLASEKDQITLIKAVSMSKHAGDIQLIIAGTGPLKQYLRFRAGRLLARKADIGFHKHADMPDLLRSGDLFVHCSIADIESVSVIEAMACGLVPVIAASELSAASQFALLGESLFPVQNAAMLARRIDWWIEHQVERAEWSEKYAEYTKAHYSVEASVHKFVDMEREAIGE

SEQ ID NO: 182 - protein, 398 aa, Bifidobacterium sp.
MPHSRNAAGDAAKPLTIVLVVDSVGNRGNGTSNSALQYAKELEREGHHVRLVGVGAPDYPVGIHHLPLISWLAAKQQMQFAEPDDAVFRHAFAGADVVHIYMPFKFGRHALAVARSMGIPVTAGFHLQPENVTYSAGPLRYIPGIPSLIYYLFDFWLYRHIGHIHAPSQMIARQLRAHGYRARLHVISNGYSSRFHPAQKQSGNKHGNNEHGKRDEDGFRIIASGRLTHEKDHETLIRAVALSRHAAQIDLTICGTGPLQRHLRRLAKRLLPRPAHIGFQPNDEMPNLLRTADLLVHPSIVDIESLSVLEGIASGLVPVIADSPLSAASQFALTDSSVFPARNAKALARRIDWWIEHPQELAEWGPKYAQEAREKYALPQSVHRFVAMEREAIADACK

SEQ ID NO: 183 - protein, 395 aa, Bifidobacterium sp.
MSDSRNAAVDADKPLTIALVVDSVGNRGNGTSNSALQYAQELERQGHHVRLIGVDAPDYPVEVHHLPFVSWLAAKQQMQFAQPDDAVFRQAFKGADIVHIYMPFKFGRCALAVARSMAIPVTAGFHLQPENVTYSAGPLRYIPGIPSLIYYLFDFWLYRHIGHIHTPSQMIARQLRAHGYRARLHVISNGYSGRFHPAKAQSCSADGDVSRRVFRIVASGRLTHEKDHETLIRAVAMSRHANQIDLTICGTGPLQRHLRRLAKRLLPRPAHIGFHANEDMPNLLREADLLVHPSIVDIESLSVLEGIASGLVPVIADSPLSAASQFALTDSSIFPARNAKALARRIDWWIEHPQKRAEWGPQYAKEAREEYALPQCVRKFIAMEREAIADERKKR

SEQ ID NO: 184 - protein, 403 aa, Bifidobacterium sp.
MPESRNVAGNAIEPLTIALVVDSVGNRGNGTSNSALQYAKELEREGHHVRLVGVGAPDYPVGIHHLPFISWLAAKQQMQFAEPDDAVFRRAFAGADVVHIYMPFKFGRHALTVARSMGIPVTAGFHLQPENVTYSAGPLRYIPGIPSLIYYLFDFWLYRHIGHIHAPSQMIARQLRAHGYRARLHVISNGYSSRFHAASTQTAETHDKNAEGAFRIIASGRLTHEKDHETLIRAVALSRHAAQIDLTICGTGPLQRHLRRLAKRLLPRPAHIGFQPNDEMPNLLRTADLLVHPSIVDIESLSVLEGIASGLVPVIADSPLSAASQFALTDSSVFPARNAKALARRIDWWIEHPQELAEWGPKYAQEAREKYALPQSVHRFVAMEREAIAGAQKTWQPAAPGRR

SEQ ID NO: 185 - protein, 579 aa, unidentified
RRPPRSTLFHYTTLFRSCFAKPDEEAYYTAFKDADIVHFYMPFRFCRRGEELARQMGIPTVAAFHVQPENITSSIFLGKNRRVNDFLYWWFYKVFYNRFDHIHCPSAFIARQLERHGYGAKLWVISNGVADAFRPAQVPRAPELEGRFTILMIGRLSGEKRQDLIIEAAKRSKYADRLQLVFAGKGPKEKAYRKLAAGLAHPPVFGFYGQDELRRLINMCDLYVHASDAEIEGISCMEALACGLVPVISDSPLSATGQFALCAESLFRAGDADDLARRIDYWVEHPEEKRAYAEQYALRQDENRVEACVARAEEMYASAIRDKRRKGYRKVPLSRWRRCTSPSCEAIRRNFCQGGTVRTLLFWLFTTLLSPILWLLDRLWLGARIEGRENLDAVQGGAVSIMNHVHPLDCTMAKVALFPYRLWFISLASNLQKPFTGWLIRFCGGVPLPDDIHGMAALERGMEARIRGGAFVHFYPEGMLVPYHEGLRAFHPGAFATAVRAGCPVVPMMLCRRPARGLWAWRKKPCFTLRIGAPLYADASLPKKQAARDLQLRAEAAMLALERGETPPLAVGEPALDAE

SEQ ID NO: 186 - protein, 398 aa, Bifidobacterium boum
MSQSSKFMRATGVNGDADRPLTIALVVDTAGNEGNGTSNSALQYARELRRQGHTVRLVGIGSPEYPIRVHHIPLVSWIAAKQQMQFAQPDRAVFHKAFAGVDVVHVYLPFKYGRVAVQTAHEMGIPATAGFHLQPENVLYSAGPLRYIPGASAFIYWLFDRMLYRHIRHIHTPSHMIAAQLRAHGYRQRLHVISNGYSPRFTPKRQREPGSPSPRPFRIVASGRLASEKDHVTLIRAVALSRHAEDIELVICGTGPLQHELRRMAGKLLPGRWSIGFHDNAQMPELLRSCDLLVHPSIVDIESLSVLEGMASGLVPVIARSTQSAAGQFALNDHSLFEAQDSAMLAQRIDWWIDHPDELSRWGAVYAEHTREEYSIASCVRKFVAMEREAIADFDGRL

SEQ ID NO: 187 - DNA, 1170 bp, Helicobacter mustelae
  source 1..1170: mol_type = genomic DNA
atgaccatcggaatagtaattgatagctacaatgatagaagcaatggcacttctatgacggcttttcgtttcgcaagagaatttgtcaaaaaggggcatgaggttcgcatcgtagcctgcaatgtaagcaagagtatgagcgatgaggaggatctcaagctctatcccgtgaagcagagatatatccccatcgttacagaggtttctaaaaaacagcatatgatttttggcgctcctgatttggaggttttgcaatctgctgtggtgggctgtgatatcgtgcatttttatatgccctttgcacttgagattgcagggatgcatctctgcagaagccttcgtatcccctatatcagtgcctttcatgtccagcctcagcatatcagctacaacatgaatatgaatttttcttggtttaatacctatcttttcaaaagattttataagcatttttatcgctacacccatcacatccattgcccgagtaaattcatcgaaaaagaactccaaagggaaaattatggaggcaaaaaatacaccattagcaatggcttttttggtggggatagggtgatggcagatccttatgaagactccttttttcacatcgcttcagtgggaagattttccaaagaaaaaaagcaagacatcatcatcaaggccatagcaaaaaatccctatgccgataaaatcaaactgcatttgcatggtgtggggccgcgggagaaatatctcaaaaatctctgcaataagctgcttatcaataagccagaatttggatttatcgataatggcgcattgcttgaaaaactcgcaaaaatgcatctttatgtgcatgcagcaaaggtagagagcgaggcaatttcttgccttgaggccatatctcttggagtggtgcctgtaatcgcagattcagagacaagcgctacggtgcaatttgccctcgatcctctaagtctttttgaggtcaataatgttgcagatttaagcaataagatcacctattggatcgagcatccaaaggagttgctagcctataagcaaaaatatgcagagtctgcgctgcagtattctctagacaaaagtattgaagaaaccctgggattgtatgaagaagcaatcagggattttcgcgatcagcctgccttgtttgatcgcatcaatgcataa

SEQ ID NO: 188 - protein, 279 aa, Eubacterium coprostanoligenes
MSTCWLQGKTVVVTGASGGMGAGIAATLIKKHGCTVIGVARNEKKMLKFVDELGETYAKQFSYELFDVSSKENWEKFAEELQEKGVKVDVLINNAGILPKFKRFDRYSYEEIERAMNINFYSCVYSVKTMLPMLLQSSTPAIINIDSSAALMTLAGTSMYSASKAALKGFTEALRVEFQGKMFVGLVCPGFTKTDIFSGQGDADMSNGAKVMDMISTDCDKMVKMIMFGIEHKTPMQVHGFDAHAMSVFNRLMPVYGSKLFSSIMRMSNVDIFKEVFSD

SEQ ID NO: 189 - protein, 252 aa, unidentified
MKTAIITGASSGLGREFARQLTDIFPEIECCWLIARREDRLEEIAREMVGVETVCLPLDLCDSMSFTTLQEKLAAEKPEVAILINNAGCGYLGRMGETETAVQTRMVDLNVRAMTAMTNLVIPYMPAGGRILNTSSIASFCPTPRMTVYGASKAYVSSFTVGLSEELKRRDITVTAVCPGPMKTEFLDVGSITGRSPAFEYLPYCDQVRVAAGALRAAKAGRTMYTPRLFYKFYRLLAKVTPVKMMVKFTKT

SEQ ID NO: 190 - protein, 252 aa, synthetic construct
MKTAIITGASSGLGREFARQLTDIFPEIECCWLIARREDRLEEIAREMVGVETVCLPLDLCDSMSFTTLQEKLAAEKPEVAILINNAGCGYLGRMGETETAVQTRMVDLNVRAMTAMTNLVIPYMPAGGRILNTSSIASFCPTPRMTVYGASKAYVSSFTVGLSEELKRRDITVTAVCPGPMKTEFLDVGSITGRSPAFEYLPYCDQVRVAAGALRAAKAGRTMYTPRLFYKFYRLLAKVAPVKLMVKFTKT

SEQ ID NO: 191 - protein, 253 aa, synthetic construct
MKTAVITGASSGLGREFVRQFYSVFPEIERVWLIARRTDRLQELAEQLEEKGISTLTLPLDLCDTMSFTAYQEHLVEEQPEIALLVNNAGCGYLGNIGEIDTVSQTRMIDLNLRALTAITNLSVPYMEAGSRILNVSSIASFCPTPRMTVYSATKAYVSAYTIGAAEELKAKGITVTAVCPGPMATEFLAVGHVVDSWPFELLPYCDQVQVAGGALRAAKAGQTIYTPRLFYKFYRLLAKLTPAKLMVKLTKT

SEQ ID NO: 192 - protein, 253 aa, Oscillibacter sp.
MKTAVITGASSGLGREFVRQFYSVFPEIERVWLIARRTDRLQELAEQLEEKGISTLTLPLDLCDTMSFTAYQEHLVEEQPEIALLVNNAGCGYLGNIGEIDTVSQTRMIDLNLRALTAITNLSVPYMEAGSRILNVSSIASFCPTPRMTVYSATKAYVSAYTIGAAEELKAKGITVTAVCPGPMATEFLAVGHVVDSRPFELLPYCDQVQVAGGALRAARAGQTIYTPRLFYKFYRLLAKLTPAKLMVKLTKT

SEQ ID NO: 193 - protein, 253 aa, unidentified; identical to SEQ ID NO: 192

SEQ ID NO: 194 - protein, 263 aa, unidentified
MSEIAVITGGTSGIGRATALYLRDAGYTVYELSRREQGVEGLHHIRCDITDEGQVRAAVAEIMDRAGRIDVLVNNAGFGISGAVEFTDTAEAQRLLDVNFFGMVRMNKAVIPHMRQAGRGRIVNLSSVAAPCPIPFQAYYSAGKAAVNAYTMALANELRPFGITVCAVQPGDIHTGFTAARVKTMEGDDAYGGRIGRSVQRMEHDEQNGMDPAKAGAFIARVAMKRRPKPIYTIRLDYQFFVFLTRILPGRTLNWLIGLLYAK

SEQ ID NO: 195 - protein, 257 aa, unidentified
MKKIAVITGASSGMGRRFAETVDSFGRFDEVWVIARHEKALEELRDQVPYPIRPLALDLTDRRSFQTYADALAEEPVEVGLLVNASGFGKFRAVVDTPLETNLNMVDLNCQAVMALCQLTVPYMPRGGQIINIASVAAFQPIPYINVYGATKAFVLSFSRALNRELRSQGVRVMALCPFWTRTAFFDRATGDGGESVVKKYVAMYEPEQLVQRAWRDAKRGKDVSQYGFVARFQAGLTKLLPHSLVMDVWMRQQKLQ

SEQ ID NO: 196 - protein, 257 aa, unidentified
MKKIAVITGASSGMGRRFAETVDTFDRFDEVWVIARHEKALEELRDRVPYPIRALALDLTDRRSFQTYADALAEEPVEVGLLVNASGFGKFRAVVDTPLEVNLNMTDLNCQAVVALCQLTAPYMPRGGQIINIASVAAFQPIPYINVYGATKAFVLSFSRALNRELRSRGVRVMALCPFWTRTAFFDRATGDGGESVVKKYVAMYEPEQLVQRAWRDARRGRDVSQFGFVARFQTALTKLLPHSLVMDVWMRQQKLQ

SEQ ID NO: 197 - protein, 263 aa, unidentified
MSEIAVITGGTSGIGRATALCLRDAGYDVYELSRREQGVEGLHHIRCDITDEDQVRAAVAEIIGQAGRIDVLINNAGFGISGAVEFTDTAEAQRLLDVNFFGMVRMNKAVIPHMRQAGRGHIVNLSSVAAPCPIPFQAYYSAGKAAVNAYTMALANELRPFGITVCAVQPGDIHTGFTAARVKTMAGDDVYQGRIGRSVQRMEHDEQTGMDPAKAGAFIARVAMKRRPKPIYTIRLDYQFFVFLTRILPGRTLNWLIGLLYAR

SEQ ID NO: 198 - protein, 257 aa, unidentified
MKKIAVITGASSGMGRRFAETVDSFGRFDEVWVIARHEKALEELRDQVPYPIRPLALDLTDRGSFQTYADALAEEPVEVGLLVNASGFGKFRAVVDTPLETNLNMVDLNCQAVMALCQLTAPYMPRGGQIINIASVAAFQPIPYINVYGATKAFVLSFSRALNRELRSRGVRVMALCPFWTRTAFFDRATGDGGESVVKKYVAMYEPEQLVQRAWRDAKRGKDVSQYGFVARFQAGLTKLLPHSLVMDVWMRQQKLQ

SEQ ID NO: 199 - protein, 263 aa, unidentified
MSEIAVITGGTSGIGRATALYLRDAGYTVYELSRREQGVEGLHHIRCDITDEDQVRAAVAEIMDRAGRIDVLVNNAGFGISGAVEFTDTAEAQRLLDVNFFGMVRMNKAVIPHMRQAGRGRIVNLSSVAAPCPIPFQAYYSAGKAAVNAYTMALANELRPFGITVCAVQPGDIHTGFTAARVKTMEGDDAYGGRIGRSVQRMEHDEQNGMDPAKAGAFIARVAMKRRPKPIYTIRLDYQFFVFLTRILPGRTLNWLIGLLYAK

SEQ ID NO: 200 - protein, 257 aa, unidentified; identical to SEQ ID NO: 196

SEQ ID NO: 201 - protein, 263 aa, unidentified; identical to SEQ ID NO: 197

SEQ ID NO: 202 - protein, 254 aa, unidentified
MKTAVITGASSGLGREFVRQFHSVFPEIQRVWLIARRAERLQELAGQLEEKGLSVLTLPLDLCDTMSFTAYHEHLVEEQPEIALLVNNAGCGYLGNIGEVDTVSQTRMIDLNLRALTALTNLTVPYMAAGSRILNVSSIASFCPNPRMTVYSATKAYVSAYTIGAAEELKAKGITVTAVCPGPMATEFLSVGRITGNSRMFERLPYCDQVQVAGAALRAAREGRTIYTPRLFYKFYRLLAKVTPAKLMVKLTKT

SEQ ID NO: 203 - protein, 263 aa, unidentified
MNTIFIVGSSSGIGKATAKLFAEKGWTVIATMRTPEKETELTAYPNVCLFPLDVTKPEQIEATVSAVLKEYDVDVLFNNAGYGMKSRFEDMTEEAMQRSLNTNLLGMVRVTQKFIPYFKAKKSGMILTTTSLAGEMGLVLDGIYAADKWAVTGLCEMLYHELYPFGIQVKTIVPGVVKTGFKMELSEMPGYDDLIKKQTNLLIPDMDSMETPEEVAQDIYAAVTDGDADRMCYVTGAITKTLYAKRQELGDEDFRRYMRGLLE

SEQ ID NO: 204 - protein, 253 aa, unidentified; identical to SEQ ID NO: 192

SEQ ID NO: 205 - protein, 263 aa, unidentified
MREIAVITGGTSGIGRATALYLRDAGYTVYELSRREQGVEGLHHIRCDITDEGQVRSAVAEIMDRAGRIDVLVNNAGFGISGAVEFTDTAEAQRLLDVNFFGMVRMNKAVIPHMRQAGRGRIVNLSSVAAPCPIPFQAYYSAGKAAVNAYTMALANELRPFGITVCAVQPGDIHTGFTAARVKTMEGDDAYGGRIGRSVQRMEHDEQTGMDPAKAGAFIARVAMKRRPKPIYTIRLDYQFFVFLTRILPGRTLNWLIGLLYAK

SEQ ID NO: 206 - protein, 257 aa, unidentified
MKKIAVITGASSGMGRRFAETVDTFGRFDEVWVIARHEKALEELRDQVPYPIRPLALDLTDRGSFQVYADALAEEPVEVGLLVNASGFGKFRAVADTPLETNLNMVDLNCQAVMALCQLTAPYMPRGGQIINIASVAAFQPIPYINVYGATKAFVLSFSRALNRELRSRGVRVMALCPFWTRTAFFARATGDGGESVVKKYVAMYEPEQLVQRAWRDAKRGKDVSQFGFVARFQTALTKLLPHSLVMDVWMRQQKLQ

SEQ ID NO: 207 - protein, 253 aa, unidentified
MKTAVITGASSGLGREFVRQFYSVFPEIERVWLIARRTDRLQELAEQLEEKGISTLTLPLDLCDTMSFTAYQEHLVEEQPEIALLVNNAGCGYLGNIGEIDTVSQTRMIDLNLRALTAITNLSVPYMEAGSQILNVSSIASFCPTPRMTVYSATKAYVSAYTIGAAEELKAKGITVTAVCPGPMATEFLAVGHVVDSRPFELLPYCDQVQVAGGALRAARAGQTIYTPRLFYKFYRLLAKLTPAKLMVKLTKT

SEQ ID NO: 208 - protein, 253 aa, unidentified; identical to SEQ ID NO: 192

SEQ ID NO: 209 - protein, 256 aa, unidentified
MRKKPIAIVTGASGGMGLEFVRLLAKKEELDEIWVIARSADKLEAVKGEIGSRLQCFPMDLSNLENIKKFGSQEGLKDCNIKYLINNAGFAKFCSHDDLSLEESINMIDLNVSGVVAMGLVCIPHMEKGGRIINIASQASFQPLPYLNIYSSTKAFVRSYTRALNVELKDRGVTATAVCPGWMKTGLFARGLIGAKKEVKNFVGMVTPDVVAKKALSDADKGKDMSVYGLYVKMCHLIAKVLPQKVMMKLWLIQQK

SEQ ID NO: 210 - protein, 266 aa, unidentified
MSERRVAIVTGGTSGIGKATALALRHAGCTVYELSRRAEGTEGLHHISADVTDERAVRDAVAQVMAAEGHIDILVNNAGFGISGAIEYTETEDAKKLFDVNFFGMVNMNRAVVPLMRQAGRGRIVNLSSVAAPVPIPFQAYYSATKAAVNAYSMALANELRPFGVTVCAVMPGDICTGFTAARKKIVTGDDIYHGRISRSVQRMEHDEETGMEPAKAGAYIASVALRDGSRHPLYAIRFDYKFFTFLAKVLPARFLNWLIYCLYGK

SEQ ID NO: 211 - protein, 257 aa, unidentified
MKKIAVITGASSGMGKRFAETVDVYGTFDEVWVIARHREQLEALRETVPFPIRVLPLDLTDRSSFDVYAAALAEEPVEVGLLMNCSGYGKFSAVLDTPLAVNLNMTDLNCQAVVAMCQLTAPYMVRGGQIINIASVAAFQPIPYIDVYSAGKAFVLSFSRALNRELRGRGIGVMAVCPFWTRTAFFDRAIKGGEEPVVKKYAAMYDPADIVKRTWRDAKRGKDVCKYGFVARAQAGLTKLLPHSVVMDVWMRQQKLR

SEQ ID NO: 212 - protein, 266 aa, unidentified; identical to SEQ ID NO: 210

SEQ ID NO: 213 - protein, 257 aa, unidentified; identical to SEQ ID NO: 211

SEQ ID NO: 214 - protein, 266 aa, unidentified; identical to SEQ ID NO: 210

SEQ ID NO: 215 - protein, 257 aa, unidentified; identical to SEQ ID NO: 211

SEQ ID NO: 216 - protein, 266 aa, unidentified; identical to SEQ ID NO: 210

SEQ ID NO: 217 - protein, 257 aa, unidentified; identical to SEQ ID NO: 211

SEQ ID NO: 218 - protein, 291 aa, unidentified
MLEERSAIILGASSGVGYGAALRFAEEGAHVIAGARSLNKLEALKAEAEKQGFSGTITPVACDVTHDNDLDHILQVCLDAYGKVDILACIAQSNLNDQHGFEDSDLENIMAFYRGGPGYTFQMIKKCLPHMKEQHYGRIITCASGAGERYTPHSCGYGMAKAAIINLTRTCAVELGQYGIVTNCFLPVIQVSHFEKQSNDAALAVPIMNALSPVGRMGDAYEDGSPMLAFLASEEARYINGQVISICGGISYINPNRARVIHPRPVWLSSAYTASYSSGTCLQTAQANCRP

SEQ ID NO: 219 - protein, 266 aa, unidentified; identical to SEQ ID NO: 210

SEQ ID NO: 220 - protein, 257 aa, unidentified; identical to SEQ ID NO: 211

SEQ ID NO: 221 - protein, 263 aa, unidentified
MGKIVIVTGGTSGIGLHTAAYLQSQGCTVYTVSRRPCDDARFHHICADVTQEADVARAVKSVLDEAGRIDILVNNAGFGISGAVEFTDLATARRQLDVNFWGMAAMTHAVLPVMRRQGSGRIVNLSSVAAPAAIPFQTYYSVSKAAINDFTLATANEVRPFGITVCAVMPGDICTGFTDARQKIPAGDEIYGGRISRSVAGMEKDERTGMAPEAAGRFVGKVALRTSHKPLYTIGFVYRCCVLLLKLLPARISNWLVGKLYAK

SEQ ID NO: 222 - protein, 257 aa, unidentified
MQKTIAVITGASSGMGAEFVRTIESFGVRLDEIWAIARRQERLEALQAPCPVRPLALDLTDPASFSEYQALLEQEQPRVALLINASGFGKFAATWQTPLAVNQAMVALNCQAVLAMCQLTLPYLGEGSCIVNIASVAAFQPIPYINVYAASKAFVLQLSRALNREVRPQGIRVMALCPFWTKTEFFNRAIDAGRKQIVKKYTAMYDPAQIVRRAWRDLARGKDVSKYGFVARFQAALCKLLPHSFVMTYWMHQQKLP

SEQ ID NO: 223 - protein, 253 aa, unidentified
MKTAVITGASSGLGLEFARQVHQVFPEIERVWLIARRVQRLEELAAQLTDEGLSVLTLPLDLCDTMSFTAYHEHLVEEQPEISLLINNAGCGYLGKIGEVDTVSQTRMIDLNLRALTAITNLSVPYMEAGSRILNVSSIAAFCPTPRMTVYSATKAYVSAYTIGAAEELKAKGITVTAVCPGPMATEFLETGHIHNSPAFELLPYCDQVRVAGGALRAAKAGQTIYTPRLFYKFYRLLAKLTPAKLMVKLTKT

SEQ ID NO: 224 - protein, 253 aa, unidentified
MKTAVITGASSGLGREFVRQFYSVFPEIERVWLIARRTDRFQELAEQLEEKGISTLTLPLDLCDTMSFTAYQEHLVEEQPEIALLVNNAGCGYLGNIGEIDTVSQTRMIDLNLRALTAITNLSVPYMEAGSRILNVSSIASFCPTPRMTVYSATKAYVSAYTIGAAEELKAKGITVTAVCPGPMATEFLAVGHVVDSRPFELLPYCDQVQVAGGALRAARAGQTIYTPRLFYKFYRLLAKLTPAKLMVKLTKT

SEQ ID NO: 225 - protein, 263 aa, unidentified
MSEIAVITGGTSGIGRATALYLRDAGYTVYELSRREQGVEGLHHIRCDITDEDQVRSAVAEIMDRAGRIDVLVNNAGFGISGAVEFTDTAEAQRLLDVNFFGMVRMNKAVIPHMRQAGRGRIVNLSSVAAPCPIPFQAYYSAGKAAVNAYTMALANELRPFGITVCAVQPGDIHTGFTAARVKTMEGDNVYGGRIGRSVQRMEHDEQNGMDPAKAGAFIARVAMKRRPKPIYTIRLDYQFFVFLTRILPGRTLNWLIGLLYAK

SEQ ID NO: 226 - protein, 263 aa, unidentified
MREIAVITGGTSGIGRATALCLRDAGYTVYELSRRPEGVEGLHHIRCDITDEDQVRSAVAEIMDRAGRIDVLVNNAGFGISGAVEFTDTAEAQRLLDVNFFGMVRMNKAVIPHMRQAGRGRIVNLSSVAAPCPIPFQAYYSAGKAAVNAYTMALANELRPFGITVCAVQPGDIHTGFTAARVKTMEGDNVYGGRIGRSVQRMEHDEQTGMDPAKAGAFIARVAMKRRPKPIYTIRLDYQFFVFLTRILPGRTLNWLIGLLYAR

SEQ ID NO: 227 - protein, 266 aa, unidentified
VTPVKKVCVITGGTSGIGLCTAQAMLEKGYTVYELSRRAEGAPGMKHIMADVTKEETLAAAVQEILKQEDHIDVLINNAGFGISGAVEFTKTEDAQHQLDVNFFGMVRMNRQVLPVMRRQCHGRIVNLSSVAGAIPIPFQAYYSASKAAINSYTMALANEVKPFGIQVCCVQPGDIRTGFTAAREKNPEGDDIYGGRIARSVAGMERDEQNGMAPEKAGAFIAHVADKKSVQPINTIGLQYKFFCFLVKILPAKTLNWLVGLIYAK

SEQ ID NO: 228 - protein, 250 aa, unidentified
MTAAVITGASAGLGAEFTRQLVREFPEIEEFWLIARRVGKLEELAQQFPEKKFVCMGLNLLDPKSFRYLGEKLAEQKADVRVLVNNAGCGTTGNIGSSASSADVMRVVNLNVRALTMVTQTVVPFMTRGAKIINVSSIAAFCPTPRMTVYSASKAYVSAFTGGIADELRPKGISVTAVCPGPMKTEFFDAAGDHDLFGNIPWCDPVKVVAGTIRAAKKGRTFYTPTAFNKFYRFVAKILPMKWMIKATRV

SEQ ID NO: 229 - protein, 247 aa, unidentified
MEKLDLHGKAGFIIGGTRGIGNAIAMRMARLGMNLTVIARKEDELAAMKAQVEPLGVRFLGIQSSNTDFDAIAAAFKTSWQTYGRLDLLVNAAGTGVMTPFEELTKSDVDTTIDVNLKGTIYAVKLSVPYFEKTGGGNILNISSMSAIRGIPDPVNNNGIYTATKFAINGFTECMQKYLLKYGIRVTALCPGSTATSWWTRWTHSFGTDAMLPVETLADIAELIVTSPEKVLFKQLQVLPTVEIDNF

SEQ ID NO: 230 - protein, 266 aa, unidentified
MNIAIVTGASSGMGREFVRQLGGYVSVDEIWAVARRASALETLKAEASVPVRPIVLDLLEASSFTRLEALLESEKPNVRLLVNAAGFGKFGAYRKIPVEDDCRMIDLNCKALLLMTRLCVPYMQPGSHILELDSLSAFQPVPYITTYAATKAFVLSYSRSMNRELKAKGIRVMAMNPGWVKTEFFNHAFQTNADNEVRYFNRLYEAKDVVKTGLNDLYHSKKDCSIHGFPVKFQVFLVKLLPHSLVMNTWLNQQKKAKNNKGLTTK

SEQ ID NO: 231 - protein, 264 aa, unidentified
MSGQSRPVALVTGATQYTGFSTAKLFAQRGFDVCVTSRSLEKAEDAAQRIRSAVPGARVLGLQMDPPSVAQTQSAFQKVEAVFGRLDVFVANACAACRFKSLLSTTEEDYDAIVNANLKGYFFGAQAAARLMIKTECKGSMILIGSVHSRGAIGNRIPYAISKGGIEVLGRNSAYELGKYGIRVNCVVCGAIVNDKFIEQTEEEKNARRANWPLGRESYPEDVAKAVFFLASDEAKTITGTSLVVDSGVSACMLKYDPNWETVG

SEQ ID NO: 232 - protein, 264 aa, unidentified
VSKVAVVTGGTSGIGRATALALKDAGYTVYELSRRAEGVEGLHHISADVTDQQAVNDAAAQIMEAEGHIDVLVNNAGFGISGAIEFTETAEAKALFDVNFFGMVNMNRAVVPLMRQAGRGRIVNLSSVAAPVPIPFQAYYSATKAAVNAYTMALANELRPFGVTVCAVMPGDIHTGFTAARRKVSAGDAIYQGRISRSVKRMEHDEETGMDPAKAGAYIASVAMREGHHHPLYAIRFDYKFFTFLAKVLPARFLNWLIYCLYGK

SEQ ID NO: 233 - protein, 257 aa, unidentified
MKKIAVITGASSGMGKRFAETVDRYGTFDEVWVIARHEAQLEALRATVPFPVRVLALDLTDRGSFDVYAAALAETPVEVGLLMNCSGYGKFSAVLDTPLAVNLNMTDLNCQAVVAMCQITAPYMPRGSQIINIASVAAFQPIPYIDIYGATKAFVLSFSRALNRELKSRGIGVMAVCPFWTKTAFFDRAIKSDETPIVKKYAAMYDPDDIVARTWRDAKRGKDVCKYGFVARVQAGLTKLLPHSVVMDVWMKQQDLH

SEQ ID NO: 234 - protein, 263 aa, unidentified
MSKVAVVTGGTSGIGKQTALALKAAGYTVYELSRRAQGVEGLHHLVADITREELVDAAIGEVLRQEGHIDVVVNNAGFGISGAIEFTKTEDAKRLFDADFFGMVNVNRAVIPHMRQAGAGRIVNLSSVAAAAPIPFQAYYSAAKAAVNSYTMALANELRPYGVTVCAVQPGDIHTGFTAAREKTIDGDDVYGGRISRSVARMEHDEQTGMDPAKAGAFIAKVVMKQRVKPIYTIRFDYQFLALLTRILPYRFLNWLIGVIYGK

SEQ ID NO: 235 - protein, 257 aa, unidentified
MKRIAVITGASSGMGRRFAETVDTFGRFDEIWVIARHGAALEGLRERVPFPVRPLTLDLTDRSSFATYAKALTEEPVEVGLLVNASGFGKFRAVVDTPLEVNLNMVDLNCQAVMALCQLTIPYMPEGGQIINIASVAAFQPIPYIDVYGASKAFVLSFSRALNRELRGRGIRVMVLCPFWTRTAFFARATVNGGESVVKKYVAMYEPEQLVQRAWRDAKRGKDVSQFGFVARFQTGLTKFLPHSLVMDVWMHQQKLK

SEQ ID NO: 236 - protein, 265 aa, unidentified
MSNKVAVVTGGTSGIGRATALALKDAGCTVYELSRRAQGVEGLHHISADVTKEESVRAAVEQIMAREGRIDILVNNAGFGISGAVEFTSTEDAKSLFDVNFFGMVNMNRQVVPIMREAGRGRIVNLSSVAAPVPIPFQTYYSATKAAVNAYTMALANELRPFGVTVCAVMPGDIHTGFTAARRKIGEGDDIYQGRISRSVARMEHDEETGMDPARAGGYVARVALREGSHHPLYAIRIDYKFFVFLTKVLPAGALNRLVYLIYGK

SEQ ID NO: 237 - protein, 263 aa, unidentified
MKKIAIVTGGSGGIGRCTAAALRDAGCTVYELSRREKPADGIVHITADVTDEAQVRAAIDEVISREGRIDILVNNAGFGISGAAELTDTKDSHAQLELNVFGTDNVTRAVLPHMRAHGGGRIVCMSSIAGIVPIPFQLWYSVSKAAIIAYVLALQNEVKPFNISVCAIMPGDIASGFTDARKKSGAGDDVYAGRIKRSVAVMEHDERTGMSPEFAGRFVAKYALKKNSRPLVAMGAAYKGAAALVKLLPRQTSNWLVGKIYAK

SEQ ID NO: 238 - protein, 253 aa, unidentified
MKIAVITGASSGMGREFVYALDRDEEFDELWVIARREDRLRELQSKCRAKVRPLALDLQDRASFAAYRALLESEKPEISVLVNAAGFGLFGMFTEMDMDKQLDIIDLNDRALTAMCHMSIPYMAAGSRIYNMGSMSSWQPVPYINVYGASKAYVLSFSRALGVELEKQGIRVMAVCPGWIKTEFFSHAIHDNTVNYFNRYYGPEQVVAKALKDMKRGKDASVLGFPERMQVRLVKLLPVKMVMNTWCRQQGKK

SEQ ID NO: 239 - protein, 252 aa, unidentified; identical to SEQ ID NO: 189

SEQ ID NO: 240 - protein, 266 aa, unidentified
VTPVKKVCVITGGTSGIGLCTAQAMLEKGYTVYELSRRAEGAPGMNHIAADVTKEETLAAAIQEILKREDHIDVLINNAGFGISGAVEFTKTEDAQHQLDVNFFGMVRMNRQVLPVMRRQCYGRIVNLSSVAGAIPIPFQTYYSASKAAINSYTMALANEVKPFGIQVCCVQPGDIRTGFTAAREKNPEGDDIYGGRIARSVAGMERDEQNGMDPEKAGAFIAHVATRKGIRPVNTIGLQYKFFCFLVKILPAKTLNWLVGLIYAK

SEQ ID NO: 241 - protein, 250 aa, unidentified
MTAAVITGASAGLGAEFTRQLVREFPEIEEFWLIARRVGKLEELAQQFPDKKFVCMGLNLLDPKSFRYLGEKLAEQKADVRVLVNNAGCGTTGNIGFGATSADVMRVVDLNVRALTMVTQTVIPFMTRGAKIINVSSIAAFCPTPRMTVYSASKAYVSAFTGGIADELRPKGISVTAVCPGPMKTEFFDAAGDHDLFGNIPWCDPVKVVAGTIRAAKKGRTFYTPTVFNKFYRFVAKILPMKWMIKATRV

SEQ ID NO: 242 - protein, 247 aa, unidentified
MEKLDLHGKTGFIIGGTRGIGNAIAMRMAGLGMNLTVIARKEDELAAMKAQVESLGVRFLGIQASNTDFDAIAAAFETSWQTYGRLDLLVNAAGTGVMTPFEELTKSDVDTTIDVNLKGTIYAVKLSVPYFEKTGGGNILNISSMSAIRGIPDPVNNNGIYTATKFATNGFTECMQKYLLKYGIRVTALCPGSTATSWWTRWTHSFGTDAMLPVDTLADIAELIVTSPEKVLFKQLQVLPTVEIDNF

SEQ ID NO: 243 - protein, 263 aa, unidentified
VSQVCVITGGTSGIGRCTAQAMLARGYTVYELSRRAEGVAGMQHIVADVTKEETLAAAVAQILQREDHIDVLINNAGFGISGAVEFTGTEEAQRQLDVNFFGMVRMNRQVLPVMRKQGYGRIVNLSSVAGAIPIPFQTYYSASKAAINSYTMALANEVKPFGIQVCCVQPGDIRTGFTAAREKNQLGDDIYGGRIARSVSGMERDEQTGMAPEQAGAFIARVATRKGVRPVNTIGLQYKFFCFLAKVLPARWLNALVGLIYAK

SEQ ID NO: 244 - protein, 250 aa, unidentified
MTAAVITGASAGLGAEFTRQLVREFPDVEEFWLIARRVGKLEELAQQFPEKKFVCIGLNLLDPKSFRYLGEKLAEQKADVRLLVNNAGCGTTGNIDASASSADVMRVVDLNVRALTMVTQTVVPFMTRGAKIINVSSIAAFCPTPRMTVYSASKAYVSAFTGGLADELRPKGITVTAVCPGPMKTEFFDAAGDHDLFGNIPWCDPVRVVAGTIRAAKKGRTFYTPTAFNKFYRFVAKILPMKWMIKATRV

SEQ ID NO: 245 - protein, 263 aa, unidentified
MSKIALVTGGTSGIGKETALYLAKNGCTVYELSRRAEGVAGLRHISADVTDEASVRRAVEQILQEAGQIDILVNNAGFGISGAVEFTDTEEAERQFNVNFFGMVRMNRAVIAAMRSRGGGRIVNLSSVAAPVPIPFQTYYSASKAAINSYTMALANELRPFGITVCAVMPGDIHTGFTAARRKVAEGDEIYAGRISRSVKRMEHDEQTGMDPAKAGAFVGRVALKTGHKPLYTIGFAYKAAVFLTKILPAGTLNWLIGKIYAS

SEQ ID NO: 246 - protein, 254 aa, unidentified
MKKIAVVTGASSGMGRDFVRAVDREFSPQEIWVIARREDRLLALQEQVHATIVPFAMDLSRDEAFRCYQEALEREQPEIVALVNAAGYGKFTPFAEMDMASQLGIVELNDRALTAMCHMSLPYMHPGSKIINLGSNSAWQPVPYMSVYGASKAYVLSFSRALGRELKGRDIQVLCVCPGWVKTEFMDRAVHDSTVSFFDRWYESEAVVEKAMRDLRKGKTVSILGFPVRMQVRLVKLLPVRMIMNIWCKQQKKP

SEQ ID NO: 247 - protein, 271 aa, unidentified
MEKMRFEGKVVIVTGASSGIGRSTARLFAMEGAKVVAAARRMKRLEDLRDKVAAENAPGVILPVKTDVRDPAQIEAMFDTCLKEFGHLDILVNNAGVLDGQLPIHETTPEVYDMIYETNQRAVFLCCQRAIRIFLEQGTPANIVNVASAASLRGLKGGAMYVTTKHAVLGLTRNISASFFERGIRCNCIEPANILTEINKVPREKGIGILEWQMRAGKAAPLHTITKPGEKKPMLGKPKDCANAIAYLADDVAARYISGAELKVDAGWLNM

SEQ ID NO: 248 - protein, 269 aa, unidentified
MFSVSEASVAVITGGSSGIGLNAARALRDRGLIVYELSRRAENAEPGVTHLQADVTDETQVNAAVAEILRREGRIDILINNAGFGISGAIEFTPPQEARRQFDVNFFGMVNMNRAVLPVMRQQGGGRIVNMSSVAAPIAIPFQAYYSASKAAVRTYTLALASEVRPFGIEVCVIMPGDIATGFTAARHKSCGGDDVYNGRIARSVAVMEHDERTGMSAQFAGQFVARRATQKHPKLICTMGRKYALFVFLMRILPTRTATRIVGRIYAS

SEQ ID NO: 249 - protein, 253 aa, unidentified
MNIAVITGASAGIGRELVYAVDKDAEYDEIWVIARRKERLEELRGKCRNPIRPIALDLSDLSSIDAYQALLEQEQPEIRMLVNAAGCGVFGPFAEADRKKLISSAQLNSLALTGMCHASLPYMHSGSNIINMGSNSAWQPVPFQAVYGASKSYVLSLSRALGRELRPQGIHVMCVCPGWIKTEFQQVAHHDEFIRYVDKWYGPDEVAAQAMKDLKKKKSVSILGHPVRRQVRLVKLLPVDTVMDIWCKQQGIE

SEQ ID NO: 250 - protein, 265 aa, unidentified
MSKKVAIVTGGTSGIGRATALALQERGYTVYELSRRAEGMPDIRHIVADITKEETLRAAVEQVLAVEGRLDLVVNNAGFGISGAIEFTDTQEAQRLFDTLFFGMVRMNRCVIPLMRQQGHGRIVNISSVAAPVPIPFQAYYSAGKAAINAYTMALANELRPFGVTVCAVMPGDIKTGFTAARHKIIDGDDIYQGRIGRSVQRMEHDELTGMDPAKVGRYIAAVASREGSHHPLYATRIDYKFFVFLTKVLPARFLNWLIYQLYGK

SEQ ID NO: 251 - protein, 256 aa, unidentified
MKKIAVITGASSGMGKRFAETIDQFGTFDEVWVIARQWDKLEALRDTVPFPIRVLAMDLTDRASFNIYKAALAEEPVQVGLLMNCSGYGKFSAVLDTPLEVNLNMTDLNCQAVVAMCQLTAPYMPRGSQIINIASVAAFQPIPYINVYGATKAFVLSFSRALNRELRRQGVGVMAVCPFWTKTAFFDRAVTDGEAIVKKYAAMYDPDDIVRRTWRDAKRGKDVCKYGFVARGQAALTKLLPHSLVMDVWMKQQQLS

SEQ ID NO: 252 - protein, 253 aa, Oscillibacter sp.; identical to SEQ ID NO: 192

SEQ ID NO: 253 - protein, 263 aa, unidentified
MAKICVITGGTSGIGKCTAEAMAQKGYTVYELSRREEGLPGMFHIPTDVTDPDACQRAIDEVVSQAGRIDVMINNAGFGISGAIEFTPVEQAKRQFDVNFFGMVNMNCAVIPVMRAQRSGRIVNLSSVAGAIPIPFQAFYSASKAAINSYTMALANEIKPFGVQVCCVQPGDIQTGFTAARQKIVVGDDIYGGRISRSVAGMEKDERTGMRPEDAGAFVCRAATRKGVRPVNTIGLSYKFFCVLQKLLPAKTLNWLVGMVYAR

SEQ ID NO: 254 - protein, 256 aa, unidentified
MKKIAIITGASSGMGKVFAETINTYDTFDEVWVIARRLDRLESLQETVPFPVRPIALDLTDRESFRHYAELLENAQANVQLLVNCSGYGKFQAACDTPLSQNLNMVDLNCEALMAMCQLTIPYMHAGAQIINIASVAACQPVPYIGVYAASKAFVLSYSRALNRELDDKDISVMAVCPFWTKTEFFDHAVIDGEKPVVKKYAAMYEPQQIVARAWRDAKRGKDVSKFGFIARAQMALVKILPHSLIMDIWLSQQKL

SEQ ID NO: 255 - protein, 266 aa, unidentified
MNSSKVAVVTGGTSGIGKATALALQKAGCTVYELSRRAEGVEGLRHISADVTDEAAVNAAVAQIMAKAGHIDLLVNNAGFGISGAIEFTAPEDAKKLFDVNFFGMVNMNRAVVPLMRAAGHGRIVNLSSVAAPVPIPFQAYYSATKAAVNAYTMALANELRPFGVTVCAVMPGDIHTGFTAARKKVAEGDAIYHGRISRSVQRMEHDEETGMDPAKAGAYIAAVALRDGNRHPLYAIRFDYKFFTFLAKVLPARFLNWLIYCLYGK

SEQ ID NO: 256 - protein, 257 aa, unidentified
MKKIAVITGASSGMGRRFAETVDRYGRFDEVWVIARHQAQLEALKATVPFPIRVLALDLTDRTSFGTYAAALAEEPVQVGLLMNCSGYGKFSAVLDTPLAVNLNMTDLNCQAVVAMCQLTAPYMPSGSQIINIASVAAFQPIPYIDIYGATKAFVLSFSRALNRELRSCGIGVMAVCPFWTKTAFFDRAIREGEQPIVKKYVAMYDVEDIVTRTWRDAKRGKDVCKYGFIARAQAGLAKILPHSLVMDVWMKQQELR

SEQ ID NO: 257 - protein, 286 aa, unidentified
MKENRNWVVTGASGGIGAVLVEKLLEKGFRVAALSRTPEKIEARHGAQEGLRAIRVDVRSEESVLKAREEAKAFLGRVDVVVNTAGYGLGGAIEEVSDQEARAVFDVNVFGALNVCRHFVTDLREQGGGCIINFACMDSVIATGYNAVYHATKYAMDALTDALNKEAGQFGIRAICVKNGPMRTDYLDNKQYAAEQLEVYQQARQENRAREARYGAAAAADPGKVAELLIRLSGEEEAPKDLYLTRESVKAIREKWDALEQEFETWKWATLCVDFPKEECYFGKRN

SEQ ID NO: 258 - protein, 252 aa, unidentified
MKTAIITGASSGLGSEFVRQLADVFPEIQCCWLIARRRDRLEKLAVNLPGWTVECLPMDLCDPMSFVALQEKLNTQKPEVALLINNAGCGYLGNLGEMETTTQTRMVDLNLRALTAVTNMVIPFMAPGGRILNVSSIASFCPNPRMTVYSASKAYVSAFTVGLAEELRPKGITATAVCPGPMKTEFLDVGGISGHSRTFDMLPYCDQVKVAGGALRAARAGRTIYTPRLFYKFYRVLAKVTPVKLMVKIAGT

SEQ ID NO: 259 - protein, 266 aa, unidentified
MNEKKVAVVTGGTSGIGRATALALKDAGCTVYELSRRAEGVEGLHHISADVTDESAVNAAVAQVVAAEGQIDILVNNAGFGISGAIEFTSTTDAKSLFDVNFFGMVNMNRAVVPLMRAAGHGRIVNLSSVAAPVPIPFQAYYSATKAAVNAYTMALANELRPFGVTVCAVMPGDIHTGFTAARRKLSEGDDIYQGRISRSVVRMEHDEQTGMDPAKAGTYIAKVALRSGSNHPLYAIRFDYKFFAFLAKVLPARFLNWLIYLLYGK

SEQ ID NO: 260 - protein, 257 aa, unidentified
MKKIAVITGASSGMGKRFAQRVNEFGTFDEIWLIARHGERLEALRETMPFPARVLALDLTDRTSFGVYEAALAEEPVEIGLLVNASGFGKFAAVADTPLQVNLNMTDLNCQAVVALCQLSIPYLGRGGQIINIASVAAFQPIPYVDVYGATKAFVLSFSRALNRELRPRGAAVMAVCPFWTKTAFFDRAVVSEEKPVVKKYVAMYDPEDIVTRAWRDAKRGKDVSKYGFIARFQAALTKILPHSLVMDVWMAQQGLE

SEQ ID NO: 261 - protein, 264 aa, unidentified
MNKKIAIVTGGTSGIGKATALALRGAGYTVYEFSRREAGVEGLHHIRADITDETQVRAAVQQVMDAEGQIDVVVNNAGFGISGAVEFTDTADAQRLFNADFFGMVRVNRAVIAHMRAAGRGRIVNLSSVAGPLPIPFQTYYSAAKAAVNAYTMALANELRPFGITVCAVQPGDIHTGFTAAREKTVTGDEVYGGRISRSVSRMEHDEQTGMDPAAAGRFIARVAQKTSHKPIYTIRLDYQFFVFLKRILPYRALNALIGLIYAK

SEQ ID NO: 262 - protein, 252 aa, unidentified
MKTAIITGASSGLGREFARQLTDVFPEIECCWLIARREDRLEEIAREMVGVETVCLPLDLCDSMSFTTLQEKLAAEKPEAAILINNAGCGYLGRMGETETAVQTRMVDLNVRAMTAVTNLVIPYMPAGGRILNTSSIASFCPTPRMTVYGASKAYVSSFTVGLSEELKRKNITVTAVCPGPMKTEFLDVGSITGRSPAFEYLPYCDQVRVAAGALRAAKAGRTIYTPRLFYKFYRLLAKVTPVKLMVKFTKT

SEQ ID NO: 263 - protein, 284 aa, Oscillibacter sp.
MKEFFGKVALITGAAHGIGYSFALEAASRGMKLALVDIDETAMLEAAKECSRFGAEVLTCTTDVSVYEEAKASVEATMARFGQIDLLFANAGIATAGSILHIPIRDWEWALAVNTMGIVHYVHEVLPIMEAQKTPAYLMCTASIAGLRAGMAINPPYFCSKHAAVSVAESVKAEVESSGSDIGVSVFCPMYVATDIDNCENHRPARFWDASDPFYNSEEYLTAREAFHKNIATGMPLKKIGKRLFKAIEDNQMYIVTHTQTIPYIESRHRAIEEDAKKELELQL

SEQ ID NO: 264 - protein, 252 aa, synthetic construct
MKTAIVTGASAGLGREIVRQIAAVFPEIEAYWLIARRAERLEELAAAMPDQQVDCMPLDLCDPMSFMTLQEKLASEQPEVALLVNNAGCGYLGNVGEVETSTQTRMIDLNLRALTAVTNITVPFMAPGSRILNVSSIAAFCPTPRMTVYSAGKSYVSAFSIGLAEELRHKGITVTAVCPAPMRTEFLEAGNIAGNSRMFERLPYCDQVQVAGGALRAARAGRVIYTPKLFYKFYRVLAKVTPVKLMVKFTKT

SEQ ID NO: 265 - protein, 253 aa, synthetic construct; identical to SEQ ID NO: 191

SEQ ID NO: 266 - protein, 253 aa, synthetic construct; identical to SEQ ID NO: 191

SEQ ID NO: 267 - protein, 253 aa, synthetic construct; identical to SEQ ID NO: 191

SEQ ID NO: 268 - protein, 253 aa, synthetic construct; identical to SEQ ID NO: 191

SEQ ID NO: 269 - protein, 253 aa, synthetic construct; identical to SEQ ID NO: 191

SEQ ID NO: 270 - protein, 253 aa, synthetic construct; identical to SEQ ID NO: 191

(19) United States
(12) Patent Application Publication
(10) Pub. No.: US 2026/0183347 A1
(43) Pub. Date: Jul. 2, 2026

(54) COMPOSITIONS AND METHODS FOR TREATING CARDIOVASCULAR DISEASE

(71) Applicants: The Broad Institute, Inc., Cambridge, MA (US); The General Hospital Corporation, Boston, MA (US)

(72) Inventors: Chenhao Li, Boston, MA (US); Martin Strazar, Cambridge, MA (US); Ahmed M. T. Mohamed, Cambridge, MA (US); Damian R. Plichta, Cambridge, MA (US); Ramnik J. Xavier, Boston, MA (US)

(73) Assignees: The Broad Institute, Inc., Cambridge, MA (US); The General Hospital Corporation, Boston, MA (US)

(21) Appl. No.: 19/562,539

(22) Filed: Mar. 10, 2026

Related U.S. Application Data
(63) Continuation of application No. PCT/US2024/047057, filed on Sep. 17, 2024.
(60) Provisional application No. 63/538,978, filed on Sep. 18, 2023.
(60) Provisional application No. 63/561,609, filed on Mar. 5, 2024.

(51) Int. Cl. A61K 35/74; A61K 45/06; A61P 3/06; C07K 14/195; C12N 1/20; C12N 9/10; C12R 1/01

(52) U.S. Cl. CPC A61K 35/74; A61K 45/06; A61P 3/06; C07K 14/195; C12N 1/20; C12N 9/1051; C12R 2001/01; C12Y 204/01173

(57) ABSTRACT

The present disclosure includes compositions and methods for treating cardiovascular disease (CVD) (e.g., cholesterol related disorders, or diseases caused or characterized by increased levels of plasma triglycerides, plasma cholesterol, or serum C-reactive protein), or symptoms thereof. The disclosure also includes compositions and methods for lowering plasma triglycerides in a subject, lowering plasma cholesterol levels in a subject, and lowering serum C-reactive protein levels in a subject.

### CROSS-REFERENCE TO RELATED APPLICATIONS

This application is a continuation under 35 U.S.C. § 111 (a) of PCT International Patent Application No. PCT/US2024/047057, filed Sep. 17, 2024, designating the United States and published in English, which claims priority to and the benefit of U.S. Provisional Application No. 63/561,609, filed Mar. 5, 2024, and U.S. Provisional Application No. 63/538,978, filed Sep. 18, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Sep. 30, 2024, is named 167741-051302PCT_SL.xml and is 335,598 bytes in size.

### BACKGROUND OF THE INVENTION

Cardiovascular disease (CVD) is a disease of the heart or blood vessels which is a leading cause of death globally. Hypercholesterolemia, or high circulating cholesterol, is strongly associated with the development and progression of CVD, which is the cause of one-fourth of all deaths in industrialized countries. Therefore, there is a pressing need for more effective treatments for hypercholesterolemia and CVD.

### SUMMARY OF THE INVENTION

As described below, the present invention features compositions and methods that are useful for treating cardiovascular disease.

In an aspect, the present disclosure provides a composition including or consisting of a CgT polypeptide and a pharmaceutically acceptable excipient.

In another aspect, the present disclosure provides a composition including or consisting of an Oscillibacter or Dysosmobacter IsmA polypeptide and a pharmaceutically acceptable excipient.

In another aspect, the present disclosure provides a composition including or consisting of an IsmA polypeptide and a CgT polypeptide.

In another aspect, the present disclosure provides a composition including an effective amount of an IsmA polypeptide having at least 85% amino acid sequence identity to a polypeptide encoded by Oscillibacter gene RJX3347_02204 or RJX3711_01778, or Dysosmobacter gene J115_02655 and an effective amount of an CgT polypeptide having at least 85%, 90%, or 95% amino acid sequence identity to a polypeptide encoded by Oscillibacter gene RJX3347_02251 or Dysosmobacter gene J115_17675.

In another aspect, the present disclosure provides composition including or consisting of an isolated Oscillibacter species and an excipient.

In another aspect, the present disclosure provides composition including or consisting of an isolated Oscillibacter species and an isolated Eubacterium species each expressing IsmA.

In another aspect, the present disclosure provides a recombinant microbial cell. The cell includes a heterologous polynucleotide encoding an IsmA polypeptide.

In another aspect, the present disclosure provides a composition including the recombinant microbial cell of any one of the above aspects, or embodiments thereof. The composition is formulated for delivery to the small intestine, the large intestine, the colon, or the rectum.

In another aspect, the present disclosure provides a therapeutic combination including the composition of any one of the above aspects, or embodiments thereof, or the recombinant microbial cell of any of the above aspects, or embodiments thereof, and a low density lipoprotein (LDL) cholesterol lowering agent.

In another aspect, the present disclosure provides a method of reducing plasma triglycerides in a subject. The method involves administering to the subject the composition of any one of the above aspects, or embodiments thereof, or the recombinant microbial cell of any one of the above aspects, or embodiments thereof.

In another aspect, the present disclosure provides a method of reducing plasma cholesterol in a subject in need thereof. The method involves administering to the subject the composition of any one of the above aspects, or embodiments thereof, or the recombinant microbial cell of any one of the above aspects, or embodiments thereof.

In another aspect, the present disclosure provides a method of reducing serum C-reactive protein (CRP) in a subject. The method involves administering to the subject the composition of any one of the above aspects, or embodiments thereof, or the recombinant microbial cell of any one of the above aspects, or embodiments thereof.

In another aspect, the present disclosure provides a method of treating cardiovascular disease, cholesterol related disorders, or diseases associated with or characterized by increased levels of plasma triglycerides, plasma cholesterol, or serum C-reactive protein in a subject or reducing the propensity of the subject to develop cardiovascular disease, cholesterol related disorders, or diseases associated with or characterized by increased levels of plasma triglycerides, plasma cholesterol, or serum C-reactive protein. The method involves administering to the subject the composition of any one of the above aspects, or embodiments thereof, or the recombinant microbial cell of any one of the above aspects, or embodiments thereof.

In another aspect, the present disclosure provides a kit including the composition of any one of the above aspects, or embodiments thereof, or the recombinant microbial cell of any one of the above aspects, or embodiments thereof, and instructions for their use in the treatment of cardiovascular disease, cholesterol related disorders, or diseases associated with or characterized by increased levels of plasma triglycerides, plasma cholesterol, or serum C-reactive protein.

In another aspect, the present disclosure provides an expression vector including a polynucleotide encoding an IsmA polypeptide or CgT polypeptide.

In another aspect, the present disclosure provides a method of treating cardiovascular disease, cholesterol related disorders, or diseases associated with or characterized by increased levels of plasma triglycerides, plasma cholesterol, or serum C-reactive protein in a subject. The method involves administering to the subject the composition of any one of the above aspects, or embodiments thereof, or the recombinant microbial cell of any one of the above aspects, or embodiments thereof, where the subject has previously been or is concurrently being administered an LDL cholesterol lowering agent.

In another aspect, the present disclosure provides a method of treating hypercholesterolemia in a subject. The method involves administering to the subject the composition of any one of the above aspects, or embodiments thereof, or the recombinant microbial cell of any one of the above aspects, or embodiments thereof, where the subject has previously been or is concurrently being administered an LDL cholesterol lowering agent.

In another aspect, the present disclosure provides a method of lowering plasma cholesterol in a subject in need thereof. The method involves administering to the subject the composition of any one of the above aspects, or embodiments thereof, or the recombinant microbial cell of any one of the above aspects, or embodiments thereof, wherein the subject has previously been or is concurrently being administered an LDL cholesterol lowering agent.

In another aspect, the present disclosure provides a method of lowering plasma cholesterol in a selected subject. The method involves administering to the subject the composition of any one of the above aspects, or embodiments thereof, or the recombinant microbial cell of any one of the above aspects, or embodiments thereof, where the selected subject is being administered an LDL cholesterol lowering agent, and where the selected subject has increased plasma cholesterol relative to a reference during the period of administration of the LDL cholesterol lowering agent.

In any of the above aspects, or embodiments thereof, the composition includes an effective amount of the CgT or IsmA polypeptide, where the effective amount is an amount sufficient to treat or prevent cardiovascular disease in a subject.

In any of the above aspects, or embodiments thereof, the IsmA polypeptide has at least about 85%, 90%, or 95% amino acid sequence identity to a polypeptide encoded by Oscillibacter gene RJX3347_02204 or RJX3711_01778, or Dysosmobacter gene J115_02655 and the CgT polypeptide has at least 85%, 90%, or 95% amino acid sequence identity to an Oscillibacter or Dysosmobacter CgT polypeptide. In any of the above aspects, or embodiments thereof, the CgT polypeptide is encoded by Oscillibacter gene RJX3347_02251 or Dysosmobacter gene J115_17675.

In any of the above aspects, or embodiments thereof, the composition is formulated for delivery to the small intestine, the large intestine, the colon, or the rectum. In any of the above aspects, or embodiments thereof, the small intestine is the duodenum, ileum, or jejunum. In any of the above aspects, or embodiments thereof, the small intestine is the ileum.

In any of the above aspects, or embodiments thereof, the pharmaceutical composition further includes a pharmaceutically acceptable excipient.

In any of the above aspects, or embodiments thereof, the Oscillibacter species expresses an IsmA polypeptide. In any of the above aspects, or embodiments thereof, the Oscillibacter IsmA polypeptide is encoded by Oscillibacter gene RJX3347_02204 or RJX3711_01778. In any of the above aspects, or embodiments thereof, the Oscillibacter species expresses a CgT polypeptide. In any of the above aspects, or embodiments thereof, the Oscillibacter CgT polypeptide is encoded by Oscillibacter gene RJX3347_02251.

In any of the above aspects, or embodiments thereof, the Oscillibacter species is RJX3347 or RJX3711.

In any of the above aspects, or embodiments thereof, the composition further includes a Eubacterium species expressing an IsmA polypeptide. In any of the above aspects, or embodiments thereof, the Eubacterium species is Eubacterium coprostanoligenes. In any of the above aspects, or embodiments thereof, the IsmA polypeptide expressed by the Eubacterium species has at least about 85%, 90%, or 95% amino acid sequence identity to a polypeptide encoded by Eubacterium coprostanoligenes gene ECOP170.

In any of the above aspects, or embodiments thereof, the composition is formulated for oral or rectal administration. In any of the above aspects, or embodiments thereof, the composition is formulated in a powder, bolus, gel, capsule, liquid, food stuff, or suppository.

In any of the above aspects, or embodiments thereof, the recombinant microbial cell is selected from the phyla Firmicutes, Bacteroidetes, Actinobacteria, Bacteroidetes, Proteobacteria, Fusobacteria, Verrucomicrobia, Euryarchaeota, and Ascomycota. In any of the above aspects, or embodiments thereof, the recombinant microbe is within a genus selected from the group consisting of Corynebacterium, Bifidobacterium, Atopobium, Faecalibacterium, Clostridium, Roseburia, Ruminococcus, Dialister, Lactobacillus, Enterococcus, Staphylococcus, Streptococcus, Sphingobacterium, Bacteroides, Tannerella, Parabacteroides, Alistipes, Prevotella, Escherichia, Shigella, Desulfovibrio, Bilophila, Helicobacter, Fusobacterium, Pediococcus, Bacillus, Leuconostoc, Akkermansia, Methanobrevibacter, Propionibacterium, Coriobacteriaceae, Actinobacteria, Rikenellaceae, Lachnospiraceae, Firmicutes, Peptostreptococcaceae, Veillonella, Oscillospira, Dialister, Slackia, Eggerthella, Gordonibacter, Geobacter Alkaliphilus, Catenibacterium, Holdemania, Marvinbryantia, Symbiobacterium, Roseburia, Erysipelotrichaceae, Butyricicoccus, Sporobacter, Blautia, Dorea, Succinivibrio, Barnesiella, Biolophila, Eubacterium, or Saccharomyces. In any of the above aspects, or embodiments thereof, the microbe is an engineered variant of a Lactobacillus, Bifidobacterium, Saccharomyces, Enterococcus, Streptococcus, Pediococcus, Leuconostoc, Bacillus, or Escherichia coli.

In any of the above aspects, or embodiments thereof, the microbe is a bacterium. In any of the above aspects, or embodiments thereof, the IsmA polypeptide has at least about 85%, 90%, or 95% amino acid sequence identity to a polypeptide encoded by Oscillibacter gene RJX3347_02204 or RJX3711_01778, or Dysosmobacter gene J115_02655.

In any of the above aspects, or embodiments thereof, the cell further includes a CgT polypeptide encoded by a heterologous polynucleotide. In any of the above aspects, or embodiments thereof, the CgT polypeptide has at least about 85%, 90%, or 95% amino acid sequence identity to a polypeptide encoded by Oscillibacter gene RJX3347_02251 or Dysosmobacter gene J115_17675.

In any of the above aspects, or embodiments thereof, the cell further includes a Eubacterium IsmA polypeptide. In any of the above aspects, or embodiments thereof, the Eubacterium IsmA polypeptide has at least about 85%, 90%, or 95% amino acid sequence identity to a polypeptide encoded by Eubacterium coprostanoligenes gene ECOP170.

In any of the above aspects, or embodiments thereof, the LDL cholesterol lowering agent is one or more of a statin, a cholesterol absorption inhibitor, a bile acid sequestrant, a PCSK9 inhibitor, an adenosine triphosphate-citrate lyase (ACL) inhibitor, or a microsomal triglyceride transfer protein (MTP) inhibitor.

In any of the above aspects, or embodiments thereof, the LDL cholesterol lowering agent is a statin. In any of the above aspects, or embodiments thereof, the statin is one or more of atorvastatin, cerivastatin, fluvastatin, lovastatin, mevastatin, pitavastatin, pravastatin, rosuvastatin, or simvastatin.

In any of the above aspects, or embodiments thereof, the LDL cholesterol lowering agent is a cholesterol absorption inhibitor. In any of the above aspects, or embodiments thereof, the cholesterol absorption inhibitor is ezetimibe.

In any of the above aspects, or embodiments thereof, the LDL cholesterol lowering agent is a bile acid sequestrant. In any of the above aspects, or embodiments thereof, the bile acid sequestrant is cholestyramine, colesevelam, or colestipol.

In any of the above aspects, or embodiments thereof, the LDL cholesterol lowering agent is a PCSK9 inhibitor. In any of the above aspects, or embodiments thereof, the PCSK9 inhibitor is alirocumab or evolocumab.

In any of the above aspects, or embodiments thereof, the LDL cholesterol lowering agent is an ACL inhibitor. In any of the above aspects, or embodiments thereof, the ACL inhibitor is bempedoic acid.

In any of the above aspects, or embodiments thereof, the LDL cholesterol lowering agent is a MTP inhibitor. In any of the above aspects, or embodiments thereof, the MTP inhibitor is lomitapide.

In any of the above aspects, or embodiments thereof, the method increases stool cholestenone levels, cholesterol alpha-D-glucoside levels, or coprostanol levels in the subject.

In any of the above aspects, or embodiments thereof, the cholesterol related disorder is hypercholesterolemia.

In any of the above aspects, or embodiments thereof, the polynucleotide comprises a polynucleotide sequence having at least about 85%, 90%, or 95% amino acid sequence identity to Oscillibacter gene RJX3347_02204 or RJX3711_01778, Dysosmobacter gene J115_02655, or Eubacterium coprostanoligenes gene ECOP170.

In any of the above aspects, or embodiments thereof, the CgT polypeptide is an Oscillibacter or Dysosmobacter CgT polypeptide. In any of the above aspects, or embodiments thereof, the CgT polypeptide has at least about 85%, 90%, or 95% amino acid sequence identity to a polypeptide encoded by Oscillibacter gene RJX3347_02251 or Dysosmobacter gene J115_17675.

In any of the above aspects, or embodiments thereof, the subject has previously been or is concurrently being administered a maximum tolerated dose of the LDL cholesterol lowering agent.

In any of the above aspects, or embodiments thereof, the subject has side effects or symptoms of toxicity associated with the LDL cholesterol lowering agent.

The invention provides compositions and methods that are useful for treating cardiovascular disease. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “administer” is meant giving, supplying, or dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), injection, intrathecal, intramuscular, dermal, intradermal, intracranial, inhalation, rectal, intravaginal, or intraocular.

By “agent” is meant any cell, small molecule chemical compound, nucleic acid molecule, polypeptide, or fragments thereof. In some embodiments, the polypeptide is an IsmA polypeptide and/or a CgT polypeptide. In some embodiments, the cell is an Oscillibacter species, an IsmA expressing cell (e.g., bacterial cell), and/or a cell (e.g., bacterial cell) expressing CgT.

By “alteration” is meant a change in the structure, expression levels or activity of a polynucleotide or polypeptide as detected by standard art known methods, such as those described herein. The alteration can be an increase or a decrease. As used herein, an alteration includes a 10% change in expression levels, a 25% change, a 40% change, and a 50% or greater change in expression levels.”

By “ameliorate” is meant decrease, suppress, attenuate, diminish, arrest, or stabilize the development or progression of a disease. In some embodiments, the disease is a cardiovascular disease associated with undesirable levels of cholesterol.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “cardiovascular disease” or “CVD” is meant any disease of the heart or blood vessels, including, but not limited to, hypercholesterolemia, coronary artery disease, atherosclerotic cardiovascular disease (ASCVD), acute coronary syndrome, and ischemic heart disease (IHD). In some embodiments, cardiovascular disease comprises cholesterol related disorders, or diseases associated with or characterized by increased levels of plasma triglycerides, plasma cholesterol, or serum C-reactive protein.

By “Cholesterol-Alpha-Glucosyltransferase (CgT) polypeptide” is meant a protein, or fragment thereof, having at least about 85% amino acid sequence identity to GenBank Accession No. ABI95890.1 (Helicobacter mustelae), CCI80972.1 (Lactobacillus hominis), or another CGT polypeptide disclosed herein, and having glucosyltransferase activity. In some embodiments, “CgT polypeptide” comprises a protein, or fragment thereof, having at least about 85% amino acid sequence identity to any of the exemplary amino acid sequences below, and having glucosyltransferase activity. The sequence of exemplary CgT polypeptides follow:

>msp257_G141073_k105_23267_3
(SEQ ID NO: 1)
MKIVLVIDQFDDANNGTTISARRFAQALKNHGNEVRVIATGKPADYKYAVRQMRFFPVVEHLITSQGMRL
AIPNRHVFEKAAAWADVVHFMMPSPLGIMGLKHVEKLGIPHTAAFHCQPENITFTLHMGNSRRVNDFVYN
RFRDTFFNRFTHIHCPSNMIANQLRQHGYTARLHVISNGISPEYIYGKREKEPWMQGLFNVLMVGRYAGE
KRQDELIDACAKSRHAREIQVILAGKGPLEKKYRRLAEKLPNPIVMEFYEPARLLEILHMADLYVHTSDA
EIEAMSCMEAFACGLVPVIADSPRSATPQFALDERSLFPAGDTDALAQRIDWWIEHPEERQAMERRYAEH
ARQYSLEESIRQTEEMFRQAIAEQRGAKA
>J115_17675 GDP-mannose-dependent alpha-mannosyltransferase
(SEQ ID NO: 2)
MKIVLVIDQFDDANNGTTISARRFAQALKNHGNEVRVIATGKPADYKYAVRQMRFFPVVEHLITSQGMRL
AIPNRHVFEKAAAWADVVHFMMPSPLGIMGLKHVEKLGIPHTAAFHCQPENITFTLHMGNSRRVNDFVYN
RFRDTFFNRFTHIHCPSNMIANQLRQHGYTARLHVISNGISPEYIYGKREKEPWMQGLFNVLMVGRYAGE
KRQDELIDACAKSRHAREIQVILAGKGPLEKKYRRLAEKLPNPIVMEFYEPARLLEILHMADLYVHTSDA
EIEAMSCMEAFACGLVPVIADSPRSATPQFALDERSLFPAGDTDALAQRIDWWIEHPEERQAMERRYAEH
ARQYSLEESIRQTEEMFRQAIAEQRGAKA
>ABI95890.1 cholesterol alpha-glucosyltransferase
[Helicobacter mustelae]
(SEQ ID NO: 3)
MTIGIVIDSYNDRSNGTSMTAFRFAREFVKKGHEVRIVACNVSKSMSDEEDLKLYPVKQRYIPIVTEVSK
KQHMIFGAPDLEVLQSAVVGCDIVHFYMPFALEIAGMHLCRSLRIPYISAFHVQPQHISYNMNMNFSWFN
TYLFKRFYKHFYRYTHHIHCPSKFIEKELQRENYGGKKYTISNGFFGGDRVMADPYEDSFFHIASVGRFS
KEKKQDIIIKAIAKNPYADKIKLHLHGVGPREKYLKNLCNKLLINKPEFGFIDNGALLEKLAKMHLYVHA
AKVESEAISCLEAISLGVVPVIADSETSATVQFALDPLSLFEVNNVADLSNKITYWIEHPKELLAYKQKY
AESALQYSLDKSIEETLGLYEEAIRDFRDQPALFDRINA
>CCI80972.1 Cholesterol alpha-glucosyltransferase
[Lactobacillus hominis DSM 23910 = CRBIP 24.179]
(SEQ ID NO: 4)
MKILIVIDDYHNNSNGMSISTQRFVKEFKKLGCDVRVLAIGDVSYSLPEMKIPFFAKLIAQQGFHFALPV
RKTTLKAVQWADYVHLDTPFPIGWQAGHLAKKMGKTVTGTFHIYPQNMTASVPILNQKWINNLIMFVFKK
ISYKNCDVIQCPTAKVKRNLQKYHFPQKLVVISNGIAQAFIDNPHKTDTQQDFTILCIGRFSNEKDQYTL
FRAMQVCKYAKNINLIFAGQGPLKDKFEALAKTLPRKPIMRYFNSKQLRKLEAKSQLVVHCANVEVEGMS
CMEAFASGCVPIIASSDLSSTASYSLTKNNQFKAEDYHELAKRIEYWYEHPLELGNMSKKYQTYARSLNV
NKCAKMALSMIRSAKKR
>RJX3347_02251
(SEQ ID NO: 5)
MKIVLVIDQFDDANNGTTISARRFAQALRDHGNEVRVIATGKPADYKYAVRQMRFFPIVEHLITSQGMRL
AIPNRHVFEKAAAWADVVHFMMPSPLAVMGLKHVEKLGIPHTAAFHCQPENITFTLHLGNSQRANDFVYN
RFRDSFFNRFTHIHCPSNMIAEQLRQHGYTAQLHVISNGISPEYFYGKQPKETWMQGFFNVLMVGRYAGE
KRQDELIAACTKCRHAKEIQVILAGRGPLEKKYRRLAEKLPNPIVMNFYEPARLLEILHMADLYVHTSDA
EIEAMSCMEAFACGLVPVIADSPRSATPQFALDERSLFPAGDTDVLAERIDWWLEHPEERREMELRYAEH
AKQYTLEHSIQQTEEMFRMAIREQRG
>WP_187031161.1 glycosyltransferase
[Pusillibacter faecalis]
(SEQ ID NO: 6)
MKIVLVIDQFDDANNGTTISARRFAQALRDHGNEVRVIATGKPADYKYAVRQMRFFPIVEHLITSQGMRL
AIPNRHVFEKAAAWADVVHFMMPSPLAVMGLKHVEKLGIPHTAAFHCQPENITFTLHLGNSQRANDFVYN
RFRDSFFNRFTHIHCPSNMIAEQLRQHGYTAQLHVISNGISPEYFYGKQPKETWMQGFFNVLMVGRYAGE
KRQDELIAACTKCRHAKEIQVILAGRGPLEKKYRRLAEKLPNPIVMSFYEPARLLEILHMADLYVHTSDA
EIEAMSCMEAFACGLVPVIADSPRSATPQFALDERSLFPAGDTDVLAERIDWWLEHPEERREMELRYAEH
AKQYTLEHSIQQTEEMFRMAIREQRG
>MBS6353921.1 MAG: glycosyltransferase
[Oscillibacter sp.]
(SEQ ID NO: 7)
MKIVLVIDQFDDANNGTTISARRFAQALKNHGNEVRVIATGKPADYKYAVRQMKFFPVVEHLITSQGMRL
AIPNRHVFEKAAAWADVVHFMMPSPLGIMGLKHVEKLGIPHTAAFHCQPENITFTLHLGNSRRVNDFVYN
RFRDTFFNRFTHIHCPSNMIANQLRQHGYTAQLHVISNGISPEYTYGKREKEPWMQGFFNVLMVGRYAGE
KRQDELIDACAKSRHAREIQVILAGKGPLEKKYRRLAEKLPNPIVMEFYEPARLLEILHMADLYVHTSDA
EIEAMSCMEAFACGLVPVIADSPRSATPQFALDERSLFPAGDTDALAQRIDWWIEHPEERQAMERRYAEH
ARQYSLEESIRQTEEMFRQAIAEQRGAKA
>HJB54007.1 MAG TPA: glycosyltransferase
[Candidatus Oscillibacter pullicola]
(SEQ ID NO: 8)
MKIVLVIDQFDDANNGTTISARRFAQALKNHGNEVRVIATGKPADYKYAVRQMKFFPVVEHLITSQGMRL
AIPNRHVFEKAAAWADVVHFMMPSPLGIMGLKHVEKLGIPHTAAFHCQPENITFTLHLGNSRRVNDFVYN
RFRDTFFNRFTHIHCPSNMIANQLRQHGYTAQLHVISNGISPEYTYGKREKEPWMQGFFNVLMVGRYAGE
KRQDELIDACAKSRHAREIQVILAGKGPLEKKYRRLAEKLPNPIVMEFYEPARLLEILHMADLYVHTSDA
EIEAMSCMEAFACGLVPVIADSPRSATPQFALDERSLFPAGDTDALAARIDWWIEHPEERQAMERRYAEH
ARQYSLEESIRQTEEMFRQAIAEQRGAKA
>WP_021750835.1 MULTISPECIES: glycosyltransferase
[Oscillospiraceae]
(SEQ ID NO: 9)
MKIVLVIDQFDDANNGTTISARRFAQALKNHGNEVRVIATGKPADYKYAVRQMRFFPVVEHLITSQGMRL
AIPNRHVFEKAAAWADVVHFMMPSPLGIMGLKHVEKLGIPHTAAFHCQPENITFTLHMGNSRRVNDFVYN
RFRDTFFNRFTHIHCPSNMIANQLRQHGYTARLHVISNGISPEYIYGKREKEPWMQGLFNVLMVGRYAGE
KRQDELIDACAKSRHAREIQVILAGKGPLEKKYRRLAEKLPNPIVMEFYEPARLLEILHMADLYVHTSDA
EIEAMSCMEAFACGLVPVIADSPRSATPQFALDERSLFPAGDTDALAQRIDWWIEHPEERQAMERRYAEH
ARQYSLEESIRQTEEMFRQAIAEQRGAKA
>HJB13548.1 MAG TPA: glycosyltransferase
[Candidatus Oscillibacter
excrementigallinarum]
(SEQ ID NO: 10)
MKIVLVIDQFDDANNGTTISARRFAQALKDNGNEVRVIATGKPADYKYAVRQLKLFPVVEHLLTSQGMRL
AVPNKHVFEKAAAWADVVHFMMPSPLGVMGLKHVEKLGIPHTAAFHCQPENITFTLHLGNSKRVNDFVYN
RFRDSFFNRFTHIHCPSNMIADQLRQHGYTAQLHVISNGISPEYTYGKRDKEPWMQGFFNVLMVGRYAGE
KRQDELIDACAKSRHAQEIQVILAGKGPLEKKYRKLAEKLPNPIVMDFYEPARLLDILHMADLYVHTSDA
EIEAMSCMEAFACGLVPVIADSPRSATPQFALDERSLFPAGDTDALAARIDWWIEHPEERREMELRYADH
ARQYSLEASIRRTEEMFRQAIAEQRGARA
>MBE5709504.1 MAG: glycosyltransferase [Oscillibacter sp.]
(SEQ ID NO: 11)
MKIVLVIDQFDDANNGTTISARRFAQALKNHGNEVRVIATGKPADYKYAVRQMRFFPVVEHLITSQGMRL
AIPNRHVFEKAAAWADVVHFMMPSPLGIMGLKHVEKLGIPHTAAFHCQPENITFTLHMGNSRRVNDFVYN
RFRDTFFNRFTHIHCPSNMIANQLRQHGYTARLHVISNGISPEYIYGKREKEPWMQGLFNVLMVGRYAGE
KRQDELIDACAKSRHAREIQVILAGKGPLEKKYRRLAEKLPNPIVMEFYEPARLLEILHMADLYVHTSDA
EIEAMSCMEAFACGLVPVIADSPRSATPQFALDERSLFPAGDTDALAQRIDWWIEHPEERQAMERRYAEH
ARQYSLEESIRQAEEMFRQAIAEQRGAKA
>WP_025545300.1 glycosyltransferase
[Dysosmobacter welbionis]
(SEQ ID NO: 12)
MKIVLVIDQFDDANNGTTISARRFAQALKNHGNEVRVIATGKPADYKYAVRQMRFFPVVEHLITSQGMRL
AIPNRHVFEKAAAWADVVHFMMPSPLGIMGLKHVEKLGIPHTAAFHCQPENITFTLHMGNSRRVNDFVYN
RFRDTFFNRFTHIHCPSNMIANQLRQHGYTARLHVISNGISPEYIYGKREKEPWMQGLFNVLMVGRYAGE
KRQDELIDACAKSRHAREIQVILAGKGPLEKKYRRLAEKLSNPIVMEFYEPARLLEILHMADLYVHTSDA
EIEAMSCMEAFACGLVPVIADSPRSATPQFALDERSLFPAGDTDALAQRIDWWIEHPEERQAMERRYAEH
ARQYSLEESIRQTEEMFRQAIAEQRGAKA
>WP_204803546.1 glycosyltransferase
[Oscillibacter valericigenes]
(SEQ ID NO: 13)
MKIVLVIDQFDDANNGTTISARRFAQALKASGNEVRVIATGKPADYKYAVRQMKFFPVVEHLITSQGMRL
AIPNKHVFEKAAAWADVVHFMMPSPLGIMGLKHVEKLGIPHTAAFHCQPENITFTLHLGNSRRVNDFVYN
RFRDTFFNRFTHIHCPSNMIADQLRSHGYTAQLHVISNGISPEYTYGKQEKEPWMQGFFNVLMVGRYAGE
KRQDELIEACAKSRHAQEIQVILAGKGPLEKKYRRLAEKLPNPIVMSFYEPARLLEILHMADLYVHTSDA
EIEAMSCMEAFACGLVPVIADSPRSATPQFALDGRSLFPAGDTDALAERIDWWIEHPAERQEMERRYAEH
AKQYSLAESIRRTEEMFRQAIAEQRGTRA
>QUO37404.1 glycosyltransferase
[Dysosmobacter sp. Marseille-Q4140]
(SEQ ID NO: 14)
MKIVLVIDQFDDANNGTTISARRFAQALRDHGNEVRVIATGKPADYKYAVRQMRFFPVVEHLITSQGMRL
AIPNRHVFEKAAAWADVVHFMMPSPLGIMGLRHVEKLGIPHTAAFHCQPENITFTLHMGNSKRANDFVYN
RFRDTFFNRFTHIHCPSNMIADQLRSHGYTAQLHVISNGISPEYTYGKRPKEPWMEGFFNVLMVGRYAGE
KRQDELIEAAARCRHAQQIQVILAGKGPLEKKYRRLAEKLPNPVVMGFYEPARLLEILHMADLYVHTSDA
EIEAMSCMEAFACGLVPVIADSPRSATPQFALDERSLFPAGDVDALAERIDYWIDHPQERAEMERRYAVH
AGQYSLRNSIFQTEEMFRQAIEEQKRA
>MCI6053654.1 MAG: glycosyltransferase [Dysosmobacter sp.]
(SEQ ID NO: 15)
MKIVLVIDQFDDANNGTTISARRFAQALRDHGNEVRVIATGKPADYKYAVRQMRFFPVVEHLITSQGMRL
AIPNRHVFEKTAAWADVVHFMMPSPLGIMGLRHVEKLGIPHTAAFHCQPENITFTLHMGNSKRANDFVYN
RFRDTFFNRFTHIHCPSNMIADQLRSHGYTAQLHVISNGISPEYTYGKRPKEPWMEGFFNVLMVGRYAGE
KRQDELIEAAARCRHAQQIQVILAGKGPLEKKYRRLAEKLPNPVVMGFYEPARLLEILHMADLYVHTSDA
EIEAMSCMEAFACGLVPVIADSPRSATPQFALDERSLFPAGDVDALAERIDYWIDHPQERAEMERRYAVH
AGQYSLRNSIFQTEEMFRQAIEEQKRA
>WP_016322903.1 glycosyltransferase [Oscillibacter sp. 1-3]
(SEQ ID NO: 16)
MKIVLVIDQFDDANNGTTISARRFASALKAHGNEVRVIATGKPADYKYAVRQMRFFPIVEHLITSQGMRL
AIPNRHVFEKAAAWADVVHFMMPSPLAIMGLRHVEKIGIPHTAAFHCQPENITFTLHMGNSRRVNDFVYG
KFRDTFFNRFTHIHCPSSMIANQLRSHGYTAQLHVISNGISPEYVYGKRPKEDWMEGFFNVLMVGRYAGE
KRQDVLIEAAARCRHAGEIQVILPGKGPLEKKYRHLAEKLPNPIVMGFYEPARLIEILHMADLYVHTSDA
EIEAMSCMEAFACGLVPVIADSPRSATPQFALDERSLFPAGDSDTLAERIDWWIEHPAERQEMERRYAEH
AKQYALDRSIQQTEEMFRQAIREQGKKN
>MCI7733339.1 MAG: glycosyltransferase [Dysosmobacter sp.]
(SEQ ID NO: 17)
MKIVLVIDQFDDANNGTTISARRFAMALKEHGNEVRVIAIGKNTDYKYAVRQMKFPPVVEHLITSQGMRL
AIPNRHVFEKAAAWADVVHFMMPSPLAVMGLKHVEKLGIPHTAAFHCQPENITFTLHMGNSRKANDLLYH
GFRDTFFNRFTHIHCPSNMIANQLRQHGYTAQLHVISNGISPEYFYDKRPKEDWMQGYFNVLMVGRYAGE
KRQDELIEACAKSRHAREIQVILAGKGPLEKKYRKLAEKLPNPIVMQFYEPSRLIEILHMADLYVHTSDA
EIEAMSCMEAFACGLVPVIANSPRSATPQFALDERSLFPAGDTDALAERIDYWIEHPQEREEMERRYAEH
AKQYALERSIEQTEDMFRMAIAEQKATTKE
>MCI9649763.1 MAG: glycosyltransferase family 4 protein
[Oscillibacter sp.]
(SEQ ID NO: 18)
MKIVLVIDQFDDANNGTTISARRFASALKAHGNEVRVIATGKPADYKYAVRQMRFFPIVEHLITSQGMRL
AIPNKHVFEKAAAWADVVHFMMPSPLAVMGLRHVEKIGIPHTAAFHCQPENITFTLHMGNSRRVNDFVYG
KFRDTFFNRFTHIHCPSAMIANQLRDHGYTAQLHVISNGISPEYVYGKRAKEDWMEGFFNILMVGRYAGE
KRQDVLIEAAAKCRHAQEIQVILPGKGPLEKKYRHLAESLPNPVVMGFYEPARLIEILHMADLYVHTSDA
EIEAMSCMEAFACGLVPVIADSPRSATPQFALDQRSLFPAGDSDMLAQRIDWWIEHPQERREMERRYAQH
AGQYTLERSIEQAEEMFRQAIRERERKN
>MCI8803278.1 MAG: glycosyltransferase family 4 protein
[Oscillibacter sp.]
(SEQ ID NO: 19)
MKIVLVIDQFDDANNGTTISARRFAAALKEHGNEVRVIATGKPTDYKYAVRQMRFLPIVEHLITSQGMRL
AIPNRHVFEKAAAWADVVHFMMPSPLAIMGLKHVERLGIPHTAAFHCQPENITYTLHLGNSKRVNDFVYV
KFRDTFFNRFTHIHCPSNMIAGQLRDHGYTARLHVISNGISPQYVYGRRPQEEWMQGKFNVLMVGRYAGE
KRQDVLIEACARCRHREEIQVILAGKGPLEKKYRRLAEKLSNPVVMEFYEPARLLEILHMADLYVHTSDA
EIEAMSCMEAFACGLVPVIADSPRSATPQFALDERSLFPAGDSAALAEKIDWWMEHPREREEMGKRYGEH
ARQYALERSIEQTEEMFRMAIAEQRG
>MCI8573839.1 MAG: glycosyltransferase family 4 protein
[Oscillibacter sp.]
(SEQ ID NO: 20)
MKIVLVIDQFDDANNGTTISARRFATALKEHGNEVRVIATGKPADYKYAVRQMKFFPIVEHLITSQGMRL
AIPNRHVFEKAAAWADVVHFMMPSPLAIMGLKHVERLNIPHTAAFHCQPENITFTLHLGNSRRANDFVYG
KFRDTFFNRFTHIHCPSNMIANQLREHGYTAQLHVISNGISPQYQYGRQPKEDWMKDRFNVLMVGRYAGE
KRQDVLIKACAQCRHGAEIQVILAGKGPLEKKYRRLARNLPNPIVMEFYEPARLLEILHMADLYVHTSDA
EIEAMSCMEAFACGLVPVIADSPRSATPQFALDERSLFPAGDSGALAERIDWWFEHPEERQEMGLRYAAH
AEQYALSRSIEQTEEMFRQAIAER
>MCI8399811.1 MAG: glycosyltransferase family 4 protein
[Oscillibacter sp.]
(SEQ ID NO: 21)
MKIVLVIDQFDDANNGTTISARRFASALKEHGNEVRVIATGKPTDYKYAVRQMKFLPIVEHLITSQGMRL
AIPNRHVFEKAAAWADVVHFMMPSPLAIMGLRHVERLGIPHTAAFHCQPENITFTLHMGNNRRVNDFVYN
RFRDTFFNRFTHIHCPSTMIANQLREHGYTAELHVISNGISPQYSYGRAPQEDWMQGRFNVLMVGRYAGE
KRQDVLIDACAKCRHKDEIQVILAGKGPLEKKYRRMAEKLSNPIVMEFYEPSRLLEILHMADLYVHTSDA
EIEAMSCMEAFACGLVPVIADSTRSATPQFALDERSLFPAGDSGALAEKIDWWLEHPEEREAMGRRYGEH
AKQYALERSIEQTEEMFRTAIKEQRGPRP
>MCI9461594.1 MAG: glycosyltransferase family 4 protein
[Oscillibacter sp.]
(SEQ ID NO: 22)
MKIVLVIDQFDDANNGTTISARRFATALKEHGNEVRVIATGKPTDYKYAVRQMKFLPIVEHLITSQGMRL
AIPNKHVFEKAAAWADVVHFMMPSPLAVMGLKHVERLGIPHTAAFHCQPENITFTLHLGNSKRVNDFVYT
KFRDTFFNRFTHIHCPSNMIADQLRQHGYTAQLHVISNGISPQYTYGRSPQEDWMKGKFNVLMVGRYAGE
KRQDVLIDACARCRHQDDIQVILAGKGPLEKKYRRLAEKLPNPIVMEFYEPGRLLEILHMADLYVHTSDA
EIEAMSCMEAFACGLVPVIADSPRSATPQFALDGRSLFPAGDPGALAEKIDWWFEHPQEREEMGKRYGEH
AKRYALDRSIQQTEEMFQTAIREQKR
>MCI8689229.1 MAG: glycosyltransferase family 4 protein
[Oscillibacter sp.]
(SEQ ID NO: 23)
MKIVLVIDQFDDANNGTTISARRFATALKEHGNEVRVIATGKPADYKYAVRQMKFFPIVEHLITSQGMRL
AIPNRHVFEKAAAWADVVHFMMPSPLAIMGLKHVERLNIPHTAAFHCQPENITFTLHLGNSRRVNDFVYG
KFRDTFFNRFTHIHCPSNMIANQLREHGYTAQLHVISNGISPQYQYGRQPKEDWMKDRFNVLMVGRYAGE
KRQDVLIKACAQCRHGAEIQVILAGKGPLEKKYRRLARNLPNPIVMEFYEPARLLEILHMADLYVHTSDA
EIEAMSCMEAFACGLVPVIADSPRSATPQFALDERSLFPAGDSGALAERIDWWFEHPEERQEMGLRYAAH
AEQYALSRSIEQTEEMFRQAIAER
>MCI8810562.1 MAG: glycosyltransferase family 4 protein
[Oscillibacter sp.]
(SEQ ID NO: 24)
MKIVLVIDQFDDANNGTTISARRFATALKEHGNEVRVIATGKPADYKYAVRQMKFFPIVEHLITSQGMRL
AIPNRHVFEKAAAWADVVHFMMPSPLSIMGLKHVERLNIPHTAAFHCQPENITFTLHLGNSRRANDFVYS
KFRDTFFNRFTHIHCPSNMIANQLREHGYTAQLHVISNGISPQYTYGRSPKEDWMKDRFQVLIVGRYAGE
KRQDVLIKACTQCRHAREIQVILAGKGPLEKKYRRLAQSLPNPAVMEFYEPARLLEILHMADLYVHTSDA
EIEAMSCMEAFACGLVPVIADSPRSATPQFALDERSLFPAGDSAALAERIDWWFEHPSEREEMGKRYAEH
AKQYALSRSIEQTEEMFRQAIAENEP
>WP_091128677.1 glycosyltransferase [Oscillibacter sp. PC13]
(SEQ ID NO: 25)
MKIVLVIDQFDDANNGTTISARRFAMALKEHGNEVRVIATGKPADYKYAVRQMKFFPIVEHLITSQGMRL
AIPNRHVFEKAAAWADVVHFMMPSPLGVMGLRHVEKLGIPHTAAFHCQPENITFTLHMGNNKRVNDFVYN
RFRDSFFNRFTHIHCPSNMIAEQLRQHGYTAKLHVISNGISPQYIYGKRPKEDWMQGMFNVLMVGRYAGE
KRQDVLIEAASKCRHAKEIQVILAGKGPLEKKYRHLAEKELPNPVVMGFYEPSRLIEILHMCDLYVHTSD
AEIEAMSCMEAFACGLVPVIANSPRSATPQFALDDRSLFPAGDAAALAEQIDWWIEHPAEREKMEHRYAE
HAKQYTLERSIELAEGMFQEAIDEQKQV
>MCI8849281.1 MAG: glycosyltransferase family 4 protein
[Oscillibacter sp.]
(SEQ ID NO: 26)
MKIVLVIDQFDDANNGTTISARRFATALKEHGNEVRVIATGKPADYKYAVRQMKFFPIVEHLITSQGMRL
AIPNRHVFEKAAAWADVVHFMMPSPLAIMGLKHVERLNIPHTAAFHCQPENITFTLHLGNSRRANDFVYG
KFRDTFFNRFTHIHCPSNMIANQLREHGYTAQLHVISNGISPQYQYGRQPKEDWMKDRFNVLMVGRYAGE
KRQDVLIKACAQCRHGAEIQVILAGKGPLEKKYRRLARNLPNPIVMEFYEPARLLEILHMADLYVHTSDA
EIEAMSCMEAFACGLVPVIADSPRSATPQFALDERSLFPAGDSGALAERIDWWFERPEERQEMGLRYAAH
AEQYALSRSIKQTEEMFRQAIAER
>MCI8739773.1 MAG: glycosyltransferase family 4 protein
[Oscillibacter sp.]
(SEQ ID NO: 27)
MKIVLVIDQFDDANNGTTISARRFAAALKAHGNEVRVIATGKPADYKYAVRQMRFFPIVEHLITSQGMRL
AIPNKHVFEKAAAWADVVHFMMPSPLAIMGLKHVEKIGIPHTAAFHCQPENITFTLHMGNSRRMNDFVYG
KFRDTFFNRFTHIHCPSNMIANQLREHGYTAQLHVISNGISSEYVYGKRPKENWMEGFFNILMVGRYAGE
KRQDVLIEAAAKCRHAGEIQVILPGKGPLEKKYRHLAEKLPNPVVMGFYEPARLIEILHMADLYVHTSDA
EIEAMSCMEAFACGLVPVIADSPRSATPQFALDERSLFPAGDSDVLAERIDWWIEHPEERREMERRYAEH
AEQYTLERSIEQTEEMFRQAIGEKSRTV
>MCI8480955.1 MAG: glycosyltransferase family 4 protein
[Oscillibacter sp.]
(SEQ ID NO: 28)
MKIVLVIDQFDDANNGTTISARRFARALKAHGNEVRVIATGKPADYKYAVRQMRFFPIVEHLITSQGMRL
AIPNRHVFEKAAAWADVVHFLMPSPLAVMGLKHVEKLGIPHTAAFHCQPENITFSLHMGNSRRVNDFVYE
KFRDTFFNRFTHIHCPSHMIAGQLEAHGYTAQLHVISNGISPEYIYGRQPKEPWMEGKFNILMVGRYAGE
KRQDVLIDAAAKCRRAGEIQVILAGKGPLEKKYRHLAENLPNPIVMQFFEPSRLLEILHMADLYVHTSDA
EIEAMSCMEAFACGLVPVIADSPRSATPQFALDGRSLFPAGDSSALAEKIDWWMEHPEERREMGLRYGEH
ARQYALERSIAQMEEMFRLAIQEQRDR
>MBR1691026.1 MAG: glycosyltransferase [Oscillibacter sp.]
(SEQ ID NO: 29)
MKIVLVIDQFDDANNGTTISARRFAMALKEHGNEVRVIAIGKPADYKYAVRQMRFFPIVEHLITSQGMRL
AIPNKHVFEKAAAWADVVHFMMPSPMAIMGLKHVERLGIPHTAAFHCQPENITFTLHLGNSTRVNDFVYN
RFRDTFFNRFTHIHCPSNMIAEQLRRHGYTAQLHVISNGISPQYVYGKRPKEEWMQGYFNVLSVGRYAGE
KRQDVLIEAAAKCRHAQEIQVILAGKGPLEKKYRKLAEKLPNPAVMGFYEPERLLDILHMADLYVHTSDA
EIEGMSCTEAFACGLVPVIAVAPRSATSQFALDMRSLFPGGDTDALAEKIDWWIEHPAEREEMEHRYAEH
AKQYTLERSIELTEEMFHQAIAEQTHG
>MBQ9330477.1 MAG: glycosyltransferase [Oscillibacter sp.]
(SEQ ID NO: 30)
MKIVLVIDQFDDANNGTTISARRFAMALKEHGNEVRVIAIGKPADYKYAVRQMRFFPIVEHLITSQGMRL
AIPNKHVFEKAAAWADVVHFMMPSPMAIMGLKHVERLGIPHTAAFHCQPENITFTLHLGNSTRVNDFVYN
RFRDTFFNRFTHIHCPSNMIAEQLRRHGYTAQLHVISNGISPQYVYGKRPKEEWMQGYFNVLSVGRYAGE
KRQDVLIEAAAKCHHAREIQVILAGKGPLEKKYRKLAEKLPNPAVMGFYEPERLLDILHMADLYVHTSDA
EIEGMSCTEAFACGLVPVIAVAPRSATSQFALDMRSLFPGGDTDALAEKIDWWIEHPAEREEMEHRYAEH
ARQYTLERSIELTEEMFRQAIAEQTH
>MCI8970940.1 MAG: glycosyltransferase family 4 protein
[Oscillibacter sp.]
(SEQ ID NO: 31)
MKIVLVIDQFDDANNGTTISARRFATALKEHGNEVRVIATGKPTDYKYAVRQMRFLPIVEHLITSQGMRL
AIPNRHVFEKAAAWADVVHFMMPSPLAVMGLKHVERLGIPHTAAFHCQPENITFTLHLGNSKRVNDFVYD
KFRDTFFNRFTHIHCPSNMIAGQLRDHGYTAQLHVISNGISPRYTYGRSPQEDWMQGKFNVLMVGRYAGE
KRQDVLIDACARCRHKDAVQVILAGKGPLEKKYRRLAEALSNPIVMEFYEPDRLLEILHMADLYVHTSDA
EIEAMSCMEAFACGLVPVIADSPRSATPQFALDGRSLFPAGDAAALAEKIDWWFEHPEEREEMGKRYGEH
AKRYALDRSIRQTEEMFRTAIREQRT
>MCI9577908.1 MAG: glycosyltransferase family 4 protein
[Oscillibacter sp.]
(SEQ ID NO: 32)
MKIVLVIDQFDDANNGTTISARRFATALKEHGNEVRVIATGKPTDYKYAVRQMKFLPIVEHLITSQGMRL
AIPNRHVFEKAAAWADVVHFMMPSPLAVMGLKHVERLGIPHTAAFHCQPENITFTLHLGNSKRVNDFVYD
KFRDTFFNRFTHIHCPSNMIAGQLRDHGYTAQLHVISNGISPRYAYGRSPQEDWMRGKFNVLMVGRYAGE
KRQDVLIDACARCRHKDAVQVILAGKGPLEKKYRRLAETLSNPIVMEFYEPDRLLEILHMADLYVHTSDA
EIEAMSCMEAFACGLVPVIADSPRSATPQFALDERSLFPAGDAAALAEKIDWWFEHPEEREEMGKRYGEH
AKRYALDRSIRQTEEMFRTAIREQRT
>MCI8819457.1 MAG: glycosyltransferase family 4 protein
[Oscillibacter sp.]
(SEQ ID NO: 33)
MKIVLVIDQFDDANNGTTISARRFATALKEHGNEVRVIATGKPADYKYAVRQMRFFPIVEHLISSQGMRL
AIPNRHVFEKAAAWADVVHFMMPSPLAIMGLKHVERLGIPHTAAFHCQPENITFTLHMGNSRRVNDFVYV
KFRDTFFNRFTHIHCPSNMIANQLRDHGYTARLHVISNGISPQYTYGRTPQEDWMRGRFNVLMVGRYAGE
KRQDVLIDACAQCRHKDEIQVILAGKGPLERKYRRQAEKLPNPIVMEFYEPSRLLEILHMADLYVHTSDA
EIEAMSCMEAFACGLVPVIADSPRSATPQFALDERSLFPAGDAAALAEKIDWWFEHPEAREEMGRRYAQH
AEQYALDRSIVQTEEMFRLAIQEQKRG
>MCI9331938.1 MAG: glycosyltransferase family 4 protein
[Oscillibacter sp.]
(SEQ ID NO: 34)
MKIVLVIDQFDDANNGTTISARRFATALKEHGNEVRVIATGKPTDYKYAVRQMKFLPIVEHLITSQGMRL
AIPNKHVFEKAAAWADVVHFMMPSPLSIMGLKHVERLGIPHTAAFHCQPENITFTLHLGNSKRVNDFVYV
KFRDTFFNRFTHIHCPSNMIANQLRQHGYTAQLHVISNGISPQYTYGRSPQEDWMRGKFNVLMVGRYAGE
KRQDVLIDACLQCRHKDAIQVILAGKGPLEKKYRHLAEKLPNPIVMEFYEPSRLLEILHMADLYVHTSDA
EIEAMSCMEAFACGLVPVIADSPRSATPQFALDERSLFPAGDAGALAEKIDWWFEHPKEREDMGRRYAKH
AGRYALDRSIGQTEEMFQMAIREQKA
>MBP3508718.1 MAG: glycosyltransferase [Oscillibacter sp.]
(SEQ ID NO: 35)
MKIVLVIDQFDDANNGTTISGRRFAAALKEHGNEVRVIAIGKPADYKYAVRQMKFPPIVEHLVTSQGMRL
AIPNKHVFEKAAAWADVVHFMMPSPMAVMGLKHVERLGIPHTAAFHCQPENITFTFHMGNNKRVNDFVYD
KFRDTFYNRFNHIHCPSEMIANQLRQHGYTAQLHVISNGISPQYTYGKLPKEDWMQGYFNVLMVGRYAGE
KRQDVLIEAAAKCRHAKEIQVILAGKGPLEKKYRQLAEKLPNPVVMGFYEPERLLDILHMADLYVHTSDA
EIEGMSCTEAFACGLVPVIAVAPRSATSQFALDERSLFPGGDSDALAERIDWWIEHPQERQEMELKYAEH
AKQYTLERSIELTEEMFRQAIEEQEKLNLTK
>MCI9644264.1 MAG: glycosyltransferase family 4 protein
[Oscillibacter sp.]
(SEQ ID NO: 36)
MKIVLVIDQFDDANNGTTISARRFATALKEHGNEVRVIATGKPTDYKYAVRQMKFLPIVEHLITSQGMRL
AIPNRHVFEKAAAWADVVHFMMPSPLSIMGLKHVERLGIPHTAAFHCQPENITYTLHLGNSKRVNDFVYT
KFRDTFFNRFTHIHCPSNMIANQLRDHGYTAQLHVISNGISPQYTYGRAPQEDWMRGKFNVLMVGRYAGE
KRQDVLINACAQCRHKEDIQVILAGKGPLEGKYRRLAEKLPNPIVMEFYEPGRLLEILHMADLYVHTSDA
EIEAMSCMEAFACGLVPLIADSPRSATPQFALDGRSLFPAGDAAALAEKIDWWFEHPQELEEMGRRYGGH
AKQYALDRSIQQTEEMFRTAIREQRK
>MBD5148621.1 MAG: glycosyltransferase family 4 protein
[Oscillibacter sp.]
(SEQ ID NO: 37)
MKIVLVIDQFDDANNGTTISARRFATALKQHGNEVRVIATGKPTDYKYAVRQMKFLPIVEHLITSQGMRL
AIPNRHVFEKAAAWADVVHFLMPSPLAIMGLKHVEKLNIPHTAAFHCQPENITFTLHLGNSRRVNDFVYS
KFRDTFFNRFTHIHCPSQMIANQLTEHGYTAQLHVISNGISPEYVYGKQPKEDWMAGKFNILMVGRYAGE
KRQDELINAAAKCRHAQEIQVILPGKGPLEHKYRKLAEKLPNPIVMQFYXPARLLXXLHMAXLYVHTSDA
EIEAMSCMEAFACGLVPVIADSPRSATPQFALDERSLFPAGDVDALAERIDYWIDHPEERKAMELRYGEH
AXQYALDRSIQLTEEMFRQAIAEQPAKRR
>WP_235221878.1 glycosyltransferase [Oscillibacter
valericigenes]
(SEQ ID NO: 38)
MKIVLVIDQFDDANNGTTISGRRFAAALKEHGNEVRVIATGKPADYKYAVRQMKFPPVVEHLVTSQGMRL
AIPNKHVFEKAAAWADVVHFMMPSPLAVMGLKHVERLGIPHTAAFHCQPENITFTFHMGNSKRANDFVYE
KFRDTFFNRFGHIHCPSKMIADQLRQHGYTSQLHVISNGISPQYTYGKLPKEDWMQGYFNVLMVGRYAGE
KRQDVLIEAAARCRHAKEIQVILAGKGPLEKKYRQMAEKLPNPVVMGFYEPERLLDILHMADLYVHTSDA
EIEGMSCTEAFACGLVPVIAVAPRSATSQFALDERSLFPGGDSGALAERIDWWIEHPRERQEMELKYAEH
AKQYTLERSIELTEEMFRQVIEEQKELNQTKP
>MCI9113072.1 MAG: glycosyltransferase family 4 protein
[Oscillibacter sp.]
(SEQ ID NO: 39)
MKIVLVIDQFDDANNGTTISARRFATALKEHGNEVRVIATGKPTDYKYAVRQMRFLPIVEHLITSQGMRL
AIPNKHVFEKAAAWADVVHFMMPSPLAIMGLKHVERLGIPHTAAFHCQPENITFTLHMGNSKRVNDFVYT
KFRDTFFNRFTHIHCPSNMIADQLRQHGYTARLHVISNGISPRYTYGRAPQEDWMRGKFNVLMVGRYAGE
KRQDVLIDACAQCRHKDGIQVILAGKGPLEKKYRRLAEKLPNPIVMEFYEPDRLLEILHMADLYVHTSDA
EIEAMSCMEAFACGLVPVIADSPRSATPQFALDGRSLFPAGDAAALAEKIDWWFEHPEEREEMGKRYGEH
AGRYALDRSIEQAEEMFRTAIQEQRK
>MCI9554799.1 MAG: glycosyltransferase family 4 protein
[Oscillibacter sp.]
(SEQ ID NO: 40)
MKIVLVIDQFDDANNGTTISARRFATALKEHGNEVRVIATGKPTDYKYAVRQMKFLPIVEHLITSQGMRL
AIPNRHVFEKAAAWADVVHFMMPSPLAIMGLRHVERLGIPHTAAFHCQPENISFTLHLGNSKRVNDFIYT
KFRDTFFNRFTHIHCPSSMIAGQLREHGYTARLHVISNGISPQYAYGRSPQEDWMRGKFNVLMVGRYAGE
KRQDVLIDACAQCRHKDEIQVILAGKGPLEKRYRRLAEKLPNPIVMEFYEPSRLLEILHMADLYVHTSDA
EIEAMSCMEAFACGLVPIIADSPRSATPQFALDGRSLFPAGDPAALAEKIDWWFDHPREREEMGRRYGEH
AKQYALDRSIEQTEEMFRTAIREQGK
>MCI9348605.1 MAG: glycosyltransferase family 4 protein
[Oscillibacter sp.]
(SEQ ID NO: 41)
MKIVLVIDQFDDANNGTTISARRFASALKAHGNEVRVIATGKPTDYKYAVRQMRFLPIVEHLITSQGMRL
AIPNKHVFEKAAAWADVVHFMMPSPLAIMGLKHVERLGIPHTAAFHCQPENITFTLHMGNSKRANDFVYN
RFRDTFFNRFTHIHCPSHMIANQLKEHGYTAQLHVISNGISPQYVYGRSPKEDWMSGMFNVLMVGRYAGE
KRQDVLIDACAECRHKDEIQVILAGKGPLERKYRKLCQKLPNPAVMEFFEPARLLEILHMADLYVHTSDA
EIEAMSCMEAFACGLVPVIADSPRSATPQFALDDRSLFPAGDSAALAEKIDWWIEHPREREEMGRRYGES
ARQYALERSIEQTEDMFRQAILETDKR
>MCI8841208.1 MAG: glycosyltransferase family 4 protein
[Oscillibacter sp.]
(SEQ ID NO: 42)
MKIVLVIDQFDDANNGTTISARRFATALKEHGNEVRVIATGKPTDYKYAVRQMRFLPIVEHLITSQGMRL
AIPNKHVFEKAAAWADVVHFMMPSPLAIMGLKHVERLGIPHTAAFHCQPENITFTLHMGNSKRVNDFVYT
KFRDTFFNRFTHIHCPSNMIADQLRQHGYTARLHVISNGISPRYTYGRAPQEDWMRGKFNVLMVGRYAGE
KRQDVLIDACAQCRHRDGIQVILAGKGPLEKKYRRLAEKLPNPIVMEFYEPDRLLEILHMADLYVHTSDA
EIEAMSCMEAFACGLVPVIADSPRSATPQFALDGRSLFPAGDAAALAEKIDWWFEHPEEREEMGKRYGEH
AGRYALDRSIEQAEEMFRTAIQEQGK
>MCI8330378.1 MAG: glycosyltransferase family 4 protein
[Oscillibacter sp.]
(SEQ ID NO: 43)
MKIVLVIDQFDDANNGTTISARRFATALKEHGNEVRVIATGKPTDYKYAVRQMRFLPIVEHLITSQGMRL
AIPNKHVFEKAAAWADVVHFMMPSPLAIMGLKHVERLGIPHTAAFHCQPENITFTLHLGNSRRVNDFVYV
KFRDTFFNRFTHIHCPSNMIANQLREHGYTAQLHVISNGISPQYTYGRSPQEDWMRGKFNVLMVGRYAGE
KRQDVLIDACAQCRCREEIQVILAGKGPLEGKYRRRAEKLLNPIVMEFYEPSRLLEILHMADLYVHTSDA
EIEAMSCMEAFACGLVPVIADSPRSATPQFALDERSLFPAGDAAALAEKIDWWHDHPQEREEMGKCYAEH
AKQYALDRSIEQTEEMFRLAIREQRV
>WP_235228730.1 glycosyltransferase [Oscillibacter
valericigenes]
(SEQ ID NO: 44)
MKIVLVIDQFDDANNGTTISARRFAKALKDHGNEVRVIAIGKPADYKYAVKQMKFLPVMEHLVTSQGMRL
AIPNKHVFEKAAAWADVVHFMMPSPLAVTGLRHVEKLGIPHTAAFHCQPENITYTFHLGRSKRANDLLYS
GFRDTFYNRFTHIHCPSNMITNQLRQHGYTAQLHVISNGISPQYFYGKLPKEDWMKGYFNVLMVGRYAGE
KRQDVLIEACARSHHAKEIQVILAGKGPLEKKYRKLAEKLQNPIVMQFYEPERLIDILHMADLYVHTSDA
EIEAMSCMEAFACGLVPIIAESSRSATPQFALDGRSLFPAGDSAALAAQIDWWIEHPEERKTMELRYAEH
AKQYAMEESIKKTEEMFRMAIAEQGAARKAVHP
>MCI8909144.1 MAG: glycosyltransferase family 4 protein
[Oscillibacter sp.]
(SEQ ID NO: 45)
MKIVLVIDQFDDANNGTTISARRFAQALKEHGNEVRVIATGKPADYKYAVRQMRFFPIVEHLITSQGMRL
AIPNRHVFEKAAAWADVVHFMMPSPLAIMGLKHVEKLGIPHTAAFHCQPENITFTLHLGNSQRVNDFVYT
RFRNTFFNRFTHIHCPSEMIADQLRRHDYTAQLHVISNGISPQYFYEKRPKEDWMQGKFNVLMVGRYAGE
KRQDVLIQAAAKSRHSGELQVILAGKGPLEKKYRRMAEVLPNPIVMDFYQPERLIEILHMADLYVHTADA
EIEAMSCMEAFACGLVPVIANSARSATPQFALDGRSLFPAGDSSALAAQMDWWIEHPEQRAAMERRYAEH
AEQYALARSIEQTEEMFRQAIAERN
>CDC68891.1 putative glycosyltransferase
[Oscillibacter sp. CAG: 155]
(SEQ ID NO: 46)
MKRMKIVLVIDQFDDANNGTTISARRFAMALKEHGNEVRVIAIGKPGPNKYAVRQMRFLPIVEHLITSQG
MRLAVPNRHVFEKAAAWADVVHFMMPSPLAVAGLKHVEKLGIPHTAAFHCQPENITFTLHLGNSKRANDF
LYNRFRDSFYNRFTHIHCPSNMIAEQLRQHGYTAQLHVISNGISPQYHYRKLPKEDWMLGKFNILMVGRY
AGEKRQDVLIDACARSRYAQQLQVILAGKGPLERKYRRLAEKLPNPIVMEFYEPERLLDIFAMCDLYVHT
SDAEIEAMSCMEAFACGLVPVIADSPRSATPQFALDERSLFPAGDSLALARRIDWWIDHPAQRQEMEVRY
AEHAKQYALERSIQLTEEMFRQAIEEQRPASR
>MCI8811696.1 MAG: glycosyltransferase family 4 protein
[Oscillibacter sp.]
(SEQ ID NO: 47)
MKIVLVIDQFDDANNGTTISARRFAMALKAHGNEVRVIATGKPADYKYAVRQMRFFPIVEHLMTSQGMRL
AIPNRHVFEKAAAWADVVHFMMPSPLAIMGLRHVEKIGIPHTAAFHCQPENITFTLHMGNSRRMNDFVYG
KFRDTFFNRFTHIHCPSGMIANQLRDHGYTAQLHVISNGISSEYVYGKRPKEDWMEGFFNILMVGRYAGE
KRQDVLIEAAARCRHASEIQVILPGKGPLEKKYRHLAKKLPNPVVMGFYEPARLIELLHMADLYVHTSDA
EIEAMSCMEAFACGLVPVIADSPRSATPQFALDGRSLFPAGDSATLAERIDWWIEHPEERREMERRYAEH
AKQYTLERSIEQTEEMFLQAIQEQGKNN
>MCI9122293.1 MAG: glycosyltransferase family 4 protein
[Oscillibacter sp.]
(SEQ ID NO: 48)
MKIVLVIDQFDDANNGTTISARRFAAALKEHGNEVRVIATGKPTDYKYAVRQMKFLPIVEHLITSQGMRL
AIPNRHVFEKAAAWADVVHFMMPSPLAIMGLKHVERLGIPHTAAFHCQPENITFTLHLGNSKRVNDFVYV
KFRDTFFNRFTHIHCPSRMIAEQLRQHGYTARLHVISNGISPQYAYGRSPQESWMKGRFNVLMVGRYAGE
KRQDLLIEACARCRCREQVQVILAGKGPLEKKYRRLARKLANPIVMEFYPPERLLELLHQADLYVHTSDA
EIEAMSCMEAFACGLVPVIADSPRSATPQFALDERSLFPAGDAGALAEKIDWWFEHPREREEMGRRYGEA
AKRYALDRSIDQTEEMFRTAIREQRG
>WP_204791568.1 glycosyltransferase [Oscillibacter sp. CU971]
(SEQ ID NO: 49)
MKIVLVIDQFDDANNGTTISARRFATALKEHGNEVRVIATGKPTDYKYAVRQMRFLPIVEHLITSQGMRL
AIPNKHVFEKAAAWADVVHFMMPSPLAIMGLKHVERLGIPHTAAFHCQPENITFTLHMGNSKRVNDFVYT
KFRDTFFNRFTHIHCPSNMIADQLRQHGYTARLHVISNGISPRYTYGRAPQEDWMRGKFNVLMVGRYAGE
KRQDVLIDACAQXRHXDGIQVILAGKGPLEKKYRRLAEKLPNPIVMEFYEPDRLLEILHMADLYVHTSDA
EIEAMSCMEAFACGLVPVIADSPRSATPQFAXDGRSLFPAGDAAALAEKIDWWFEHPEEREEMGKRYGEH
AGRYALDRSIEQAEEMFRTAIQEQGK
>WP_040659422.1 glycosyltransferase
[Oscillibacter ruminantium]
(SEQ ID NO: 50)
MKIVLVIDQFDDANNGTTISAQRFARALIQHGNQVRVIACGKPADYKYAVRQMKFLPVVEHLITSQGMRL
AIPNRHVFEKAAAWADVVHFMMPSPLGIMGLRHVEKLGIPHTAAFHCQPENITFSVHLGNSRRVNDFVYN
RFRDTFFNRFTHIHCPSNMIADQLRQHGYTAQLHVISNGISPAYFYHKREKEAFLQEKFTILMVGRYSGE
KRQDVLIDACAKSRHAAHIQLILAGKGPMEKKLRRQCEAQLANPAILEFYSPQRLIEIISQCDLYVHTSD
AEIEAMSCMEAFACGLVPVIAQSPRSATPQFALDERSLFPAGDSRLLAKRIDYWYEHPQERQEMEKRYAE
SAKDYSLEASICQTEEMFRAAIREMRH
>MCI2056908.1 MAG: glycosyltransferase
[Oscillibacter sp.]
(SEQ ID NO: 51)
MKIVLVVDQFDDANNGTTISGQRFARALVAHGNQVRVIACGKPAEYKYAVHQLHFPFFVEHLITSQGMRL
AVPNRHVFEKAAAWADVVHFMVPTPLGVMGLRHVEKLGIPHTAAFHCQPENITFSVHLGNNKKVNDWIYN
RFRDSFYNRFTHIHCPSNMIANQLRDHGYTAQLHVISNGIGPAYHYCKNPKEDWMKEKFAIIMVGRYSGE
KRQDELIEACKRSRHARRIQLILAGKGPLEKKYRKLCESLPNPVVMDFYSPERLIEILSQCDLYVHTSDA
EIEAMSCMEAFACGRVPVIADSPRSATPQFAIDERSLFPAGDVDALAKRIDYWIEHPDEREEMEHRYAEH
AKQWSLSHSIEMTEEMFRTAIREQGKGTAE
>BAL00170.1 putative glycosyltransferase
[Oscillibacter valericigenes
Sjm18-20]
(SEQ ID NO: 52)
MKIVLVIDQFDDANNGTTISAQRFARALMAHGNQVRVIACGKPADYKYAVRQMRFLPVVEHLITSQGMRL
AIPNRHVFEKAAAWADVVHFMMPSPLGIVGLRHVEKVGIPHTAAFHCQPENITFSVHLGNSRRANDFVYN
RFRDTFFNRFTHIHCPSRMIADQLRQHGYTAQLHVISNGISPAFFYQKREKEDFLKGKFAVLMVGRYSGE
KRQDVLIDACSKSRHASQLQLILAGKGPTEKKLRRRCEERLQNPVVMEFYSPQRLIEIIDQCDLYVHTSD
AEIEAMSCMEAFASGLVPVIAQSPRSATPQFALDERSLFPAGDSTVLAKRIDYWFEHPEERREMERRYAE
SAKNYSLENSILQTEEMFGMAIREIWP
>MBS6866450.1 MAG: glycosyltransferase
[Oscillospiraceae bacterium]
(SEQ ID NO: 53)
MRLLIVVDQFDSGNNGTTISAQRFARALRERGHEVRVAAAGKPAEGKYALPRIHFLPLVDNIIKSQGMCF
ARPRRATLRAAMEWADVVHFMMPLPLEQVGRKMACQMNKPHTAAFHVQPENITSTLGLRRARQVNEALYA
WFRDSFYNHFTHVHCPSEFIAGQLRAHGYTARLHVISNGVDDCFYPRREEKQGPLAGKYAVLMIGRFSEE
KRQEVLLQAVRESRHAKQIQLVLAGQGPREKHLRKLGERLPNPPIMGFYSTRQLCRLMAMTDLYVHTAVA
EIEAIACLEAVASGLVPVIADSPLSATPQFALDGRSLFPADDAAALARKIDYWLENGAEREAMGRRYAES
AARYRLASSAQKAEEMFHEAIAEARG
>PWL87947.1 MAG: glycosyltransferase
[Oscillospiraceae bacterium]
(SEQ ID NO: 54)
MRLLIVVDQFDSGNNGTTISAQRFARALRERGHEVRVAAAGKPAEGKYALPRIHFLPLVDNIIKSQGMCF
ARPRRATLRAAMEWADVVHFMMPLPLEQVGRKMACQMNKPHTAAFHVQPENITSTLGLRRAKPVNEALYA
WFRDSFYNHFTHVHCPSEFIAGQLRAHGYTARLHVISNGVDDCFYPRREEKQGPLAGKYVVLMIGRFSEE
KRQEVLLQAVRESRHAKQIQLVLAGQGPREKHLRKLGERLPNPPIMGFYSTRQLCRLMAMTDLYVHTAVA
EIEAIACLEAVASGLVPVIADSPLSATPQFALDGRSLFPADDAAALACKIDYWLENGGEREAMGRRYAES
AARYRLAGSAQKAEEMFHEAIAEARG
>EEG28714.1 glycosyltransferase, group 1 family protein
[ [Clostridium] methylpentosum DSM 5476]
(SEQ ID NO: 55)
MVDIPGEKGKIMRILLVMDQLDDKNNGTTISAQRLAVTLREHGNEVRTVSVGEASHDRYSLKELKLLPGV
RGIVHNQGMLFAYPDRVVLEKAIRWADVVHFLMPFWVSSKGRRIAQRLGVPHTAAFHVQPENVSYNIGLG
KYEAVNSTIYAYFRQFYNQFTHVHCPSKFIARELKRHGYTAQLHVISNGVDRDFVYRKSPKPKQLEGKFV
ILMVGRLSNEKRQDVLIEAVRKSKYENRIQLLLAGRGPKQKKYESLAQGLTNRPIIGFYTKQELLDLLSI
CDLYAHAADIEIEAISCIEAFSSGLVPVIADSPKSATPQFALDGRSLFRAGDSGDLAAKIDYWIEHEEER
KRMEIVYSEHGKQYNIDACVGQMEEMFTAAIEENKCAR
>MCL2859922.1 MAG: glycosyltransferase
[Oscillospiraceae bacterium]
(SEQ ID NO: 56)
MKILLVLDQYDDCNNGTTVSAQRFAEGLTKRGHEVFIASTGKPAQNKFIVKPLPLPPGISWLIKSQGMVF
ALPTESVLKEAISKVDIVHFYLPFFLSRGGVKIAEELHIPHTAAFHSQPENITYTLGLGRSQKANDIIFN
YYRSFYNKFSHVHCPSQFIADELKSHNYKSQLHVISNGIQPEFKYIKSNKTDDLKDKFVITMVGRYSNEK
RQDLLFEAVNKSKYRDKIQVVLAGKGPNTKKYAKLAKTLTNEPVMKFFSKDDLVKMLSMTDLYVHASDAE
IEGISCIEAIACGIVPIIAKGKKAATPQFALDERSLFEAGNASDLADKIDYWLENPDERKKMEFEYAKSA
DKYSMNKSMEQIEEMFYEAIKECKEEQSDITQPAF
>HIU41948.1 MAG TPA: glycosyltransferase [Candidatus Egerieicola
faecale]
(SEQ ID NO: 57)
MKILLVSDQYYAANNGMTISARRFAGVLRQHGHEVRIMSYGTPDMVEDTDSAYLLDKYYVPIFNRLVTSQ
GMVFAKRTRKVVEAAVDWADLIHILSPFFLSHKTIRIAQKKNKPYTAAFHVQPQNITSSIYLGKVNWIND
LLYHFFHRYIYRYCTHIHCPSRFIAQQLVRTGYTEQLHVISNGIDPDFHYFKRHKPRELRDKFVILQTGR
LSIEKRPDVLIRAVAMSRHADQIQLVLAGKGPRKKKLQKLADKLLKNPVIIQFYSKPELIQLLGYCDLYV
HAADVEIEAMSCMEAFASGLVPVIANSPTSATPQFALDERSLFEVGNSKELAEKIDWWLEHPQEREQMEF
RYAELGKKYALEDCVRQAEAMFEQAVRENHGG
>MBD5102631.1 MAG: glycosyltransferase family 4 protein
[Subdoligranulum sp.]
(SEQ ID NO: 58)
MNYLFVLDQYGAENNGVSVSARRYAAVLRARGHGVRILSTGDCGPDGYSVPELRIPVFDKLIKSHGMVFG
KPEKRVLEQALAWADHVHFLMPFTLSIVTAQRARKMGKPATAAFHVQPQNISYSIGMGRWTPTNAFIYWL
FRTDFYGRFRYIHCPSAFIAKQLKAHGYGAECRVISNGITPEFTFRRDGKEAKWRGRFLILMIGRLSGEK
RQDVLIEAVKRSRHAQDIQLIFAGQGPLHDAYAKQARGLAHPLQMRFLSQEELRSVIAQCDLYVHTADAE
IEAMSCMEAFACGRVPVIANSPFSATPQFALDERSLFCPGDASDLAEKIDYWIEHPDERLAMERAYSESA
KKYAIENCVTQFEQLAAEAKEKGSWA
>MBD5094004.1 MAG: glycosyltransferase family 4 protein
[Subdoligranulum sp.]
(SEQ ID NO: 59)
MNYLFVLDQYGAENNGVSVSARRYAAVLRARGHGVRILSTGDCGPDGYSVPELRIPVFDKLIKSHGMVFG
KPEKRVLEQALAWADHVHFLMPFTLSIVTAQRARKMGKPATAAFHVQPQNISYSIGMGRWTPTNAFIYWL
FRTDFYGRFRYIHCPSAFIAKQLKAHGYGAECRVISNGITPEFTFRRDXKEAKWRGRFLILMIGRLSGEK
RQDVLIEAVKRSRHAQDIQLIFAGQGPLHDAYAKQARGLAHPLQMRFLSQEELRSVIAQCDLXVHTADAE
IEAMSCMEAFACGRVPVIANSPFSATPQFALDERSLFCPGDASDLAEKIDYWIEHPDERLAMERAYSESA
KKYAIENCVTQFEQLAAEAKEKGSWA
>WP_278885919.1 glycosyltransferase
[Ruthenibacterium lactatiformans]
(SEQ ID NO: 60)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLAHPLLRRRAAAG
>WP_205456931.1 glycosyltransferase [
Ruthenibacterium lactatiformans]
(SEQ ID NO: 61)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLERHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPNCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFVHFYPEGMLVPYHEGLRAFHPGAFTTAVRAGCPVVPMML
CRRPARGLWAWRKKPCFTLRIGAPLYADASLPKKQAARDLQLRAEAAMLALERGETPPLAVGEPALEAE
>WP_055080686.1 glycosyltransferase
[Ruthenibacterium lactatiformans]
(SEQ ID NO: 62)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFVHFYPEGMLVPYHEGLRAFHPGAFATAVRAGCPVVPMML
CRRPARGLWAWRKKPCFTLRIGAPLYADASLPKKQAARDLQLRAEAAMLALERGETPPLAVGEPALDAE
>EHL65771.1 hypothetical protein HMPREF1032_00988
[Subdoligranulum sp. 4_3_54A2FAA]
(SEQ ID NO: 63)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPEDIHGMAALERGMEARIRGGAFVHFYPEGMLVPYHEGLRAFHPGAFATAVRAGCPVVPMML
CRRPARGLWAWRKKPCFTLRIGAPLYADASLPKKQAARDLQLRAEAAMLALERGETPPLAVGEPALDAE
>WP_050005615.1 glycosyltransferase
[Ruthenibacterium lactatiformans]
(SEQ ID NO: 64)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFVHFYPEGMLVPYHEGLRAFHPGAFTTAVRAGCPVVPMML
CRRPARGLWAWRKKPCFTLRIGAPLYADASLPKKQAARDLQLRAEAAMLALERGETPPLAVGEPALDAE
>WP_155201477.1 glycosyltransferase
[Ruthenibacterium lactatiformans]
(SEQ ID NO: 65)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFVHFYPEGMLVPYHEGLRAFHPGAFTTAVRAGCPVVPMML
CRRPARGLWAWRKKPCFTLRIGAPLYADASLPKKQAARDLQLRAEAAMLALERGETPPLAVGEPALEAE
>MBD9256102.1 MAG: glycosyltransferase
[Ruthenibacterium lactatiformans]
(SEQ ID NO: 66)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPEDIHGMAALERGMEARIRGGAFVHFYPEGMLVPYHEGLRAFHPGAFTTAVRAGCPVVPMML
CRRPARGLWAWRKKPCFTLRIGAPLYADASLPKKQAARDLQLRAEAAMLALERGETPPLAVGEPALDAE
>RGC99060.1 glycosyltransferase
[Subdoligranulum sp. AM16-9]
(SEQ ID NO: 67)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPEDIHGMAALERGMEARIRGGAFVHFYPEGMLVPYHEGLRAFHPGAFATAVRAGCPVVPMML
CRRPARGLWAWRKKPCFTLRIGAPLYADAALPKKQAARDLQLRAEAAMLALERGETPPLAVGEPALDAE
>WP_270849115.1 glycosyltransferase
[Ruthenibacterium lactatiformans]
(SEQ ID NO: 68)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGIPTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYVSAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFMHFYPEGMLVPYHEGLRAFHPGAFATAVRAGCPVVPMML
CRRPARGLWAWRKKPCFTLRIGAPLYADASLPKKQAARDLQLRAEAAMLALERGETPPLAVGEPALDAE
>WP_205488614.1 glycosyltransferase
[Ruthenibacterium lactatiformans]
(SEQ ID NO: 69)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGISTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPSCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFVHFYPEGMLVPYHEGLRAFHPGAFATAVRAGCPVVPMML
CRRPARGLWAWRKKPCFTLRIGAPLYADASLPKKQAARDLQLRAEAAMLALERGETPPLAVGEPALDAE
>WP_119981398.1 glycosyltransferase
[Ruthenibacterium lactatiformans]
(SEQ ID NO: 70)
MKIVLVIDQFDDSNNGTTVTARRFAGQLRRRGHEVVILAGGAPCEGKICAPVHRIPVFQKLIESQGMCFA
KPDEEAYYTAFKDADIVHFYMPFRFCRRGEELARQMGISTVAAFHVQPENITSSIFLGKNRRVNDFLYWW
FYKVFYNRFDHIHCPSAFIARQLESHGYGAKLWVISNGVADAFRPAQVPRAPELEGRFTILMIGRLSGEK
RQDLIIEAAKRSKYADRLQLVFAGKGPKEKAYRKLAAGLAHPPVFGFYGQDELRRLINMCDLYVHASDAE
IEGISCMEALACGLVPVISDSPLSATGQFALCAESLFRAGDADDLARRIDYWVEHPEEKRAYAEQYALRQ
DENRVEACVARAEEMYASAIRDKRRKGYRKVPLSRWRRCTSPNCEAIRRNFCQGGTVRTLLFWLFTTLLS
PILWLLDRLWLGARIEGRENLDAVQGGAVSIMNHVHPLDCTMAKVALFPYRLWFISLASNLQKPFTGWLI
RFCGGVPLPDDIHGMAALERGMEARIRGGAFVHFYPEGMLVPYHEGLRAFHPGAFATAVRAGCPVVPMML
CRRPARGLWAWRKKPCFTLRIGAPLYADASLPKKQAARDLQLRAEAAMLALERGETPPLAVGEPALDAE
>MBS1326536.1 MAG: glycosyltransferase
[Oscillospiraceae bacterium]
(SEQ ID NO: 71)
MNIVLVIDQFDNGNNGTTITARRYAEQLRRLGHRVTILAGGEAAEGKICAPVHKIPVFQPLVEKQGFGFA
KPDEEAYYQAFRDADVVHFYLPFRFCRRGEEIARQMRVPTVAAFHMQPENVTYSIGMGKSKRINDFLYHW
CYRKFYNRFRYIHCPSEFIAGQLKAHGYDAELRVISNGVDPLFRPVDTPRPPEFEGKFVILMVGRLSGEK
RQDLIIEAAKKSRYADKIQLVFAGKGPKEKEYRRLSAGLAHPPIMGFYGQEELLRLLNSCDLYVHASDAE
IEGISCMEALACGLVPVIANSPLSATPQFALDDRSLFQAGNAGDLADKIDYWIEHPEERAAQGRAYAARG
DEMRVDACVARAEDMYREAIHDCRFHGYKPPRQGRLRRLTHPDPDKANQQFYHASALKKTVFGAVTNFLT
PLLLFIDGLFFGLRVEGRQHLRGIRGGAVTVMNHVHPMDCTMAKIAAFPHRQYFVSLRRNLELPFTGWLV
KLCGGLPLPPTASTMVPYQRHLEQAIRQGDFVHFYPEGLLVRYHKGLRPFHGGAFLTAARAGCPIVPMAV
VFRKPTGLRALFRRGDDMTLRIGEPLYPNPALGPKAAAQALQLRTRYAMEMLLGDSAGGYPLPATDWEEA
EDNI
>HIR53044.1 MAG TPA: glycosyltransferase
[Candidatus Onthovicinus
excrementipullorum]
(SEQ ID NO: 72)
MTIVIVIDTLALNNGTTMTAYRFANMLRAHGHTVRFVSTGPQEEGKFVVRERYFPLATPIARKQGIIFAR
SDRRVFRQAFEGADVVHLMMPFPFEHNAMRVAREMGIPCSTAFHVQPENITYITHTDALPRMNESVYRLL
YRMFYKDFHHIHCPSKFIAGQLEKNGYDAKLYVISNGVDAAFRPAEKKDTMHDDLFRILMIGRLSPEKRQ
NILIKAVSLSRHRDRIQLYLAGKGPRAEALRNMGKTLPHPPVIGFYSQEELIRLIHSCDLYVHASVVEIE
AISCMESFACGLVPVICDAPQSATRQFALDERSLFKPDDPRDLAAKIDYWIEHPEERQKMSVQYAREGDE
YRVERSVEKAEQMFREVIEDYRNGIL
>MCI8500750.1 MAG: glycosyltransferase [Oscillospiraceae bacterium]
(SEQ ID NO: 73)
MNIVLVIDQFDDGNNGTTVTARRYAHELRRLGHRVTILAGGEPAAHKICAPAHKIPFFEKLIESQGMQFA
KPDDRAYYEAFKDADVVHFYMPFRFCRRGEELARQLHIPTVAAFHVQPENITSSIGLGKRKNVNRFLYWW
FYHVFYNRFRLIHCPSRFIAGQLAAHNYDAELCVISNGVDDAFIPHPEWKAERKPEDVLNVLMIGRLSGE
KRQDLIIEAAKRSRYRDRIQLHFAGKGPKEAEYRRLSKGLPRPPVFGFYSTEELVGLINRCDLYIHSSDA
EIEGISCMEALACGLVPVISDSVLSATRQFALDERSLFKAGDPSDLARRMDYWFEHPEERLKMEAAYAQK
GEEMRVSACVRQADAMYCKAVEEQRKNGYVRPKELKIRRITHPDADRINKRYAKHSPVRRGLTRAFTNLL
AVILSVFDSLFLGFSIKGKEHLSSVEGGAVTVCNHVHPMDCTMVKIALFPRLVRYVSLRRNLELPLIGWI
LKACGVLPLPEHPIRIARFQKELEKGIAAGEWVHYYPEGMLVKYYEGLRPFQPGAFLTAVRANCPVIPLR
INYKQPHGPCALWRKRPFLELVVKAPLYADQTLPQKQAALDLMQRTLRAMGGVEEKDPAPGYLSEPEDAP
IAGTVT
>NLU26646.1 MAG: glycosyltransferase [Hungateiclostridium
thermocellum]
(SEQ ID NO: 74)
MIITLVNDTFNINNNGTTISAMRFAEALSQRGHQIRIITCGDPLKSGKDPDTGFEMFYLPELKIPIASRL
AHKQNTLFAKPVRSILKKAISGSDVVHIYQPWPLGSAAQRVARQMNIPAIAAFHIQPENITENIGLKRFS
PAAHLTYFLFYLFFYRRFSHIHCPSKFIAAQLRSHGYKARLHVISNGVHPAFCAPAKPREHTFKPIKILM
IGRLSPEKRQDVLIRAVMKSRYADRIQLYFAGSGPWEKKLRRLGNKLPNPPVFGYYNRDELIKLIHECDL
YVHASDAEIEGISLIEAFACGLVPIISDSKQSAAAQFALGPQNLFKAGSPESLAKKIDYWLDHPEQLKEA
EKKYAQLGKQYALEHSIRKIEKVYSSMTKNHKNEYHRSIFFRLSTRLFQIVIACPILLLWTRFVLGAKVY
GRENIRGLKSGVTVCNHVHLLDSALIGVTFFPRRVVFPTLTQNVKTLWPGKLVRILGGFAIPDNIMELKA
FFDEMEFLLMKNCIVHFFPEGELRPYDTGLQNFKKGAFYLAAQAQVPIVPMLITFEPPKGLIKIIRKKPV
MRLHIGKPIHPMSKDIEIDSELRMKAVCKKIEAITSV
>NJP40167.1 glycosyltransferase family 4 protein [Oscillospiraceae
bacterium HV4-5-C5C]
(SEQ ID NO: 75)
MHIVFVVDMYDVQKNGTTMTARRMAEALARRGHEVRVIYAASSDQDCIRPQAGDPVRLFSVRQLKVPVLY
QFMKAQHVTMGRPDEAVIRQALSGADVVHLFLPFPLEKQALRVAKELKIPVTAAFHLQPENVSYNIGLGR
SKSFNRAIYNYFNRYLYQYVSRVHCPSEFIARQLVAHQYKARLYVVSNGVPDQFKPAILPHPEPEAGAEI
PVIMVGRLSPEKNQALLLKAVLKSRFAKQIRVTLAGQGPDEAKLRRLGSRLPLAPEIGYYTQLELLQKLQ
TSILYIHTASAEIEAIACLEAVACGLVPLISNSDQSATPQFALDERSLFMPDSVDDLAQKLDYWLEHPGE
RQRMEQQYAKSAQAYRLDKVTAKLEQMLTEAVEYQHEPEAAGYL
>MBR4701844.1 MAG: glycosyltransferase [Oscillospiraceae bacterium]
(SEQ ID NO: 76)
MIICVVCDVLGKANNGTTMAALDLIQALKERGHLVRVVCPDLDRMGQENFYVVPVRNLGPLNNYLQKNGV
ELAKPERRILEQAMHGADHVHIMLPFPLGIKALKVAKEQGLPVTAGFHCQAENFTSHIFMKDFKPANTIT
YKTFYRLFYRHVDAVHYPTEFIRETFENATGKTNAYVISNGVRSAFQPAPSLRPKELEGRFVILFIGRYS
KEKAHKVLIDAAALSRHAKELQLIFAGSGPLREKLEKRSRKLANPPIFRFFSRDELVRAINYADLYVHPS
EVEIEAISCLEAISCGLVPVISDSYRSATRFFALDRKNLFTCNDPQALADRIDYWLEHPAERQERSRAYL
GYTVQFNYENCMDRMEQMIVETHDRVRSGKR
>MCI5886300.1 MAG: glycosyltransferase [Oscillospiraceae bacterium]
(SEQ ID NO: 77)
MDTFDRESGRDLPSLPGKAAQPIPQRRTAAMKVAMICDVMGQPNNGTTLAALNLIRYLRDAGHTVTVVAP
GGEASDGYLPVRVWHAGPLIDRILRMNGVELAVPDKQLLEAVIREADVVHLLIPLPLARAALKIARRLGK
PVTASFHCQAENITAHLGMMNAGWLNRLIYRNFYRKVYRWCTAVHYPTEFIREVFETATHPTPAHVISNG
VNDMFRLPDSRPENGKFTVVCSGRYSREKAQQQLLRAAALCRHRDDIRLILAGDGPRRKHYLRLAKQYGL
DCQFAFFPRQELLHILQTADLYVHTAIIEIEAIACTEAICCGLVPVICNSDRSATRFFACGDHTLFEPGD
VRALADLLDYWYEHPAARVERAAEYADLRHSFDQTACMQQMEQMLKEAAGL
>MBQ6431120.1 MAG: glycosyltransferase [Oscillospiraceae bacterium]
(SEQ ID NO: 78)
MTICVVCDVLGRENNGTTIAAMNLIRSLHAKGHTVRIVCPDKERKGEPDYFVVPTLKLGPFDPYVRKNGV
VIAKPRHKVLEAALDGVDHLHLMVPFMVSRFVLKMARKKGISVTAGFHCQAENLTSHLFLKNNRLANRLV
YRNFYRRFYRYVDGVHFPSEFIHGVFERYGGKTNAYVISNGVNKQFKPMEVERPKELEGKRVILFTGRES
KEKSHTVLIDAAKMSRHVDELQLVFAGDGPLKKKLEKRGRGLRNPPIFRFFSREELLRVINSADLYVHPA
EIEIEAISCLEAISCGLVPVISNSERSATRFFALSERNLFPCNDSAALAERIDYWLEHPEEKEACSREYL
GYTKQFDFDVCMDRMEQMILETYENKHKA
>MBQ1404831.1 MAG: glycosyltransferase [Oscillospiraceae bacterium]
(SEQ ID NO: 79)
MTICIVCDVLGRENNGTTIAAMNLIRSLRAKGHSVRIVCPDRERSGEPDCYIVPTLPLGPLNGYVRKNGV
VIARPSRKVLEAALSGVDHVHLMLPFPVSCAMLRLARERGISVTAGFHCQAENFTSHIFMKDSRLVNRLV
YKAFYRHCYRYVDAVHYPTEFIREVFERYGGKTNAWVISNGVNRQFRPIPVSRPEELEGKYVILFIGRLS
KEKSHRVLIDAAKQSRHAEELQLVFAGDGPLKEKLMKRSKALKNPPIFRFFSREELVKTINSADLYVHPA
EIEIEAISCLEAISCGLVPVISDSPRSATRFFALGEQNLFHSNDSKALAERIDWWLEHPAEQEACSKAYL
GYTKQFDFDACMDRMEQMILETHAAKHKEG
>MBQ2178194.1 MAG: glycosyltransferase [Oscillospiraceae bacterium]
(SEQ ID NO: 80)
MTICIVCDVLGRENNGTTIAAMNLIRSLRAKGHSVRIVCPDRERSGEPDCYIVPTLPLGPLNGYVRKNGV
VIARPSRKVLEAALSGVDHVHLMLPFPVSCAMLRLARERGISVTAGFHCQAENFTSHIFMKDSRLVNRLV
YKAFYRHCYRYVDAVHYPTEFIREVFERYGGKTNAWVISNGVNRQFRPIPVSRPEELEGKYVILFIGRLS
KEKSHRVLIDAAKQSRHAEELQLVFAGDGPLKEKLMKRSKALKNPPIFRFFSREELVKTINSADLYVHPA
EIEIEAISCLEAISCGLVPVISDSPRSATRFFALGEQNLFRCNDSKALAERIDWWLEHPAEQETCSKAYL
GYTKQFDFDACMDRMEQMILETHAAKHKEG
>MCR5652922.1 MAG: glycosyltransferase [Ruminococcus sp.]
(SEQ ID NO: 81)
MKITVVCDVLGAENNGTTIAAMNLIRAMNKRGHEVTVVCSDEDKKGKEGYVVMPKLNLGPLNNYVSKNGV
SLSRANKKVLEPVIKNSDVVHIMIPFMLGAAAVKLCRKYNIPVTAGFHCQAENFTSHIFMKDNSLANQIT
YNSIYRSVYRYVDAVHYPTEFIRNVFEQAVHKRTNGYVISNGVSERFKPIKTEKPPEFSNRFVILFSGRY
SKEKSHSVLIDAAALSKHSREIQLVFAGDGPLKQKLKEQAKKLPNRPLFNFFPHAQMLNVLNYADLYVHP
AEIEIEAIACMEALACGQVPIISDSPRSATVKFALDERNLFKNRDPEDLAKKIDFFIDNPDVLEEYRERY
KGIVAEFSQKVCMDKMEKMFYEALR
>MBQ3356221.1 MAG: glycosyltransferase [Oscillospiraceae bacterium]
(SEQ ID NO: 82)
MTICVVCDVLGRENNGTTIAAMNLIRSLRAKGHAVRVVCPDAERKGEPDCFVVPTLNLGPINAYVRKNGV
VIAKPERKVLEAALDGVDHVHLMVPFAVSRAALKLARERGLSVTAGFHCQAENFTSHIFMKNNRLANLLT
YRNFYQHCYRWVDGVHYPSEFIRGVFERYGGKTNAYVISNGVNRQFRPMQVERPKELEGKRVILFTGRYS
KEKSHTVLIDAAKLSRHADELQLVFAGDGPLKKKLEKRSRGLKHPPIFRFFSREELLQVINSADLYVHPA
EIEIEAISCLEAISCGLVPVISDSERSATRFFALREENLFPCNDSRTLATRIDYWLEHPQEKEKCSRAYL
GYTKQFDFDVCMDRMEQMILETHENKHRA
>MBR3185621.1 MAG: glycosyltransferase [Oscillospiraceae bacterium]
(SEQ ID NO: 83)
MTIAVICDVLGKENNGTTIAAMNLIRSLREKGYDVRVVCSDPERAGQKDFYVVPTLNLGPLNAYVAKNGV
ALANPDRNILTAALNGVDHVHVMMPFALGSAAARLAHELGLPLTAGFHCQAENFTGHIFMKNFSPANRIA
YHVFYRNLYRYCDCIHYPSQFICDTFEAIVGPTQHRIISNGVNQIFQPKPAEKPKELKGRFVVLFTGRYS
PEKSHKVLIEAVARSRYKDEIQLIFAGDGPLRQSLARQAKRRGVHDPIMRFYSREELVNVINYADLYVHP
AEIEIEAIACLEAIACGKVPLIADSPRSATRYFALTDRNLFRYNDPQDLADRMDWWLEHPEERLSCSKSY
LGYAAQFDFTSCMDQMEKMILDVSEARHES
>MBQ1589099.1 MAG: glycosyltransferase [Oscillospiraceae bacterium]
(SEQ ID NO: 84)
MTIAVICDVLGKENNGTTIAAMNLIRSLREKGYDVRVVCSDPERAGQKDFYVVPTLNLGPLNAYVAKNGV
ALANPDRNILTAALNGVDHVHVMMPFALGSAAARLAHELGLPLTAGFHCQAENFTGHIFMKNFSPANRIA
YHVFYRNLYRYCDCIHYPSQFICDTFEAIVGPTQHRIISNGVNQIFQPKPAEKPKELKGRFVVLFTGRYS
PEKSHKVLIEAVARSRYKDEIQLIFAGDGPLRQSLARQAKRRGVHDPILRFYSREELVKVINYADLYVHP
AEIEIEAIACLEAIACGKVPLIADSPRSATRYFALTDRNLFRYNDPQDLADRMDWWLEHPEERLSCSKSY
LGYAAQFDFTSCMDQMEKMILDVSEARHES
>MCD8116392.1 MAG: glycosyltransferase [Oscillospiraceae bacterium]
(SEQ ID NO: 85)
MNILFVINNLYSDGNGLCASARRTIGLLRERGPEVRVLSACGPDGQTPDYPLPDWHMPLFGPLVARQGYR
FAARDKAVIRQALEWADLVHLEEPFFLQMTVCRMARSMGVPCVATYHLHPENFFASVGLQRSRYFNAATL
WVWRKYVYDHCAIVQCPTERVRERLARRGFRAELRVISNGLLPEERPGAGPHIHTPGEPYNILCVGRYSE
EKDQMTLLRAMEYTKHAHEIRLILAGRGPKEEKLRAAAEKLCRDGVLTIPPVFGFYSAAGLEELFAQADL
YIHCATVEVEGLSCMEAVRTGIVPLIADGPLTATAQFALSRESRFPVGDAKALAAGIDYWLSDDEARRRE
AARYTALGEQYAIARSIDALVQMYRDALTAPGVR
>MBQ6755393.1 MAG: glycosyltransferase [Oscillospiraceae bacterium]
(SEQ ID NO: 86)
MRIAVICDVLGDKNNGTSIVAYNLIDFLRQKGHEVRIVCPDEEHTGEEGYWVVPKMHFGIFQSYVDRNGV
APAKLDKRILYDAIHDVDLIHVMIPFALGKAAAKYANQHGIPLTAGFHCQAENITSHIFLKDSRFANRVT
YKILNERLYKYCDGIHYPTEFIRDTFERIVGTTPHFVISNGVGDEFRPMKAERPEALRDKYILLFTGRLS
REKSHNVLIDAVAKSKHRDKIQLFFAGEGPLERELLAHAERVGISAPVIKFYSRAKLLNIINMADLYVHP
AEIEIEAISCLEAIACGKVPLIADSPRSATRFFALSEKNLFKSGDAVDLSHKLDFWLDHPDERKKCSEEY
LGYAERFRLKDCMEAMEDMLKKVANEKKKDCLVR
>MBQ9046124.1 MAG: glycosyltransferase [Oscillospiraceae bacterium]
(SEQ ID NO: 87)
MKIAVICDVLGKENNGTTIAAMNLIRSLRAKGHNVRVVCADPERAGQQDYYIVPKMNFGPINGYVAKNGV
APARVDRAILEAAIADVDLIHVMMPFAVGCAAAAYAHEHHIPLTAGFHCQAENVTGHIFMMDFPLANTIA
YKTFYHKLYRYCDCIHYPSQFICDTFEGIVGPTPHRIISNGVSSTFQPIPVEKPEALRGRFVVLFTGRYS
PEKSHKVLIDAVALSQHKDEIQLIFAGTGPLKLTLQRQARRRGIPQPIMEFYDRNTLIDMINSADLYVHP
AEIEIEAISCLEAIACGRVPLIADSPRSATRYFALSDDNLFRYDDPADLAQKMDWWLEHPEERERCSQAY
LGYAKQFDFQTCMDAMENMMLDTVEKKRHEK
>MCI8619273.1 MAG: glycosyltransferase family 4 protein
[Oscillospiraceae bacterium]
(SEQ ID NO: 88)
MILTVVCDVLGEENNGTTIAAMNLIRSMRQKGYTVRVVCPDESRQGQPGFFIVPTYNLRVFNGYVQKNGV
TLAKPVQSILEAAIVGADVVHIIVPFALGRAALSLAKAQGIPVTAGFHCQAENITNHIFLMNANLANRLI
YKVFYRSFYQYCDCIHYPTQFICDLFEKETKPTNHYVISNGVNRSFVRRDCPKPEELQDKFVILSTGRYS
KEKSQQILLRAVALSKFKDKIQLILAGKGPRQAFLEQEAEKLGLLPPIFRFFSREALIDVINYADLYVHP
AEIEIESIACLEAIACGKVPIIADSPRSAARNFALTPENLFACNDPADLAKRIDGWLSDPQARAACSEKY
LGYAQAFAFDRCMEQMEQMLLDAVEGKRHG
>MBQ2211465.1 MAG: glycosyltransferase [Ruminococcus sp.]
(SEQ ID NO: 89)
MKITVVCDVLGVENNGTTISAMNLIRAMAKRGHEVTVVCSDEDKRGKDGYVVMPTLSLGPLNNYVSKNGV
SLSRANKRVLKPVIQNSDVVHVMMPFMLGTAAVKLCKKYDIPVTAGFHMQAENFTSHVEMKDNPLANQIT
YHTIYRNVYRHVDAVHYPTEFIRNVFEQAVHHRTNGYVISNGVGERFKPIKIEKPAEFANRFVIIFSGRY
SKEKCHSVLIDAAALSKHSRDIQLIFAGEGPLKQKLRERAKKLYNKPLFNFFPHDQMLNVLNYADLYVHP
AEIEIEAIACMEALACGQVPIIADSPRSATVKFALDERNLFKNRDAQDLADKIDFFIEHPDVLEEYRERY
KGIVADFSQEACMDKMEKMFYEAMK
>MBR4472306.1 MAG: glycosyltransferase [Oscillospiraceae bacterium]
(SEQ ID NO: 90)
MTIAVICDVLGKENNGTTIAAMNLIRSLREKGYDVRVVCSDPERAGQKDFYVVPTLNLGPLNAYVAKNGV
ALANPDRNILTAALNGVDHVHVMMPFALGSAAARLAHELGLPLTAGFHCQAENFTGHIFMKDFPLANRIA
YHVFYRNLYRYCDCIHYPSQFICDTFEAIVGPTPHRIISNGVNHIFQPKPAEKPKELKDRFVVLFTGRYS
PEKSHKVLIEAVARSRYKDEIQLIFAGDGPLRQRLARQAKRQGVHDPIMRFYSREELVNVINYADLYVHP
AEIEIEAIACLEAIACGKVPLIADSPRSATRHFALTDHNLFHYNDPQDLADRMDWWLEHPEERLSCSKSY
LGYAAQFNFTSCMDQMEKMILDVSEGRHES
>MBQ2427448.1 MAG: glycosyltransferase [Ruminococcus sp.]
(SEQ ID NO: 91)
MKITVVCDVLGVENNGTTISAMNLIRAMAKRGHEVTVVCSDEDKRGKDGYVVMPTLSLGPLNNYVSKNGV
SLSRANKRVLKPVIQNSDVVHVMMPFMLGTAAVKLCKKYDIPVTAGFHMQAENFTSHVEMKDNPLANQIT
YHTIYRNVYRHVDAVHYPTEFIRNVFEQAVHHRTNGYVISNGVGERFKPIKIEKPAEFANRFVIIFSGRY
SKEKCHSVLIDAAALSKHSRDIQLIFAGEGPLKQKLRERAKKLYNKPLFNFFPHDQMLNVLNYADLYVHP
AEIEIEAIACMEALACGQVPIIADSPRSATVKFALDERNLFKNRDAQDLADKIDFFIKHPDVLEEYRERY
KGIVADFSQEACMDKMEKMFYEAMK
>MBQ6316523.1 MAG: glycosyltransferase [Oscillospiraceae bacterium]
(SEQ ID NO: 92)
MTIAVICDVLGKENNGTTIAAMNLIRSLREKGYDVRVVCSDPERAGQKDFYVVPTLNLGPLNAYVAKNGV
ALANPDRNILTAALNGVDHVHVMMPFALGSAAARLAHELGLPLTAGFHCQAENFTGHIFMKDFPLANWIA
YHVFYRNLYRYCDCIHYPSQFICDTFEAIVGPTPHQIISNGVNHIFQPKPAEKPKELKDRFVVLFTGRYS
PEKSHKVLIEAVARSRYKDEIQLIFAGDGPLRQSLARQAKRRGVHDPIMRFYSREELVKVINYADLYVHP
AEIEIEAIACLEAIACGKVPLIADSPRSATRHFALTDHNLFHYNDPQDLADKMDWWLEHPEERLSCSKSY
LGYAAQFNFTSCMDQMEKMILDVSEGRHES
>MBR6424427.1 MAG: glycosyltransferase [Oscillospiraceae bacterium]
(SEQ ID NO: 93)
MIVAVICDVLGEENNGTTIAAMNLIRSLQAKGHEVRVVCADEARAGQAGFFLVPKFNFGIFNAYVQKNGV
APARVDHKVLEQAIHDADIIHVMLPFGLGKAAAQYASEHGIPLTAGFHCQAENITSHIFLKDFAWGNRAV
YRILYRRLYRYCDCIHYPTQFICDTFEQVVGPTPHRIISNGVDRAFQPMTVPRPKTLEGKFVVLFTGRFS
REKAHSVLIRAAAKSRHRDKLQLIFAGSGPLEAELRQEAEQAGILRPIMRFYSRAELIELINQANLYVHP
AEIEIEAISCLEAISCGKVPLIADSPRSATRYFALSDRNLFRNRDPQDLADKLDWWIEHPEEIEQAQKAY
LGYARQFDFDLCMDNMERMLLETWEAKHAAG
>MBQ9679709.1 MAG: glycosyltransferase [Ruminococcus sp.]
(SEQ ID NO: 94)
MKITVVCDVLGAENNGTTIAAMNLIRALKAKGHDVIVVCSDEDKKGKNGYVVMPQLNLGPLNGYVKKNGV
ALSRADNKVLEPVIWDSDVVHIMLPFALGRAALKLCLKHNIPVTAGFHMQAENFTSHVFMKDNPVANNLT
YRYIYNNLYKYVNAVHYPTDFIREVFENAVGHKTNAYVISNGVSDRFKPNGAKKPPEFRNKFVILFSGRY
SKEKSHSVLIDAAALSKHSSEIQLIFAGDGPLKDMLKERSKKLPNKPVFSFFPHHQMLNVLNYADLYVHP
AEIEIEAIACMEALSCGQVPIIANSPRCATKKFALDERNLFENRNPQDLADKIDYFIEHPEAIEEYRERY
KGIVAELSQEACMNRMEEMLYEARG
>ABN52751.1 glycosyl transferase group 1 [Acetivibrio thermocellus
ATCC 27405]
(SEQ ID NO: 95)
MLFLLTVFFFVFEKLKVNAARKERGVKFLMIITLVNDTFNINNNGTTISAMRFAEALSQRGHQIRIITCG
DPLKSGKDPDTGFEMFYLPELKIPIASRLAHKQNTLFAKPVRSILKKAISGSDVVHIYQPWPLGSAAQRV
ARQMNIPAIAAFHIQPENITFNIGLKRFSPAAHLTYFLFYLFFYRRFSHIHCPSKFIAAQLRSHGYKARL
HVISNGVHPAFCAPAKPREHTFKPIKILMIGRLSPEKRQDVLIRAVMKSRYADRIQLYFAGSGPWEKKLR
RLGNKLPNPPVFGYYNRDELIKLIHECDLYVHASDAEIEGISLIEAFACGLVPIISDSKQSAAAQFALGP
QNLFKAGSPESLAEKIDYWLDHPEQLKEAEKKYAQLGKQYALEHSIRKIEKVYSSMTKNHKNEYHRSIFF
RLSTRLFQIVIACPILLLWTRFVLGAKVYGRENIRGLKSGVTVCNHVHLLDSALIGVTFFPRRVVFPTLT
QNVKTLWPGKLVRILGGFAIPDNIMELKAFFDEMEFLLMKNCIVHFFPEGELRPYDTGLQNFKKGAFYLA
AQAQVPIVPMLITFEPPKGLIKIIRKKPVMRLHIGKPIHPMSKDIEIDSELRMKAVCKKIEAITSV
>WP_000237258.1 glycosyltransferase family 4 protein [Helicobacter
pylori]
(SEQ ID NO: 96)
MVIVLVVDSFKDTSNGTSMTAFRFFEALKKRGHAMRVVAPYVDNLGSEEEGYYNLKERYIPLVTEISHKQ
HILFAKPDEKILRKAFKGADMIHTYLPFLLEKTAVKIAREMQVPYIGSFHLQPEHISYNMKLGQFSWFNM
MLFSWFKSSHYRYIHHIHCPSKFIVEELEKYNYGGKKYAISNGFDPMFKCEHPQKSLFDTIPFKIAMVGR
YSNEKNQSVLIKAVALSRYKQDIVLLLKGKGPDEKKIKLLAQKLGVKTEFGFVNSNELLEILKTCTLYVH
AANVESEAIACLEAISVGIVPVIANSPLSATRQFALDERSLFEPNNAKDLSTKIDWWLENKLERERMQNK
YAKSTLNYTLENSVIQIEKVYEEAIRDFKNNPHLFKTLS
>WP_003517717.1 glycosyltransferase [Acetivibrio thermocellus]
(SEQ ID NO: 97)
MVITLVNDTFNINNNGTTISAMRFAEALSQRGHQIRIITCGDPLKSGKDPDTGFEMFYLPELKIPIASRL
AHKQNTLFAKPVRSTLKKAISGSDVVHIYQPWPLGSAAQRVARQMNIPAIAAFHIQPENITENIGLKRFS
PAAHLTYFLFYLFFYRRFSHIHCPSKFIAAQLRSHGYKARLHVISNGVHPAFCAPAKPREHTFKPIKILM
IGRLSPEKRQDVLIRAVMKSRYADRIQLYFAGSGPWEKKLCHLGNKLPNPPVFGYYNRDELIKLIHECDL
YVHASDAEIEGISLIEAFACGLVPIISDSKQSAAAQFALGPQNLFKAGSPESLAEKIDYWLDHPEQLKEA
EKKYAQLGKQYALEHSIRKIEKVYSSMTKNHKNEYHRSIFFRLSTRLFQIVIACPILLLWTRFVLGAKVY
GRENIRGLKSGVTVCNHVHLLDSALIGVTFFPRRVVFPTLTQNVKTLWPGKLVRILGGFAIPDNIMELKA
FFDEMEFLLMKNCIVHFFPEGELRPYDTGLQNFKKGAFYLAAQAQVPIVPMLITFEPPKGLIKIIRKKPV
MRLHIGKPIHPMSKDIEIDSELRMKAVCKKIEAITSV
>WP_003813888.1 glycosyltransferase [Bifidobacterium bifidum]
(SEQ ID NO: 98)
MRDNKNEEPRPQDRSVLDRPMTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAAGQFALLDESLFPARDVAMLARRID
WWIDHPHELAVWGGRYAEHAKADYSVEASVAKFVAMEREAIADAKA
>WP_003815502.1 glycosyltransferase [Bifidobacterium bifidum]
(SEQ ID NO: 99)
MRDNKNEEPRPQDRSVLDRPMTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_003819341.1 glycosyltransferase [Bifidobacterium bifidum]
(SEQ ID NO: 100)
MRDNKNEEPRPQDRSVLDRPLTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_007057736.1 glycosyltransferase [Bifidobacterium longum]
(SEQ ID NO: 101)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVARQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVIASGRLASEKDQITLIKAVSMSRHAGDIQLVIAGTGPLKQYLKFRAGRLLARKADIGFHKHADMPDLL
RSGDLFVHCSIADIESVSVIEAMACGLVPVIAASELSAASQFALIDESLFPVRDAALLARRIDWWIAHQA
ERAEWGEKYAEYTKAHYSVEASVHKFVDMERKAIGE
>WP 013363698.1 MULTISPECIES : glycosyltransferase [Bifidobacterium]
(SEQ ID NO: 102)
MRDNKNEEPHPQDRSVLDRPMTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_013390098.1 glycosyltransferase [Bifidobacterium bifidum]
(SEQ ID NO: 103)
MRDNKNEEPRPQDRSALDRPMTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_015359397.1 glycosyltransferase [Thermoclostridium stercorarium]
(SEQ ID NO: 104)
MGMRIVFVIDSFNLNNGTAATARRYAEELRKRGHEVTILAAGSADGNKTGIRKLRIPFFQPLIEKQGFCF
ARPNDEAYYRAFRNADIIHFFLPTWFCRRGEFIARQMRIPTVSAFHLQPENVTYSIGLGKSKRANDVIYR
YFYKCFYNRFRFIHCPSEMIAEQLMQHGYDAECRVISNGVNDLFRPAEVRRPEYLKGKIVLLMVGRLSGE
KRQDLLIEAVQYSKYRDKIQLVFAGMGPKEKKYRKLSRNLKNKPIFRFFTQEELLQMYNICDLYIHTSDA
EIEGISCIEAMACGAVPVISDSHLSATKSFALHPNCLFKAGDARSLAEKIDYWIEHPEERSKMSRIYAEK
AETIRVSKCVAEMERLYKDVISDYHKNGYKQPEKSRLRKLLHPDTDAVNVAYSKRTPARQALFYVFTNLI
AIIIYFIDTVFFGLVIEGKDKLKKVRGGAVTVMNHIHPMDCTMVKLAVFPRPIYFTSLVNNLELPLVGWL
IRFCGALPVPNGKGKLVGFMKHIKHGIQNGDLVHFYPEGMLIRNYEGLREFQPGAFYTAVHTGCPVIPMV
LVNIHPQGIWRIAGGRRMHLFIGEPQYPNSELSPKESVTELKNRTWQIMNEMMNEPVEAERISFSVAVRI
ACILYIASQVVRIIAIRL
>WP_021648620.1 glycosyltransferase [Bifidobacterium bifidum]
(SEQ ID NO: 105)
MRDNKNEEPHPQDRSVLDRPMTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIVCGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_023062492.1 glycosyltransferase [Acetivibrio thermocellus]
(SEQ ID NO: 106)
MIITLVNDTFNINNNGTTISAMRFAEALSQRGHQIRIITCGDPLKSGKDPDTGFEMFYLPELKIPIASRL
AHKQNTLFAKPVRSILKKAISGSDVVHIYQPWPLGSAAQRVARQMNIPAIAAFHIQPENITENIGLKRFS
PAAHLTYFLFYLFFYRRFSHIHCPSKFIAAQLRSHGYKARLHVISNGVHPAFCAPAKPREHTFKPIKILM
IGRLSPEKRQDVLIRAVMKSRYADRIQLYFAGSGPWEKKLRRLGNKLPNPPVFGYYNRDELIKLIHECDL
YVHASDAEIEGISLIEAFACGLVPIISDSKQSAAAQFALGPQNLFKAGSPESLAEKIDYWLDHPEQLKEA
EKKYAQLGKQYALEHSIRKIEKVYSSMTKNHKNEYHRSIFFRLSTRLFQIVIACPILLLWTRFVLGAKVY
GRENIRGLKSGVTVCNHVHLLDSALIGVTFFPRRVVFPTLTQNVKTLWPGKLVRILGGFAIPDNIMELKA
FFDEMEFLLMKNCIVHFFPEGELRPYDTGLQNFKKGAFYLAAQAQVPIVPMLITFEPPKGLIKIIRKKPV
MRLHIGKPIHPMSKDIEIDSELRMKAVCKKIEAITSV
>WP_026642518.1 glycosyltransferase [Bifidobacterium tsurumiense]
(SEQ ID NO: 107)
MNRDREVPSGQRENGDIDVRPLTIALVVDTIGNHGNGTSNSALQYAQELERQGHHVRLVGIGSQEYPAKV
NRIPLVSHIAARQQMQFAKPSDTLFRTAFAGVDVVHIYMPFSFGRRARIIAQEMGIPVTAGFHVQPENVL
YSAGPLRFVPGASRFIYWLFKRWLYRYVDHIHTPTQMIAEELRKHHYDAKLHAISNGYSPRFVPRQPRAA
ADIRPPYRVAASGRLSLEKSQITLIKAISLCRHGSDIELSICGTGPMRRYLQMRAKRLLNSPVRIGFQPN
DRMPGLLRSQDLLVHASVVDIESLSVMEGIGSGVVPIIARSGLSAASQFALTDQSLFEARNAQQLADRID
WWLDHPMELVSWERKYARFAAQRYSVESSVRQFIEMERESIAEYESSH
>WP_029576019.1 glycosyltransferase [Bifidobacterium
thermacidophilum]
(SEQ ID NO: 108)
MKNQEFSQAAGSTEDAGRPLVIALVVDTAGNEGNGTSNSALQYAKELRRQGHTVRLVGIGSPEYPIRVRH
IPLVSWIAAKQQMQFAQPDRAMFRKAFEGADVVHVYLPFKYGRMAVRTAHEMGIPATAGFHLQPENVLYS
AGPLRYIPGASAFIYWLFDRILYRHIRHIHTPSHMIAAQLRAHGYRQRLHVISNGYSPRFTAKQQREPGS
PSPRPFRIVASGRLASEKDHVTLIRAVALSRHAEDIELSICGTGPLQHELRRMAAQLLPGRWSIGFHDNA
QMPVFLRSCDLLVHPSIVDIESLSVLEGMASGLVPVIARSTQSAAGQFALCDHSLFEAQDPAMLAQRIDW
WIDHPDELSRWGAIYAEHTREEYSIESCVRKFVAMEREAIADFDSGSRMTGAI
>WP_029679177.1 glycosyltransferase [Bifidobacterium longum]
(SEQ ID NO: 109)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVAHQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVIASGRLASEKDQITLIKAVSMSRHAGDIQLVIAGTGPLKQYLKFRAGRLLARKADIGFHKHADMPDLL
RSCDLFVHCSIADIESVSVIEAMACGLVPVIAASELSAAGQFALIDESLFPVRDAALLARRIDWWIAHQA
ERAEWGEKYAEYTKVHYSVEASVHKFVDMERKAIGE
>WP_032684134.1 glycosyltransferase [Bifidobacterium longum]
(SEQ ID NO: 110)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVAHQMGIPVTAGFHLQPENVLYSAGPLRR
IPGISSFLYWLFKHWLYKHIDHIHVPTEMTASLLRAHGYKAKLHVISNGYSPVYSPKKPADPDAPAPVPF
RVVASGRLASEKDQITLIKAVSMSRHAGDIQLVIAGTGPLKQYLKFRAGRLLARKADIGFHKHADMPDLL
RSCDLFVHCSIADIESVSVIEAMACGLVPVIAASELSAAGQFALIDESLFPVRDAALLARRIDWWIAHQA
ERAEWGEKYAEYTKVHYSVEASVHKFVDMERKAIGE
>WP_033490312.1 glycosyltransferase [Bifidobacterium boum]
(SEQ ID NO: 111)
MSQSSKFTRATGVNGDADRPLTIALVVDTAGNEGNGTSNSALQYARELRRQGHTVRLVGIGSPEYPIRVH
HIPLVSWIAAKQQMQFAQPDRAVFRKAFVGVDVVHVYLPFKYGRVAVQTAHEMGIPATAGFHLQPENVLY
SAGPLRYIPGASAFIYWLFDRMLYRHIRHIHTPSHMIAAQLRAHGYRQRLHVISNGYSPRFTPKRQREPG
SPSPRPFRIVASGRLASEKDHVTLIRAVALSRHAEDIELVICGTGPLQHELRRMAGKLLPGRWSIGFHDN
AQMPELLRSCDLLVHPSIVDIESLSVLEGMASGLVPVIARSTQSAAGQFALNDHSLFEAQDSAMLAQRID
WWIDHPDELSRWGAVYAEHTREEYSIASCVRKFVAMEREAIADFDGRL
>WP_033889606.1 glycosyltransferase [Bifidobacterium saguini]
(SEQ ID NO: 112)
MRENGSEESIDRPLTIALVVDTVGNQGNGTSNSALQWAAELERQGHHVRLVGVGAPEYPARVNKVPLVSW
VASKQLMQFAQPSDTLFRTAFQGVDVVHIYMPFKFGRHAAKVARQMGIPVTAGFHLQPENVLYSAGPLRH
IPGVSSFLYWLFKHWLYRRIDHIHVPTEMTASLLRAHGYKAKLHVISNGYSPAYKPLKSVDPAAPTPVPF
RIIASGRLAREKDQITLIKAISMSRHAADIQLVIAGTGPLRRYLSFRAGRRLARKADIGFHKHAEMPELL
RSGDLFVHCSIADIESVSVIEAMACGLVPVIAASELSAASQFALLDESLFPVRDAALLARRIDWWISHQQ
ERAEWSAKYAENTKAHYSVEASVHKFVDMEREAIRE
>WP_034526977.1 glycosyltransferase [Bifidobacterium
stellenboschense]
(SEQ ID NO: 113)
MREQENAEEFGKTSIVEEDIDRPLTIALVVDTVGNQGNGTSNSALQWAAELERQGHHVRLVGVGAPDYPA
RVNRVPLVSWVAAKQQMQFAEPSDTLFRTAFQGVDVVHIYMPFKFGRRAAKIARQMHIPVTAGFHLQPEN
VLYSAGPLRYIPGMEKFLYWLFRQWLYKRVDHIHTPTEMTASLLREHGYKGTMHVISNGYSPRFTARKPL
DSDAPAHVPFRVVASGRLAHEKDQITLIKAISMSRHASDIQLVIAGTGPLKRYLTFRANRRLKRKADIGF
HRNADMPALLRSCDLFVHCSIADIESVSVIEAMACGLVPVIAASELSAASQFALTDASLFPVRDAAMLAR
RIDWWIDHPDELARWGEIYAEHTREHYSVEASVHRFVDMEREAIATAGR
>WP_044089922.1 MULTISPECIES: glycosyltransferase [Bifidobacterium]
(SEQ ID NO: 114)
MHENGTGEESIDRPLTIALVVDTVGNQGNGTSNSALQWAAELERQGHHVRLVGIGAPEYPARVNKVPLVS
WVASKQLMQFAQPSDTLFRTAFRGVDVVHVYMPFKFGRHAAKVARQMGIPVTAGFHLQPENVLYSAGPLR
HIPGISGFLYWLFKHWLYKRIDHIHVPTEMTASLLRSHGYQAKLHVISNGYSPVYTPKPPAESSEDPDAA
SPVPFRIIASGRLAREKDQITLIKAVSMSRHAADIQLVIAGTGPLKHYLKFRAGRLLARKADIGFHKHAD
MPELLRSGDLFVHCSIADIESVSVIEAMACGLVPVIAASELSAASQFALLDESLFPVRDAALLARRIDWW
IAHQRERVAWGARYAQNTKEHYSVEASVHKFVDMEREAIEE
>WP_044098601.1 glycosyltransferase [Bifidobacterium porcinum]
(SEQ ID NO: 115)
MKNQEFSVTAGSTEDAGRPLVIALVVDTAGNEGNGTSNSALQYAGELRRQGHTVRLVGIGSPEYPIRVRH
IPLVSWIAAKQQMQFAQPDRAMFRKAFEGADVVHVYLPFKYGRMAVRTAHEMGIPATAGFHLQPENVLYS
AGPLRYIPGASAFIYRLFDRMLYRHIRHIHTPSHMIATQLRAHGYRQRLHVISNGYSPRFTAKQQREPGS
PSPRPFRIVASGRLASEKDHVTLIRAVALSRHAEDIELSICGTGPLQHELRRMAAQLLPGRWSIGFHDNA
QMPVFLRSCDLLVHPSIVDIESLSVLEGMASGLVPVIARSTQSAAGQFALCDHSLFEAQDPAMLAQRIDW
WIDHPDELSRWGAVYAEHTREEYSIESCVRKFVAMEREAIADFDGGSRMTGAI
>WP_044282439.1 glycosyltransferase [Bifidobacterium thermophilum]
(SEQ ID NO: 116)
MKNQEFSVAAGSTEDAGRPLVIALVVDTAGNEGNGTSNSALQYAKELRRQGHTVRLVGIGSPEYPIRVRH
IPLVSWIAAKQQMQFAQPDRAMFRKAFEGADVVHVYLPFKYGRMAVRTAHEMGIPATAGFHLQPENVLYS
AGPLRYIPGASAFIYWLFDRMLYRHIRHIHTPSHMIAAQLRAHGYRQRLHVISNGYSPRFTAKQQREPGS
PSPRPFRIVTSGRLASEKDHVTLIRAVALSRHAEDIELSICGTGPLQHELRRMAAQLLPGRWSIGFHDNA
QMPAFLRSCDLLVHPSIVDIESLSVLEGMASGLVPVIARSTQSAAGQFALCDHSLFEAQDPAMLAQRIDW
WIDHPDELSRWGAIYAEHTREEYSIESCVRKFVAMEREAIADFDGRL
>WP_047289411.1 glycosyltransferase [Bifidobacterium bifidum]
(SEQ ID NO: 117)
MRDNKNEEPRPQDRSVLDRPMTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_049185285.1 glycosyltransferase [Bifidobacterium scardovii]
(SEQ ID NO: 118)
MLENGNEGNGGLPEIDRPLTIVLVMDTIGNQGNGTSNSALQYAHELERQGHHVRLVGIGAPEYPVRVNKV
PLVSWVAAKQQMQFAEPSETLFRTAFAGADVVHIYLPFKFGRCAAKVARRMGIAVTAGFHLQPENVTYSA
GPIKYIPGISNFLYALFRHWLYRNIGHIHVPTEMIAGQLRAHGYTAKLHVISNGYVPRFTPKRQRRADAP
APVPFRIVASGRLSHEKDHITLIKAIARCRHAKDIELTICGTGPLRRYLRFRAKMLLSRPASIGFHKNAE
MPALLRSCDLFAHPSIVDIESLSVIEGMASGLVPVIASAELSAAGQFALLDESLFPAHDTVTLARRIDWW
IDHPAELAEWGARYAAHTKAHYSVEASVAQFVAMEREAIADNARDAR
>WP_051126230.1 glycosyltransferase [Bifidobacterium minimum]
(SEQ ID NO: 119)
MRDDGRGTPHDGRPLTIVMVVDTIGRNGNGTSNSAMQYAGRLRELGHRVRLVGIGSEDYPAASHHVPIAS
WASAKQQIQFARPDADLMRRAFAGADVVHIYLPFAFGRCARAVAREMDVPVTAGFHLQPENVTYSAGPLR
YVPGVSAFLYRLFRRWLYAGIGHVHAPTRMIADQLRSHGYDNEIHVISNGFSPMFSPSHGGEPDGRDGRD
RGRRFRIVASGRLSHEKDHITLIRAVARCRHAADIDLAICGTGPLDSYLRAQARRLLPRTRWSIGFHSHD
EMPRLLRACDLIVHPSIVDIESLSVLEGIACGLVPVIAESDLSAARQFALTDESLFPARDVQALADGIDW
WVEHAEDRRYWAARYADEARSRYSLERCVDRFVDMERAAIADHQALAISRPVEAAS
>WP_061085917.1 glycosyltransferase [Bifidobacterium bifidum]
(SEQ ID NO: 120)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APASVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_061870441.1 glycosyltransferase [Bifidobacterium bifidum]
(SEQ ID NO: 121)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_065470626.1 glycosyltransferase [Bifidobacterium breve]
(SEQ ID NO: 122)
MRENGIEKSIDRPLTIALVVDTVGNQGNGTSNSALQWAAELERQGHHVRLVGVGAPEYPARVNKVPLVSW
VAAKQLMQFAEPSDTLFRTAFQGVDVVHVYMPFKFGRRAAKVAHQMGIPVTAGFHLQPENVLYSAGPLRH
IPGISSFLYWLFKHWLYKRIDHIHVPTEMTASLLRAHGYKAKLHVISNGYSPVYSPKKPVDPEASAPVPF
RVIASGRLASEKDQITLIKAVSMSKHAGGIQLIIAGTGPLEQYLRFRAGRLLARKADIGFHKHADMPDLL
RSGDLFVHCSIADIESVSVIEAMACGLVPVIAASELSAASQFALLDESLFPVQNAAMLARRIDWWIEHQV
ERAEWGEKYAEYTKANYSVEASVHKFVDMEREAIGE
>WP_072726496.1 glycosyltransferase [Bifidobacterium lemurum]
(SEQ ID NO: 123)
MLKNASEHVEQPLTIALVVDSIGNQGNGTSNSALQWASELERQGHHVRLVGVGAPEYPARVNKVPLVSWV
AAKQQMQFAEPSDTLFRTAFAGADVVHIYMPFKFGRRAAKIARDMGIPVTAGFHLQPENVLYSAGPLRHI
PGMQNLLYRLFHWWLYRRIDHIHTPTEMTASLLRSHGYRGTLHVISNGYSPRFEAREPLSAQEAVRTPLR
IVASGRLAHEKDQITLIKAISMSRYASDIQLIIAGTGPLRRYLQFRADLLLARKAQIGFHKNADMPALLR
SCNLFVHCSIADIESVSVIEAMACGLVPVIAASELSAASQFALLDESLFPVRNAALLARRIDWWFDHPEE
LAAWGARYAEHTESHYSVESSVRRFVAMEREAIADAAK
>WP_082440323.1 glycosyltransferase [Bifidobacterium aesculapii]
(SEQ ID NO: 124)
MREQEVEEDRIVSGRDARTVKSGDDAGVTEEGIDRPLTIALVVDTIGNQGNGTSNSALQWAAELRRQGHH
VRLVGIGAPEYPARVNHVPLVSWVAAKQQMQFAEPSDTLFRTAFRGVDVVHIYMPFKFGRRAAKIARQMG
IPVTAGFHLQPENVLYSAGPLRYIPGMERFLYWLFRQWLYKRVDHIHTPTEMTASLLREHGYKGVMHVIS
NGYAPRFTARAPLAPDAAAHVPFRVIASGRLAHEKDQITLIKAISMSRHASDIQLVIAGTGPLRRYLTFR
ANRRLKRKADIGFHRNADMPALLRSCDLFVHCSIADIESVSVIEAMACGLVPVIAASELSAASQFALLDE
SLFPVRDATMLARRIDWWIDHPDELARWGATYARHTREHYSVEASVHRFVDMEREAMAQGAR
>WP_094636880.1 glycosyltransferase [Bifidobacterium eulemuris]
(SEQ ID NO: 125)
MLKNASEHVEQPLTIALVVDSIGNQGNGTSNSALQWASELERQGHHVRLVGVGAPEYPARVNKVPLVSWV
AAKQQMQFAEPSDTLFRTAFAGADVVHIYMPFKFGRRAAKIARDMGIPVTAGFHLQPENVLYSAGPLRHI
PGMQNLLYRLFHLWLYRRVDHIHTPTEMTASLLRSHGYRGTLHVISNGYSPRFEAREPLSAQEAVRMPLR
IVASGRLAHEKDQITLIKAVSMSRYASDIQLIIAGTGPLRRYLQFRADLMLARKAQIGFHRNSDMPALLR
SCNLFVHCSIADIESVSVIEAMACGLVPVIAASELSAASQFALLDESLFPVRNAALLARRIDWWFDHPEE
LAAWGARYAEHTESHYSVESSVRRFVAMEREAIADAAK
>WP_100496701.1 glycosyltransferase [Bifidobacterium scaligerum]
(SEQ ID NO: 126)
MRENGSEESLDRPLTIALVVDTVGNQGNGTSNSALQWATELERQGHHVRLVGIGAPEYPARVNKVPLVSW
VAAKQLMQFAQPSDTLFRTAFQGVDVVHIYMPFKFGRHAAKVARQMGIPVTAGFHLQPENVLYSAGPLRH
IPGISTFLYWLFKHWLYKRVDHIHVPTEMTASLLRSHGYQATLHVISNGYSPEYQPGELPDPKAASPVPF
RIIASGRLAHEKDQITLIKAIAMSRHAEDIQLVIAGTGPLRHYLRFRAGRLLARKAEIGFHRHAEMPSLL
RSGDLFVHCSIADIESVSVIEAMACGLVPVIAASELSAASQFALLDESLFPVRDVALLARRIDWWIAHPR
ERAEWSAKYAESTKEHYSVEASVHKFVDMEREAIEG
>WP_100988792.1 glycosyltransferase [Bifidobacterium longum]
(SEQ ID NO: 127)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVARQMGIPVTAGFHLQPENVLYSAGPLRR
IPGISSFLYWLFKHWLYKHIDHIHVPTEMTASLLRAHGYKAKLHVISNGYSPVYSPKKPADPDASASVPF
RVIASGRLASEKDQITLIKAVSMSRHAGDIQLVIAGTGPLKQYLKFRAGRLLARKADIGFHKHADMPDLL
RSGDLFVHCSIADIESVSVIEAMACGLVPVIAASELSAASQFALIDESLFPVRDAALLARRIDWWIAHQA
ERAEWGEKYAEYTKAHYSVEASVHKFVDMERKAIGE
>WP_101027530.1 glycosyltransferase [Bifidobacterium longum]
(SEQ ID NO: 128)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVAHQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVVASGRLASEKDQITLIKAVSMSRHAGDIQLVIAGTGPLKQYLKFRAGRLLARKADIGFHKHADMPDLL
RSCDLFVHCSIADIESVSVIEAMACGLVPVIAASELSAAGQFALIDESLFPVRDAALLARRIDWWIAHQA
ERAEWGEKYAEYTKAHYSVEASVHKFVDMERKAIGE
>WP_101452535.1 glycosyltransferase [Bifidobacterium thermophilum]
(SEQ ID NO: 129)
MKNQEFSVAAGSTEDAGRPLVIALVVDTAGNEGNGTSNSALQYAKELRRQGHTVRLVGIGSPEYPIRVRH
IPLVSWIAAKQQMQFAQPDRAMFRKAFEGADVVHVYLPFKYGRMAVRTAHEMGIPATAGFHLQPENVLYS
AGPLRYIPGASAFIYWLFNRMLYRHIWHIHTPSHMIAAQLRAHGYRQRLHVISNGYSPRFTAKQQREPGS
PSPRPFRIVASGRLASEKDHVTLIRAVALSRHAEDIELTICGTGPLQHELRRMAGKLLPGRWSIGFHDNA
QMPELLRSCDLLVHPSIVDIESLSVLEGMASGLVPVIARSTQSAAGQFALNDHSLFEAQDPAMLAQRIDW
WIDHPDELSRWGAIYAEHTREEYSIASCVRKFVAMEREAIADFDGRL
>WP_101455202.1 glycosyltransferase [Bifidobacterium thermophilum]
(SEQ ID NO: 130)
MKNQEFSVAAGSTEDAGRPLVIALVVDTAGNEGNGTSNSALQYAKELRRQGHTVRLVGIGSPEYPIRVRH
IPLVSWIAAKQQMQFAQPDRAMFRKAFEGADVVHVYLPFKYGRMAVRTAHEMGIPATAGFHLQPENVLYS
AGPLRYIPGASAFIYWLFNRMLYRHIWHIHTPSHMIAAQLRAHGYRQRLHVISNGYSPRFTAKQQREPGS
PSPRPFRIVASGRLASEKDHVTLIRAVALSRHTEDIELTICGTGPLQHELRRMAAQLLPGRWSIGFHDNA
QMPAFLRSCDLLVHPSIVDIESLSVLEGMASGLVPVIARSTQSAAGQFALCDHSLFEAQDPAILAQRIDW
WIDHPDELSRWGAIYAEHTREEYSIASCVRKFVAMEREAIADFDGRL
>WP_101625806.1 glycosyltransferase [Bifidobacterium imperatoris]
(SEQ ID NO: 131)
MRENGSEESIDRPLTIALVVDTVGNQGNGTSNSALQWAAELERQGHHVRLVGVGAPEYPARVNKVPLVSW
VASKQLMQFAQPSDTLFRTAFQGVDVVHIYMPFKFGRHAAKVARQMGIPVTAGFHLQPENVLYSAGPLRH
IPGISSFLYWLFKHWLYKRIDHIHVPTEMTASLLRAHGYKAKLHVISNGYSPAYKPLKSVDPAAPTPVPF
RIIASGRLAREKDQITLIKAISMSRHAADIQLVIAGTGPLRRYLSFRAGRRLARKADIGFHKHAEMPELL
RSGDLFVHCSIADIESVSVIEAMACGLVPVIAASELSAASQFALLDESLFPVRDAALLARRIDWWISHQQ
ERAEWSAKYAENTKAHYSVEASVHKFVDMEREAIRE
>WP_106628192.1 glycosyltransferase [Bifidobacterium breve]
(SEQ ID NO: 132)
MRENGIEKSIDRPLTIALVVDTVGNQGNGTSNSALQWAAELERQGHHVRLVGVGAPEYPARVNKVPLVSW
VAAKQLMQFAEPSDTLFRTAFQGVDVVHVYMPFKFGRRAAQVARQMGIPVTAGFHLQPENVLYSAGPLRH
IPGISSFLYWLFKHWLYKRIDHIHVPTEMTASLLRAHGYKAKLHVISNGYSPVYSPKKSVDPEASAPVPF
RVIASGRLASEKDQITLIKAVSMSKHAGDIQLIIAGTGPLKQYLRFRAGRLLARKADIGFHKHADMPDLL
RSGDLFVHCSIADIESVSVIEAMACGLVPVIAASELSAASQFALLDESLFPVQNAAMLARRIDWWIEHQV
ERAEWGEKYAEYTKSHYSVEASVHKFVDMEREAIGE
>WP_107041170.1 glycosyltransferase [Bifidobacterium callitrichos]
(SEQ ID NO: 133)
MRERAIEAIEEDHEERIDRPLTIALVVDTVGNQGNGTSNSALQWAAELKRQGHHVRLVGIGAPDYPARVN
KVPLVSWVAAKQQMQFAEPSDTLFRTAFRGVDVVHIYMPFKFGRRAAKVARQMGIPVTAGFHLQPENVLY
SAGPLRYIPGMEKFLYWLFRQWLYKRVDHIHTPTEMTASLLREHGYRGVMHVISNGYSPRFTAREPIDPQ
APARVPFRVVASGRLAHEKDQITLIKAISMSRHASDIQLIIAGTGPLKRYLTFRANRRLKRRADIGFHNN
ADMPALLRSCDLFVHCSIADIESVSVIEAMASGLVPVIAASELSAASRFALLDESLFPVRDAAMLARRID
WWIDHPAELARWGARYATHTREHYSVEASVHKFVDMEREAIDAKATAAR
>PWM36879.1 MAG: glycosyltransferase [Oscillospiraceae bacterium]
(SEQ ID NO: 134)
MDTFDRESGRDLPSLPGRAAQPIPQRRTAAMKVATICDVMGQPNNGTTLAALNLIRYLRDAGHTVTVVAP
GGEASDGYLPVRVWHAGPLIDRILRMNGVELAVPDKQLLEAVIREADVVHLLIPLPLARAALKIARRLGK
PVTASFHCQAENITAHLGMMNAGWLNRLIYRNFYRKVYRWCTAVHYPTEFIREVFETATHPTPAHVISNG
VNDMFRLPDSRPENGKFTVVCSGRYSREKAQQQLLRAAALCRHRDDIRLILAGDGPRRKHYLRLAKQYGL
DCQFAFFPRQELLHILQTADLYVHTAIIEIEAIACTEAICCGLVPVICNSDRSATRFFACGDHTLFEPGD
VRALADLLDYWYEHPAARVERAAEYADLRHSFDQTACMQQMEQMLKEAAGL
>WP_115954881.1 glycosyltransferase [Bifidobacterium bifidum]
(SEQ ID NO: 135)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_117631659.1 glycosyltransferase [Bifidobacterium bifidum]
(SEQ ID NO: 136)
MRDNKNEEPRLQDRSALDRPLTIVLVMDTIGNKGNGTSNSALQYAHELERQGHRVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQCSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_117658361.1 glycosyltransferase [Bifidobacterium bifidum]
(SEQ ID NO: 137)
MRDNKNEEPRPQDRSVLDRPLTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_117726135.1 glycosyltransferase [Bifidobacterium bifidum]
(SEQ ID NO: 138)
MRDNKNEEPHPQDRSVLDRPMTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_125980622.1 glycosyltransferase [Bifidobacterium goeldii]
(SEQ ID NO: 139)
MAENESREEDARPPQIERPLTIVLVMDTIGNKGNGTSNSALQYAHELERQGHHVRLVGIGAPEYPARINK
VPLVSWVAAKQQMQFAQPSETLFRTAFAGADIVHIYLPFKFGRQAYRIARSMGIPVTAGFHLQPENVTYS
AGPLKYVPGISSFLYWLFNMWLYRRVDHIHAPTEMIAEQLRSHGYKAKLHVISNGYTARFTPKQQRSANA
PTPVPFRVVASGRLTHEKDHITLIKAIARCRHARDIELTICGTGPLAHYLRFRAKRLLTRPASIGFHDNA
DMPALLRSCDLFVHPSIVDIESLSVIEGMASGLVPVIAASELSAASQFALLDESLFPARDATLLARRIDW
WIDHPDELAKWGERYARHTKEHYSVEASVTQFVDMERQAIADNH
>WP_134994128.1 glycosyltransferase [Bifidobacterium bifidum]
(SEQ ID NO: 140)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGLYAEHAKADYSVEASVAKFVAMEREAIADAKA
>WP_143246563.1 glycosyltransferase [Bifidobacterium breve]
(SEQ ID NO: 141)
MRENGIEKSIDRPLTIALVVDTVGNQGNGTSNSALQWAAELERQGHHVRLVGVGAPEYPARVNKVPLVSW
VAAKQLMQFAEPSDTLFRTAFQGVDVVHVYMPFKFGRRAAKVAHQMGIPVTAGFHLQPENVLYSAGPLRH
IPGISSFLYWLFKHWLYKRIDHIHVPTEMTASLLRAHGYKAKLHVISNGYSPVYSPKKPVDPEASAPVPF
RVIASGRLASEKDQITLIKAVSMSKHAGDIQLIIAGTGPLKQYLRFRAGRLLARKADIGFHKHADMPDLL
RSGDLFVHCSIADIESVSVIEAMACGLVPVIAASELSAASQFALLDESLFPVQNAAMLARRIDWWIEHQV
ERAEWGEKYAEYTKAHYSVEASVHKFVDMEREAIGE
>WP_150335554.1 glycosyltransferase [Bifidobacterium reuteri]
(SEQ ID NO: 142)
MRENGTGEESIDRPLTIALVVDTVGNQGNGTSNSALQWAAELERQGHHVRLVGIGAPEYPARVNKVPLVS
WVASKQLMQFAQPSDTLFRTAFRGVDVVHVYMPFKFGRHAAKVARQMGIPVTAGFHLQPENVLYSAGPLR
HIPGISGFLYWLFKHWLYKRIDHIHVPTEMTASLLRSHGYQAKLHVISNGYSPVYTPKPPAESSEDPDAA
SPVPFRIIASGRLAREKDQITLIKAVSMSRHAADIQLVIAGTGPLKHYLKFRAGRLLARKADIGFHKHAD
MPELLRSGDLFVHCSIADIESVSVIEAMACGLVPVIAASELSAASQFALLDESLFPVRDAALLARRIDWW
IAHQRERVAWGAKYAQNTKEHYSVEASVHKFVDMEREAITDNAYSSAMS
>WP_150383086.1 glycosyltransferase [Bifidobacterium rousetti]
(SEQ ID NO: 143)
MRERAIEAIEEDHEERIDRPLTIALVVDTVGNQGNGTSNSALQWAAELKRQGHHVRLVGIGAPDYPARVN
KVPLVSWVAAKQQMQFAEPSDTLFRTAFRGVDVVHIYMPFKFGRRAAKVARQMGIPVTAGFHLQPENVLY
SAGPLRYIPGMEKFLYWLFRQWLYKRVDHIHTPTEMTASLLREHGYRGVMHVISNGYSPRFTAREPVDPQ
APARVPFRVVASGRLAHEKDQITLIKAISMSRHASDIQLIIAGTGPLKHYLTFRANRRLKRRADIGFHNN
ADMPALLRSCDLFVHCSIADIESVSVIEAMASGLVPVIAASELSAASQFALLDESLFPVRDAAMLARRID
WWIDHPAELARWGARYATHTREHYSVEASVHKFVDMEREAIDAKATAAR
>WP_151901892.1 glycosyltransferase [Bifidobacterium bifidum]
(SEQ ID NO: 144)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPRELAVWGGRYAEHAKADYSVEASVAKFVAMEREAIADAKA
>WP_152029623.1 glycosyltransferase [Bifidobacterium bifidum]
(SEQ ID NO: 145)
MRDNKNEEPHPQDRSVLDRPMTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASGAKFVAMEREAIADAKA
>WP_152233706.1 glycosyltransferase [Bifidobacterium leontopitheci]
(SEQ ID NO: 146)
MLANRGKENDGRTQAPQIEAPLTIALVVDTIGNKGNGTSNSALQYARELERQGHHVRLVGVGAPEYPAKV
NKVPLVSWVAAKQQMQFARPSDTLFRTAFAGADVVHVYLPFKFGRRACKVAHDMGIPVTAGFHLQPENVM
YSAGPLKYLPGMSAFLYALFRFWLYRHVRHIHAPTEMIAGQLRAHGYKAKLHVISNGYVPRFTPKRQPAA
DAPVPVPFRIVASGRLTHEKDHITLIKAIARCRHAKDIELVICGTGPLQRYLRFRAKRLLEREAQIGFHR
NADMPALLRSCDLLVHPSIVDIESLSVIEGMASGLVPVIAASDLSAASQFALLDESLFPARDVTMLARRI
DWWIDHPDELRRWGAQYARHAKEHYSVEASVTKFVAMEREAVAEGV
>WP_152350488.1 glycosyltransferase [Bifidobacterium avesanii]
(SEQ ID NO: 147)
MPEIEDEARETHEAGDRPLTIALVMDTIGTQGNGTSNSALQYARELERQGHHVRLVGLGSEEWPARENHV
PLVSWVAKKQHMRFAKPSDTLFRTAFRGVDVVHIYLPFSFGRRAWKIAREMGIPVTAGFHLQPENVSYNA
GPIKWIPGVNDFMYALFRFWLYRRVGHIHTPTEMIATQLREHDYKAKLHVISNGYSPRFTPKTQRKAGAR
EPWRYRVVASGRLAREKDHATLIRAVAMCRHAERIELHIAGTGPRERYLKRRAKRMLPNPAYIGFHRNAD
MPAFLKTCDLFVHPSIVDIESLSVIEGMASGLVPVIAESELSAAGQFALLDESLFPAGDAAALAERIDWW
LDHPRELATWGAKYAGHTREHYSIESCVRRFVAMEREAIADAA
>WP_152355176.1 glycosyltransferase [Bifidobacterium apri]
(SEQ ID NO: 148)
MKNQGSNQGSVAAGNIADAGRPLVIALVVDTAGNEGNGTSNSALQYAKELRRQGHTVRLVGIGSPEYPIR
VHHVPLVSWIAAKQQMQFAQPDRSMFRKAFAGADVVHVYLPFKYGRVAVRTAHEMGIPATAGFHLQPENV
LYSAGPLRYIPGASAFIYWLFDRMLYRHIRHIHTPSHMIAAQLRAHGYRQRLHVISNGYAPRFTAKRQRE
PGSPVPRPFRIVASGRLASEKDHVTLIRAVALSRHAEDIELVICGTGPLQHELRRMAGKLLPGRWSIGFH
DNAQMPQLLRSCDLLVHPSIVDIESLSVLEGMASGLVPVIARSAQSAAGQFALNDHSLFEAQDPAMLAQR
IDWWIDHPDELSRWGAIYAEHTREEYSIASCVRKFVAMEREAIADFDGRL
>WP_152358124.1 glycosyltransferase [Bifidobacterium ramosum]
(SEQ ID NO: 149)
MREQQEDGVAADAAASQPQLDRPLTIVLVMDTIGNQGNGTSNSALQYAHELERQGHHVRLVGIGAPEYPA
RVSHVPLVSWVAAKQQMQFAEPSETLFRTAFRGADVVHIYLPFRFGRHAAKVARAMGIPVTAGFHLQPEN
VTYSAGPLRHVPGMSDFLYWLFRMWLYRGVGHIHAPTEMIAGQLRAHGYRAKLHVISNGYTARFTPKRQR
EPGTPSPVPFRVVASGRLTHEKDHITLIKAIARCRHAKDIELTICGTGPLQRYLRFRAKMLLGRPASIGF
HQNADMPALLRSCDLFVHPSIVDIESLSVIEGMASGLVPVIASAELSAAGQFALLDKSLFPVRDAAALAQ
RIDWWIDHPDELAEWGARYAEHTKEHYSVEASVAKFVDMEREAIADVG
>WP_154055943.1 glycosyltransferase [Bifidobacterium bifidum]
(SEQ ID NO: 150)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFHFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLQHYLRFRADRLLERKAQIGFHKN
AEMPTLLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_154568573.1 glycosyltransferase [Bifidobacterium tsurumiense]
(SEQ ID NO: 151)
MNRDREVPSGQRENGDIDVRPLTIALVVDTIGNHGNGTSNSALQYAQELERQGHHVRLVGIGSQEYPAKV
NRIPLVSHIAARQQMQFAKPSDTLFRTAFAGVDVVHIYMPFSFGRRARIIAQEMGIPVTAGFHVQPENVL
YSAGPLRFVPGASRFIYWLFKRWLYRYVDHIHTPTQMIAEELRKHHYDAKLHAISNGYSPRFVPRQPRAA
ADIRPPYRVAASGRLSLEKSQITLIKAISLCRHGADIELSICGTGPMRRYLQMRAKRLLNSPVRIGFQPN
DRMPGLLRSQDLLVHASVVDIESLSVMEGIGSGVVPIIARSGLSAASQFALTDQSLFEARNAQQLADRID
WWLDHPMELVSWEQKYARFAAQRYSVESSVRQFIEMERESIAEYESSH
>WP_159438164.1 glycosyltransferase [Massiliimalia massiliensis]
(SEQ ID NO: 152)
MAGKIPGGRNQDLELGGSMVITFVIDMYDVKKNGTTMTAQRFAHYLQQLGHEIRVVSTGDPGPGKYPVPQ
WKIPIVQHFSSKQSFIIAKPDDEQIRRAIEGSDLVHLFLPFPLEVRAYHIAREMGIPCSAAFHLQPENIT
YNIHMGRWRGLSDFVYYLFREYFYKNFEHIHCPSYFMAEQLKAHGYKAKLHVISNGIIPEFCPGQATRQD
ELIHILMVGRLSPEKRQDLLIKAAGMSRHAEKIQLHFAGSGPWKQMLAHRGKKLKHPPVFGFYSKPELIE
LIRSCDLYVHASDAESEAIACLEAIACGLVPVISDSKLSATGQFALDERSLFHAGDAASLAEQIDYWIDH
PEEKARMSSEYAKSTEKYRIEHCVREAETMFEEAVRDARGTGRPA
>WP_163195972.1 glycosyltransferase [Bifidobacterium platyrrhinorum]
(SEQ ID NO: 153)
MREHESERNGRRPAGIEGFGADGVGFEEDIDRPLTIALVVDTVGNQGNGTSNSALQWAAELERQGHHVRL
VGIGAPEYPARVNKVPLVSWVAAKQQMQFAQPSDTLFRTAFQGVDVVHVYMPFKFGRRAAKVARQMGIPV
TAGFHLQPENVLYSAGPLRYVPGMERFLYWLFRQWLYKRVDHIHTPTEMTAGLLREHGYRGVTHVISNGY
SPRFTAREPLDPDAPAHVPFRVVASGRLAHEKDQITLIKAVSMSRHAGDIHLTIAGTGPLKRYLTFRAHR
RLRRNADIGFHRNADMPALLRSCDLFVHCSIADIESVSVIEAMACGLVPVIAASELSAASQFALTDESLF
PVRDAAMLARRIDWWIDHPDELARWGAKYAEHTREHYSVEASVHRFVDMEREAIATAGR
>WP_168973571.1 glycosyltransferase [Bifidobacterium boum]
(SEQ ID NO: 154)
MSQSSKFTRATGVNGDADRPLTIALVVDTAGNEGNGTSNSALQYARELRRQGHTVRLVGIGSPEYPIRVH
HIPLVSWIAAKQQMQFAQPDQAVFHKAFAGVDVVHVYLPFKYGRVAVQTAHEMGIPATAGFHLQPENVLY
SAGPLRYIPGASAFIYWLFDRMLYRHIRHIHTPSHMIAAQLRAHGYRQRLHVISNGYSPRFTPKRQREPG
SPSPRPFRIVASGRLASEKDHVTLIRAVALSRHAEDIELVICGTGPLQHELRRMAGKLLPGRWSIGFHDN
AQMPQLLRSCDLLVHPSIVDIESLSVLEGMASGLVPVIARSTQSAAGQFALNDHSLFEAQDPAMLAQRID
WWIDHPDELSHWGAIYAEHTHEEYSIASCVRKFVAMEREAIADFDGRL
>WP_168983778.1 glycosyltransferase [Bifidobacterium thermophilum]
(SEQ ID NO: 155)
MKNQGFSVAAGSTEDAGRPLVIALVVDTAGNEGNGTSNSALQYAKELRRQGHTVRLVGIGSPEYPIRVRH
IPLVSWIAAKQQMQFAQPDRAMFRKAFEGADVVHVYLPFKYGRVAVRTAHEMGIPATAGFHLQPENVLYS
AGPLRYIPGASAFIYWLFDRILYRHIRHIHTPSHMIAAQLRAHGYRQRLHVISNGYSPRFTAKQQREPGS
PSPRPFRIVASGRLASEKDHVTLIRAVALSRHAEDIELSICGTGPLQHELRRMAAQLLPGRWSIGFHDNA
QMPAFLRSCDLLVHPSIVDIESLSVLEGMASGLVPVIARSTQSAAGQFALCDHSLFEAQDPAMLAQRIDW
WIDHPDELSRWGAIYAEHTREEYSIASCVRKFVAMEREAIADFDGRL
>WP_169172223.1 glycosyltransferase [Bifidobacterium sp. DSM 109957]
(SEQ ID NO: 156)
MVEQPLTIALVIDTLANRGNGTSNSALQFADELQRQGHHVRLVGVGSPDYPARINKVPLVSWVASKQLMQ
FAEPSDTLFRTAFAGVDVVHIYMPFKFGRRAAKIARDMGIPVTAGFHLQPENVLYSAGPLRHIPGLSDFL
YWLFRQWLYRRIDHIHVPTQMTASLLRAHGYKAKLHVISNGYSPDYTPGAPRDPNAPNPVPFRIIASGRL
AREKDQITLIKAVALSKHAADIQLVIAGTGPLKRYLKFRSGRLLARKADIGFYKHADMPELLRSGDLFVH
CSIADIESVSVIEAMACGLVPVIAASELSAASQFAMLDESLFPVHDAALLARRIDWWIDHPAERAQWSAE
YAAHTKAHYSVEASVHKFADMEREAVTAAR
>WP_169275146.1 glycosyltransferase [Bifidobacterium sp. DSM 109958]
(SEQ ID NO: 157)
MAKNRENGAVAADGGRALTIVLVMDTIGTQGNGTSNSALQYARELERQGHRVRLVGLGSEEYPARENHVP
LVSWVAAKQHMQFAKPSDTLFRTAFQGADVVHLYLPFAFGRRAWKVAREMGIPVTAGFHLQPENVSYNAG
PIKAIPGVNDFMYLLFRHWLYRHVGHIHAPTEMIAEQLRAHGYKAKLHVISNGYSPRFTPKSQRKPTDRE
PLPLRVVASGRLAREKDHITLIRAVAMCRHAERIELHIAGTGPRQRYLRRRAKRLLPRPASIGFHRNADM
PAFLKSCDLFVHPSIVDIESLSVIEGMASGLVPVIAQSELSAASQFALLDESLFPARDAAALAERIDWWL
DHPRELAEWGARYAEHTREHYSIESCVRAFVAMEREAIADAAR
>WP_172145072.1 glycosyltransferase [Bifidobacterium sp. DSM 109963]
(SEQ ID NO: 158)
MREQSREKTTDRPLVIALVVDTVGNQGNGTSNSALQWAAELRRQGHTVRLVGIGSAEYPARVRRVPLVSW
VAAKQQMQFAEPSDTLFRKAFEGADVVHIYTPFAFGRRAAHVARTMGVPVTAGYHVQPENVLYSAGPLRL
IPGAQRFIYWLFNRWLYHDIAHIHTPTEMTASLLREHGYRAKLHVISNGYAPRFTARETSEPQADVQAEP
HVPFRIIASGRLAHEKDHITLIKAVAMSRHAGDIQLIIAGTGPLKRYLTFRANRLLARKADIGFHPNAEM
PELLRSCDLFVHCSIADIESISVIEAMACGLVPVIATSELSAASQFALLDESLFGVRDVAMLARRIDWWI
DHPCELAEWGARYAAHTREHYSVASSVRQFVQMEREAAS
>WP_195235442.1 glycosyltransferase [Bifidobacterium bifidum]
(SEQ ID NO: 159)
MRDNKNEEPRPQDRSVLDRPMTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APASVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_195261742.1 glycosyltransferase [Bifidobacterium longum]
(SEQ ID NO: 160)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVARQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVIASGRLASEKDQITLIKAVSMSRHAGDIQLVIAGTGPLKQYLKFRAGRLLARKADIGFHKHADMPDLL
RSGDLFVHCSIADIESVSVIEAMACGLVPVIAASELSAASQFALIDESLFPVRDAALLARRIDWWIAHQA
ERAEWGEKYAEYTKAHYSVEASVLKFVDMERKAIGE
>WP_195272662.1 glycosyltransferase [Bifidobacterium bifidum]
(SEQ ID NO: 161)
MRDNKNEEPRPQDRSALDRPMTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APASVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_197308541.1 glycosyltransferase [Bifidobacterium longum]
(SEQ ID NO: 162)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVAHQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVVASGRLASEKDQITLIKAVSMSRHAGDIQLVIAGTGPLEQYLKFRAGRLLARKADIGFHKHADMPDLL
RSGDLFVHCSIADIESVSVIEAMACGLVPVIAASELSAAGQFALIDESLFPVRDAALLARRIDWWIAHQA
ERAEWGEKYAEYTKVHYSVEASVHKFVDMERKAIGE
>WP_200488207.1 glycosyltransferase [Bifidobacterium longum]
(SEQ ID NO: 163)
MRENGIEESIDRPLTIALVVDTVGNQGNGTSNSALQWAAELERQGHHVRLVGIGAPEYPARVNRVPLVSW
VAAKQLMQFAEPSDTLFRTAFQGVDVVHIYMPFKFGRRAAKVAHQMGIPVTAGFHLQPENVLYSAGPLRR
IPGISSFLYWLFKHWLYKHIDHIHVPTEMTASLLRAHGYKAKLHVISNGYSPMYSPKKPADPDASAPVPF
RVIASGRLASEKDQITLIKAVSMSRHAGDIQLVIAGTGPLKQYLKFRAGRLLARKADIGFHKHADMPDLL
RSGDLFVHCSIADIESVSVIEAMACGLVPVIAASELSAASQFALIDESLFPVRDAALLARRIDWWIAHQA
ERAEWGEKYAEYTKAHYSVEASVHKFVDMERKAIGE
>WP_202570263.1 glycosyltransferase [Bifidobacterium longum]
(SEQ ID NO: 164)
MRENGIEESIDRPLTIALVVDTVGNQGNGTSNSALQWAAELERQGHHVRLVGIGAPEYPARVNKVPLVSW
VAAKQLMQFAEPSDTLFRTAFQGVDVVHIYMPFKFGRRAAKVAHQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVVASGRLASEKDQITLIKAVSMSRHAGDIQLVIAGTGPLKQYLKFRAGRLLARKADIGFHKHADMPDLL
RSGDLFVHCSIADIESVSVIEAMACGLVPVIAASELSAAGQFALIDESLFPVRNAALLARRIDWWIAHQA
ERAEWGEKYAEYTKVHYSVEASVHKFVDMERKAIGE
>WP_204467051.1 glycosyltransferase [Bifidobacterium pullorum]
(SEQ ID NO: 165)
MRENENERRIDGPLTIALVVDTVGNQGNGTSNSALQWAQELERQGHHVRLVGVGAPEYPARVNKVPLVSW
VAAKQQMQFAEPSDTLFRAAFAGCDVVHIYMPFKFGRHAAKVARRMGIPVTAGFHLQPENVLYSAGPLRF
IPGMQSLLYALFRHWLYRKVDHIHVPTEMTASLLRGHGYQARLHVISNGYSTRFTAREPRDPEAAAPVPF
RIVASGRLAREKDQVTLVKAVAMSRHASNIQLVIAGTGPLKHYLKFRAGWLPRHAEIGFHRNADMPALLR
SCDLFVHCSIADIESVSVIEAMACGLVPVIAASELSAASQFALTDRSLFPVRDAAILANRIDWWIDHPGE
LARWGAVYGEHTKEHYSVEASVHRFVDMEREAIADAG
>WP_214312604.1 glycosyltransferase [Bifidobacterium sp. CP2]
(SEQ ID NO: 166)
MREHGDEAEHEESIDRPLTIALVVDTVGNQGNGTSNSALQWAAELERQGHHVRLVGIGAPEFPARVNRVP
LVSWVAAKQQMQFAEPSDTLFRAAFDGVDVVHIYMPFKFGRRAAKVARQMGIPVTAGFHLQPENVLYSAG
PLRYIPGMERFLYWLFRQWLYKRVGHVHTPTEMTASLLREHGYKGVMHVISNGYSPRFTARRPLDPSAPA
HVPFRVVASGRLAHEKDQITLIKAISMSRHASDIQLVIAGTGPLKRYLTFRANRRLKRRADIGFHRNADM
PALLRSCDLFVHCSIADIESVSVIEAMACGLVPVIAASELSAASQFALLDESLFPVRDAAILARRIDWWI
DHPDELARWGARYAEHTCEHYSVEASVHRFVDMEREAIADS
>WP_214356220.1 glycosyltransferase [Bifidobacterium sp. SO4]
(SEQ ID NO: 167)
MREKGNEESIDRPLTIALVVDTVGNQGNGTSNSALQWAAELERQGHRVRLVGIGAPEYPARVNKVPLVSW
VASKQLMQFAEPSDTLFRTAFQGVDVVHIYMPFKFGRHAAKVARQMGIPVTAGFHLQPENVLYSAGPLRH
IPGVSGFLYWLFKHWLYKRVDHIHVPTEMTASLLRSHGYKARLHVISNGYSPAYSPKEPVDPQAPTPVPF
RIIASGRLAHEKDQITLIKAISMSRHAGDIQLVIAGTGPLRRYLTFRAGRLLARKAEIGFHKHSEMPELL
RSGDLFVHCSIADIESVSVIEAMACGLVPIIAASELSAASQFALLDESLFPVRDAALLARRIDWWIAHQT
ERAEWGAKYAEHTKEHYSVEASVHKFVDMEREAIAE
>WP_214357607.1 glycosyltransferase [Bifidobacterium santillanense]
(SEQ ID NO: 168)
MREHGVEEAHGEYGQEEGIDRPLTIALVVDTVGNQGNGTSNSALQWAAELERQGHHVRLVGIGAPEYPAR
VNRVPLVSWVAAKQQMQFAEPSDTLFRTAFRGVDVVHVYMPFKFGRRAAKVARQMGIPVTAGFHLQPENV
LYSAGPLRYVPGAERFLYWLFRQWLYKRIDHIHTPTEMTASLLREHGYRGTLHVISNGYSPRFTAREPAD
PSAPAHVPFRVVASGRLAHEKDQITLIKAISMSRHAGDIQLVIAGTGPLKRYLTFRAHRRLKRRADIGFH
RNADMPALLRSCDLFVHCSIADIESVSVIEAMASGLVPVIAASELSAASQFALLDESLFPVRDAALLSRR
IDWWIDHPSELARWGAEYATHTREHYSVEASVHRFVDMEREAIATAAR
>WP_214375870.1 glycosyltransferase [Bifidobacterium colobi]
(SEQ ID NO: 169)
MRENGKEESIDRPLTIALVVDTVGNQGNGTSNSALQWAAELERQGHHVRLVGVGAPEYPARVNKVPLVSW
VASKQLMQFAQPSDTLFRTAFQGVDVVHIYMPFKFGRHAAKVARQMGIPVTAGFHLQPENVLYSAGPLRH
IPGVSAFLYWLFKHWLYKRIDHIHVPTEMTASLLRSHGYKAKLHVISNGYSPVYKPVKPVDPDAATPVPF
RIVASGRLAREKDQITLIKAISMSRHAADIQLVIAGTGPLRRYLTFRAGRLLARKADIGFHKHVEMPQLL
RSGDLFVHCSIADIESVSVIEAMACGLVPVIAASELSAASQFALLDESLFPVRDAALLARRIDWWITHQR
ERAEWSAKYAQNTKEHYSVEASVHKFVDMEREAIGE
>WP_217049481.1 glycosyltransferase [Bifidobacterium bifidum]
(SEQ ID NO: 170)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRAAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRLDG
APAPVPFRIVASGRLSHEKDQITLIKAISRCRHAKDIELIICGTGPLQHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGRYAEHAKADYSVEASVAKFVAMEREAIADAKA
>WP_217296494.1 glycosyltransferase [Bifidobacterium breve]
(SEQ ID NO: 171)
MRENGIEKSIDRPLTIALVVDTVGNQGNGTSNSALQWAAELERQGHHVRLVGVGAPEYPARVNKVPLVSW
VAAKQLMQFAEPSDTLFRTAFQGVDVVHVYMPFKFGRRAAKVAHQMGIPITAGFHLQPENVLYSAGLLRH
IPGISSFLYWLFKHWLYKRIDHIHVPTEMTASLLRAHGYKAKLHVISNGYSPVYSPKKPVDPEASAPVPF
RVIASGRLASEKDQITLIKAVSMSKHAGGIQLIIAGTGPLEQYLRFRAGRLLARKADIGFHKHADMPDLL
RSGDLFVHCSIADIESVSVIEAMACGLVPVIAASELSAASQFALLDESLFPVQNAAMLARRIDWWIEHQV
ERAEWGEKYAEYTKANYSVEASVHKFVDMEREAIGE
>WP_217738406.1 glycosyltransferase [Bifidobacterium longum]
(SEQ ID NO: 172)
MRENGIEESIDRPLTIALVVDTVGNQGNGTSNSALQWAAELERQGHHVRLVGIGAPEYPARVNRVPLVSW
VAAKQLMQFAEPSDTLFRTAFQGVDVVHIYMPFKFGRRAAKVARQMGIPVTAGFHLQPENVLYSAGPLRR
IPGISSFLYWLFKHWLYKHIDHIHVPTEMTASLLRAHGYKAKLHVISNGYSPVYSPKKPADPDASAPVPF
RVIASGRLASEKDQITLIKAVSMSRHASDIQLVIAGTGPLKQYLKFRAGRLLARKADIGFHKHADMPDLL
RSGDLFVHCSIADIESVSVIEAMACGLVPVIAASELSAASQFALIDESLFPVRDAALLARRIDWWIAHQA
ERAEWGEKYAEYTKAHYSVEASVHKFVDMERKAIGE
>WP_219080545.1 glycosyltransferase [Bifidobacterium phasiani]
(SEQ ID NO: 173)
MQENKVKVEDPLTIAMVVDTSGNRGNGTSNSALQWARELERQGHRIRLVGIGAPDFPAKVNHVPLVSWVA
RKQQMQFAEPSDTLFRAAFLGADVVHIYTPFRFGQHACRVARQMGVPVTAGYHVQPENITYSAGPLKYVP
GIDSFIYWLFRHWLYRNVGHVHVPTELGAELLRSHGYTSKLHVISNGYEPRFTAKRQRPGDEPPDWPIRI
IASGRLSNEKDQITLIRAVALSRHADDIQLTIAGTGPLKRRLQREAARLLPRPASIGFHRNAEMPALLRS
ADLFVHPSIADLESVSVLEGMASGLVPIIASSPLSAAGHFALREESLFPVGDAQALADRIDWWLDHPGEL
NAWGARYAEHTREHYSVEESVRRFVAMEREAIADARGHELTAA
>WP_219131603.1 glycosyltransferase [Bifidobacterium saguinibicoloris]
(SEQ ID NO: 174)
MREHGDEAEHEESIDRPLTIALVVDTVGNQGNGTSNSALQWAAELERQGHHVRLVGIGAPEFPARVNRVP
LVSWVAAKQQMQFAEPSDTLFRAAFDGVDVVHIYMPFKFGRRAAKVARQMGIPVTAGFHLQPENVLYSAG
PLRYIPGMERFLYWLFRQWLYKRVGHVHTPTEMTASLLREHGYKGVMHVISNGYSPRFTARRPLDPSAPA
HVPFRVVASGRLAHEKDQITLIKAISMSRHASDIQLVIAGTGPLKRYLTFRANRRLKRRADIGFHRNADM
PALLRSCDLFVHCSIADIESVSVIEAMACGLVPVIAASELSAASQFALLDESLFPVRDAAILARRIDWWI
DHPDELARWGARYAEHTREHYSVEASVHRFVDMEREAIADS
>WP_235342183.1 glycosyltransferase [Bifidobacterium boum]
(SEQ ID NO: 175)
MSQSSKFTRATGVNGDADRPLTIALVVDTAGNEGNGTSNSALQYARELRRQGHTVRLVGIGSPDYPVPVH
WIPLVSWIAAKQQMQFAQPDRAVFHKAFVGVDVVHVYLPFKYGRVAVQTAHEMGIPATAGFHLQPENVLY
SAGPLRYIPGASAFIYWLFDRMLYRHIRHIHTPSHMIAAQLRAHGYRQRLHVISNGYAPRFTAKQQRAPG
SPVPKPFRVVASGRLASEKDHVTLIRAVALSRHAEDIELVICGTGPLQHELRRMAGKLLPGRWSIGFHDN
AQMPQLLRSCDLLVHPSIVDIESLSVLEGMASGLVPVIARSTQSAAGQFALNDHSLFEAQDSAMLAQRID
WWIDHPDELSRWGAVYAEHTREEYSIASCVRKFVAMEREAIVDFDGRL
>WP_235715181.1 glycosyltransferase [Acetivibrio thermocellus]
(SEQ ID NO: 176)
MLFLLTVFFFVFEKLKVNAARKERGVKFLMVITLVNDTFNINNNGTTISAMRFAEALSQRGHQIRIITCG
DPLKSGKDPDTGFEMFYLPELKIPIASRLAHKQNTLFAKPVRSTLKKAISGSDVVHIYQPWPLGSAAQRV
ARQMNIPAIAAFHIQPENITFNIGLKRFSPAAHLTYFLFYLFFYRRFSHIHCPSKFIAAQLRSHGYKARL
HVISNGVHPAFCAPAKPREHTFKPIKILMIGRLSPEKRQDVLIRAVMKSRYADRIQLYFAGSGPWEKKLC
HLGNKLPNPPVFGYYNRDELIKLIHECDLYVHASDAEIEGISLIEAFACGLVPIISDSKQSAAAQFALGP
QNLFKAGSPESLAEKIDYWLDHPEQLKEAEKKYAQLGKQYALEHSIRKIEKVYSSMTKNHKNEYHRSIFF
RLSTRLFQIVIACPILLLWTRFVLGAKVYGRENIRGLKSGVTVCNHVHLLDSALIGVTFFPRRVVFPTLT
QNVKTLWPGKLVRILGGFAIPDNIMELKAFFDEMEFLLMKNCIVHFFPEGELRPYDTGLQNFKKGAFYLA
AQAQVPIVPMLITFEPPKGLIKIIRKKPVMRLHIGKPIHPMSKDIEIDSELRMKAVCKKIEAITSV
>WP_249293896.1 glycosyltransferase [Fumia xinanensis]
(SEQ ID NO: 177)
MTIVLVVDVFDNLTNGTTMTAYRFAQSLRENGHEVRAVAIGDKEENPYALNEAQLPVATKIAHKQGFAFA
KADVTTFERAFQGADVVHFLVPLFMDHKALKVAKKMDIPICGAFHLQPENVTFILHVSKAQWLARSIYRF
FNRVFYRNFDHIHCPSYFIANQLAKNGYTAKLHVISNGVDDDFCPGEVRERFDDGLIHILMVGRLSPEKR
QRILIDAVAKSQYADRIQLHFAGKGPCREKLIEQGKILPHPPTFAFYSKRELISLIRRCDLYVHASVVEI
EAISCLEAFSCGLVPVICDSDMSATVQFALDERSLFRPDDAKDLAAKIDYWLSHPEEKARMEQEYAKQGD
SYRISASVKKAEEMFLDVIRDYREKHRVSRY
>WP_252891671.1 glycosyltransferase [Thermoclostridium stercorarium]
(SEQ ID NO: 178)
MRIPTVSAFHLQPENVTYSIGLGKSKRANDVIYRYFYKCFYNRFRFIHCPSEMIAEQLMQHGYDAECRVI
SNGVNDLFRPAEVRRPEYLKGKIVLLMVGRLSGEKRQDLLIEAVQYSKYRDKIQLVFAGMGPKEKKYRKL
SRNLKNKPIFRFFTQEELLQMYNICDLYIHTSDAEIEGISCIEAMACGAVPVISDSHLSATKSFALHPNC
LFKAGDARSLAEKIDYWIEHPEERSKMSRIYAEKAETIRVSKCVAEMERLYKDVISDYHKNGYKQPEKSR
LRKLLHPDTDAVNVAYSKRTPARQALFYVFTNLIAIIIYFIDTVFFGLVIEGKDKLKKVRGGAVTVMNHI
HPMDCTMVKLAVFPRPIYFTSLVNNLELPLVGWLIRFCGALPVPNGKGKLVGFMKHIKHGIQNGDLVHFY
PEGMLIRNYEGLREFQPGAFYTAVHTGCPVIPMVLVNIHPQGIWRIAGGRRMHLFIGEPQYPNSELSPKE
SVTELKNRTWQIMNEMMNEPVEAERISFSVAVRIACILYIASQVVRIIAIRL
>WP_265975751.1 glycosyltransferase [Thermoclostridium stercorarium]
(SEQ ID NO: 179)
MRIPTVSAFHLQPENVTYSIGLGKSKRANDVIYRYFYKCFYNRFRFIHCPSEMIAEQLMQHGYDAECRVI
SNGVNDLFRPAEVRRPEYLKGKIVLLMVGRLSGEKRQDLLIEAVQYSKYRDKIQLVFAGMGPKEKKYRKL
SRNLKNKPIFRFFTQEELLQMYNICDLYIHTSDAEIEGISCIEAMACGAVPVISDSHLSATKSFALHPNC
LFKAGDARSLAEKIDYWIEHPEERSKMSRIYAEKAETIRVSKCVAEMERLYKDVISDHHKNGYKQPEKSR
LRKLLHPDTDAVNVAYSKRTPARQALFYVFTNLIAIIIYFIDTVFFGLVIEGKDKLKKVRGGAVTVMNHI
HPMDCTMVKLAVFPRPIYFTSLVNNLELPLVGWLIRFCGALPVPNGKGKLAGFMKHIKHGIQNGDLVHFY
PEGMLIRNYEGLREFQPGAFYTAVHTGCPVIPMVLVNIHPQGIWRIAGGRRMHLFIGEPQYPNSELSPKE
SVTELKNRTWQIMNEMMNEPVEAERISFSVAVRIACILYIASQVVRIIAIRL
>WP_270268159.1 glycosyltransferase [Bifidobacterium bifidum]
(SEQ ID NO: 180)
MRDNKNEEPRPQDRSALDRPLTIVLVMDTIGNKGNGTSNSALQYAHELERQGHHVRLVGIGAPEYPARVN
KVPLVSWVAAKQQMQFAKPSDTLFRTAFAGADVVHLYLPFKFGRCAYKVARSMGIPVTAGFHLQPENVMY
SAGPLKYLPGMSGFLYALFRFWLYRRIGHIHAPTEMIAGQLRAHGYKAKLHVISNGYVPRFTPKRQRSDG
APAPVPFRIVASGRLSHEKDQITLIKAISRCRHAKDIELIICGTGPLRHYLRFRADRLLERKAQIGFHKN
AEMPALLRSCDLFVHPSIVDIESLSVIEGMASGLVPVIASAELSAASQFALLDESLFPARDVAMLARRID
WWIDHPHELAVWGGLYAEHAKADYSVEASVAKFVAMEREAIADAKA
>WP_271723644.1 glycosyltransferase [Bifidobacterium breve]
(SEQ ID NO: 181)
MRENGIEKSIDRPLTIALVVDTVGNQGNGTSNSALQWAAELERQGHHVRLVGVGAPEYPARVNKVPLVSW
VAAKQLMQFAEPSDTLFRTAFQGVDVVHVYMPFKFGRRAAKVAHQMGVPVTAGFHLQPENVLYSAGPLRH
IPGISSFLYWLFKHWLYKRIDHIHVPTEMTASLLRAHGYKAKLHVISNGYSPVYSPKKPVDPEASAPVPF
RVIASGRLASEKDQITLIKAVSMSKHAGDIQLIIAGTGPLKQYLRFRAGRLLARKADIGFHKHADMPDLL
RSGDLFVHCSIADIESVSVIEAMACGLVPVIAASELSAASQFALLGESLFPVQNAAMLARRIDWWIEHQV
ERAEWSEKYAEYTKAHYSVEASVHKFVDMEREAIGE
>WP_277156009.1 glycosyltransferase [Bifidobacterium sp. ESL0798]
(SEQ ID NO: 182)
MPHSRNAAGDAAKPLTIVLVVDSVGNRGNGTSNSALQYAKELEREGHHVRLVGVGAPDYPVGIHHLPLIS
WLAAKQQMQFAEPDDAVFRHAFAGADVVHIYMPFKFGRHALAVARSMGIPVTAGFHLQPENVTYSAGPLR
YIPGIPSLIYYLFDFWLYRHIGHIHAPSQMIARQLRAHGYRARLHVISNGYSSRFHPAQKQSGNKHGNNE
HGKRDEDGFRIIASGRLTHEKDHETLIRAVALSRHAAQIDLTICGTGPLQRHLRRLAKRLLPRPAHIGFQ
PNDEMPNLLRTADLLVHPSIVDIESLSVLEGIASGLVPVIADSPLSAASQFALTDSSVFPARNAKALARR
IDWWIEHPQELAEWGPKYAQEAREKYALPQSVHRFVAMEREAIADACK
>WP_277161713.1 glycosyltransferase [Bifidobacterium sp. ESL0682]
(SEQ ID NO: 183)
MSDSRNAAVDADKPLTIALVVDSVGNRGNGTSNSALQYAQELERQGHHVRLIGVDAPDYPVEVHHLPFVS
WLAAKQQMQFAQPDDAVFRQAFKGADIVHIYMPFKFGRCALAVARSMAIPVTAGFHLQPENVTYSAGPLR
YIPGIPSLIYYLFDFWLYRHIGHIHTPSQMIARQLRAHGYRARLHVISNGYSGRFHPAKAQSCSADGDVS
RRVFRIVASGRLTHEKDHETLIRAVAMSRHANQIDLTICGTGPLQRHLRRLAKRLLPRPAHIGFHANEDM
PNLLREADLLVHPSIVDIESLSVLEGIASGLVPVIADSPLSAASQFALTDSSIFPARNAKALARRIDWWI
EHPQKRAEWGPQYAKEAREEYALPQCVRKFIAMEREAIADERKKR
>WP_277174872.1 glycosyltransferase [Bifidobacterium sp. ESL0704]
(SEQ ID NO: 184)
MPESRNVAGNAIEPLTIALVVDSVGNRGNGTSNSALQYAKELEREGHHVRLVGVGAPDYPVGIHHLPFIS
WLAAKQQMQFAEPDDAVFRRAFAGADVVHIYMPFKFGRHALTVARSMGIPVTAGFHLQPENVTYSAGPLR
YIPGIPSLIYYLFDFWLYRHIGHIHAPSQMIARQLRAHGYRARLHVISNGYSSRFHAASTQTAETHDKNA
EGAFRIIASGRLTHEKDHETLIRAVALSRHAAQIDLTICGTGPLQRHLRRLAKRLLPRPAHIGFQPNDEM
PNLLRTADLLVHPSIVDIESLSVLEGIASGLVPVIADSPLSAASQFALTDSSVFPARNAKALARRIDWWI
EHPQELAEWGPKYAQEAREKYALPQSVHRFVAMEREAIAGAQKTWQPAAPGRR
>WP_278659680.1 glycosyltransferase, partial [Ruthenibacterium
lactatiformans]
(SEQ ID NO: 185)
RRPPRSTLFHYTTLFRSCFAKPDEEAYYTAFKDADIVHFYMPFRFCRRGEELARQMGIPTVAAFHVQPEN
ITSSIFLGKNRRVNDFLYWWFYKVFYNRFDHIHCPSAFIARQLERHGYGAKLWVISNGVADAFRPAQVPR
APELEGRFTILMIGRLSGEKRQDLIIEAAKRSKYADRLQLVFAGKGPKEKAYRKLAAGLAHPPVFGFYGQ
DELRRLINMCDLYVHASDAEIEGISCMEALACGLVPVISDSPLSATGQFALCAESLFRAGDADDLARRID
YWVEHPEEKRAYAEQYALRQDENRVEACVARAEEMYASAIRDKRRKGYRKVPLSRWRRCTSPSCEAIRRN
FCQGGTVRTLLFWLFTTLLSPILWLLDRLWLGARIEGRENLDAVQGGAVSIMNHVHPLDCTMAKVALFPY
RLWFISLASNLQKPFTGWLIRFCGGVPLPDDIHGMAALERGMEARIRGGAFVHFYPEGMLVPYHEGLRAF
HPGAFATAVRAGCPVVPMMLCRRPARGLWAWRKKPCFTLRIGAPLYADASLPKKQAARDLQLRAEAAMLA
LERGETPPLAVGEPALDAE
>WP_278768024.1 glycosyltransferase [Bifidobacterium boum]
(SEQ ID NO: 186)
MSQSSKFMRATGVNGDADRPLTIALVVDTAGNEGNGTSNSALQYARELRRQGHTVRLVGIGSPEYPIRVH
HIPLVSWIAAKQQMQFAQPDRAVFHKAFAGVDVVHVYLPFKYGRVAVQTAHEMGIPATAGFHLQPENVLY
SAGPLRYIPGASAFIYWLFDRMLYRHIRHIHTPSHMIAAQLRAHGYRQRLHVISNGYSPRFTPKRQREPG
SPSPRPFRIVASGRLASEKDHVTLIRAVALSRHAEDIELVICGTGPLQHELRRMAGKLLPGRWSIGFHDN
AQMPELLRSCDLLVHPSIVDIESLSVLEGMASGLVPVIARSTQSAAGQFALNDHSLFEAQDSAMLAQRID
WWIDHPDELSRWGAVYAEHTREEYSIASCVRKFVAMEREAIADFDGRL

CgT is the glycosyltransferase that forms cholesteryl-α-d-glucopyranoside (CGL), a major cell wall glycolipid of certain bacterial species. CGL was originally discovered as one of three major cholesteryl glucosides (CGs) that account for 25% of total cell wall lipids of Helicobacter species, including H. pylori. In H. pylori, CgT catalyzes the conversion of membrane cholesterol to cholesteryl glucosides, which can be incorporated into the bacterial cell wall, facilitating evasion from immune defense and colonization in the host.

By “Cholesterol alpha-glucosyltransferase polynucleotide” is meant a nucleic acid molecule encoding a CgT polypeptide. The sequence of an exemplary CgT polynucleotide follows:

>DQ865239.1 Helicobacter mustelae cholesterol alpha-glucosyltransferase
(CHLaGcT) gene, complete cds
(SEQ ID NO: 187)
ATGACCATCGGAATAGTAATTGATAGCTACAATGATAGAAGCAATGGCACTTCTATGACGGCTTTTCGTT
TCGCAAGAGAATTTGTCAAAAAGGGGCATGAGGTTCGCATCGTAGCCTGCAATGTAAGCAAGAGTATGAG
CGATGAGGAGGATCTCAAGCTCTATCCCGTGAAGCAGAGATATATCCCCATCGTTACAGAGGTTTCTAAA
AAACAGCATATGATTTTTGGCGCTCCTGATTTGGAGGTTTTGCAATCTGCTGTGGTGGGCTGTGATATCG
TGCATTTTTATATGCCCTTTGCACTTGAGATTGCAGGGATGCATCTCTGCAGAAGCCTTCGTATCCCCTA
TATCAGTGCCTTTCATGTCCAGCCTCAGCATATCAGCTACAACATGAATATGAATTTTTCTTGGTTTAAT
ACCTATCTTTTCAAAAGATTTTATAAGCATTTTTATCGCTACACCCATCACATCCATTGCCCGAGTAAAT
TCATCGAAAAAGAACTCCAAAGGGAAAATTATGGAGGCAAAAAATACACCATTAGCAATGGCTTTTTTGG
TGGGGATAGGGTGATGGCAGATCCTTATGAAGACTCCTTTTTTCACATCGCTTCAGTGGGAAGATTTTCC
AAAGAAAAAAAGCAAGACATCATCATCAAGGCCATAGCAAAAAATCCCTATGCCGATAAAATCAAACTGC
ATTTGCATGGTGTGGGGCCGCGGGAGAAATATCTCAAAAATCTCTGCAATAAGCTGCTTATCAATAAGCC
AGAATTTGGATTTATCGATAATGGCGCATTGCTTGAAAAACTCGCAAAAATGCATCTTTATGTGCATGCA
GCAAAGGTAGAGAGCGAGGCAATTTCTTGCCTTGAGGCCATATCTCTTGGAGTGGTGCCTGTAATCGCAG
ATTCAGAGACAAGCGCTACGGTGCAATTTGCCCTCGATCCTCTAAGTCTTTTTGAGGTCAATAATGTTGC
AGATTTAAGCAATAAGATCACCTATTGGATCGAGCATCCAAAGGAGTTGCTAGCCTATAAGCAAAAATAT
GCAGAGTCTGCGCTGCAGTATTCTCTAGACAAAAGTATTGAAGAAACCCTGGGATTGTATGAAGAAGCAA
TCAGGGATTTTCGCGATCAGCCTGCCTTGTTTGATCGCATCAATGCATAA

A “codon-optimized” nucleic acid molecule (polynucleotide) refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

By coprostanol is meant a compound having the following structure:

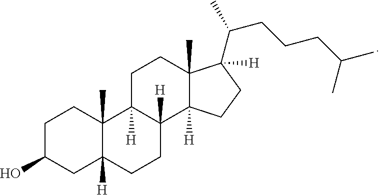

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight. “Detect” refers to identifying the presence, absence or amount of an analyte, compound, agent, or substance to be detected. In embodiments, the analyte is cholesterol or a metabolite thereof.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cardiovascular disease, cholesterol related disorders, or diseases associated with or characterized by increased levels of plasma triglycerides, plasma cholesterol, or serum C-reactive protein.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In an embodiment, an effective amount of a compound, cell, or probiotic composition is an amount sufficient to reduce the level of cholesterol in a biological sample of a subject by at least about 1%, 2%, 5%, 10%, 20%, 25%, 35%, 50%, 75% or more.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increase” is meant to alter positively relative to a reference. An increase may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “Intestinal Sterol Metabolism A (IsmA) polypeptide” is meant a protein or fragment thereof having at least 85% amino acid sequence identity to an IsmA amino acid sequence associated with GenBank Accession No. WP_078769004.1 or any of the IsmA amino acid sequences provided herein, or fragments thereof, and that has cholesterol dehydrogenase activity. For example, IsmA proteins metabolize cholesterol to cholestenone metabolic activity. The sequence of an exemplary IsmA protein from Eubacterium coprostanoligenes, ECOP170, which is described by Kenny et al., Cell Host Microbe. 2020 Aug. 12; 28(2): 245-257.e6, follows:

>WP_078769004. 1 SDR family NAD(P)-dependent oxidoreductase [Eubacterium
coprostanoligenes]
(SEQ ID NO: 188)
MSTCWLQGKTVVVTGASGGMGAGIAATLIKKHGCTVIGVARNEKKMLKFVDELGETYAKQFSYELFDVSS
KENWEKFAEELQEKGVKVDVLINNAGILPKFKRFDRYSYEEIERAMNINFYSCVYSVKTMLPMLLQSSTP
AIINIDSSAALMTLAGTSMYSASKAALKGFTEALRVEFQGKMFVGLVCPGFTKTDIFSGQGDADMSNGAK
VMDMISTDCDKMVKMIMFGIEHKTPMQVHGFDAHAMSVFNRLMPVYGSKLFSSIMRMSNVDIFKEVFSD.

In some embodiments, IsmA polypeptide may instead mean a protein or fragment thereof having at least 85% amino acid identity to any of the exemplary IsmA equivalent protein sequences below, and that has cholesterol dehydrogenase activity.

>MSP257_G141039_k105_18263_1
(SEQ ID NO: 189)
MKTAIITGASSGLGREFARQLTDIFPEIECCWLIARREDRLEEIAREMVGVETVCLPLDLCDSMSFTTLQEKLAAEK
PEVAILINNAGCGYLGRMGETETAVQTRMVDLNVRAMTAMTNLVIPYMPAGGRILNTSSIASFCPTPRMTVYGASKA
YVSSFTVGLSEELKRRDITVTAVCPGPMKTEFLDVGSITGRSPAFEYLPYCDQVRVAAGALRAAKAGRTMYTPRLFY
KFYRLLAKVTPVKMMVKFTKT
>J115_02655 hypothetical protein
(SEQ ID NO: 190)
MKTAIITGASSGLGREFARQLTDIFPEIECCWLIARREDRLEEIAREMVGVETVCLPLDLCDSMSFTTLQEKLAAEK
PEVAILINNAGCGYLGRMGETETAVQTRMVDLNVRAMTAMTNLVIPYMPAGGRILNTSSIASFCPTPRMTVYGASKA
YVSSFTVGLSEELKRRDITVTAVCPGPMKTEFLDVGSITGRSPAFEYLPYCDQVRVAAGALRAAKAGRTMYTPRLFY
KFYRLLAKVAPVKLMVKFTKT
>RJX3711_01778 hypothetical protein
(SEQ ID NO: 191)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT
>MSP384_G139985_k105_38511_6
(SEQ ID NO: 192)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>0262AD_0917_052_G4|00637 Sulfoacetaldehyde reductase 2
(SEQ ID NO: 193)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>0471HH_0918_059_C7|01044 Cyclopentanol dehydrogenase
(SEQ ID NO: 194)
MSEIAVITGGTSGIGRATALYLRDAGYTVYELSRREQGVEGLHHIRCDITDEGQVRAAVAEIMDRAGRIDVLVNNAG
FGISGAVEFTDTAEAQRLLDVNFFGMVRMNKAVIPHMRQAGRGRIVNLSSVAAPCPIPFQAYYSAGKAAVNAYTMAL
ANELRPFGITVCAVQPGDIHTGFTAARVKTMEGDDAYGGRIGRSVQRMEHDEQNGMDPAKAGAFIARVAMKRRPKPI
YTIRLDYQFFVFLTRILPGRTLNWLIGLLYAK
>0471HH_0918_059_C7|02227 Serine 3-dehydrogenase
(SEQ ID NO: 195)
MKKIAVITGASSGMGRRFAETVDSFGRFDEVWVIARHEKALEELRDQVPYPIRPLALDLTDRRSFQTYADALAEEPV
EVGLLVNASGFGKFRAVVDTPLETNLNMVDLNCQAVMALCQLTVPYMPRGGQIINIASVAAFQPIPYINVYGATKAF
VLSFSRALNRELRSQGVRVMALCPFWTRTAFFDRATGDGGESVVKKYVAMYEPEQLVQRAWRDAKRGKDVSQYGFVA
RFQAGLTKLLPHSLVMDVWMRQQKLQ
>1462QI_0319_071_A08|01307 Serine 3-dehydrogenase
(SEQ ID NO: 196)
MKKIAVITGASSGMGRRFAETVDTFDRFDEVWVIARHEKALEELRDRVPYPIRALALDLTDRRSFQTYADALAEEPV
EVGLLVNASGFGKFRAVVDTPLEVNLNMTDLNCQAVVALCQLTAPYMPRGGQIINIASVAAFQPIPYINVYGATKAF
VLSFSRALNRELRSRGVRVMALCPFWTRTAFFDRATGDGGESVVKKYVAMYEPEQLVQRAWRDARRGRDVSQFGFVA
RFQTALTKLLPHSLVMDVWMRQQKLQ
>1462QI_0319_071_A08|02518 3-oxoacyl- [acyl-carrier-protein] reductase FabG
(SEQ ID NO: 197)
MSEIAVITGGTSGIGRATALCLRDAGYDVYELSRREQGVEGLHHIRCDITDEDQVRAAVAEIIGQAGRIDVLINNAG
FGISGAVEFTDTAEAQRLLDVNFFGMVRMNKAVIPHMRQAGRGHIVNLSSVAAPCPIPFQAYYSAGKAAVNAYTMAL
ANELRPFGITVCAVQPGDIHTGFTAARVKTMAGDDVYQGRIGRSVQRMEHDEQTGMDPAKAGAFIARVAMKRRPKPI
YTIRLDYQFFVFLTRILPGRTLNWLIGLLYAR
>1462QI_0319_071_A09|00528 Serine 3-dehydrogenase
(SEQ ID NO: 198)
MKKIAVITGASSGMGRRFAETVDSFGRFDEVWVIARHEKALEELRDQVPYPIRPLALDLTDRGSFQTYADALAEEPV
EVGLLVNASGFGKFRAVVDTPLETNLNMVDLNCQAVMALCQLTAPYMPRGGQIINIASVAAFQPIPYINVYGATKAF
VLSFSRALNRELRSRGVRVMALCPFWTRTAFFDRATGDGGESVVKKYVAMYEPEQLVQRAWRDAKRGKDVSQYGFVA
RFQAGLTKLLPHSLVMDVWMRQQKLQ
>1462QI_0319_071_A09|00875 Cyclopentanol dehydrogenase
(SEQ ID NO: 199)
MSEIAVITGGTSGIGRATALYLRDAGYTVYELSRREQGVEGLHHIRCDITDEDQVRAAVAEIMDRAGRIDVLVNNAG
FGISGAVEFTDTAEAQRLLDVNFFGMVRMNKAVIPHMRQAGRGRIVNLSSVAAPCPIPFQAYYSAGKAAVNAYTMAL
ANELRPFGITVCAVQPGDIHTGFTAARVKTMEGDDAYGGRIGRSVQRMEHDEQNGMDPAKAGAFIARVAMKRRPKPI
YTIRLDYQFFVFLTRILPGRTLNWLIGLLYAK
>1462QI_0319_071_A11|01307 Serine 3-dehydrogenase
(SEQ ID NO: 200)
MKKIAVITGASSGMGRRFAETVDTFDRFDEVWVIARHEKALEELRDRVPYPIRALALDLTDRRSFQTYADALAEEPV
EVGLLVNASGFGKFRAVVDTPLEVNLNMTDLNCQAVVALCQLTAPYMPRGGQIINIASVAAFQPIPYINVYGATKAF
VLSFSRALNRELRSRGVRVMALCPFWTRTAFFDRATGDGGESVVKKYVAMYEPEQLVQRAWRDARRGRDVSQFGFVA
RFQTALTKLLPHSLVMDVWMRQQKLQ
>1462QI_0319_071_A11|02332 3-oxoacyl- [acyl-carrier-protein] reductase FabG
(SEQ ID NO: 201)
MSEIAVITGGTSGIGRATALCLRDAGYDVYELSRREQGVEGLHHIRCDITDEDQVRAAVAEIIGQAGRIDVLINNAG
FGISGAVEFTDTAEAQRLLDVNFFGMVRMNKAVIPHMRQAGRGHIVNLSSVAAPCPIPFQAYYSAGKAAVNAYTMAL
ANELRPFGITVCAVQPGDIHTGFTAARVKTMAGDDVYQGRIGRSVQRMEHDEQTGMDPAKAGAFIARVAMKRRPKPI
YTIRLDYQFFVFLTRILPGRTLNWLIGLLYAR
>1462YY_0218_030_D6|01408 Sulfoacetaldehyde reductase 2
(SEQ ID NO: 202)
MKTAVITGASSGLGREFVRQFHSVFPEIQRVWLIARRAERLQELAGQLEEKGLSVLTLPLDLCDTMSFTAYHEHLVE
EQPEIALLVNNAGCGYLGNIGEVDTVSQTRMIDLNLRALTALTNLTVPYMAAGSRILNVSSIASFCPNPRMTVYSAT
KAYVSAYTIGAAEELKAKGITVTAVCPGPMATEFLSVGRITGNSRMFERLPYCDQVQVAGAALRAAREGRTIYTPRL
FYKFYRLLAKVTPAKLMVKLTKT
>3050YG_0918_062_E10|02102 3-oxoacyl- [acyl-carrier-protein] reductase FabG
(SEQ ID NO: 203)
MNTIFIVGSSSGIGKATAKLFAEKGWTVIATMRTPEKETELTAYPNVCLFPLDVTKPEQIEATVSAVLKEYDVDVLF
NNAGYGMKSRFEDMTEEAMQRSLNTNLLGMVRVTQKFIPYFKAKKSGMILTTTSLAGEMGLVLDGIYAADKWAVTGL
CEMLYHELYPFGIQVKTIVPGVVKTGFKMELSEMPGYDDLIKKQTNLLIPDMDSMETPEEVAQDIYAAVTDGDADRM
CYVTGAITKTLYAKRQELGDEDFRRYMRGLLE
>3790QQ_0218_002_G9|01148 Sulfoacetaldehyde reductase 2
(SEQ ID NO: 204)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>4324HL_0218_028_E7|00180 Cyclopentanol dehydrogenase
(SEQ ID NO: 205)
MREIAVITGGTSGIGRATALYLRDAGYTVYELSRREQGVEGLHHIRCDITDEGQVRSAVAEIMDRAGRIDVLVNNAG
FGISGAVEFTDTAEAQRLLDVNFFGMVRMNKAVIPHMRQAGRGRIVNLSSVAAPCPIPFQAYYSAGKAAVNAYTMAL
ANELRPFGITVCAVQPGDIHTGFTAARVKTMEGDDAYGGRIGRSVQRMEHDEQTGMDPAKAGAFIARVAMKRRPKPI
YTIRLDYQFFVFLTRILPGRTLNWLIGLLYAK
>4324HL_0218_028_E7|02464 Serine 3-dehydrogenase
(SEQ ID NO: 206)
MKKIAVITGASSGMGRRFAETVDTFGRFDEVWVIARHEKALEELRDQVPYPIRPLALDLTDRGSFQVYADALAEEPV
EVGLLVNASGFGKFRAVADTPLETNLNMVDLNCQAVMALCQLTAPYMPRGGQIINIASVAAFQPIPYINVYGATKAF
VLSFSRALNRELRSRGVRVMALCPFWTRTAFFARATGDGGESVVKKYVAMYEPEQLVQRAWRDAKRGKDVSQFGFVA
RFQTALTKLLPHSLVMDVWMRQQKLQ
>4324HL_0218_028_E9|00882 Sulfoacetaldehyde reductase 2
(SEQ ID NO: 207)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSQILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>4324HL_0218_028_F5|00714 Sulfoacetaldehyde reductase 2
(SEQ ID NO: 208)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>4452YQ_0918_057_F10|00956 putative oxidoreductase
(SEQ ID NO: 209)
MRKKPIAIVTGASGGMGLEFVRLLAKKEELDEIWVIARSADKLEAVKGEIGSRLQCFPMDLSNLENIKKFGSQEGLK
DCNIKYLINNAGFAKFCSHDDLSLEESINMIDLNVSGVVAMGLVCIPHMEKGGRIINIASQASFQPLPYLNIYSSTK
AFVRSYTRALNVELKDRGVTATAVCPGWMKTGLFARGLIGAKKEVKNFVGMVTPDVVAKKALSDADKGKDMSVYGLY
VKMCHLIAKVLPQKVMMKLWLIQQK
>5399RU_0319_069_B05|00631 Serine 3-dehydrogenase
(SEQ ID NO: 210)
MSERRVAIVTGGTSGIGKATALALRHAGCTVYELSRRAEGTEGLHHISADVTDERAVRDAVAQVMAAEGHIDILVNN
AGFGISGAIEYTETEDAKKLFDVNFFGMVNMNRAVVPLMRQAGRGRIVNLSSVAAPVPIPFQAYYSATKAAVNAYSM
ALANELRPFGVTVCAVMPGDICTGFTAARKKIVTGDDIYHGRISRSVQRMEHDEETGMEPAKAGAYIASVALRDGSR
HPLYAIRFDYKFFTFLAKVLPARFLNWLIYCLYGK
>5399RU_0319_069_B05|02025 putative oxidoreductase
(SEQ ID NO: 211)
MKKIAVITGASSGMGKRFAETVDVYGTFDEVWVIARHREQLEALRETVPFPIRVLPLDLTDRSSFDVYAAALAEEPV
EVGLLMNCSGYGKFSAVLDTPLAVNLNMTDLNCQAVVAMCQLTAPYMVRGGQIINIASVAAFQPIPYIDVYSAGKAF
VLSFSRALNRELRGRGIGVMAVCPFWTRTAFFDRAIKGGEEPVVKKYAAMYDPADIVKRTWRDAKRGKDVCKYGFVA
RAQAGLTKLLPHSVVMDVWMRQQKLR
>5399RU_0319_069_C10|00788 Serine 3-dehydrogenase
(SEQ ID NO: 212)
MSERRVAIVTGGTSGIGKATALALRHAGCTVYELSRRAEGTEGLHHISADVTDERAVRDAVAQVMAAEGHIDILVNN
AGFGISGAIEYTETEDAKKLFDVNFFGMVNMNRAVVPLMRQAGRGRIVNLSSVAAPVPIPFQAYYSATKAAVNAYSM
ALANELRPFGVTVCAVMPGDICTGFTAARKKIVTGDDIYHGRISRSVQRMEHDEETGMEPAKAGAYIASVALRDGSR
HPLYAIRFDYKFFTFLAKVLPARFLNWLIYCLYGK
>5399RU_0319_069_C10|02031 putative oxidoreductase
(SEQ ID NO: 213)
MKKIAVITGASSGMGKRFAETVDVYGTFDEVWVIARHREQLEALRETVPFPIRVLPLDLTDRSSFDVYAAALAEEPV
EVGLLMNCSGYGKFSAVLDTPLAVNLNMTDLNCQAVVAMCQLTAPYMVRGGQIINIASVAAFQPIPYIDVYSAGKAF
VLSFSRALNRELRGRGIGVMAVCPFWTRTAFFDRAIKGGEEPVVKKYAAMYDPADIVKRTWRDAKRGKDVCKYGFVA
RAQAGLTKLLPHSVVMDVWMRQQKLR
>5399RU_0319_069_D10|00785 Serine 3-dehydrogenase
(SEQ ID NO: 214)
MSERRVAIVTGGTSGIGKATALALRHAGCTVYELSRRAEGTEGLHHISADVTDERAVRDAVAQVMAAEGHIDILVNN
AGFGISGAIEYTETEDAKKLFDVNFFGMVNMNRAVVPLMRQAGRGRIVNLSSVAAPVPIPFQAYYSATKAAVNAYSM
ALANELRPFGVTVCAVMPGDICTGFTAARKKIVTGDDIYHGRISRSVQRMEHDEETGMEPAKAGAYIASVALRDGSR
HPLYAIRFDYKFFTFLAKVLPARFLNWLIYCLYGK
>5399RU_0319_069_D10|01996 putative oxidoreductase
(SEQ ID NO: 215)
MKKIAVITGASSGMGKRFAETVDVYGTFDEVWVIARHREQLEALRETVPFPIRVLPLDLTDRSSFDVYAAALAEEPV
EVGLLMNCSGYGKFSAVLDTPLAVNLNMTDLNCQAVVAMCQLTAPYMVRGGQIINIASVAAFQPIPYIDVYSAGKAF
VLSFSRALNRELRGRGIGVMAVCPFWTRTAFFDRAIKGGEEPVVKKYAAMYDPADIVKRTWRDAKRGKDVCKYGFVA
RAQAGLTKLLPHSVVMDVWMRQQKLR
>5399RU_0319_069_D12|00785 Serine 3-dehydrogenase
(SEQ ID NO: 216)
MSERRVAIVTGGTSGIGKATALALRHAGCTVYELSRRAEGTEGLHHISADVTDERAVRDAVAQVMAAEGHIDILVNN
AGFGISGAIEYTETEDAKKLFDVNFFGMVNMNRAVVPLMRQAGRGRIVNLSSVAAPVPIPFQAYYSATKAAVNAYSM
ALANELRPFGVTVCAVMPGDICTGFTAARKKIVTGDDIYHGRISRSVQRMEHDEETGMEPAKAGAYIASVALRDGSR
HPLYAIRFDYKFFTFLAKVLPARFLNWLIYCLYGK
>5399RU_0319_069_D12|02046 putative oxidoreductase
(SEQ ID NO: 217)
MKKIAVITGASSGMGKRFAETVDVYGTFDEVWVIARHREQLEALRETVPFPIRVLPLDLTDRSSFDVYAAALAEEPV
EVGLLMNCSGYGKFSAVLDTPLAVNLNMTDLNCQAVVAMCQLTAPYMVRGGQIINIASVAAFQPIPYIDVYSAGKAF
VLSFSRALNRELRGRGIGVMAVCPFWTRTAFFDRAIKGGEEPVVKKYAAMYDPADIVKRTWRDAKRGKDVCKYGFVA
RAQAGLTKLLPHSVVMDVWMRQQKLR
>5399RU_0319_069_E08|01796 3-oxoacyl-[acyl-carrier-protein] reductase FabG
(SEQ ID NO: 218)
MLEERSAIILGASSGVGYGAALRFAEEGAHVIAGARSLNKLEALKAEAEKQGFSGTITPVACDVTHDNDLDHILQVC
LDAYGKVDILACIAQSNLNDQHGFEDSDLENIMAFYRGGPGYTFQMIKKCLPHMKEQHYGRIITCASGAGERYTPHS
CGYGMAKAAIINLTRTCAVELGQYGIVTNCFLPVIQVSHFEKQSNDAALAVPIMNALSPVGRMGDAYEDGSPMLAFL
ASEEARYINGQVISICGGISYINPNRARVIHPRPVWLSSAYTASYSSGTCLQTAQANCRP
>5399RU_0319_069_G09|00785 Serine 3-dehydrogenase
(SEQ ID NO: 219)
MSERRVAIVTGGTSGIGKATALALRHAGCTVYELSRRAEGTEGLHHISADVTDERAVRDAVAQVMAAEGHIDILVNN
AGFGISGAIEYTETEDAKKLFDVNFFGMVNMNRAVVPLMRQAGRGRIVNLSSVAAPVPIPFQAYYSATKAAVNAYSM
ALANELRPFGVTVCAVMPGDICTGFTAARKKIVTGDDIYHGRISRSVQRMEHDEETGMEPAKAGAYIASVALRDGSR
HPLYAIRFDYKFFTFLAKVLPARFLNWLIYCLYGK
>5399RU_0319_069_G09|02005 putative oxidoreductase
(SEQ ID NO: 220)
MKKIAVITGASSGMGKRFAETVDVYGTFDEVWVIARHREQLEALRETVPFPIRVLPLDLTDRSSFDVYAAALAEEPV
EVGLLMNCSGYGKFSAVLDTPLAVNLNMTDLNCQAVVAMCQLTAPYMVRGGQIINIASVAAFQPIPYIDVYSAGKAF
VLSFSRALNRELRGRGIGVMAVCPFWTRTAFFDRAIKGGEEPVVKKYAAMYDPADIVKRTWRDAKRGKDVCKYGFVA
RAQAGLTKLLPHSVVMDVWMRQQKLR
>5399RU_0319_069_H12|00177 Cyclopentanol dehydrogenase
(SEQ ID NO: 221)
MGKIVIVTGGTSGIGLHTAAYLQSQGCTVYTVSRRPCDDARFHHICADVTQEADVARAVKSVLDEAGRIDILVNNAG
FGISGAVEFTDLATARRQLDVNFWGMAAMTHAVLPVMRRQGSGRIVNLSSVAAPAAIPFQTYYSVSKAAINDFTLAT
ANEVRPFGITVCAVMPGDICTGFTDARQKIPAGDEIYGGRISRSVAGMEKDERTGMAPEAAGRFVGKVALRTSHKPL
YTIGFVYRCCVLLLKLLPARISNWLVGKLYAK
>5399RU_0319_069_H12|00412 Sulfoacetaldehyde reductase
(SEQ ID NO: 222)
MQKTIAVITGASSGMGAEFVRTIESFGVRLDEIWAIARRQERLEALQAPCPVRPLALDLTDPASFSEYQALLEQEQP
RVALLINASGFGKFAATWQTPLAVNQAMVALNCQAVLAMCQLTLPYLGEGSCIVNIASVAAFQPIPYINVYAASKAF
VLQLSRALNREVRPQGIRVMALCPFWTKTEFFNRAIDAGRKQIVKKYTAMYDPAQIVRRAWRDLARGKDVSKYGFVA
RFQAALCKLLPHSFVMTYWMHQQKLP
>8131GG_1118_056_H6|00424 putative oxidoreductase
(SEQ ID NO: 223)
MKTAVITGASSGLGLEFARQVHQVFPEIERVWLIARRVQRLEELAAQLTDEGLSVLTLPLDLCDTMSFTAYHEHLVE
EQPEISLLINNAGCGYLGKIGEVDTVSQTRMIDLNLRALTAITNLSVPYMEAGSRILNVSSIAAFCPTPRMTVYSAT
KAYVSAYTIGAAEELKAKGITVTAVCPGPMATEFLETGHIHNSPAFELLPYCDQVRVAGGALRAAKAGQTIYTPRLF
YKFYRLLAKLTPAKLMVKLTKT
>9573HE_0319_068_B09|02110 Sulfoacetaldehyde reductase 2
(SEQ ID NO: 224)
MKTAVITGASSGLGREFVRQFYSVFPEIERVWLIARRTDRFQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>msp_069|G140109_k105_26829_8
(SEQ ID NO: 225)
MSEIAVITGGTSGIGRATALYLRDAGYTVYELSRREQGVEGLHHIRCDITDEDQVRSAVAEIMDRAGRIDVLVNNAG
FGISGAVEFTDTAEAQRLLDVNFFGMVRMNKAVIPHMRQAGRGRIVNLSSVAAPCPIPFQAYYSAGKAAVNAYTMAL
ANELRPFGITVCAVQPGDIHTGFTAARVKTMEGDNVYGGRIGRSVQRMEHDEQNGMDPAKAGAFIARVAMKRRPKPI
YTIRLDYQFFVFLTRILPGRTLNWLIGLLYAK
>msp_069|SM-ITLFT_k103_17414_2
(SEQ ID NO: 226)
MREIAVITGGTSGIGRATALCLRDAGYTVYELSRRPEGVEGLHHIRCDITDEDQVRSAVAEIMDRAGRIDVLVNNAG
FGISGAVEFTDTAEAQRLLDVNFFGMVRMNKAVIPHMRQAGRGRIVNLSSVAAPCPIPFQAYYSAGKAAVNAYTMAL
ANELRPFGITVCAVQPGDIHTGFTAARVKTMEGDNVYGGRIGRSVQRMEHDEQTGMDPAKAGAFIARVAMKRRPKPI
YTIRLDYQFFVFLTRILPGRTLNWLIGLLYAR
>msp_099|G140906_k105_25884_7
(SEQ ID NO: 227)
VTPVKKVCVITGGTSGIGLCTAQAMLEKGYTVYELSRRAEGAPGMKHIMADVTKEETLAAAVQEILKQEDHIDVLIN
NAGFGISGAVEFTKTEDAQHQLDVNFFGMVRMNRQVLPVMRRQCHGRIVNLSSVAGAIPIPFQAYYSASKAAINSYT
MALANEVKPFGIQVCCVQPGDIRTGFTAAREKNPEGDDIYGGRIARSVAGMERDEQNGMAPEKAGAFIAHVADKKSV
QPINTIGLQYKFFCFLVKILPAKTLNWLVGLIYAK
>msp_099|SM-GRF5P_k103_52182_1
(SEQ ID NO: 228)
MTAAVITGASAGLGAEFTRQLVREFPEIEEFWLIARRVGKLEELAQQFPEKKFVCMGLNLLDPKSFRYLGEKLAEQK
ADVRVLVNNAGCGTTGNIGSSASSADVMRVVNLNVRALTMVTQTVVPFMTRGAKIINVSSIAAFCPTPRMTVYSASK
AYVSAFTGGIADELRPKGISVTAVCPGPMKTEFFDAAGDHDLFGNIPWCDPVKVVAGTIRAAKKGRTFYTPTAFNKF
YRFVAKILPMKWMIKATRV
>msp_099|SM-GREZ2_k103_6461_1
(SEQ ID NO: 229)
MEKLDLHGKAGFIIGGTRGIGNAIAMRMARLGMNLTVIARKEDELAAMKAQVEPLGVRFLGIQSSNTDFDAIAAAFK
TSWQTYGRLDLLVNAAGTGVMTPFEELTKSDVDTTIDVNLKGTIYAVKLSVPYFEKTGGGNILNISSMSAIRGIPDP
VNNNGIYTATKFAINGFTECMQKYLLKYGIRVTALCPGSTATSWWTRWTHSFGTDAMLPVETLADIAELIVTSPEKV
LFKQLQVLPTVEIDNF
>msp_108|G139968_k105_17260_3
(SEQ ID NO: 230)
MNIAIVTGASSGMGREFVRQLGGYVSVDEIWAVARRASALETLKAEASVPVRPIVLDLLEASSFTRLEALLESEKPN
VRLLVNAAGFGKFGAYRKIPVEDDCRMIDLNCKALLLMTRLCVPYMQPGSHILELDSLSAFQPVPYITTYAATKAFV
LSYSRSMNRELKAKGIRVMAMNPGWVKTEFFNHAFQTNADNEVRYFNRLYEAKDVVKTGLNDLYHSKKDCSIHGFPV
KFQVFLVKLLPHSLVMNTWLNQQKKAKNNKGLTTK
>msp_108|2140596562_1_k103_28412_5
(SEQ ID NO: 231)
MSGQSRPVALVTGATQYTGFSTAKLFAQRGFDVCVTSRSLEKAEDAAQRIRSAVPGARVLGLQMDPPSVAQTQSAFQ
KVEAVFGRLDVFVANACAACRFKSLLSTTEEDYDAIVNANLKGYFFGAQAAARLMIKTECKGSMILIGSVHSRGAIG
NRIPYAISKGGIEVLGRNSAYELGKYGIRVNCVVCGAIVNDKFIEQTEEEKNARRANWPLGRESYPEDVAKAVFFLA
SDEAKTITGTSLVVDSGVSACMLKYDPNWETVG
>msp_121|SM-F6B63_k103_86534_2
(SEQ ID NO: 232)
VSKVAVVTGGTSGIGRATALALKDAGYTVYELSRRAEGVEGLHHISADVTDQQAVNDAAAQIMEAEGHIDVLVNNAG
FGISGAIEFTETAEAKALFDVNFFGMVNMNRAVVPLMRQAGRGRIVNLSSVAAPVPIPFQAYYSATKAAVNAYTMAL
ANELRPFGVTVCAVMPGDIHTGFTAARRKVSAGDAIYQGRISRSVKRMEHDEETGMDPAKAGAYIASVAMREGHHHP
LYAIRFDYKFFTFLAKVLPARFLNWLIYCLYGK
>msp_121|G141055_k105_39005_2
(SEQ ID NO: 233)
MKKIAVITGASSGMGKRFAETVDRYGTFDEVWVIARHEAQLEALRATVPFPVRVLALDLTDRGSFDVYAAALAETPV
EVGLLMNCSGYGKFSAVLDTPLAVNLNMTDLNCQAVVAMCQITAPYMPRGSQIINIASVAAFQPIPYIDIYGATKAF
VLSFSRALNRELKSRGIGVMAVCPFWTKTAFFDRAIKSDETPIVKKYAAMYDPDDIVARTWRDAKRGKDVCKYGFVA
RVQAGLTKLLPHSVVMDVWMKQQDLH
>msp_179_2140257829_1_k103_34263_2
(SEQ ID NO: 234)
MSKVAVVTGGTSGIGKQTALALKAAGYTVYELSRRAQGVEGLHHLVADITREELVDAAIGEVLRQEGHIDVVVNNAG
FGISGAIEFTKTEDAKRLFDADFFGMVNVNRAVIPHMRQAGAGRIVNLSSVAAAAPIPFQAYYSAAKAAVNSYTMAL
ANELRPYGVTVCAVQPGDIHTGFTAAREKTIDGDDVYGGRISRSVARMEHDEQTGMDPAKAGAFIAKVVMKQRVKPI
YTIRFDYQFLALLTRILPYRFLNWLIGVIYGK
>msp_179|G140919_k105_31346_2
(SEQ ID NO: 235)
MKRIAVITGASSGMGRRFAETVDTFGRFDEIWVIARHGAALEGLRERVPFPVRPLTLDLTDRSSFATYAKALTEEPV
EVGLLVNASGFGKFRAVVDTPLEVNLNMVDLNCQAVMALCQLTIPYMPEGGQIINIASVAAFQPIPYIDVYGASKAF
VLSFSRALNRELRGRGIRVMVLCPFWTRTAFFARATVNGGESVVKKYVAMYEPEQLVQRAWRDAKRGKDVSQFGFVA
RFQTGLTKFLPHSLVMDVWMHQQKLK
>msp_188|G141131_k105_87743_14
(SEQ ID NO: 236)
MSNKVAVVTGGTSGIGRATALALKDAGCTVYELSRRAQGVEGLHHISADVTKEESVRAAVEQIMAREGRIDILVNNA
GFGISGAVEFTSTEDAKSLFDVNFFGMVNMNRQVVPIMREAGRGRIVNLSSVAAPVPIPFQTYYSATKAAVNAYTMA
LANELRPFGVTVCAVMPGDIHTGFTAARRKIGEGDDIYQGRISRSVARMEHDEETGMDPARAGGYVARVALREGSHH
PLYAIRIDYKFFVFLTKVLPAGALNRLVYLIYGK
>msp_242|2140247930_1_k103_14216_45
(SEQ ID NO: 237)
MKKIAIVTGGSGGIGRCTAAALRDAGCTVYELSRREKPADGIVHITADVTDEAQVRAAIDEVISREGRIDILVNNAG
FGISGAAELTDTKDSHAQLELNVFGTDNVTRAVLPHMRAHGGGRIVCMSSIAGIVPIPFQLWYSVSKAAIIAYVLAL
QNEVKPFNISVCAIMPGDIASGFTDARKKSGAGDDVYAGRIKRSVAVMEHDERTGMSPEFAGRFVAKYALKKNSRPL
VAMGAAYKGAAALVKLLPRQTSNWLVGKIYAK
>msp_242|2140247930_1_k103_32115_23
(SEQ ID NO: 238)
MKIAVITGASSGMGREFVYALDRDEEFDELWVIARREDRLRELQSKCRAKVRPLALDLQDRASFAAYRALLESEKPE
ISVLVNAAGFGLFGMFTEMDMDKQLDIIDLNDRALTAMCHMSIPYMAAGSRIYNMGSMSSWQPVPYINVYGASKAYV
LSFSRALGVELEKQGIRVMAVCPGWIKTEFFSHAIHDNTVNYFNRYYGPEQVVAKALKDMKRGKDASVLGFPERMQV
RLVKLLPVKMVMNTWCRQQGKK
>msp_257|G141039_k105_18263_1
(SEQ ID NO: 239)
MKTAIITGASSGLGREFARQLTDIFPEIECCWLIARREDRLEEIAREMVGVETVCLPLDLCDSMSFTTLQEKLAAEK
PEVAILINNAGCGYLGRMGETETAVQTRMVDLNVRAMTAMTNLVIPYMPAGGRILNTSSIASFCPTPRMTVYGASKA
YVSSFTVGLSEELKRRDITVTAVCPGPMKTEFLDVGSITGRSPAFEYLPYCDQVRVAAGALRAAKAGRTMYTPRLFY
KFYRLLAKVTPVKMMVKFTKT
>msp_282|SM-J7DNM_k103_18055_3
(SEQ ID NO: 240)
VTPVKKVCVITGGTSGIGLCTAQAMLEKGYTVYELSRRAEGAPGMNHIAADVTKEETLAAAIQEILKREDHIDVLIN
NAGFGISGAVEFTKTEDAQHQLDVNFFGMVRMNRQVLPVMRRQCYGRIVNLSSVAGAIPIPFQTYYSASKAAINSYT
MALANEVKPFGIQVCCVQPGDIRTGFTAAREKNPEGDDIYGGRIARSVAGMERDEQNGMDPEKAGAFIAHVATRKGI
RPVNTIGLQYKFFCFLVKILPAKTLNWLVGLIYAK
>msp_282|SM-GREZZ_k103_34274_7
(SEQ ID NO: 241)
MTAAVITGASAGLGAEFTRQLVREFPEIEEFWLIARRVGKLEELAQQFPDKKFVCMGLNLLDPKSFRYLGEKLAEQK
ADVRVLVNNAGCGTTGNIGFGATSADVMRVVDLNVRALTMVTQTVIPFMTRGAKIINVSSIAAFCPTPRMTVYSASK
AYVSAFTGGIADELRPKGISVTAVCPGPMKTEFFDAAGDHDLFGNIPWCDPVKVVAGTIRAAKKGRTFYTPTVFNKF
YRFVAKILPMKWMIKATRV
>msp_282|G141817_k105_72636_7
(SEQ ID NO: 242)
MEKLDLHGKTGFIIGGTRGIGNAIAMRMAGLGMNLTVIARKEDELAAMKAQVESLGVRFLGIQASNTDFDAIAAAFE
TSWQTYGRLDLLVNAAGTGVMTPFEELTKSDVDTTIDVNLKGTIYAVKLSVPYFEKTGGGNILNISSMSAIRGIPDP
VNNNGIYTATKFATNGFTECMQKYLLKYGIRVTALCPGSTATSWWTRWTHSFGTDAMLPVDTLADIAELIVTSPEKV
LFKQLQVLPTVEIDNF
>msp_283|G140039_k105_56422_1
(SEQ ID NO: 243)
VSQVCVITGGTSGIGRCTAQAMLARGYTVYELSRRAEGVAGMQHIVADVTKEETLAAAVAQILQREDHIDVLINNAG
FGISGAVEFTGTEEAQRQLDVNFFGMVRMNRQVLPVMRKQGYGRIVNLSSVAGAIPIPFQTYYSASKAAINSYTMAL
ANEVKPFGIQVCCVQPGDIRTGFTAAREKNQLGDDIYGGRIARSVSGMERDEQTGMAPEQAGAFIARVATRKGVRPV
NTIGLQYKFFCFLAKVLPARWLNALVGLIYAK
>msp_283|2140697321_1_k103_60069_1
(SEQ ID NO: 244)
MTAAVITGASAGLGAEFTRQLVREFPDVEEFWLIARRVGKLEELAQQFPEKKFVCIGLNLLDPKSFRYLGEKLAEQK
ADVRLLVNNAGCGTTGNIDASASSADVMRVVDLNVRALTMVTQTVVPFMTRGAKIINVSSIAAFCPTPRMTVYSASK
AYVSAFTGGLADELRPKGITVTAVCPGPMKTEFFDAAGDHDLFGNIPWCDPVRVVAGTIRAAKKGRTFYTPTAFNKF
YRFVAKILPMKWMIKATRV
>msp_315|2140215031_1_k103_77694_43
(SEQ ID NO: 245)
MSKIALVTGGTSGIGKETALYLAKNGCTVYELSRRAEGVAGLRHISADVTDEASVRRAVEQILQEAGQIDILVNNAG
FGISGAVEFTDTEEAERQFNVNFFGMVRMNRAVIAAMRSRGGGRIVNLSSVAAPVPIPFQTYYSASKAAINSYTMAL
ANELRPFGITVCAVMPGDIHTGFTAARRKVAEGDEIYAGRISRSVKRMEHDEQTGMDPAKAGAFVGRVALKTGHKPL
YTIGFAYKAAVFLTKILPAGTLNWLIGKIYAS
>msp_315|2140758809_1_k103_66051_26
(SEQ ID NO: 246)
MKKIAVVTGASSGMGRDFVRAVDREFSPQEIWVIARREDRLLALQEQVHATIVPFAMDLSRDEAFRCYQEALEREQP
EIVALVNAAGYGKFTPFAEMDMASQLGIVELNDRALTAMCHMSLPYMHPGSKIINLGSNSAWQPVPYMSVYGASKAY
VLSFSRALGRELKGRDIQVLCVCPGWVKTEFMDRAVHDSTVSFFDRWYESEAVVEKAMRDLRKGKTVSILGFPVRMQ
VRLVKLLPVRMIMNIWCKQQKKP
>msp_319|2140813589_1_k103_29674_3
(SEQ ID NO: 247)
MEKMRFEGKVVIVTGASSGIGRSTARLFAMEGAKVVAAARRMKRLEDLRDKVAAENAPGVILPVKTDVRDPAQIEAM
FDTCLKEFGHLDILVNNAGVLDGQLPIHETTPEVYDMIYETNQRAVFLCCQRAIRIFLEQGTPANIVNVASAASLRG
LKGGAMYVTTKHAVLGLTRNISASFFERGIRCNCIEPANILTEINKVPREKGIGILEWQMRAGKAAPLHTITKPGEK
KPMLGKPKDCANAIAYLADDVAARYISGAELKVDAGWLNM
>msp_319|2140511497_1_k103_21708_26
(SEQ ID NO: 248)
MFSVSEASVAVITGGSSGIGLNAARALRDRGLIVYELSRRAENAEPGVTHLQADVTDETQVNAAVAEILRREGRIDI
LINNAGFGISGAIEFTPPQEARRQFDVNFFGMVNMNRAVLPVMRQQGGGRIVNMSSVAAPIAIPFQAYYSASKAAVR
TYTLALASEVRPFGIEVCVIMPGDIATGFTAARHKSCGGDDVYNGRIARSVAVMEHDERTGMSAQFAGQFVARRATQ
KHPKLICTMGRKYALFVFLMRILPTRTATRIVGRIYAS
>msp_319|SM-F1KPA_k103_35872_8
(SEQ ID NO: 249)
MNIAVITGASAGIGRELVYAVDKDAEYDEIWVIARRKERLEELRGKCRNPIRPIALDLSDLSSIDAYQALLEQEQPE
IRMLVNAAGCGVFGPFAEADRKKLISSAQLNSLALTGMCHASLPYMHSGSNIINMGSNSAWQPVPFQAVYGASKSYV
LSLSRALGRELRPQGIHVMCVCPGWIKTEFQQVAHHDEFIRYVDKWYGPDEVAAQAMKDLKKKKSVSILGHPVRRQV
RLVKLLPVDTVMDIWCKQQGIE
>msp_335|G140039_k105_18457_4
(SEQ ID NO: 250)
MSKKVAIVTGGTSGIGRATALALQERGYTVYELSRRAEGMPDIRHIVADITKEETLRAAVEQVLAVEGRLDLVVNNA
GFGISGAIEFTDTQEAQRLFDTLFFGMVRMNRCVIPLMRQQGHGRIVNISSVAAPVPIPFQAYYSAGKAAINAYTMA
LANELRPFGVTVCAVMPGDIKTGFTAARHKIIDGDDIYQGRIGRSVQRMEHDELTGMDPAKVGRYIAAVASREGSHH
PLYATRIDYKFFVFLTKVLPARFLNWLIYQLYGK
>msp_335|G140039_k105_113541_3
(SEQ ID NO: 251)
MKKIAVITGASSGMGKRFAETIDQFGTFDEVWVIARQWDKLEALRDTVPFPIRVLAMDLTDRASFNIYKAALAEEPV
QVGLLMNCSGYGKFSAVLDTPLEVNLNMTDLNCQAVVAMCQLTAPYMPRGSQIINIASVAAFQPIPYINVYGATKAF
VLSFSRALNRELRRQGVGVMAVCPFWTKTAFFDRAVTDGEAIVKKYAAMYDPDDIVRRTWRDAKRGKDVCKYGFVAR
GQAALTKLLPHSLVMDVWMKQQQLS
>msp_384|G139985_k105_38511_6
(SEQ ID NO: 252)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSRPFELLPYCDQVQVAGGALRAARAGQTIYTPRLF
YKFYRLLAKLTPAKLMVKLTKT
>msp_389|2140583655_1_k103_24582_14
(SEQ ID NO: 253)
MAKICVITGGTSGIGKCTAEAMAQKGYTVYELSRREEGLPGMFHIPTDVTDPDACQRAIDEVVSQAGRIDVMINNAG
FGISGAIEFTPVEQAKRQFDVNFFGMVNMNCAVIPVMRAQRSGRIVNLSSVAGAIPIPFQAFYSASKAAINSYTMAL
ANEIKPFGVQVCCVQPGDIQTGFTAARQKIVVGDDIYGGRISRSVAGMEKDERTGMRPEDAGAFVCRAATRKGVRPV
NTIGLSYKFFCVLQKLLPAKTLNWLVGMVYAR
>msp_389|2140583655_1_k103_54238_28
(SEQ ID NO: 254)
MKKIAIITGASSGMGKVFAETINTYDTFDEVWVIARRLDRLESLQETVPFPVRPIALDLTDRESFRHYAELLENAQA
NVQLLVNCSGYGKFQAACDTPLSQNLNMVDLNCEALMAMCQLTIPYMHAGAQIINIASVAACQPVPYIGVYAASKAF
VLSYSRALNRELDDKDISVMAVCPFWTKTEFFDHAVIDGEKPVVKKYAAMYEPQQIVARAWRDAKRGKDVSKFGFIA
RAQMALVKILPHSLIMDIWLSQQKL
>msp_409|SM-ITLFT_k103_11120_15
(SEQ ID NO: 255)
MNSSKVAVVTGGTSGIGKATALALQKAGCTVYELSRRAEGVEGLRHISADVTDEAAVNAAVAQIMAKAGHIDLLVNN
AGFGISGAIEFTAPEDAKKLFDVNFFGMVNMNRAVVPLMRAAGHGRIVNLSSVAAPVPIPFQAYYSATKAAVNAYTM
ALANELRPFGVTVCAVMPGDIHTGFTAARKKVAEGDAIYHGRISRSVQRMEHDEETGMDPAKAGAYIAAVALRDGNR
HPLYAIRFDYKFFTFLAKVLPARFLNWLIYCLYGK
>msp_409|2140231489_1_k103_16108_4
(SEQ ID NO: 256)
MKKIAVITGASSGMGRRFAETVDRYGRFDEVWVIARHQAQLEALKATVPFPIRVLALDLTDRTSFGTYAAALAEEPV
QVGLLMNCSGYGKFSAVLDTPLAVNLNMTDLNCQAVVAMCQLTAPYMPSGSQIINIASVAAFQPIPYIDIYGATKAF
VLSFSRALNRELRSCGIGVMAVCPFWTKTAFFDRAIREGEQPIVKKYVAMYDVEDIVTRTWRDAKRGKDVCKYGFIA
RAQAGLAKILPHSLVMDVWMKQQELR
>msp_443|SM-GRF5H_k103_6007_2
(SEQ ID NO: 257)
MKENRNWVVTGASGGIGAVLVEKLLEKGFRVAALSRTPEKIEARHGAQEGLRAIRVDVRSEESVLKAREEAKAFLGR
VDVVVNTAGYGLGGAIEEVSDQEARAVFDVNVFGALNVCRHFVTDLREQGGGCIINFACMDSVIATGYNAVYHATKY
AMDALTDALNKEAGQFGIRAICVKNGPMRTDYLDNKQYAAEQLEVYQQARQENRAREARYGAAAAADPGKVAELLIR
LSGEEEAPKDLYLTRESVKAIREKWDALEQEFETWKWATLCVDFPKEECYFGKRN
>msp_443|SM-GRF5H_k103_14038_1
(SEQ ID NO: 258)
MKTAIITGASSGLGSEFVRQLADVFPEIQCCWLIARRRDRLEKLAVNLPGWTVECLPMDLCDPMSFVALQEKLNTQK
PEVALLINNAGCGYLGNLGEMETTTQTRMVDLNLRALTAVTNMVIPFMAPGGRILNVSSIASFCPNPRMTVYSASKA
YVSAFTVGLAEELRPKGITATAVCPGPMKTEFLDVGGISGHSRTFDMLPYCDQVKVAGGALRAARAGRTIYTPRLFY
KFYRVLAKVTPVKLMVKIAGT
>msp_464|2140295410_1_k103_60112_3
(SEQ ID NO: 259)
MNEKKVAVVTGGTSGIGRATALALKDAGCTVYELSRRAEGVEGLHHISADVTDESAVNAAVAQVVAAEGQIDILVNN
AGFGISGAIEFTSTTDAKSLFDVNFFGMVNMNRAVVPLMRAAGHGRIVNLSSVAAPVPIPFQAYYSATKAAVNAYTM
ALANELRPFGVTVCAVMPGDIHTGFTAARRKLSEGDDIYQGRISRSVVRMEHDEQTGMDPAKAGTYIAKVALRSGSN
HPLYAIRFDYKFFAFLAKVLPARFLNWLIYLLYGK
>msp_464|2140295410_1_k103_83442_3
(SEQ ID NO: 260)
MKKIAVITGASSGMGKRFAQRVNEFGTFDEIWLIARHGERLEALRETMPFPARVLALDLTDRTSFGVYEAALAEEPV
EIGLLVNASGFGKFAAVADTPLQVNLNMTDLNCQAVVALCQLSIPYLGRGGQIINIASVAAFQPIPYVDVYGATKAF
VLSFSRALNRELRPRGAAVMAVCPFWTKTAFFDRAVVSEEKPVVKKYVAMYDPEDIVTRAWRDAKRGKDVSKYGFIA
RFQAALTKILPHSLVMDVWMAQQGLE
>msp_482|2140339903_1_k103_44722_3
(SEQ ID NO: 261)
MNKKIAIVTGGTSGIGKATALALRGAGYTVYEFSRREAGVEGLHHIRADITDETQVRAAVQQVMDAEGQIDVVVNNA
GFGISGAVEFTDTADAQRLFNADFFGMVRVNRAVIAHMRAAGRGRIVNLSSVAGPLPIPFQTYYSAAKAAVNAYTMA
LANELRPFGITVCAVQPGDIHTGFTAAREKTVTGDEVYGGRISRSVSRMEHDEQTGMDPAAAGRFIARVAQKTSHKP
IYTIRLDYQFFVFLKRILPYRALNALIGLIYAK
>msp_579|G139969_k105_64033_72
(SEQ ID NO: 262)
MKTAIITGASSGLGREFARQLTDVFPEIECCWLIARREDRLEEIAREMVGVETVCLPLDLCDSMSFTTLQEKLAAEK
PEAAILINNAGCGYLGRMGETETAVQTRMVDLNVRAMTAVTNLVIPYMPAGGRILNTSSIASFCPTPRMTVYGASKA
YVSSFTVGLSEELKRKNITVTAVCPGPMKTEFLDVGSITGRSPAFEYLPYCDQVRVAAGALRAAKAGRTIYTPRLFY
KFYRLLAKVTPVKLMVKFTKT
>RJX3347|00904 3-phenylpropionate-dihydrodiol/cinnamic acid-dihydrodiol
dehydrogenase
(SEQ ID NO: 263)
MKEFFGKVALITGAAHGIGYSFALEAASRGMKLALVDIDETAMLEAAKECSRFGAEVLTCTTDVSVYEEAKASVEAT
MARFGQIDLLFANAGIATAGSILHIPIRDWEWALAVNTMGIVHYVHEVLPIMEAQKTPAYLMCTASIAGLRAGMAIN
PPYFCSKHAAVSVAESVKAEVESSGSDIGVSVFCPMYVATDIDNCENHRPARFWDASDPFYNSEEYLTAREAFHKNI
ATGMPLKKIGKRLFKAIEDNQMYIVTHTQTIPYIESRHRAIEEDAKKELELQL
>RJX3347|02204 hypothetical protein
(SEQ ID NO: 264)
MKTAIVTGASAGLGREIVRQIAAVFPEIEAYWLIARRAERLEELAAAMPDQQVDCMPLDLCDPMSFMTLQEKLASEQ
PEVALLVNNAGCGYLGNVGEVETSTQTRMIDLNLRALTAVTNITVPFMAPGSRILNVSSIAAFCPTPRMTVYSAGKS
YVSAFSIGLAEELRHKGITVTAVCPAPMRTEFLEAGNIAGNSRMFERLPYCDQVQVAGGALRAARAGRVIYTPKLFY
KFYRVLAKVTPVKLMVKFTKT
>TM34_A01|01778 hypothetical protein
(SEQ ID NO: 265)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT
>TM34_A02|00902 hypothetical protein
(SEQ ID NO: 266)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT
>TM34_B02|02211 hypothetical protein
(SEQ ID NO: 267)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT
>TM34_C02|01483 hypothetical protein
(SEQ ID NO: 268)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT
>TM34_D02|01557 hypothetical protein
(SEQ ID NO: 269)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT
>TM34_G01|02337 hypothetical protein
(SEQ ID NO: 270)
MKTAVITGASSGLGREFVRQFYSVFPEIERVWLIARRTDRLQELAEQLEEKGISTLTLPLDLCDTMSFTAYQEHLVE
EQPEIALLVNNAGCGYLGNIGEIDTVSQTRMIDLNLRALTAITNLSVPYMEAGSRILNVSSIASFCPTPRMTVYSAT
KAYVSAYTIGAAEELKAKGITVTAVCPGPMATEFLAVGHVVDSWPFELLPYCDQVQVAGGALRAAKAGQTIYTPRLF
YKFYRLLAKLTPAKLMVKLTKT

IsmA is a cholesterol dehydrogenase that acts in the metabolic pathway which forms coprostanol form cholesterol. IsmA was first discovered as the enzyme responsible for the metabolism of cholesterol to cholestenone in E. coprostanoligenes. The majority of coprostanol formation in diverse human populations can be attributed to a clade of highly prevalent, IsmA-encoding bacterial species.

By “IsmA polynucleotide” is meant a nucleic acid molecule that encodes an IsmA polypeptide or an IsmA equivalent protein. Exemplary IsmA polynucleotides are known in the art and described, for example, by Kenny et al.

The terms “isolated,” “purified,” or “biologically pure” refer to material that is free to varying degrees from components which normally accompany it as found in its native state.

“Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. In some embodiments, a cell (e.g., bacterial cell or other microbe) present in a biological sample (e.g., stool sample) is isolated.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein, polynucleotide, analyte, or clinical indicator having an alteration in expression level or activity that is associated with a developmental state, condition, disease, or disorder (e.g., associated with a cardiovascular disease). In some embodiments, the marker is cholesterol or a metabolite thereof. Cholesterol metabolites include, for example, cholestenone, coprostanone, and coprostanol. In some embodiments, an increase in intestinal coprostanol denotes a reduction in serum cholesterol. In other embodiments, an increase in coprostanol present in stool denotes a reduction in serum cholesterol.

As used herein, “obtaining” as in “obtaining an agent” includes synthesizing, purchasing, or otherwise acquiring the agent.

By “Oscillibacter species” is meant any member of the Oscillibacter genus. Exemplary Oscillibacter species include Oscillibacter valerigens, Oscillibacter valericigenes, Oscillibacter sp. 57_20, or any species associated with Global Microbiome Conservancy Isolate IDs 0471HH_0918_059_C7, 1462QI_0319_071_A08, 1462QI_0319_071_A09, 1462QI_0319_071_A11, 1462YY_0218_030_D6, 3050YG_0918_062_E10, 3790QQ_0218_002_G9, 4324HL_0218_028_E7, 4324HL_0218_028_E9, 4324HL_0218_028_F5, 4452YQ_0918_057_F10, 5399RU_0319_069_B05, 5399RU_0319_069_C10, 5399RU_0319_069_D10, 5399RU_0319_069_D12, 5399RU_0319_069_E08, 5399RU_0319_069_G09, 5399RU_0319_069_H12, 8131GG_1118_056_H10, 8131GG_1118_056_H2, 8131GG_1118_056_H6, 8144JZ_0517_029_D1, 9573HE_0319_068_B09, 9573HE_0319_068_H06, 9734KT_0319_070_B03, or 9734KT_0319_070_C10, or NCBI RefSeq assembly Nos. GCF_018408705 or GCF_005121165.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g.

Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively relative to a reference. A reduction may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In embodiments, a reference level is the level of cholesterol or a cholesterol metabolite (e.g., cholestenone, coprostanone, coprostanol) present in an untreated control subject or present in a subject at an earlier time point during the course of treatment with an agent described herein.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, about less than about 500 mM NaCl and 50 mM trisodium citrate, or about less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. Any compositions or methods provided herein can be combined with one or more of any of the other compositions and methods provided herein.

### BRIEF DESCRIPTION OF THE DRAWINGS

FIG. 1 provides a scatter plot showing an example of Oscillibacter-blood measurement associations. The line with the shaded area represents the fitted linear regression line with 95% confidence intervals. P values were adjusted for multiple comparisons with the Benjamini-Hochberg method. TPM denotes Transcript-Per-Million.

FIG. 2. provides chemical structures illustrating the pathway for biotransformation from cholesterol to coprostanol. An Intestinal Sterol Metabolism A (IsmA) encoded protein catalyzes the first and third steps.

FIG. 3 is a graph showing Spearman correlation coefficients for metagenomic species pangenomes (MSPs) in most represented genera (Alistipes, Bacteroides, Blautia, Roseburia and Oscillibacter), IsmA encoders and other. The “+” sign shows the mean coefficient value.

FIG. 4 provides a graph showing the abundance of stool cholesterol in samples stratified by the presence of selected Oscillibacter MSPs and IsmA encoders.

FIG. 5 provides scatter plots showing the relationship between Oscillibacter genus abundance and stool cholesterol in ismA+ and ismA− samples. Line with shaded area represents the fitted linear regression line with 95% confidence intervals FIG. 6 provides a graph showing that Oscillibacter and IsmA encoders were associated with a decrease of plasma cholesterol in a combinatorial manner.

FIG. 7 provides a graph showing predicted structural distance (PROSE distance) of genes from each Oscillibacter isolate with key cholesterol metabolism genes. Oscillibacter genes that also show sequence alignment similarity (amino acid level) are highlighted in black.

FIG. 8A-8D FIG. 8A provides a phylogenetic tree of metagenomic species pangenomes, RJX3347, RJX3711, J115 and references from the Genome Taxonomy Database for the main genera negatively associated with stool cholesterol. A bubble plot outside the tree shows the amino acid sequence identity and coverage compared to the cholesterol metabolism candidates from RJX3347 (A.ond: Alistipes onderdonkii, B.the: Bacteroides thetaiotaomicron, B.obe: Blautia obeum, E.cop: Eubacterium coprostanoligenes, P.cop: Prevotella copri, R.hom: Roseburia homini). FIG. 8B provides a schematic showing predicted protein structures of candidates RJX3347_02204 and RJX3711_01178 superimposed on IsmA from Eubacterium coprostanoligenes. FIG. 8C provides a zoomed in view highlighting conserved catalytic triad Ser-Tyr-Lys. Residues from reference sequence ECOP170 are highlighted. FIG. 8D provides a schematic showing predicted protein structures of candidate RJX3347_02251 and cholesterol-alpha-glucosyltransferase (CgT) from Helicobacter pylori superimposed on an experimentally solved structure from the Protein Data Bank.

FIG. 9A-9B provides confocal microscopy images of live (FIG. 9A) Oscillibacter sp. RJX3711, Dysosmobacter welbionis J115, Oscillibacter sp. RJX3347 and (FIG. 9B) Escherichia coli RJX1193 with membrane dye and fluorescent-labeled cholesterol.

FIG. 10 provides chemical structures and graphs showing intensity in cell pellets for cholesterol and Oscillibacter-produced derivatives in media with cholesterol (CHO) supplementation experiments . . .

FIG. 11 provides chemical structures and graphs showing intensity in cell pellets for cholesterol and Oscillibacter-produced derivatives in media with ¹³C isotope-labeled cholesterol (CHO*) supplementation experiments. Stars in the chemical structures indicate the isotope-labeled carbon atoms.

### DETAILED DESCRIPTION OF THE INVENTION

The disclosure features compositions and methods that are useful for treating cardiovascular disease (e.g., cholesterol related disorders, or diseases associated with or characterized by increased levels of plasma triglycerides, plasma cholesterol, or serum C-reactive protein) in a subject.

The present disclosure is based, at least in part, on the discovery that species from the Oscillibacter genus were associated with decreased fecal and plasma cholesterol, plasma triglycerides, and serum C-reactive protein. Further, it was discovered that the enzymes IsmA and CgT, and bacterial cells expressing such enzymes were associated with decreased plasma cholesterol, plasma triglycerides, and serum C-reactive protein. Using functional prediction and in vitro characterization of multiple representative human gut Oscillibacter isolates, conserved cholesterol-metabolizing capabilities, including glycosylation and dehydrogenation were uncovered. These findings suggest that cholesterol metabolism is a broad property of phylogenetically diverse Oscillibacter species and reveal the beneficial potential for lipid homeostasis and cardiovascular health by using Oscillibacter species.

Accumulating evidence suggests that cardiovascular disease (CVD) is associated with an altered gut microbiome. Understanding the underlying mechanisms has been hindered by lack of matched multi-omic data with diagnostic biomarkers. To comprehensively profile gut microbiome contributions to CVD, stool metagenomics and metabolomics from 1,429 Framingham Heart Study participants was generated and a variety of blood lipids and cardiovascular health measurements associated with microbiome and metabolome composition were identified, including 129 species pangenomes and 7,479 metabolic features. The combined profiles revealed microbial pathways implicated in CVD, including flavonoid, γ-butyrobetaine and cholesterol metabolism.

Accordingly, the disclosure provides therapeutic compositions comprising a bacterial cell expressing an IsmA protein (e.g., Oscillibacter species, Eubacterium, Lactobacillus or other benign bacterial cell of a human microbiome that endogenously expresses IsmA or that is engineered to express IsmA), and methods of using such bacterial cells to reduce levels of proinflammatory lipids, to reduce plasma triglycerides, or to otherwise benefit the cardiovascular health of a subject. In other embodiments, the disclosure provides therapeutic compositions comprising a bacterial cell expressing a CgT polypeptide (e.g., Oscillibacter species, Eubacterium, Lactobacillus or other benign bacterial cell of a human microbiome that endogenously expresses CgT or that is engineered to express CgT). In embodiments, the composition is a probiotic for oral or rectal administration. In other embodiments, the composition isa pharmaceutical composition comprising a bacterial cell expressing an IsmA and/or CgT polypeptide for administration (e.g., oral, rectal) administration to a subject having or having a propensity to develop cardiovascular disease (e.g., cholesterol related disorders, or diseases associated with or characterized by increased levels of plasma triglycerides, plasma cholesterol, or serum C-reactive protein) in a subject, or symptoms thereof. In yet another embodiment, the disclosure provides a method of treating a subject having or having a propensity to develop cardiovascular disease (e.g., cholesterol related disorders, or diseases associated with or characterized by increased levels of plasma triglycerides, plasma cholesterol, or serum C-reactive protein) in a subject, or symptoms thereof including administering to the subject an amount of one or more agents disclosed herein, such as Oscillibacter species, cells expressing an IsmA and/or CgT polypeptide, wherein the agent is administered in an amount sufficient to treat the cardiovascular disease (e.g., cholesterol related disorder, or disease associated with or characterized by increased levels of plasma triglycerides, plasma cholesterol, or serum C-reactive protein) in a subject or symptom thereof, under conditions such that the disease or disorder is treated. In some embodiments, methods of the present disclosure include the treatment of any cholesterol related disorder in a subject by compositions including agents disclosed herein.

### Cholesterol Related Disorders

As used herein, “a cholesterol related disorder” includes any one or more of the following: hypercholesterolemia, heart disease, metabolic syndrome, diabetes, coronary heart disease, stroke, cardiovascular diseases, and Alzheimer's disease, which can be manifested, for example, by an elevated total serum cholesterol, elevated LDL, elevated triglycerides, elevated VLDL, and/or low HDL. Agents of the present disclosure can also be useful in preventing or treating atherosclerotic diseases, such as, for example, coronary heart disease, coronary artery disease, peripheral arterial disease, stroke (ischaemic and hemorrhagic), angina pectoris, or cerebrovascular disease and acute coronary syndrome, myocardial infarction. In certain embodiments, the agents of the invention are useful in reducing the risk of: nonfatal heart attacks, fatal and non-fatal strokes, certain types of heart surgery, hospitalization for heart failure, chest pain in patients with heart disease, and/or cardiovascular events because of established heart disease such as prior heart attack, prior heart surgery, and/or chest pain with evidence of clogged arteries. In certain embodiments, the agents of the invention and methods described herein can be used to reduce the risk of recurrent cardiovascular events.

In some embodiments, the invention provides methods and compositions for treating and/or preventing cardiovascular diseases and related disorders. Cardiovascular diseases and related disorders referred to herein are diseases and disorders that involve the heart or blood vessels (e.g., arteries and veins). Cardiovascular diseases and related disorders include atherosclerosis, cardiac dysrhythmia, cardiomyopathy, coronary heart disease, hypertension, dyslipidemia, myocardial infarction, myocarditis, congestive heart failure, valvular heart disease, and vascular disease.

In some embodiments, the invention provides a method and/or composition for treating and/or preventing myocardial infarction, coronary heart disease, atherosclerosis or dyslipidemia. Myocardial infarction (MI) or acute myocardial infarction (AMI), commonly known as a heart attack, is the interruption of blood supply to part of the heart, causing myocardial cellular death. Classical symptoms of acute myocardial infarction include sudden chest pain, shortness of breath, nausea, vomiting, palpitations, sweating, anxiety, weakness, a feeling of indigestion, and fatigue. Myocardial infarctions are commonly a result of atherosclerosis, but are also associated with severe infections, intense psychological stress or physical exertion, coronary heart disease, and diabetes.

Coronary heart disease refers to any condition in which there is the narrowing or blockage of the coronary arteries, usually caused by atherosclerosis. Examples of coronary heart disease include, but are not limited to coronary artery disease.

Atherosclerosis is the buildup of cholesterol and fatty deposits, called plaques, on the inner walls of the arteries. Plaque formation causes thickening of the blood vessel walls, which obstructs blood flow and leads to diminished amounts of oxygen and nutrients reaching the target organ. Atherosclerosis can lead to ischemia, myocardial infarction, coronary heart disease, and/or congestive heart failure. Examples of atherosclerosis include, but are not limited to arteriosclerosis and ateriolosclerosis. In some embodiments, the disease is an atherosclerosis related disease, which includes, but is not limited to, atherosclerotic heart disease and acute coronary syndrome.

Vascular disease includes diseases affecting the arteries, veins, lymph vessels, and blood disorders that affect circulation. Most commonly, vascular disease is associated atherosclerosis. Examples of vascular disease include, but are not limited to, cerebrovascular disease, peripheral artery disease, aneurysm, renal artery disease, Raynaud's Phenomenom, Buerger's Disease, peripheral venous disease, varicose veins, blood clotting disorders, blood clots in the veins, and lymphedema.

Congestive heart failure (CHF), or heart failure, is a condition in which the heart is restricted from pumping enough blood to the body's other organs. This can result from narrowed arteries that supply blood to the heart muscle (e.g., coronary artery disease), past myocardial infarction having scar tissue that interferes with the heart muscle's normal work, high blood pressure, heart valve disease due to past rheumatic fever or other causes, cardiomyopathy, congenital heart defects, endocarditis and/or myocarditis.

Hypertension, or high blood pressure, is a chronic medical condition in which the blood pressure in the arteries is elevated. Hypertension increases the risk for ischemic heart disease, strokes, peripheral vascular disease, heart failure, aortic aneurysm, diffuse atherosclerosis, pulmonary embolism, hypertensive retinopathy, and hypertensive nephropathy.

### Gut Microbiome Impacts on Cardiovascular Health

Intestinal microbiota-host interactions play a role in the regulation of human health. Alterations to fecal microbial composition, an accessible proxy for the gut microbiome, have been associated with numerous diseases. Cross-sectional studies have uncovered gut microbial composition shifts (dysbiosis) in patients with cardiometabolic diseases such as diabetes mellitus (Zhao et al., Science 359, 1151-1156, 2018), obesity (Turnbaugh et al., Nature 444, 1027-1031, 2006), and non-alcoholic fatty liver disease (NAFLD) (Loomba et al., Cell Metabolism 25, 1054-1062.e5). Intestinal dysbiosis has also been linked to cardiovascular disease (CVD), a leading cause of death globally. Several cohort studies have identified changes in bacterial diversity and individual microbes in patients with atherosclerotic cardiovascular disease (ASCVD) (Jie et al., Nat. Commun. 8, 845, 2017), acute coronary syndrome (Talmor-Barkan et al., Nat. Med. 28, 295-302, 2022), and ischemic heart disease (IHD) (Fromentin et al., Nat. Med. 28, 303-31, 20224).

Although some associations between the gut microbiome and CVD are of great interest, the discovery of clinical actionable mechanisms of these associations remain challenging due to the limited understanding of gut microbiome metabolism. The most studied CVD-related microbial metabolic pathways are the trimethylamine (TMA) pathways, which converts dietary choline or L-carnitine to TMA (Wang et al. (2011) Nature 472, 57-63; Tang et al., (2013). N. Engl. J. Med. 368, 1575-1584; Koeth et al. (2013). Nat. Med. 19, 576-585; Yoo et al. (2021). Science 373, 813-818; Li et al. (2022) Gut 71, 724-733; Rajakovich et al., (2021) Proc. Natl. Acad. Sci. U.S.A. 118. 10.1073/pnas.2101498118; Buffa et al. (2022). Nat Microbiol 7, 73-86). TMA is oxidized by the liver into atherosclerosis-promoting agent trimethylamine N-oxide (TMAO), which contributes to platelet hyperreactivity and enhanced thrombosis potential in mouse models and is associated with increased risks of heart failure and mortality in humans. The bile acid pool can also be modified by the microbiome (Sato et al. (2021) Nature. 10.1038/s41586-021-03832-5), impacting host lipid homeostasis through nuclear receptors and G protein-coupled receptors (Keitel et al., (2019). Bile Acid-Activated Receptors: GPBAR1 (TGR5) and Other G Protein-Coupled Receptors. In Bile Acids and Their Receptors, S. Fiorucci and E. Distrutti, eds. (Springer International Publishing), pp. 19-49; Chiang et al., (2020). Bile Acid and Cholesterol Metabolism in Atherosclerotic Cardiovascular Disease and Therapy. Cardiol Plus 5, 159-170.

In addition, the microbiome metabolizes cholesterol, an essential lipid whose homeostasis is related to cardiovascular health. The IsmA enzyme that participates in converting cholesterol to coprostanol was previously discovered, and a group of uncultured Eubacterium species carrying the IsmA gene that was associated with significantly lower stool and plasma cholesterol was previously identified (Kenny, et al. (2020). Cell Host Microbe 28, 245-257.e6). More recently, the Bacteroides thetaiotaomicron Bt_0416 sulfotransferase was shown to be involved cholesterol sulfate production and associated with increased plasma cholesterol and cholesterol sulfates (Le et al., (2022) Nat Microbiol 7, 1390-1403; Yao et al. (2022) Nat Microbiol 7, 1404-1418). Potential cholesterol metabolism has been identified in other gut microbes including Bifidobacterium, Enterococcus, Lactobacillus and Parabacteroides(23), pointing to a largely unexplored space of cholesterol metabolic activity.

### Oscillibacter Species, IsmA and CVD

Understanding how gut microbial metabolic activity influences the host cardiovascular health requires extensive profiling of the gut metagenome and metabolome within the context of cardiovascular diseases. To address this gap, the Framingham Heart Study (FHS) was used, the longest running observational cohort study on the epidemiology of CVD which has identified many CVD risk factors, including plasma lipid (cholesterol, triglyceride) levels and hypertension. 16S rRNA gene sequencing data generated from the stool samples from over 1,000 FHS participants was previously used to identify taxa associated with CVD risk factors, CVD-related medications and dietary information. Here, to comprehensively profile the gut microbiome and elucidate metabolic functions related to cardiovascular health, shotgun metagenomic and paired untargeted metabolomic data were generated for stool samples collected from FHS Gen3/Omni2 cohort individuals. The integrative analysis uncovered over 16,000 associations between microbes and metabolic features, including the strong negative association between Oscillibacter spp, and host cholesterol. Molecular networking and protein language models were combined to search for potential genes involved in cholesterol metabolic pathways in Oscillibacter spp. Efficient cholesterol uptake and further biotransformation into cholestenone, glycosylated cholesterol and hydroxycholesterol was also demonstrated by multiple human gut Oscillibacter spp. isolates. These findings mechanistically link associations between gut microbes, microbial metabolic pathways, and markers of cardiovascular health to the cholesterol-metabolizing capabilities of the largely uncharacterized Oscillibacter genus.

The IsmA enzyme converts cholesterol to coprostanol, and a group of uncultured Eubacterium species carrying the ismA gene that was associated with significantly lower stool and plasma cholesterol was identified (Kenny et al., supra). More recently, the Bacteroides thetaiotaomicron Bt_0416 sulfotransferase was shown to be involved in the production of cholesterol sulfate and associated with increased plasma levels of cholesterol and cholesterol sulfates (Le et al., supra, Yao et al., supra). Potential cholesterol metabolism has been identified in other gut microbes including Bifidobacterium, Enterococcus, Lactobacillus and Parabacteroides, pointing to a largely unexplored space of cholesterol metabolic activity.

Recombinant Cells Expressing IsmA and/or CgT

In addition to the use of Oscillibacter cells that endogenously express IsmA and CgT polypeptides, the disclosure provides for a variety of other microbial cells that are engineered to express these polypeptides. Methods for engineering microbial cells are known in the art and described, for example, by Reeves et al., ACS Synthetic Biology 2015 4 (5), 644-654. Such bacterial cells are selected for either promoting gut health or for being benign bacteria that normally inhabit the gut of healthy human subjects. In some embodiments, the recombinant microbial cells belong to a genus selected from Firmicutes, Bacteroidetes, Actinobacteria, Bacteroidetes, Proteobacteria, Fusobacteria, Verrucomicrobia, Euryarchaeota, and Ascomycota. In some embodiments, the recombinant microbial cells belong to a genus selected from Corynebacterium, Bifidobacterium, Atopobium, Faecalibacterium, Clostridium, Roseburia, Ruminococcus, Dialister, Lactobacillus, Enterococcus, Staphylococcus, Streptococcus, Sphingobacterium, Bacteroides, Tannerella, Parabacteroides, Alistipes, Prevotella, Escherichia, Shigella, Desulfovibrio, Bilophila, Helicobacter, Fusobacterium, Pediococcus, Bacillus, Leuconostoc, Akkermansia, Methanobrevibacter, Propionibacterium, Coriobacteriaceae, Actinobacteria, Rikenellaceae, Lachnospiraceae, Firmicutes, Peptostreptococcaceae, Veillonella, Oscillospira, Dialister, Slackia, Eggerthella, Gordonibacter, Geobacter Alkaliphilus, Catenibacterium, Holdemania, Marvinbryantia, Symbiobacterium, Roseburia, Erysipelotrichaceae, Butyricicoccus, Sporobacter, Blautia, Dorea, Succinivibrio, Barnesiella, Biolophila, Eubacterium, or Saccharomyces. In embodiments, the engineered microbe is an engineered variant of a Lactobacillus, Bifidobacterium, Saccharomyces, Enterococcus, Streptococcus, Pediococcus, Leuconostoc, Bacillus, or Escherichia coli.

### Probiotic Formulations

The disclosure further provides probiotic formulations that include composition comprising a IsmA or CgT polypeptide, a microbial cell that endogenously expresses IsmA or CgT or a microbial cell that is engineered to express said proteins. In one embodiment, a probiotic formulation comprises an Oscillibacter cell that endogenously expresses IsmA and CgT. In another embodiment, the probiotic formulation comprises a Eubacterium expressing IsmA and/or CgT. In other embodiments, any one of the following cells is included in the probiotic formulation: B. subtilis, B. coagulans, B. subtilis, or B. cereus. In another embodiment, the probiotic formulation comprises a cell of the genus Bifidobacterium or a Lactobacillus (e.g., Lactobacillus acidophilus, L. casei, L. paracasei, L. rhamnosus, L. delbrueckii subsp. bulgaricus, L. brevis, L. johnsonii, L. plantarum and L. fermentum). The genome of select Lactobacillus is described, for example, by Makarova K, et al. Comparative genomics of the lactic acid bacteria. Proc. Natl. Acad. Sci. USA. 2006; 103:15611-15616. doi: 10.1073/pnas.0607117103.

In some embodiments, the probiotic is administered in a single bolus. In other embodiments, repeated (e.g., daily, weekly, monthly) dosing is required.

### Probiotic Formulations

Probiotic formulations comprising a CgT or IsmA polypeptide, a microbial cell that endogenously expresses such polypeptides (e.g., Oscillobacter, Eubacterium), or recombinant microbial cells (e.g., cells expressing a heterologous polynucleotide encoding an Oscillobacter or Eubacterium derived CgT or IsmA) are useful in probiotic applications. In particular embodiments, the probiotic is formulated for oral delivery, optionally a powder, bolus gel, capsule, liquid, or foodstuff. In embodiments, the foodstuff can be formulated as described, for example, in U.S. Pat. No. 6,787,151. The one or more microbial cells (e.g., Oscillobacter, Eubacterium, Lactobacillus, or other benign bacterial cell) that expresses or is engineered to express IsmA and/or CgT can be provided in a probiotic composition in an effective amount to reduce triglycerides and/or total cholesterol levels in the gut and/or blood. In some embodiments, the probiotic can further comprise a prebiotic, see, e.g., Ooi, L.-G. & Liong, M.-T. Cholesterol-lowering effects of probiotics and prebiotics: a review of in vivo and in vitro findings. Int. J. Mol. Sci. 11, 2499-2522 (2010). Exemplary prebiotics include oligosaccharides (isomaltooligosaccharides, lactosucrose, xylooligosaccharides and glucooligosaccharides), sugar alcohols and polysaccharides (starch, resistant starch and modified starch), fructooligosaccharides, inulin, oligofructose, lactulose, and galactooligosaccharides. Probiotic compositions comprising one or more bactera encoding one or more Cgt or IsmA enzymes are provided herein. The one or more bacteria of the probiotic composition can be from the phylum Firmicutes.

Examples of engineered probiotics using a safe host bacterium to deliver a protein or contribute a metabolic activity may comprise E. coli Nissle 1917, see, e.g. Kurtz et al., Science Transl. Med. 11:475, 16 Jan. 2019; DOI: 10.1126/scitranslmed.aau7975. Lactococcus lactis has also been utilized for probiotic administration, see, e.g. Cook et al, Front. Immun. “Lactococcus lactis as a Versatile Vehicle for Tolerogenic Immunotherapy,” 17 Jan. 2018; DOI: 10.3389/fimmu.2017.01961. Lactobacillus gasseri has been engineered to express an anti-inflammatory protein from S. thermophilus to reduce colitis symptoms and is thus a useful species for probiotic applications. See, e.g. Carroll et al., Am J. Phys., Oct. 1, 2007; DOI: 10.1152/ajpgi.00132.2007. Engineered probiotics, natural microbes producing enzyme and/or purified enzymes have been administered in treatment of C. difficile infection, see, e.g. Mullish et al, Gut 2019:0:1-10. DOI: 10.1136/gutnjl-2018-317842, and those approaches can be adapted for use with the microbes, COR proteins and probiotics of the current disclosure. Similarly, administration of L. rhamnosus as a probiotic has been used in regulation of cholesterol metabolism. See, Park S, Kang J, Choi S, Park H, Hwang E, Kang Y, et al. (2018) Cholesterol-lowering effect of Lactobacillus rhamnosus BFE5264 and its influence on the gut microbiome and propionate level in a murine model. PLoS ONE 13(8): e0203150; doi: 10.1371/journal.pone.0203150.

The engineered microbes may be engineered to inducibly or constitutively express one or more CgT or IsmA polypeptides, and optionally one or more enzymes in the cholesterol metabolic pathway described herein. In embodiments, the engineered bacteria can be designed with a kill switch that responds to environmental cues. See, e.g. Stirling et al., Rational Design of Evolutionarily Stable Microbial Kill Switches Molecular Cell 68:686-697, Nov. 16, 2017. Temperature sensitive mutants can be utilized to control growth in a temperature-dependent manner. See, e.g. Stirling at 691-692. In one embodiment, the engineered are engineered using CRISPR systems.

### Genome Editing

In other aspects, a genome of a cell of a subject is edited to express a CgT or IsmA polynucleotide. Genome editing is a major focus of biomedical research. The development of novel “gene editing” tools provides the ability to manipulate the DNA sequence of a cell at a specific chromosomal locus, without introducing mutations at other sites of the genome. This technology effectively enables the researcher to manipulate a bacterial cell in vitro or in vivo.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule is introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. Importantly, if the donor DNA molecule differs from the chromosomal sequence, HR-mediated DSB repair will introduce the donor sequence (e.g., introducing a CgT or IsmA polynucleotide) into the chromosome, resulting in gene conversion/gene correction of the chromosomal locus. By targeting the nuclease to a genomic site of interest, the concept is to use DSB formation to stimulate HR and to thereby replace target sequence with a desired sequence, which might include a gene having a deletion or mutation (e.g., insertion, point mutation, frame shift).

Current genome editing tools use the induction of double strand breaks (DSBs) to enhance gene manipulation of cells. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for ˜18 bp sequences in the genome.

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121): 823-6). Unlike other gene editing methods, which add a functional, or partially functional, copy of a gene to a subject's cells but retain the original copy of the gene, this system can remove and replace the target gene. Genetic editing using engineered nucleases has been demonstrated in tissue culture cells and rodent models of rare diseases.

CRISPR has been used in a wide range of organisms including bacteria, bakers yeast (S. cerevisiae), zebra fish, nematodes (C. elegans), plants, mice, and several other organisms. Additionally CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (.about.30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096): 816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 2I promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

In one approach, one or more cells of a subject are altered to express a heterologous gene (e.g., IsmA, CgT) using a CRISPR-Cas system. Cas9 can be used to introduce a desired sequence (e.g., IsmA, CgT polynucleotide) to a target bacterial genome. Upon target recognition, Cas9 induces double strand breaks in the target genome. Homology-directed repair (HDR) at the double-strand break site can allow insertion of a desired sequence (e.g., IsmA, CgT polynucleotide).

The following US patents and patent publications are incorporated herein by reference: U.S. Pat. No. 8,697,359, 20140170753, 20140179006, 20140179770, 20140186843, 20140186958, 20140189896, 20140227787, 20140242664, 20140248702, 20140256046, 20140273230, 20140273233, 20140273234, 20140295556, 20140295557, 20140310830, 20140356956, 20140356959, 20140357530, 20150020223, 20150031132, 20150031133, 20150031134, 20150044191, 20150044192, 20150045546, 20150050699, 20150056705, 20150071898, 20150071899, 20150071903, 20150079681, 20150159172, 20150165054, 20150166980, and 20150184139.

### Delivery of Microbial Cells to a Subject

In embodiments, a microbial strain suitable for use as a probiotic and/or for oral delivery is modified to express IsmA and/or CgT. In embodiments, the engineered bacterial cell is suitable for delivery to the gastrointestinal tract, of a subject, by, for example, fecal microbiota transplant (FMT). In particular embodiments, the FMT may comprise bacteria cultured from the stool of encoders. As used herein, encoders are subjects that encode enzymes with the catalytic capabilities needed to perform transformations on cholesterol or related molecules, in some embodiments, encoders have the capability of metabolizing cholesterol to coprosterol. Encoders may be identified by a reduced amount of cholesterol content in the stool, an increase in cholestenone, coprostanone and/or coprostanol in the stool relative to non-encoders.

Targeted delivery may use materials with particular properties, for example, enteric, colon-targeting, omniphobic, mucoadhesive, or mucus-penetrating properties. Microbes can be further engineered with tissue targeting properties. In some embodiments, compositions including the microbial cells of the present disclosure are formulated to deliver the microbial cells to one at least one region of the gastrointestinal tract, such as, the small intestine, the large intestine, the colon, or the rectum. In some embodiments, the compositions including the microbial cells are formulated to deliver the microbial cells to the small intestine, such as to the duodenum, the ileum, or the jejunum. In an embodiment, the compositions including the microbial cells are formulated to deliver the microbial cells to the ileum.

Enteric coatings with buffering or protective compositions may be used for targeted delivery of the microbial cells of the present disclosure. The enteric coating may be formulated to deliver the microbial cells to at least one region of the gastrointestinal tract, such as, the small intestine, the large intestine, the colon, or the rectum. In some embodiments, the enteric coating is formulated to deliver the microbial cells to the small intestine, such as to the duodenum, the ileum, or the jejunum. In an embodiment, the enteric coating is formulated to deliver the microbial cells to the ileum. Whether enteric coatings are necessary for delivery of the microbial cells is dependent on the particular strains used, and may be determined by the person of skill in the art. For example, some bacteria strains (e.g, Lactobacillus acidophilus, Bifidobacterium Streptococcus mutants, etc.) partially resist the acidic environment of the stomach and the high bile salt conditions of the intestine, but others (e.g., Lactobacillus delbrueckii, Streptococcus thermophiles, Escherichia coli Nissle 1917, etc.) require their protection using enteric coatings. Examples of materials used in enteric coatings include copolymers of methyl acrylate, methyl methacrylate, and methacrylic acid. Exemplary enteric coatings to be used with the microbial cells of the present disclosure may be found in Yus et al., Polymers 2019, 11, 1668, the contents of which are hereby incorporated by reference.

In an embodiment, the microbial cells of the present disclosure are administered and/or delivered in the form of microbial spores. In some embodiments, the microbial cells comprise purified spore populations. These purified spore populations may be obtained from biological materials obtained from mammalian subjects, including humans. Exemplary biological materials include fecal materials such as feces or materials isolated from the various segments of the small and large intestines. Purification of the spore populations may be through methods known in the art, including solvent treatments, chromatography treatments (i.e., contacting the biological sample with a solid medium containing a hydrophobic interaction chromatographic (HIC) medium or an affinity chromatographic medium), mechanical treatments (i.e., physical disruption), thermal treatments (e.g., subjecting the biological material mixed in a solution to a heated environment), or irradiation treatments (e.g., subjecting the biological material to ionizing radiation, typically gamma irradiation, ultraviolet irradiation or electron beam irradiation provided at an energy level sufficient to kill pathogenic materials while not substantially damaging the desired spore populations). Exemplary methods of purification of spore populations and formulations for microbial spore administration may be found, for example, in U.S. Application Pub. No. 2021/0244774 A1, the contents of which are hereby incorporated by reference.

### Engineering a Bacterial Cell

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g. bacterial vectors having a bacterial origin of replication). A vector can be a replicon, such as a plasmid, phage, or cosmid, into which another DNA segment may be inserted so as to bring about the replication of the inserted segment.

### Pharmaceutical Compositions

The invention also encompasses the use of pharmaceutical compositions comprising a IsmA polypeptide (e.g., an Oscillibacter IsmA polypeptide) and/or a CgT polypeptide, and/or a microbial cell of the disclosure (e.g., Oscillibacter species, species expressing IsmA, and/or bacterial cells expressing IsmA and/or CgT) to practice the methods of the invention. Such a pharmaceutical composition may comprise said polypeptides and/or cells in a form suitable for administration to a subject and one or more pharmaceutically acceptable carriers, one or more additional ingredients, or some combination of these.

In embodiments, the pharmaceutical composition is administered enterically. This preferentially includes oral administration, or by an oral or nasal tube (including nasogastric, nasojejunal, oral gastric, or oral jejunal). In other embodiments, administration includes rectal administration (including enema, suppository, or colonoscopy). The composition may be administered to at least one region of the gastrointestinal tract, including the mouth, esophagus, stomach, small intestine, large intestine, and rectum. In some embodiments, it is administered to all regions of the gastrointestinal tract. The compositions may be administered orally in the form of medicaments such as powders, capsules, tablets, gels or liquids. The compositions may also be administered in gel or liquid form by the oral route or through a nasogastric tube, or by the rectal route in a gel or liquid form, by enema or instillation through a colonoscope or by a suppository.

If the composition is administered colonoscopically and, optionally, if the composition is administered by other rectal routes (such as an enema or suppository) or even if the subject has an oral administration, the subject may have a colonic-cleansing preparation. The colon-cleansing preparation can facilitate proper use of the colonoscope or other administration devices, but even when it does not serve a mechanical purpose it can also maximize the proportion of the composition, such as compositions comprising microbial cells of the present disclosure, relative to the other organisms previously residing in the gastrointestinal tract of the subject. Any ordinarily acceptable colonic cleansing preparation may be used such as those typically provided when a subject undergoes a colonoscopy.

In some embodiments, compositions of the present disclosure are formulated for targeted delivery to one at least one region of the gastrointestinal tract, such as, the small intestine, the large intestine, the colon, or the rectum. In some embodiments, the compositions are formulated for targeted delivery to the small intestine, such as to the duodenum, the ileum, or the jejunum. In an embodiment, the compositions are formulated for targeted delivery to the ileum. Enteric coatings with buffering or protective compositions may be used for targeted delivery in pharmaceutical compositions of the present disclosure. The enteric coating may be formulated to deliver the composition to at least one region of the gastrointestinal tract, such as, the small intestine, the large intestine, the colon, or the rectum. In some embodiments, the enteric coating is formulated to deliver the composition to the small intestine, such as to the duodenum, the ileum, or the jejunum. In an embodiment, the enteric coating is formulated to deliver the composition to the ileum. Examples of materials used in enteric coatings include, but are not limited to, copolymers of methyl acrylate, methyl methacrylate, and methacrylic acid. In embodiments, compositions of the present disclosure may include enteric coatings in combination with other buffers or protective compositions.

Solid dosage forms for oral administration include capsules, tablets, caplets, pills, troches, lozenges, powders, and granules. Alternatively, powders or granules embodying the compositions disclosed herein can be incorporated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

Compositions of the present disclosure may be prepared in lyophilized form. In short, compositions of the present disclosure may be suspended in lyophilization medium, the medium optionally comprising cryoprotectants, and/or biological or chemical oxygen scavengers. The compositions of the present disclosure are then transferred into a lyophilizer, optionally under anaerobic conditions, for lyophilization. In embodiments, compositions including microbial cells of the present disclosure may comprise freshly thawed liquid microbial suspensions or refrigerated gelatin capsules filled with lyophilized microbial biomass. Exemplary lyophilization methods and formulations may be found in U.S. Patent Application Publication Nos. US 2016/0331791 A1 and US 2019/0105359 A1, each of which are hereby incorporated by reference.

In some embodiments, the food product can be a solid foodstuff. Suitable examples of a solid foodstuff include without limitation a food bar, a snack bar, a cookie, a brownie, a muffin, a cracker, an ice cream bar, a frozen yogurt bar, and the like.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 500 mg/kg/day.

The relative amounts of the agents and the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, parenteral, or another route of administration.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it may include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A non-limiting preservative is a combination of about 0.5% to 2.0% benzyl alcohol and 0.05% to 0.5% sorbic acid.

The composition preferably includes an antioxidant and a chelating agent which inhibits degradation. Exemplary antioxidants are BHT, BHA, alpha-tocopherol and ascorbic acid in the preferred range of about 0.01% to 0.3%, for example BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, for example in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

In some embodiments, the pharmaceutical composition further comprises a viscosity enhancing agent. In some embodiments, the viscosity enhancing agent includes methylcellulose, hydroxyethylcellulose, hydroxypropylmethylcellulose and smart hydrogel. In some embodiments, the viscosity enhancing agent is hydroxyethylcellulose. In some embodiments, the pharmaceutical composition comprises 0.01-1.0% (w/v) viscosity enhancing agent. In other embodiments, the intranasal pharmaceutical composition comprises 0.05% (w/v) hydroxyethylcellulose.

In some embodiments, the pH of the pharmaceutical composition is from 4.0 to 7.5. In other embodiments, the pH of the pharmaceutical composition is from 4.0 to 6.5. In another embodiment the pharmaceutical composition has a pH of from 5.5 to 6.5. In further embodiments, the pharmaceutical composition has a pH of from 6.0 to 6.5. In various implementations, the pH of said aqueous solution or liquid formulation is from pH 3 to pH 7, from pH 3 to pH 6, from pH 4 to pH 6, or from pH 5 to pH 6. These pH ranges may be achieved through the incorporation of one or more pH modifying agents, buffers, and the like. In some embodiments, a pH modifier such as acetic acid, is present in a final concentration of at least 0.001%, preferably at least 0.01%, more preferably between 0.01%-0.2% by weight of the composition.

In some embodiments, compositions comprising an Oscillibacter species, bacteria expressing IsmA and/or CgT, and/or recombinant bacterial cells expressing IsmA and/or CgT, is administered as a maximum-tolerated dose (MTD). In some embodiments, MTD is the dose with estimated probability of dose limiting toxicity (DLT) closest to the target toxicity rate of 20%. In some embodiments, compositions comprising an Oscillibacter species, bacteria expressing IsmA and/or CgT, and/or recombinant bacterial cells expressing IsmA and/or CgT, is administered in a therapeutically effective dose for a mammal. In some embodiments, the mammal is a mouse. In some embodiments, a mouse is administered a dose of 0.5 million to 15 million cells. In some embodiments, the mammal is a human. In some embodiments, a human is administered a dose of at least about 0.25×10⁶ cells/kg, at least about 0.5×10⁶ cells/kg, at least about 1×10⁶ cells/kg, or at least about 1.5×10⁶ cells/kg.

### Combination Therapies

Optionally, a IsmA polypeptide (e.g., an Oscillibacter IsmA polypeptide) and/or a CgT polypeptide, and/or a microbial cell of the disclosure (e.g., Oscillibacter species, species expressing IsmA, and/or bacterial cells expressing IsmA and/or CgT) described herein may be administered in combination with any other agent having low density lipoprotein (LDL) cholesterol lowering effects (i.e., an LDL cholesterol lowering agent).

In an embodiment, the administration is part of a method of treating cardiovascular disease, cholesterol related disorders, or diseases associated with or characterized by increased levels of plasma triglycerides, plasma cholesterol, or serum C-reactive protein in a subject, or a method of lowering plasma cholesterol in a subject. The IsmA polypeptide (e.g., an Oscillibacter IsmA polypeptide) and/or a CgT polypeptide, and/or a microbial cell of the disclosure (e.g., Oscillibacter species, species expressing IsmA, and/or bacterial cells expressing IsmA and/or CgT) described herein may be advantageously used in treating subjects which have previously been, or are concurrently being administered an LDL cholesterol lowering agent, particularly where the subject is currently, or has previously been administered a maximum tolerated dose of the LDL cholesterol lowering agent, or where the subject has side effects or symptoms of toxicity associated with the LDL cholesterol lowering agent. The IsmA polypeptide (e.g., an Oscillibacter IsmA polypeptide) and/or a CgT polypeptide, and/or a microbial cell of the disclosure (e.g., Oscillibacter species, species expressing IsmA, and/or bacterial cells expressing IsmA and/or CgT) described herein can also be formulated as a combination therapy with any other agent having LDL cholesterol lowering effects.

Non-limiting examples of an LDL cholesterol lowering agent include one or more of a statin, a cholesterol absorption inhibitor, a bile acid sequestrant, a PCSK9 inhibitor, an adenosine triphosphate-citrate lyase (ACL) inhibitor, or a microsomal triglyceride transfer protein (MTP) inhibitor. Examples of statins include, but are not limited to, atorvastatin, cerivastatin, fluvastatin, lovastatin, mevastatin, pitavastatin, pravastatin, rosuvastatin, or simvastatin. Examples of cholesterol absorption inhibitors include, but are not limited to, ezetimibe. Examples of bile acid sequestrants include, but are not limited to, cholestyramine, colesevelam, or colestipol. Examples of PCSK9 inhibitors include, but are not limited to, alirocumab or evolocumab. Examples of ACL inhibitors include, but are not limited to, bempedoic acid. Examples of MTP inhibitors include, but are not limited to, lomitapide.

In some embodiments, the IsmA polypeptide (e.g., an Oscillibacter IsmA polypeptide) and/or a CgT polypeptide, and/or a microbial cell of the disclosure (e.g., Oscillibacter species, species expressing IsmA, and/or bacterial cells expressing IsmA and/or CgT) described herein is administered simultaneously or sequentially with the LDL cholesterol lowering agent.

### Methods of Delivery

The regimen of administration of the IsmA and/or CgT polypeptides, microbial cell endogenously expressing the same, or a microbial cell engineered to express the same may be provided to a subject in an effective amount. Further, several divided dosages, as well as staggered dosages may be administered daily or sequentially, or the dose may be continuously infused, or may be a bolus injection. Further, the dosages of the formulations may be proportionally increased or decreased as indicated by the exigencies of the therapeutic or prophylactic situation. In embodiments, the IsmA and/or CgT polypeptides, microbial cell endogenously expressing the same, or a microbial cell engineered to express the same are provided in a powder, bolus gel, capsule, liquid, or foodstuff.

In embodiments, a polypeptide (e.g., IsmA, CgT) is delivered to the gut by virtually any method known in the art. See, for example, Kreitz et al, Nature. 2023; 616(7956): 357 364; and El-Sherbiny, Carbohydrate Polymers, Volume 80, Issue 4, 16 May 2010, Pages 1125-113; and U.S. Pat. No. 11,505,583. Methods for the targeted delivery of probiotics and related agents is described, for example, by Yoha et al., Probiotics Antimicrob Proteins. 2022; 14(1): 15-48. See also, Enck et al., Curr Pharm Des. 2020; 26(26): 3134-3140, and US Patent Publication No. 20180296582, as well as U.S. Pat. No. 11,633,436.

An effective amount of the therapeutic composition necessary to achieve a therapeutic effect may vary according to factors such as the activity of the particular composition employed; the time of administration; the rate of excretion of the composition; the duration of the treatment; other drugs, compounds or materials used in combination with the active ingredient; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic composition without undue experimentation.

The composition can be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the composition in dosage unit form for ease of administration and uniformity of dosage. Dosage unit form as used herein refers to physically discrete units suited as unitary dosages for the patients to be treated; each unit containing a predetermined quantity of therapeutic composition calculated to produce the desired therapeutic effect in association with the required pharmaceutical vehicle. The dosage unit forms of the invention are dictated by and directly dependent on (a) the unique characteristics of the therapeutic composition and the particular therapeutic effect to be achieved, and (b) the limitations inherent in the art of formulating such a therapeutic composition for the treatment of CVD or cholesterol related disorders in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It will be readily apparent to one skilled in the art that the frequency of administration of the various compositions of the invention will vary from subject to subject depending on many factors including, but not limited to, age, disease or disorder to be treated, gender, overall health, and other factors. Thus, the invention should not be construed to be limited to any particular dosage regimen and the precise dosage and composition to be administered to any patient will be determined by the attending physical taking all other factors about the patient into account.

Compositions of the invention for administration may be in the range of from about 1 mg to about 7,500 mg, about 20 mg to about 7,000 mg, about 40 mg to about 6,500 mg, about 80 mg to about 6,000 mg, about 100 mg to about 5,500 mg, about 200 mg to about 5,000 mg, about 400 mg to about 4,000 mg, about 800 mg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween. In certain preferred embodiments, the compositions of the invention can be administered to a subject in a dosage from about 0.1 mg/kg body weight to about 10 mg/kg body weight.

In some embodiments, the dose of a composition of the invention is from about 0.5 mg and about 5,000 mg. In some embodiments, a dose of a composition of the invention used herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a composition of the invention, alone or in combination with a second pharmaceutical agent; and instructions for using the compound to treat, prevent, or reduce one or more symptoms of CVD in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing cancer in a patient.

### Routes of Administration

Routes of administration of IsmA and/or CgT polypeptides, microbial cell endogenously expressing the same, or a microbial cell engineered to express the same, or any of the other compositions described herein include oral, rectal, parenteral, and (trans) rectal), intravesical, intraduodenal, and intragastrical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application of IsmA and/or CgT polypeptides, microbial cell endogenously expressing the same, or a microbial cell engineered to express the same, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compositions useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a IsmA and/or CgT polypeptides, microbial cell endogenously expressing the same, or a microbial cell engineered to express the same includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution or as a lyophilized cake which can be reconstituted by the addition of a solvent. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Kits

The present compositions (e.g., compositions comprising IsmA and/or CgT polypeptides, microbial cell endogenously expressing the same, or a microbial cell engineered to express the same) can be assembled into kits or pharmaceutical systems for treating a CVD or a symptom thereof. Kits or pharmaceutical systems comprise a carrier means, such as a box, carton, tube or the like, having in close confinement therein one or more container means, such as vials, tubes, ampoules, bottles and the like. The kits or pharmaceutical systems can also comprise associated instructions for using the agents of the presently disclosed embodiments. In some embodiments, kits include therapeutic compositions disclosed herein (e.g., compositions including IsmA, CgT, Oscillibacter species, species expressing IsmA, and/or cells expressing IsmA and/or CgT).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Characterization of the FHS Gut Microbiome Using Shotgun Metagenomics

The Framingham Heart Study (FHS) monitors anthropometric measurements (e.g. body mass index or BMI), blood measurements (e.g. plasma cholesterol, triglycerides and glucose), relevant prescription medications (e.g. statin and insulin) and diagnoses of clinical cardiovascular disease (CVD) and diabetes across multiple generations of participants. The present analysis focuses on the most recently recruited FHS cohorts involving 1,429 individuals (Generation 3 recruited in 2002, OMNI2, and New Offspring Spouse recruited in 2003). Overall, the samples are well balanced in sex (796 women and 633 men) and span a wide age range (32-89 years, with a mean of 55.3). A notable fraction of the individuals exhibited markers of CVD risk on the basis of plasma measurements of key risk factors (e.g. 315 with fasting glucose >100 mg/dL, 174 with triglycerides >150 mg/dL and 434 with cholesterol >200 mg/dL), despite not having been diagnosed with clinical CVD or diabetes, or taking any related medications at the time of collection.

To investigate the link between the gut microbiome and cardiovascular health in this cohort, fecal samples were collected for metagenomic and metabolomic characterization. Shotgun metagenomic libraries were generated from the fecal samples (N=1,345 after quality control), resulting in an average of 16 million high-quality microbial reads per sample. The reads were de novo assembled into a non-redundant gene catalog (32) of over 3 million genes and 687 species-level assemblies (referred as metagenomic species pangenomes or MSPs (30)) distributed across 231 known species and 107 genera. The assembled MSPs carry an average of 1,038 core genes (shared by all the subspecies of the MSP) and 667 accessory genes (not present in all the subspecies of the MSP). Notably, an average of 36% of the genes were poorly characterized, with 19% not mapped to any Clusters of Orthologous Groups (COG) gene families and 17% mapped to families with unknown function. To find remote homologs (low sequence level similarity) of the proteins encoded by these genes, the uncharacterized sequences were further annotated using a protein language model searching structurally similar proteins against a customized database containing more than 1.4 million characterized proteins, yielding an additional 3,994 annotated gene families spanning 42,842 genes.

The broad microbiome profiles denoted by the first two low dimensional projection coordinates (determined by principal coordinates analysis (PCoA) using Bray-Curtis dissimilarity) revealed a continuous spectrum. It ranged from samples with low α-diversity and enriched in Ruminococcus gnavus, Clostridium bolteae, Bacteroides vulgatus and Flavonifractor plautii (msp_130) to samples with high α-diversity and enriched with genera Alistipes and Oscillibacter. Notably, several Oscillibacter MSPs were associated with overall microbiome diversity, which is not reported in previous studies relying on reference based analysis workflow due to the incompleteness of reference databases. In addition, Prevotella copri (msp_016) was present in a small fraction of samples, albeit at high abundance. Subjects with high α-diversity and enrichment of Alistipes and Oscillibacter showed lower plasma levels of triglycerides, glucose, and higher plasma levels of HDL in the cohort. These overall trends are also observed in other ethnically distinct cohorts, indicating an important role of the gut microbiome in cardiovascular health.

### Example 2: Untargeted Metabolomic Profiling Reveals Overall Association Between the Stool Metabolites and Microbiome

To gain further insights into the functional implications of the host-microbiome interactions, untargeted metabolomic profiles from 899 matched stool samples were generated by performing liquid chromatography-mass spectrometry (LC-MS) using four methods, measuring polar metabolites, lipids, free fatty acids, and bile acids, respectively. This yielded 130,877 LC-MS peaks, of which 568 were aligned with standards (referred as “known metabolites” hereafter). The remaining unaligned peaks were matched with chemical formulas in the Human Metabolome Database (HMDB) (within 5 ppm tolerance (33, 34)), yielding 62,439 candidate annotations. Among all the candidate molecules per peak, the predicted molecular class was selected using the majority rule. While this approach cannot be used for exact compound identification, it yielded the correct class prediction for 76% of known metabolites and enabled survey of broader changes in the stool metabolomes.

The overall metabolomic profile was significantly associated with the MSP profile (Procrustes analysis, P<0.05). Major metabolomic classes with the strongest associations with microbiome α-diversity were fatty acyls, steroids and derivatives, glycerol lipids and prenol lipids. Notably, the number of unknown peaks was higher in samples enriched with Alistipes and Oscillibacter compared to other samples, indicating a large uncharacterized metabolomic space in gut microbiomes of these participants that potentially related to the associations between the gut microbiome and lower blood lipid levels observed.

### Example 3: Identification of Microbial Species Associated with Blood Measurements

The relationship between the gut microbiome and cardiovascular health related blood measurements was next analyzed (plasma: triglycerides, cholesterol, high-density lipoprotein (HDL), glucose; serum: alanine transaminase (ALT), aspartate aminotransferase (AST), albumin, creatinine and C-reactive protein (CRP); whole blood: hemoglobin A1c (HbA1c); low-density lipoprotein (LDL) estimated using Friedwald equation; blood pressure), as most of the participants were reported to be healthy during the sample collection. The overall gut microbiome of FHS participants was significantly associated with blood triglycerides, HbA1c, cholesterol, liver enzyme ALT and systolic blood pressure (PERMANOVA test, Padj <0.1), and the percentage of explained variations were similar to reports from other studies (Talmor-Barkan et al., Nat. Med. 28, 295-302, 2022, Fromentin et al., Nat. Med. 28, 303-314, 2022). Specifically, 129 pairs of significant associations between individual MSPs and blood measurements were identified (two-sided t-test, Padj <0.1) using generalized linear models controlling for various host factors also associated with the microbiome (age, sex, BMI and CVD-relevant prescriptions; Methods). Overall, the associations with triglycerides and cholesterol were concordant with the findings from the PREDICT1 cohort using a different analysis workflow (Spearman correlation >0.56, P<0.05).

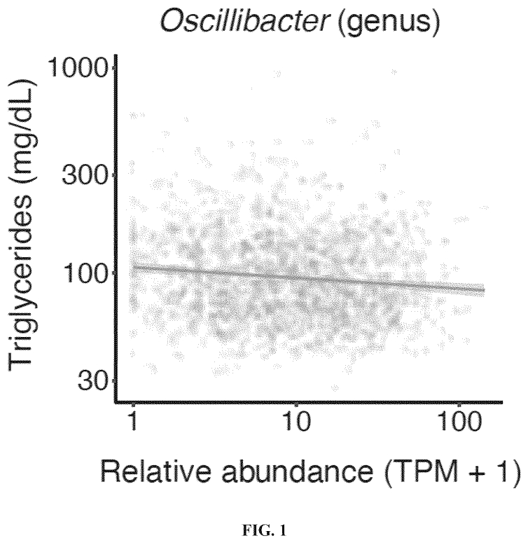

Several MSPs were associated with multiple CVD risk factors, including F. plautii (msp_130) and C. bolteae. Both of the MSPs were associated with higher plasma levels of triglycerides and glucose, and F. plautii was also associated with higher plasma cholesterol. The abundance of Oscillibacter, a group of poorly characterized slow-growing microbes, was associated with lower plasma triglyceride levels (FIG. 1). These specific associations with blood cholesterol and triglycerides were also validated in the PREDICT1 cohort.

In addition to validated associations, associations were uncovered that had not previously been reported. For example, a positive association was observed between the serum concentrations of the inflammation marker CRP and Parabacteroides merdae. A negative association was also detected between Alistipes obesi with both plasma cholesterol and triglycerides. The reference-free analysis facilitated discovery of unreported associations for poorly characterized microbes without high-quality reference genomes. For example, an MSP from Firmicutes (msp_120) showed the strongest positive association with plasma cholesterol, which warrants further investigation.

The de novo assembly workflow also allowed investigation of microbial associations between subspecies and blood measurements using the accessory genes. 5,132 pairs of significant associations were identified (accessory genes with blood measurements; two-sided t-test, Padj <0.05) involving 153 MSPs. Notably, the dominant human gut archaeon, Methanobrevibacter smithii, carried 120 accessory genes associated with plasma cholesterol levels (46 negative and 74 positive). The two subspecies (based on the presence-absence of the two groups of accessory genes) were consistent with published M. smithii genomes and corresponded to the two clades identified previously showing different growth behaviors in vitro. The two subspecies carry different MtrC and MtrD genes, which form the Mtr complex critical for reducing hydrogen levels in the gut through the methane production pathway.

### Example 4: Stool Metabolomes Reveal Associations Between Blood Measurements and Lipids, Including Steroids and Derivatives

To examine the relationship between stool metabolites and blood measurements, 2,368 co-abundance clusters of LC-MS peaks were defined, following the principles of molecular networking which can elucidate pathway-dependent changes in human samples. A total of 1,375 clusters harbored at least one peak associated with cardiovascular health after adjustment for age, sex, BMI and medications (Padj <0.05). The blood measurements with the largest number of associations with stool metabolites were plasma HDL (3,005), plasma triglycerides (1,706), plasma cholesterol (467) and serum CRP (1,672).

Using the predicted metabolites classes from HMDB (majority rule), it was observed that the largest number of associated peaks were among prenol lipids and fatty acyls (with HDL and CRP), while plasma triglycerides were disproportionately associated with steroids and steroid derivatives, highlighting the importance of this class for lipid homeostasis of the host.

The analysis identified numerous individual metabolites related to microbial metabolism associated with known health parameters or dietary patterns. Plasma triglyceride levels were strongly associated with carnitines and trimethyllysine, which are converted by gut microbes to trimethylamine (TMA), a precursor to the known CVD risk factor TMAO. Another group of triglyceride-associated metabolites were N-acylethanolamines (palmitoyl-EA and linoleoyl-EA), host-produced lipids which promote growth of inflammation-associated bacteria R. gnavus and E. coli.

Potential microbial tryptophan (Trp) derivatives and G-protein coupled receptor (GPCR) ligands (e.g. tryptamine, kynurenic acid, indoleacetate and serotonin) were associated with different measurements. Proinflammatory metabolite tryptamine produced by microbes (e.g., R. gnavus) from tryptophan was correlated with increased plasma triglycerides, while kynurenic acid was associated with decreased plasma triglyceride levels. Indoleacetate was associated with increased CRP levels. Elevated serotonin and melatonin precursor N-acetylserotonin levels were associated with decreased plasma triglyceride levels.

Primary bile acids including chenodeoxycholate, glycodeoxycholate and taurodeoxycholate in the stool were positively associated with plasma triglycerides. Conversely, secondary bile acid isoallolithocholic acid (isoalloLCA), a microbial product with anti-inflammatory and antimicrobial (particularly against Gram-positive pathogens) functions showed negative correlation with plasma triglycerides. Moreover, a cluster of metabolites including short-chain fatty acids (butyrate and propionate) was positively associated with the levels of HDL, consistent with results from a recent clinical trial on attenuating atherosclerosis with propionate.

Finally, stool cholesterol was among the strongest correlates to multiple CVD-relevant blood measurements, such as levels of plasma cholesterol, plasma triglycerides and serum CRP. Consistent with previous findings, coprostanol, a microbial product of cholesterol metabolism, showed the expected reverse trend with the above measurements. In addition, increased levels of cholesteryl esters (CEs) were associated with elevated levels of LDL and decreased HDL cholesterol. Taken together, these metabolites are potentially contributed by the microbes associated with cardiovascular health identified above.

### Example 5: Characterization of MSP-Metabolite Associations with Integrated Analysis

Next, significant associations between gut microbes and stool metabolites in the context of cardiovascular health were characterized. It was observed that two clusters of known metabolites correlated with multiple MSPs (>5), forming two blocks of distinct MSP-metabolite associations. The two clusters are referred to by their representative MSPs, Oscillibacter (also includes Alistipes) and R. gnavus (also includes C. bolteae, F. plautii/msp_130 and B. obeum), respectively. The Oscillibacter cluster was positively correlated with dicarboxylic acids (e.g. 3-methyladipate, azelate, suberate and dodecanedioic acid), isoalloLCA and coprostanol, while the R. gnavus cluster was positively correlated with polyunsaturated fatty acids (e.g. docosapentaenoate, eicosatrienoate and adrenate), acylcarnitines, N-acylethanolamines (NEAs; e.g. linoleoyl ethanolamide (LEA) and palmitoylethanolamide (PEA)), tryptamine, primary bile acids (cholate and chenodeoxycholate), cholesterol, phytosterols (campesterol and sitosterol) and neurosteroids (dehydroepiandrosterone sulfate and pregnenolone sulfate). These differences between the Oscillibacter and R. gnavus clusters indicate a gut environmental spectrum in healthy individuals defined by abundances of inflammatory molecules and specific MSPs. Notably, the metabolites signifying the inflammatory extreme of such a spectrum (i.e. high PUFA, tryptamine and N-acylethanolamines, and low dicarboxylic acids) match the metabolites enriched in the gut of patients with immune disorders compared to healthy controls (Fornelos et al., Nature Microbiology 5, 486-497, 2020, Franzosa et al., Nat Microbiol 4, 293-305, 2019, Bowerman et al., Nat. Commun. 11, 5886, 2020).

Using the paired multi-omics data, the plausible molecular functions of the MSPs correlated with cardiovascular health related measurements was investigated. This began with MSPs that produce γ-butyrobetaine (γBB), an intermediate in carnitine metabolism (strongly correlated with carnitine, Spearman correlation=0.51, P<10-15), the major contributor to atherosclerosis-promoting agent TMAO (15,16). Five MSPs F. plautii (msp_130), C. bolteae, B. vulgatus, C. clostridioforme and R. gnavus were positively associated with stool γ-butyrobetaine levels (Spearman correlation >0.2). The E. coli caiTABCDE operon responsible for the production of γ-butyrobetaine from L-carnitine was queried against the MSP genes, and found that only C. clostridioforme possessed the full set of six genes (five were found in C. bolteae).

To validate the production of γ-butyrobetaine by these MSPs, the metabolomic profiling data of bacterial isolate cultures from a published dataset was reanalyzed. It was confirmed that an 8.4- to 11.06-fold increase of γ-butyrobetaine for C. clostridioforme and C. bolteae in spent medium compared to controls. The canonical precursor L-carnitine remained largely unchanged, suggesting the presence of an unknown bio-transformation to produce γ-butyrobetaine in these strains. Choline, another known precursor of TMA, showed more than 100-fold decrease in the culture of C. clostridioforme and C. bolteae.

Next, the metabolic potential of F. plautii (msp_130) was examined, due to its association with several CVD-related blood measurements. F. plautii degrades flavonoids into phenolic acids, which have antioxidant and anti-inflammatory properties that are expected to have a beneficial role on human health. To uncover potential explanations for such inconsistencies between metabolic function and association with blood measurements, the association between F. plautii (msp_130) and phenolic acids was examined. The abundance of F. plautii (msp_130) was negatively correlated with phenyl-propionic acid (QI21021), dihydroxy-phenyl-propionic acid (QI12828, QI49585) and dihydroxy-phenyl-acetic acid (QI19271), but positively correlated with hydroxy-phenyl-propionic acid (QI19217) and hydroxy-phenyl-acetic acid (QI4944, QI20809). Potential candidate flavonoid degradation intermediates (chalcones or dihydrochalcones, predicted) that correlated with F. plautii (msp_130) abundances (absolute correlation >0.2) were also identified. In contrast, another flavonoid degrader Eubacterium ramulus (58) did not show a comparable correlation with the metabolites above (max absolute correlation <0.2). Therefore, F. plautii's varied efficiency in degrading different dietary flavonoids (59) might contribute to a phenolic acid profile (e.g. less dihydroxyl-phenolic acids) that is less effective in attenuating inflammatory responses (60).

To further mine novel metabolic reactions carried out by the gut microbiome, the associations between MSPs and the unknown LC-MS peaks were also explored. In total, 13,398 positive and 2,626 negative pairs of strong correlations (absolute Spearman correlation >0.4) were found. Among them, two MSPs negatively associated with plasma triglycerides, Ruminococcus bicirculans and Eubacterium siraeum were strongly associated with unknown peaks (QI29995 and QI28613, respectively). Firmicutes msp_120, an MSP with the strongest positive association with plasma cholesterol, was positively correlated with an unknown peak (QI16816) approximately matched to a glycerophosphocholine. Moreover, using MS/MS data and SIRIUS, Bifidobacterium adolescentis-associated peak QI29637 was predicted as a bile acid derivative that showed similarity (GNPS cosine >0.7) to other predicted bile acids. The majority of peaks, especially the unknown ones, were strongly associated with only a small number of MSPs. These include 756 unknown peaks belonging to the co-abundance clusters significantly associated with blood measurements. Further characterization of these peaks and MSPs that potentially produce or consume them, as presented for Oscillibacter spp. below, could lead to the discovery of novel CVD-related gut microbial metabolic reactions.

### Example 6: Oscillibacter MSPs are Strongly Associated with Stool Cholesterol and its Derivatives

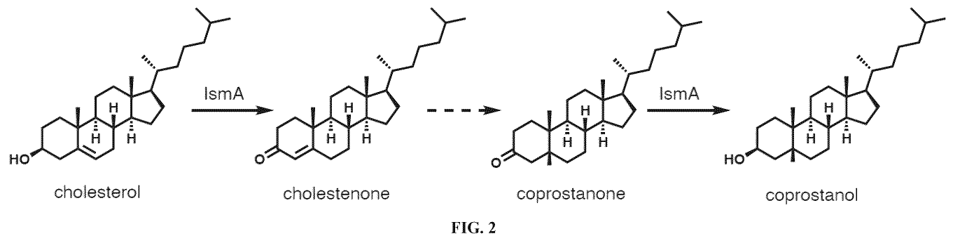

Cholesterol levels in the stool were associated with multiple CVD-related blood measurements. Recently, a protein family of 3ß-hydroxysteroid dehydrogenases (3β-HSD) encoded by intestinal sterol metabolism A (ismA) genes harbored by uncultured gut bacteria in cluster IV Clostridium was discovered. The IsmA enzyme first converts cholesterol to cholestenone (4-cholesten-3-one) and then transforms the final intermediate coprostanone to coprostanol, which is poorly absorbed by the host and largely excreted in feces (FIG. 2). The presence of microbes carrying the ismA genes in the gut microbiome was also shown to be associated with decreased stool and plasma cholesterol levels in humans (Kenny et al., Cell Host Microbe 28, 245-257.e6, 2020). To build on these findings, this framework was applied to identify additional gut microbes and enzymes that may metabolize cholesterol and modulate its levels in human hosts.

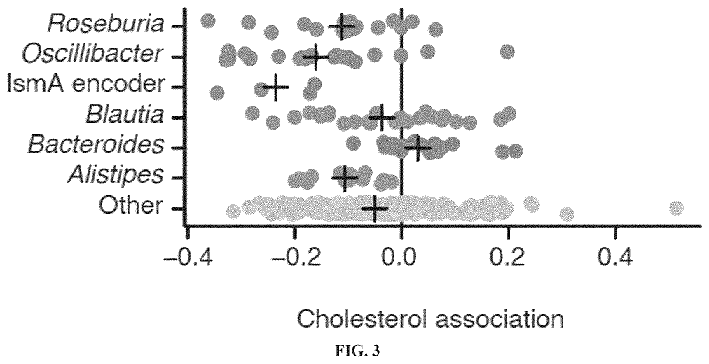

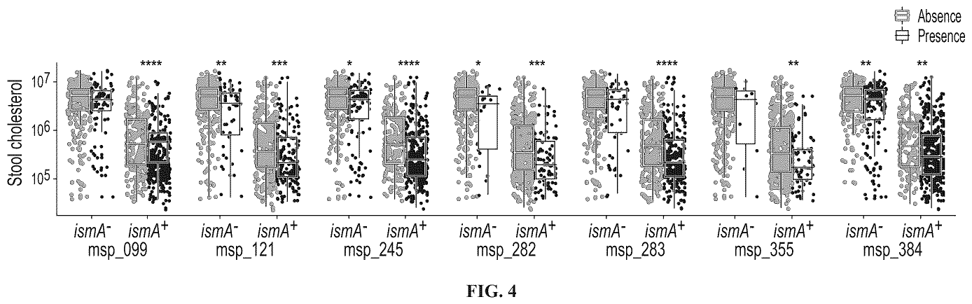

It was hypothesized that the abundances of gut microbes metabolizing cholesterol should be correlated with stool cholesterol level. 180 MSPs negatively associated with stool cholesterol were found (Padj <0.05 FIG. 3), including 4 MSPs encoding ismA gene homologs (IsmA encoders). Consistent with the previous report, the presence of IsmA encoders in 470 (out of 830) samples was associated with an increase of cholestenone and coprostanol. However, detection of cholestenone and coprostanol in the remaining 360 samples lacking IsmA encoders suggests that other microbes might also be able to produce these cholesterol derivatives. Among the MSPs associated with stool cholesterol, a significant enrichment of MSPs annotated to the Oscillibacter genus was noted (FIG. 3; Fisher's exact test, P<10-5), in which no strong sequence similarity to IsmA was found (no more than 50% amino acid identity and 50% coverage; Methods). These Oscillibacter spp. were highly prevalent regardless of the presence or absence of the IsmA encoders and exhibited diverse abundance patterns across the samples. In addition, among the top 30 MSPs associated with decreased stool cholesterol, MSPs annotated to the Oscillibacter genus had the highest cumulative relative abundance (median 1.4%, IQR 4.3%), prompting an in-depth follow-up. To control for the effect of IsmA encoders, the samples were stratified based on IsmA encoder detection (ismA+ and ismA−) and prioritized Oscillibacter MSPs using a sparse regression model. The presence of seven Oscillibacter MSPs was associated with further decreased stool cholesterol levels in ismA+ samples regardless of the inclusion of dietary variables (except for msp_282), and several of them were associated with significant decrease of stool cholesterol levels (and increase of stool cholestenone or coprostanol) in ismA− samples (FIG. 4).

It was hypothesized that IsmA encoders and Oscillibacter MSPs act additively or synergistically in metabolizing cholesterol, resulting in the decreased stool cholesterol levels. To uncover potential metabolic pathways of cholesterol and its derivatives in Oscillibacter spp., a molecular network seeded by seven LC-MS peaks was examined that had a mass corresponding to that of cholesterol (C27H460; theoretical m/z 386.3554; including the standard-annotated peak) and significantly correlated to plasma cholesterol. The cholesterol peaks induced a network consisting of metabolites separated by mass shifts corresponding to common microbial enzymatic reactions (62,63) and retention times within 0.5 min. This yielded 232 peaks representing predicted cholesterol derivatives (some peaks might redundantly correspond to the same metabolite) and a total of 239 peaks of interest. The targeted MS/MS fragmentation confirmed that most of identifiable peaks indeed correspond to steroids and their derivatives. Out of 239 targeted peaks, 84 MS/MS spectra were collected and for 56 an HMDB identifier could be predicted with SIRIUS (41 steroids and steroid derivatives, 9 prenol lipids, 4 homogenous other non-metal compounds, 1 phenol). Putative representatives of original molecules were selected using highest CSI: Finger ID scores. Two of the original seed peaks had cholesterol as a top prediction and two others agreed in the molecular formula predicted from the spectrum confirming the utility of the approach and the initial selection. Within all the selected peaks, 183 showed positive associations (two-sided t-test Padj <0.05) with at least one Oscillibacter MSP, while 63 showed at least one significant negative association.

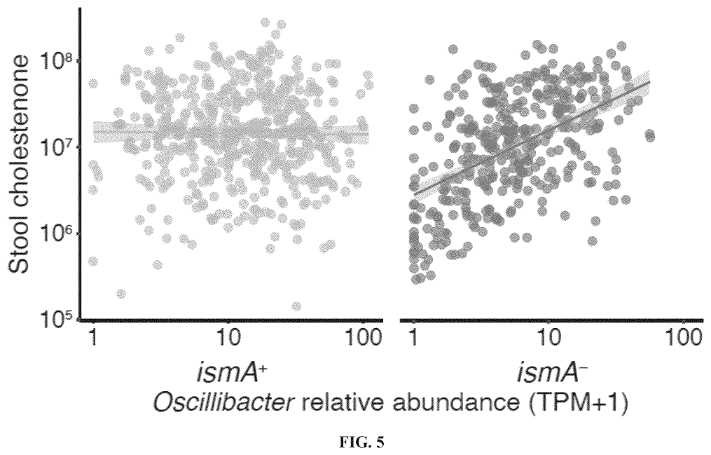

To correct for the potential effects on cholesterol derivatives by IsmA encoders, the correlation between the nodes and Oscillibacter abundances in the ismA− samples were re-examined. Most peaks connecting to the verified cholesterol peak have a stronger correlation with Oscillibacter in ismA− compared to ismA+ samples, confirming that the correlation is independent from the effect of IsmA encoders. The derivative with the strongest positive correlation with Oscillibacter in ismA− samples was cholestenone (Spearman correlation=0.50, P<10-21; FIG. 5), which was also positively correlated with Oscillibacter in ismA− samples from two independent cohorts (Spearman correlation of 0.23 and 0.21 in iHMP and PRISM, respectively; P<10-5). Other prominent peaks included coprostanol, desmosterol, oxysterols, beta-sitostenone and numerous peaks with increased mass in relation to cholesterol. Presence of both the IsmA encoders and Oscillibacter also exhibited an additive effect on lowering plasma cholesterol levels. Notably, a further decrease of plasma cholesterol and triglycerides was observed when an Oscillibacter MSP (msp_384; FIG. 6) was present together with the IsmA encoders. Reassuringly, such an additive effect was also observed in an ethnically distinct cohort collected from a hospital in Guangdong, China. The above in-depth exploration of untargeted metabolomic profiles and stool metagenomics thus revealed MSPs from the Oscillibacter clade as novel candidate cholesterol metabolizers. Its association with cholesterol and its derivatives is particularly notable in samples that lack the known cholesterol-reducing bacteria which suggest it may independently or synergistically contribute to reduced stool cholesterol levels.

### Example 7: Structural Similarity Search Using a Protein Language Model Identifies Candidate Oscillibacter Genes Involved in Cholesterol Metabolism

To seek candidate Oscillibacter proteins that engage with cholesterol, the genomes of Oscillibacter MSPs were analyzed together with isolates O. sp. RJX3711, Dysosmobacter welbionis (formerly named Oscillibacter welbionis) J115 and O. sp. RJX3347. RJX3711 was obtained from the stool of an FHS participant, and was shown to be the same species as msp_384 (genome-wide average nucleotide identity or ANI=98.0%). J115 is a commercial isolate almost identical to msp_257 (ANI=99.2%) and was previously shown to improve host lipid and glucose homeostasis in a mouse model. RJX3347 was isolated from the stool of a healthy individual and is phylogenetically bounded by the other two isolates and was shown to be more similar to msp_257 (ANI=82.7%). These three isolates belong to a group containing the two most prevalent Oscillibacter MSPs (msp_257 and msp_384) in the FHS cohort (>47% of participants), and therefore they were all analyzed as representatives hereafter.

The Oscillibacter assemblies and the genomes of Oscillibacter isolates were queried utilizing PROtein Sequence Embedding (PROSE), a pre-trained deep learning model which leverages tertiary structural information and thus improves functional protein queries over sequence comparison. Briefly, the 41,934 proteins making up 26 Oscillibacter MSPs were represented by a PROSE model with 6,165 variables. To enable functional queries in practice, this approach requires establishing Euclidean distance thresholds analogous to percentage identities in the sequence-based search. Analysis of 13,696 annotated enzymes in Oscillibacter MSPs showed that the Euclidean distance (dPROSE) in this PROSE model was predictive of a pair of proteins sharing an enzymatic function, with increasing sensitivity across Enzyme Commission number (EC) levels (area under ROC curve for EC level 4 separation=0.89). This established the method's utility and an interpretable range of distance values (median distance between equivalent enzymes dPROSE=1.95, for random pairs dPROSE=5.43).

For each of the three Oscillibacter isolates, a large fraction of proteins from the genome mapped to proteins from Oscillibacter MSPs (3,042 of 3,407 for RJX3347; 3,302 of 3,314 for RJX3711; and 3,198 of 3,529 for J115; dPROSE <1.95). The MSPs with most mapped proteins were msp_384 for RJX3711 (n=1,116 of 1,327 in msp_384), and msp_257 for both RJX3347 and J115 (n=516 and 1,652 of 1,843, respectively in msp_257), consistent with the phylogenetic placement of the isolates and MSPs.

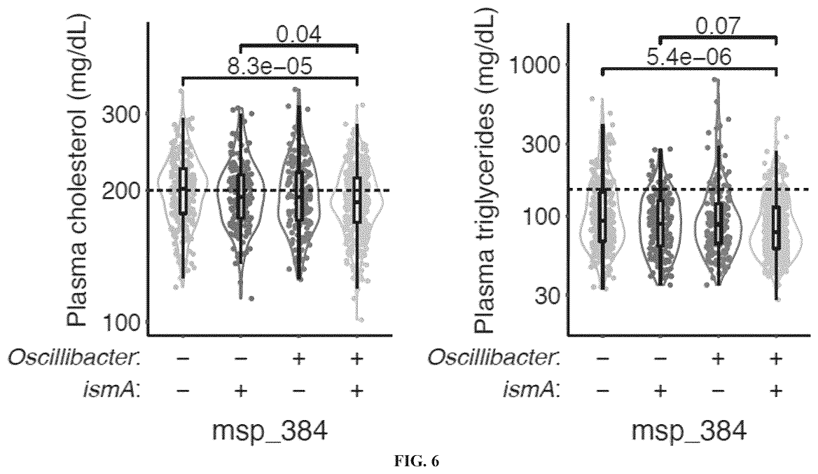

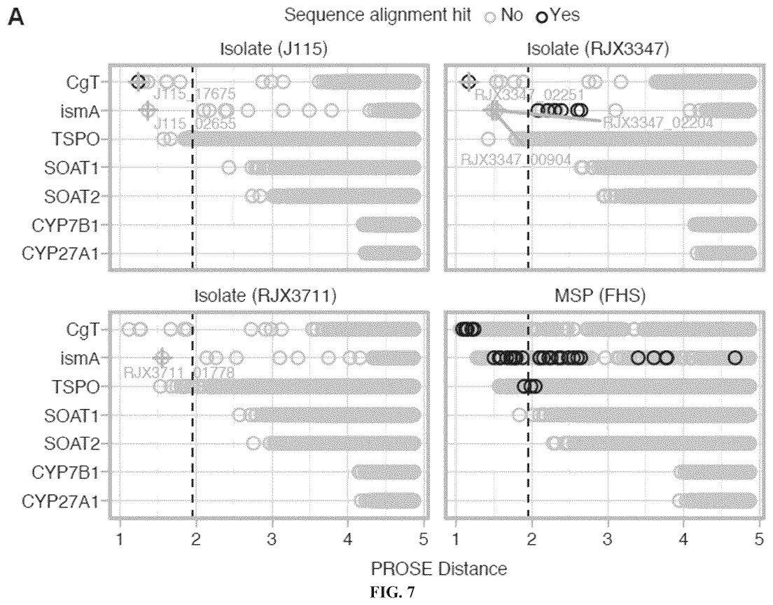

PROSE was then used to screen Oscillibacter isolate genomes for functional analogs to a curated list of proteins in human and microbial cholesterol pathways. Proteins were found that were predicted to structurally resemble cholesterol-α-glucosyltransferase (CgT), IsmA and translocator protein (TSPO) (FIG. 7). CgT catalyzes the synthesis of glycosylated cholesterol cholesteryl-α-D-glucopyranoside by Helicobacter pylori, IsmA is involved in converting cholesterol to coprostanol via cholestenone, and TSPO is responsible for cholesterol translocation from outer to inner membrane in human mitochondria. Neither the isolates nor Oscillibacter MSPs had proteins with high similarity to human enzymes for production of cholesteryl esters associated with CVD markers (SOAT1, min dPROSE=1.83; SOAT2 dPROSE =2.28), or production of oxygen-dependent oxysterols associated with Oscillibacter MSP abundance (CYP27A1, dPROSE=3.94; CYP27A1, dPROSE=3.96).

Of the top CgT hits, RJX3347_02251 (dPROSE=1.16) and J115_17675 (dPROSE=1.23) were almost identical (>90% identity). In addition, they had a high sequence similarity to the query in H. pylori (>70% identity) and were annotated to the corresponding function (“GDP-mannose-dependent alpha-mannosyltransferase”). However, the top hit from RJX3711 (RJX3711_02717, dPROSE=1.11) did not match the CgT query by sequence and was annotated to “D-inositol-3-phosphate glycosyltransferase”; and it is therefore likely to function on substrates other than cholesterol. For the IsmA hits, RJX3347_02204 (dPROSE=1.53), RJX3711_01778 (dPROSE=1.55) and J115_02655 (dPROSE-1.36) shared high sequence similarity (>70% identity) and likely have a similar function. IsmA hit RJX3347_00904 (dPROSE=1.48) was unique to this isolate and not found by sequence similarity search in the MSPs, and is therefore likely not relevant for the association of Oscillibacter spp. with cholesterol in the cohort. Finally, the three TSPO hits (RJX3347_01966, dPROSE=1.42; RJX3711_02962, dPROSE=1.53; J115_17465, dPROSE=1.57) had weaker similarity (>50% identity), but likely belong to the same protein family.

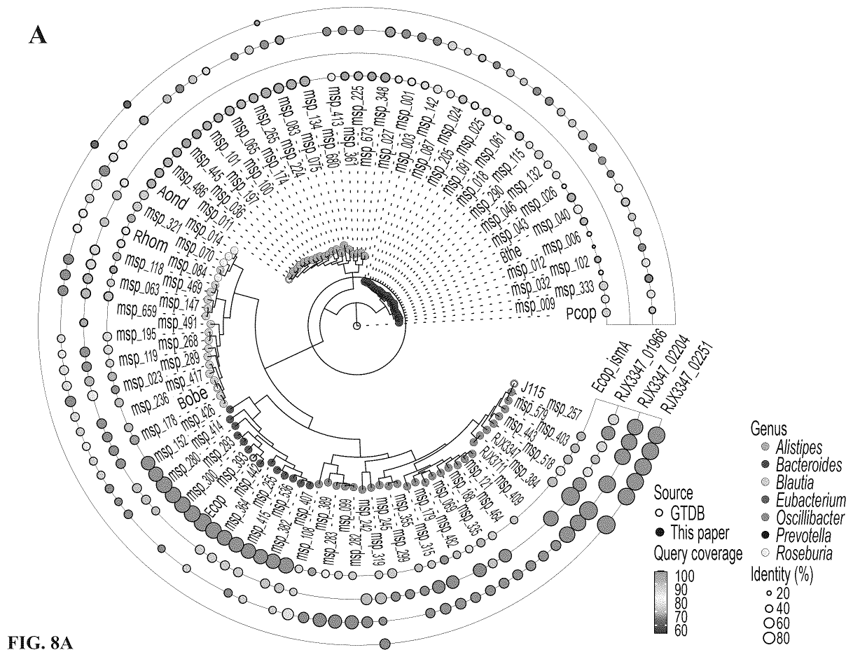

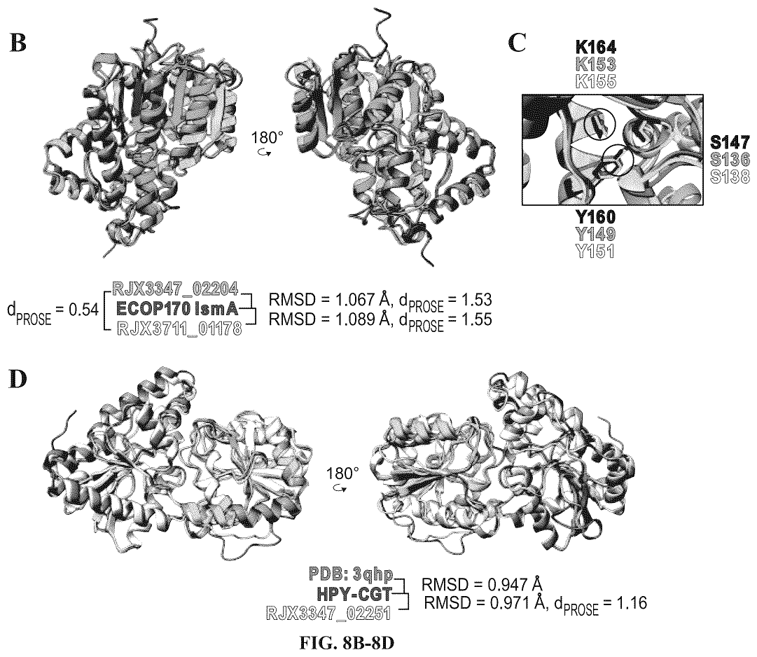

Based on the results above, one representative protein for each protein family (RJX3347_02204 for IsmA and RJX3347_02251 for CgT and RJX3347_01966 for TSPO) was used to search for corresponding hits in the Oscillibacter MSPs by sequence similarity (FIG. 8A; identity >50% and query coverage >50%). The CgT hits were only present in the MSPs phylogenetically bounded by RJX3347 and J115. The IsmA hit was found broadly in multiple MSPs negatively associated with stool cholesterol (e.g. msp_384, msp_099, msp_282, msp_283). Detection of the predicted proteins related to cholesterol metabolism in multiple Oscillibacter genomes suggested that cholesterol metabolism could be a property of many members from this diverse clade, consistent with their association with stool cholesterol levels.

To further support the functional relevance of the candidates, the three-dimensional structural properties of IsmA and CgT queries were compared and the two putative cholesterol metabolizing proteins from D. welbionis J115, RJX3347 and RJX3711 (IsmA only) were compared. As X-ray crystal structures of these proteins were unavailable, predicted structures were generated using AlphaFold2. All predictions displayed high confidence and low predicted alignment errors. Oscillibacter IsmA hits RJX3347_02204, RJX3711_01178 and J115_02546 were predicted to fold similarly to the E. coprostanoligenes IsmA (ECOP170) with low root mean-square deviation (RMSD) upon superimposition (RMSD-1.067 Å, 1.089 Å and 0.975 Å with 146, 140 and 143 pruned atom pairs, respectively) (FIG. 8B). Structural protein alignment also confirmed the conservation of the catalytic triad of Ser-Tyr-Lys required for HSD activity in the IsmA candidates from Oscillibacter (FIG. 8C). At the sequence level, the catalytic triad was also confirmed in the homologs in Oscillibacter MSPs. In the case of H. pylori CgT, the predicted structure of full-length CgT was first superimposed to the crystal structure of its catalytic domain (PDB: 3qhp) with RMSD of 0.947 Å across all pairs, which confirmed the high accuracy of the predicted structures. The predicted structure of the RJX3347_02251 candidate (90% sequence identity to the J115 candidate) was then superimposed to the predicted CgT structure with RMSD 0.971 Å (335 pruned atom pairs) between 335 pruned atom pairs (FIG. 8D). Overall, evidence from structural and sequence homology search, tertiary structure prediction and gene conservation supported the potential for cholesterol metabolism by Oscillibacter spp. via multiple genes.

### Example 8: Oscillibacter sp. Metabolizes Cholesterol Via Multiple Pathways

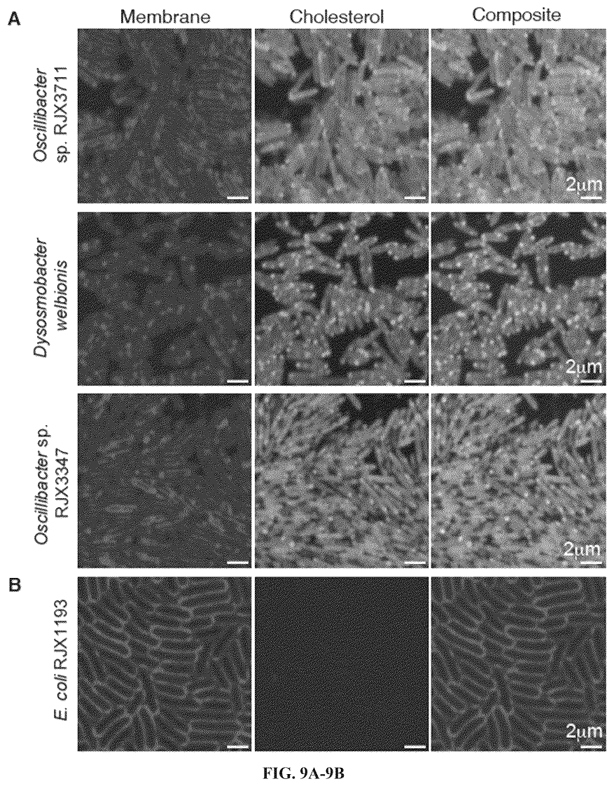

To test the hypothesis that Oscillibacter metabolizes cholesterol, the ability of Oscillibacter spp. to uptake exogenous cholesterol was first investigated. Confocal microscopy was used to examine the uptake of fluorescently labeled cholesterol by the three Oscillibacter isolates (TopFluor Cholesterol). Fluorescence was detected within the cytoplasm of all the three Oscillibacter isolates (FIG. 9A) but not in the cells of an E. coli strain isolated from the gut of a healthy donor (FIG. 9B). These data suggested that cholesterol was actively taken up and internalized by the Oscillibacter spp. tested.

To study the metabolic transformation of cholesterol, untargeted metabolomic profiling of bacterial isolate cultures was conducted. The three isolates RJX3711, J115 and RJX3347 were grown in media with or without added cholesterol. Bacteroides thetaiotaomicron (VPI-5482), a common human gut commensal which can uptake and metabolize cholesterol, was used as a control. After confirming stable growth, cell supernatants and washed cells were separated and lipid profiles were measured with LC-MS. Among the masses detected in conditioned media, 801 of the masses showed at least a ten-fold increase in intensity when one of the three Oscillibacter spp. was grown in the medium, whereas 489 masses decreased in intensity by the same relative amount. Annotated lipids with reference standards detected in conditioned media corresponded to triglycerides, phosphoethanolamines, phosphocholines, Lanosta-8,24-dien-3-ol, (3beta,5alpha,6beta,24R)-Stigmastane-3,5,6-triol and cholestenone, among which cholesterol reached high levels when added to unspent media, as expected (YCFAC). Cholesterol reduction in spent media after bacterial exposure was substantially more notable for all three Oscillibacter isolates (below detection limit for J115, fold change 0.01% for RJX3347 and 0.01% for RJX3711, relative to unspent media) than for B. thetaiotaomicron (fold change 2%).

In cell pellets of Oscillibacter spp., 1,689 metabolite peaks were detected whose intensity increased more than tenfold upon addition of cholesterol (CHO). Out of these, 221 masses were predicted to be “steroids and steroid derivatives” class based on a molecular formula match in HMDB. The metabolic profile, especially the predicted steroids, showed high concordance across the three Oscillibacter isolates (Spearman correlation >0.49 and >0.67 for all peaks and predicted steroids, respectively). Notably, the predicted steroids from Oscillibacter pellets showed an overall increase compared to B. thetaiotaomicron, which was further manifested when cholesterol was added to the media. This indicated a unique shift in steroid metabolomic profile in response to cholesterol for Oscillibacter spp., which was consistently observed for all the three isolates.

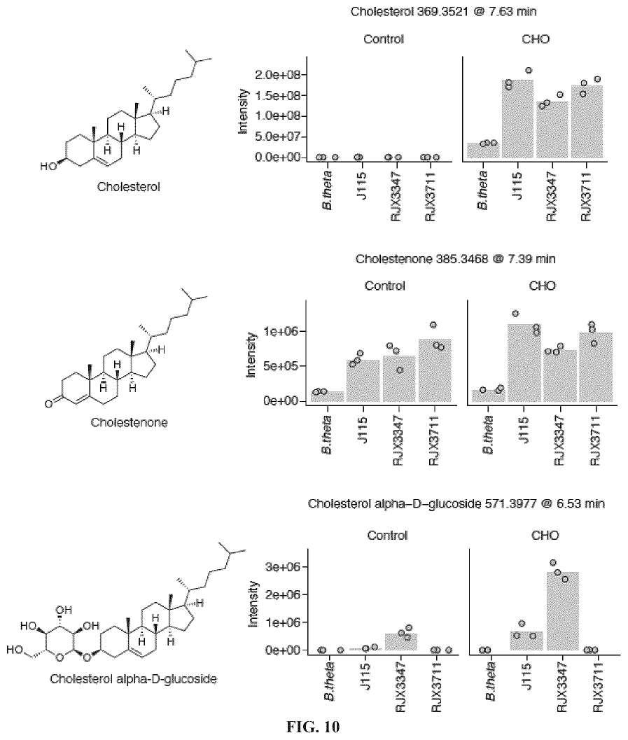

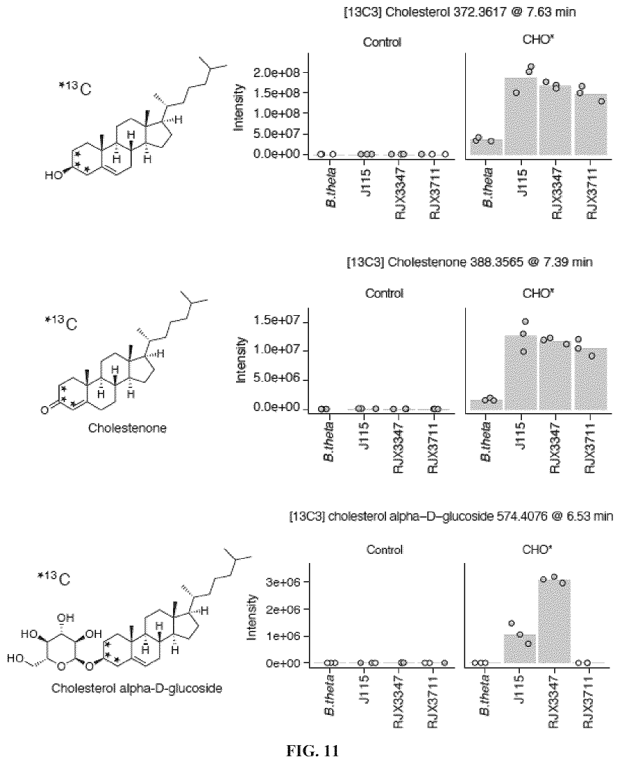

Among the most increased peaks annotated with reference standards were cholestenone (4-cholesten-3-one), 5-cholesten-3-one, 7alpha-hydroxycholesterol and a glycosylated cholesterol, whose fragmentation pattern matched cholesterol alpha-D-glucoside or its diastereomer alpha-mannosyl cholesterol (FIG. 10). Furthermore, the glycosylated cholesterol was exclusively found in the pellets of isolate J115 and RJX3347, consistent with the detection of the CgT gene (J115_17675 and RJX3347_02251) in the genomes of the isolates but not RJX3711 (FIG. 10). The above metabolic products were also detected in the controls without the addition of cholesterol, likely due to the presence of cholesterol in the media.

To track the fate of cholesterol, media was supplemented with ¹³C-labeled cholesterol. Overall, it was confirmed that the predicted steroids from Oscillibacter pellets were again systematically increased compared to B. thetaiotaomicron. 210 LC-MS peak pairs were detected consisting of an unlabeled mass and a ¹³C-labeled counterpart (having the same retention time and a mass shift of 3.0093 units) and thus likely deriving from cholesterol. Indeed, matching to an internal library of 229 diverse lipids (69 of which are sterols), annotated 14 peaks. All were exclusively steroids, including cholesterol, cholestenone and cholesterol alpha-D-glucoside (FIG. 11), which validated the peak matching strategy and increased confidence in genuine changes in steroid metabolism. These data corroborated the observations from (1) association analysis of human stool samples showing cholestenone as a prominent Oscillibacter-associated metabolite and (2) comparative bacterial genomics which hypothesized the presence of CgT and ismA homologs in the Oscillibacter spp.

Revisiting the initial cholesterol-induced mass shift network, 20 out of 148 masses in the network were also found in pellets of at least one Oscillibacter spp, and had a corresponding mass peak in ¹³C-labeled cholesterol experiments. In stool, 15 out of 20 had a stronger correlation with Oscillibacter abundance in ismA− samples than in ismA+ samples. Most masses (7) matched dehydrogenated cholesterols (C27H440), including confirmed cholestenone, and MS/MS-predicted cholesta-5,25-dien-3-beta-ol and desmosterol. Also, an MS/MS-predicted oxysterol (24(R)-Hydroxycholesterol or 25-Hydroxycholesterol, 425.3391 m/z, 7.32 min), a precursor to 7a-HC, showed >2.7-fold increase in culture and 0.39 Spearman correlation in stool for ismA− samples and no correlation for ismA+ samples (Spearman correlation=−0.01). Together, the isotope labeling of cholesterol confirmed that cholestenone, cholesterol alpha-D-glucoside and a number of predicted oxysterols were produced from uptake of cholesterol in the media by the Oscillibacter isolates.

In summary, these experiments suggested the presence of diverse types of cholesterol metabolism in multiple representative Oscillibacter strains isolated from the human gut. Metabolite networking in stool metabolomic samples predicted potential microbially-mediated fates of cholesterol in the gut and was supported by detection of dehydrogenated cholesterol derivatives or oxysterols in microbial cultures. Moreover, adding cholesterol to growth media did not inhibit the growth of Oscillibacter isolates tested, suggesting that the observed negative association between Oscillibacter and cholesterol in vivo was not due to the inhibitory effect of cholesterol on the growth of the bacteria. Finally, detection of genes encoding related proteins (IsmA and CgT) in multiple MSPs from the genus strengthened the hypothesis that Oscillibacter spp. contribute to the decrease of cholesterol in the human gut. The metabolites (e.g. cholestenone) produced from cholesterol could potentially be used by the IsmA-encoding Eubacterium, supporting the proposed additive or synergistic effect in further reducing cholesterol in the gut. In effect, lowered gut cholesterol could consequently influence cholesterol uptake and lipid homeostasis of the host, leading to the observed decreased levels of plasma cholesterol and triglycerides.

The large-scale characterization of the gut microbiome and metabolome of FHS participants provided a unique opportunity to mine and validate human gut microbiome metabolism in the context of cardiovascular health. In the examples above, using de novo metagenomics assembly and the extensive metadata collected from FHS participants, associations between 129 MSPs and cardiovascular health related measurements were identified. These include findings robustly validated in other cohorts, such as the positive associations between plasma triglycerides and F. plautii and C. bolteae, negative association between plasma triglycerides and the Oscillibacter genus, and those not previously reported, such as positive association between P. merdae and CRP, and negative associations between A. obesi and blood lipids (cholesterol and triglycerides). In addition, two M. smithii subspecies associated with plasma cholesterol levels were found. The two M. smithii subspecies exhibit different growth characteristics in vitro and carry different mtr genes, which are critical for methane production.

For stool metabolites associated with higher CVD risk, the enrichment of cholesterol, primary bile acids, polyunsaturated fatty acids (PUFAs), N-acylethanolamines (NEAs) coupled with the depletion of coprostanol and dicarboxylic acids was noted.

Beyond detecting these associations between the gut microbiome and cardiovascular health related measurements, integrated analyses in the above examples allowed proposal of potential mechanisms for them. For example, F. plautii was linked to multiple CVD related blood measurements, but the underlying metabolic mechanisms had not been explored. It was found that the abundance of F. plautii was associated with higher gut polyunsaturated fatty acid (PUFA) and N-acylethanolamines levels, suggesting its growth might be regulated by lipid composition. As a flavonoid degrader, F. plautii could interact with the host by shaping the composition of phenolic acid profiles, although flavonoids are not the sole source of phenolic acids. Another observation closely related to CVD is the positive association between γ-butyrobetaine and Clostridia, such as the opportunistic colonizers C. clostridioforme and C. bolteae associated with unfavorable environmental exposures. Published in vitro culture data confirmed the production of γ-butyrobetaine by these Clostridia species. The detection of cutC/D gene homologs in the two MSPs and observation of reduced choline levels in their cultures indicates that it is likely that they contribute to elevated CVD risks via the TMAO pathway.

In the above examples, the large uncharacterized metagenomic and metabolomic space of the human gut environment, represented by MSPs with unmapped taxonomy, microbial genes with undetermined functions and tens of thousands of unknown metabolite mass spectrometry peaks was also clarified. The large-scale profiling and extensive annotation of stool metabolites paired with the metagenomic data made it possible to gain insights into this uncharacterized space. An unknown Firmicutes (msp_120, annotated more specifically to Acutalibacteraceae in the Genome Taxonomy Database (GTDB)) was found which exhibited the strongest positive associations with both plasma and stool cholesterol. Intriguingly, it was also the top positively associated MSP with stool cholesterol sulfate, which was recently discovered to be produced from cholesterol in B. thetaiotaomicron. A gene with significant, albeit low, sequence similarity to the human SULT2B1 cholesterol sulfotransferase (33.3% identity and 14% coverage) was found in the genome of msp_120, suggesting potential existence of microbial phyla in addition to Bacteroides with cholesterol sulfonation activity. Moreover, it was found that a large number of unknown metabolites were strongly associated with specific MSPs, and therefore were likely to be produced or consumed by the MSPs. This includes a metabolite (QI28613) positively associated with E. siraeum (Spearman correlation=0.66), an MSP negatively associated with plasma triglycerides. As these microbe-metabolite associations can yield novel consumer or producer relationships, they provide a resource for targeted characterization of unknown microbial metabolites prioritized based on correlations with disease markers and in vivo abundance.

Further, in the above examples, cholesterol metabolism by the gut microbiome was focused on. Although host cholesterol homeostasis can be influenced by multiple factors such as genetics, diet, and lifestyle, the gut microbiome has also been implicated in decreasing the intestinal absorption of cholesterol by converting it to coprostanol in this environment. A clade of prevalent and abundant Oscillibacter MSPs negatively associated with stool cholesterol levels was discovered. Consistently, Oscillibacter spp. abundance was associated with high overall microbiome diversity, low abundances of proinflammatory lipids and low level of plasma triglycerides, suggesting their potential beneficial role to the host cardiovascular health. Despite the sporadic report of associations between Oscillibacter and host health indicators including low blood triglycerides and insulin homeostasis (Liu et al., Nat. Genet. 54, 52-61, 2022, Asnicar Nat. Med. 10.1038/s41591-020-01183-8, 2021), limited functional characterization of Oscillibacter spp. has been reported. A recent study on Oscillibacter showed supplementing mice fed on a high-fat diet with live Dysosmobacter welbionis strain J115T (member of the Oscillibacter clade) reduced host body weight and fat mass gain, improved glucose homeostasis, increased non-shivering thermogenesis and the number of mitochondria in the brown adipose tissue. However, none of these studies have investigated the metabolic mechanisms of the beneficial roles of Oscillibacter.

Employing molecular networking and protein structural embedding, a set of potential cholesterol derivatives produced by Oscillibacter spp. was prioritized, including cholestenone and glycosylated cholesterol together with a set of Oscillibacter candidate genes involved in cholesterol metabolism. The above examples confirmed that three human gut Oscillibacter sp. isolates demonstrated the uptake of cholesterol, and production of cholestenone, cholesterol glucoside and hydroxycholesterol in vitro, validating the existence of multiple pathways for cholesterol metabolism by Oscillibacter. Cholesterol metabolizing activity has been reported in other bacteria. For example, cholestenone was proposed to be an intermediate in the conversion of cholesterol to coprostanol by E. coprostanoligenes and other IsmA encoders (Kenny, supra), and H. pylori synthesizes glycosylated cholesterol as a critical component of its cell wall. Hydroxycholesterol does not appear to be produced by any prokaryotes. It is anticipated that further investigation of the diverse routes of cholesterol utilization by Oscillibacter will reveal the complete cholesterol metabolic network in Oscillibacter and provide broader insight into microbial cholesterol metabolism.

In the above examples, decrease in host blood cholesterol levels was also observed in samples with Oscillibacter and Eubacterium containing the ismA gene across different cohorts. Cholestenone, which is strongly associated with the abundance of Oscillibacter in stool samples and was efficiently produced by all three Oscillibacter isolates tested in vitro, can potentially be utilized for coprostanol production by IsmA-encoding Eubacterium, leading to the observed synergistic or additive interaction between the two clades. This suggests a key role for Oscillibacter and IsmA-encoding species in coprostanol production and excretion, which reduces the level of accessible cholesterol in the gut.

### METHODS OF THE EXAMPLES

The following methods were employed in the above examples.

### Bacterial Strains

Stool samples from subjects were obtained under a protocol approved by the institutional review board at MIT. Participants provided informed consent and all experiments adhered to the regulations of the review board. The stool samples were cycled into a Coy anaerobic chamber (5% H2, 20% CO2, and 75% N2) within 1 hour of collection to maintain microbial viability. All materials and reagents were reduced in the anaerobic chamber 24 hours prior to experimentation. 1 g of stool was homogenized in 1 mL of 1×PBS, serially diluted in PBS, plated onto YCFA+0.1% taurocholate agar plates, and incubated in the chamber for 1 week at 37° C. Well isolated and morphologically distinct colonies were re-streaked to ensure purity. Freezer stocks per each species isolated were established by scraping biomass into 1×PBS, 40% glycerol, 0.001% L-cysteine solution and storing at −80° C. Oscillibacter spp. (RJX3347 and RJX3711) and E. coli (RJX1193) were the species that resulted from the isolation effort. Dysosmobacter welbionis J115 was acquired from Creative Biolabs Live Biotherapeutics (Cat No. LBSX-0522-GF115).

### Human Subjects

The stool metagenomic samples of Framingham Heart Study (FHS) participants have been previously described (Kenny et al. Cell Host Microbe 28, 245-257.e6) where a subset of the samples were analyzed. The microbiome and metabolome data were generated for the Generation 3 and the Omni 2 cohorts. The study protocol was approved by the Massachusetts General Hospital/Partners Human Research Committee (2016P001079/MGH) and the Institutional Review Board of the Boston University Medical Center (H-32132, H-33166). All experiments adhered to the regulations of these review boards. All study procedures were performed in compliance with all relevant ethical regulations. Each participant signed an informed consent prior to participation.

### Metagenomic Library Preparation and Sequencing

Stool was collected in 100% ethanol for nucleic acid extraction as previously described (Lloyd-Price, et al. Nature 569, 655-662). For DNA extraction, a combination of the QIAamp 96 PowerFecal Qiacube HT Kit (Qiagen Cat No./ID: 51531), the Allprep DNA/RNA 96 Kit (Qiagen Cat No./ID: 80311), and IRS solution (Qiagen Cat No./ID: 26000-50-2) kits were used with a custom protocol as previously described (Lavoie, S et al., Elife 8. 10.7554/eLife.39982). Briefly, approximately 100 mg of stool was transferred into individual wells of the PowerBead plate, with 0.1 mm glass beads (Cat No./ID: 27500-4-EP-BP) prior to bead beating on a TissueLyzer II at 20 Hz for a total of 10 minutes. Samples were transferred into AllPrep 96-well DNA plates and processed as per manufacturer's instructions. Purified DNA was stored at −20° C.

For metagenomic library construction, DNA samples were first quantified by Quant-iT PicoGreen dsDNA Assay (Life Technologies) and normalized to a concentration of 50 μg/μL. Illumina sequencing libraries were prepared from 100-250 μg of DNA using the Nextera XT DNA Library Preparation kit (Illumina) according to the manufacturer's recommended protocol, with reaction volumes scaled accordingly. Prior to sequencing, libraries were pooled by collecting equal volumes (200 nL) of each library from batches of 96 samples. Insert sizes and concentrations for each pooled library were determined using an Agilent Bioanalyzer DNA 1000 kit (Agilent Technologies). Libraries were sequenced on HiSeq 2500 2×101 to yield ˜10 million paired end reads per sample. De-multiplexing and BAM and FASTQ file generation were performed using the Picard suite (https://broadinstitute.github.io/picard).

### Metabolomic Library Preparation and Mass Spectrometry Analysis

### LC-MS Profiling.

A combination of four LC-MS methods were used to profile metabolites in the fecal homogenates, as previously published (Kostic, Cell Host Microbe 17, 260-273, 2015): two methods that measure polar metabolites, a method that measures metabolites of intermediate polarity (e.g., fatty acids and bile acids), and a lipid profiling method. Ethanol-preserved stool samples were thawed on ice, spun in a swing bucket rotor at 4° C. at 5,000 g for 5 minutes and dried under a constant stream of nitrogen gas in batches of 54 samples using a TurboVap evaporator (Biotage, Charlotte, NC). Dried samples were stored at −80° C. until all samples in the study had been dried. Aqueous homogenates were then generated by sonicating each dried sample in 900 μl of H2O using an ultrasonic probe homogenizer (Branson Sonifier 250) set to a duty cycle of 25% and output control of 2 for 3 minutes (samples were kept on ice during the homogenization process). The homogenate for each sample was divided into two 10 μL and two 30 μL aliquots in 1.5 mL centrifuge tubes for LC-MS sample preparation. 30 μL of homogenate from each sample was transferred into a 50 ml conical tube on ice to create a pooled reference sample. Subjects were randomized in the analysis queue in each method. Additionally, pairs of pooled reference samples were inserted into the queue at intervals of approximately 20 samples for quality control and data standardization. Samples were prepared for each method using extraction procedures that are matched for use with the chromatography conditions. Data were acquired using LC-MS systems comprised of Nexera X2 U-HPLC systems (Shimadzu Scientific Instruments) coupled to Q Exactive/Exactive Plus orbitrap mass spectrometers (Thermo Fisher Scientific). The method details are summarized below.

LC-MS Method 1: HILIC-pos (positive ion mode MS analyses of polar metabolites). LC-MS samples were prepared from stool homogenates (10 μl) by protein precipitation with the addition of nine volumes of 74.9:24.9:0.2 v/v/v acetonitrile/methanol/formic acid containing stable isotope-labeled internal standards (valine-d8, Isotec; and phenylalanine-d8, Cambridge Isotope Laboratories). The samples were centrifuged (10 min, 9,000 g, 4° C.), and the supernatants injected directly onto a 150×2-mm Atlantis HILIC column (Waters). The column was eluted isocratically at a flow rate of 250 μl/min with 5% mobile phase A (10 mM ammonium formate and 0.1% formic acid in water) for 1 min followed by a linear gradient to 40% mobile phase B (acetonitrile with 0.1% formic acid) over 10 min. MS analyses were carried out using electrospray ionization in the positive ion mode using full scan analysis over m/z 70-800 at 70,000 resolution and 3-Hz data acquisition rate. Additional MS settings are: ion spray voltage, 3.5 kV; capillary temperature, 350° C.; probe heater temperature, 300° C.; sheath gas, 40; auxiliary gas, 15; and S-lens RF level 40.

LC-MS Method 2: HILIC-neg (negative ion mode MS analysis of polar metabolites). LC-MS samples were prepared from stool homogenates (30 μl) by protein precipitation with the addition of four volumes of 80% methanol containing inosine-15N4, thymine-d4 and glycocholate-d4 internal standards (Cambridge Isotope Laboratories). The samples were centrifuged (10 min, 9,000 g, 4° C.) and the supernatants were injected directly onto a 150×2.0-mm Luna NH2 column (Phenomenex). The column was eluted at a flow rate of 400 μl/min with initial conditions of 10% mobile phase A (20 mM ammonium acetate and 20 mM ammonium hydroxide in water) and 90% mobile phase B (10 mM ammonium hydroxide in 75:25 v/v acetonitrile/methanol) followed by a 10-min linear gradient to 100% mobile phase A. MS analyses were carried out using electrospray ionization in the negative ion mode using full scan analysis over m/z 60-750 at 70,000 resolution and 3 Hz data acquisition rate. Additional MS settings are: ion spray voltage, −3.0 kV; capillary temperature, 350° C.; probe heater temperature, 325° C.; sheath gas, 55; auxiliary gas, 10; and S-lens RF level 40.

LC-MS Method 3: C18-neg (negative ion mode analysis of metabolites of intermediate polarity; for example, bile acids and free fatty acids). Stool homogenates (30 μl) were extracted using 90 μl methanol containing PGE2-d4 as an internal standard (Cayman Chemical Co.) and centrifuged (10 min, 9,000 g, 4° C.). The supernatants (10 μl) were injected onto a 150×2.1-mm ACQUITY BEH C18 column (Waters). The column was eluted isocratically at a flow rate of 450 μl/min with 20% mobile phase A (0.01% formic acid in water) for 3 min followed by a linear gradient to 100% mobile phase B (0.01% acetic acid in acetonitrile) over 12 min. MS analyses were carried out using electrospray ionization in the negative ion mode using full scan analysis over m/z 70-850 at 70,000 resolution and 3 Hz data acquisition rate. Additional MS settings are: ion spray voltage, −3.5 kV; capillary temperature, 320° C.; probe heater temperature, 300° C.; sheath gas, 45; auxiliary gas, 10; and S-lens RF level 60.

LC-MS Method 4: C8-pos. Lipids (polar and nonpolar) were extracted from stool homogenates (10 μl) using 190 μl isopropanol containing 1-dodecanoyl-2-tridecanoyl-sn-glycero-3-phosphocholine as an internal standard (Avanti Polar Lipids; Alabaster, AL). After centrifugation (10 min, 9,000 g, ambient temperature), supernatants (10 μl) were injected directly onto a 100×2.1-mm ACQUITY BEH C8 column (1.7 μm; Waters). The column was eluted at a flow rate of 450 μl/min isocratically for 1 min at 80% mobile phase A (95:5:0.1 v/v/vl 10 mM ammonium acetate/methanol/acetic acid), followed by a linear gradient to 80% mobile phase B (99.9:0.1 v/v methanol/acetic acid) over 2 min, a linear gradient to 100% mobile phase B over 7 min, and then 3 min at 100% mobile phase B. MS analyses were carried out using electrospray ionization in the positive ion mode using full scan analysis over m/z 200-1,100 at 70,000 resolution and 3 Hz data acquisition rate. Additional MS settings are: ion spray voltage, 3.0 kV; capillary temperature, 300° C.; probe heater temperature, 300° C.; sheath gas, 50; auxiliary gas, 15; and S-lens RF level 60.

### Metabolomics Data Processing.

Raw LC-MS data were acquired using the data acquisition computer interfaced to each LC-MS system and then stored on a robust and redundant file storage system (Isilon Systems) accessed via the internal network at the Broad Institute. Nontargeted data were processed using Progenesis QIsoftware (v 2.0, Nonlinear Dynamics) to detect and de-isotope peaks, perform chromatographic retention time alignment, and integrate peak areas. To remove redundant adducts and peaks, LC-MS peaks were clustered based on intensities and retention times. Clusters were generated containing peaks occurring within a retention time window of 0.025 minutes (0.015 min for C8-pos) and with intensities correlating with Spearman rank correlation coefficients above 0.8 and 0.85 for negative mode and positive methods respectively. The peak with the highest mean abundance within each cluster was kept as the representative ion and all other cluster members removed from the final dataset. Unclustered peaks (singlets) were kept in the final data set. Peaks of unknown identity were tracked by method, m/z and retention time. Identification of the resulting 130,877 nontargeted metabolite LC-MS peaks was conducted by: i) matching measured retention times and masses to mixtures of reference metabolites analyzed in each batch; and ii) matching an internal database of >600 compounds that have been characterized using the Broad Institute methods, yielding 568 peaks with a match. Temporal drift was monitored and normalized with the intensities of peaks measured in the pooled reference samples.

### MS/MS Peak Annotation Prediction.

Study QC pools generated during the sample acquisition were used to generate MS-MS for unknown features of interest at collision energies ranging from 10 to 50V in 10V increments in a Thermo IDX mass spectrometer (Thermo Fisher Scientific; Waltham, MA) with electrospray ionization in the positive ion mode using full scan analysis at 60,000 resolution followed by five MS-MS scans at 30,000 resolution and an isolation width of +/−0.2 mass units. Additional MS settings were: sheath gas 40, sweep gas 2, spray voltage 3.5 kV, capillary temperature 330° C., S-lens RF 40, heater temperature 300° C., microscans 1, automatic gain control target 1e6, and maximum ion time 250 ms. Parsed MS-MS data (* ms) were loaded into SIRIUS CSI-Finger ID version 4.7.2. Molecular formula predictions generated with Orbitrap-specific settings (MS-MS isotope scorer: ignore, mass deviation: 5 ppm, Candidates: 10, Candidates per ion: 1, possible ionizations: [M+H]+, [M+K]+, [M+Na]+). Structure elucidations were done using all included databases and the adducts [M+H]+, [M+K]+, [M+Na]+). Predictions were exported and the top three structure elucidations parsed for each peak. MS-MS-based networks were built using the Global Natural Products Social Molecular Networking and resulting networks visualized with Cytoscape v. 3.8.2.

### Metagenomics Data Analysis

### Metagenomic Sequencing Data Processing.

The de novo assembly steps used in Kenny et al were followed to construct metagenomic species pangenomes (MSPs) (Kenny, supra). Briefly, raw sequencing reads were trimmed to remove sequencing adapters with Trim Galore! (v0.4.4, default parameters). KneadData (http://huttenhower.sph.harvard.edu/kneaddata, v0.7.2, HEADCROP: 15 SLIDINGWINDOW: 1:20) was used to remove host DNA contamination and trim low-quality bases. Processed reads from each sample were then assembled into contigs with MegaHIT (v1.1.4, default parameters). Full-length genes were predicted from the contigs using Prodigal (v2.6.3, default parameters) and a non-redundant gene catalog was created by clustering the genes using CD-HIT (v4.7, 95% identity and 90% coverage). A count matrix was created by mapping the reads to the gene catalog with BWA (v0.7.17, keeping reads with >95% identity) and calculating the transcript-per-million (TPM) value for each gene in each sample. Contigs were then binned into MSPs (including core and accessory genes) with MSPminer (30) (default parameters) using the TPM matrix. The gene catalog and MSPs were annotated following the methods as described previously (Li, Nat. Biotechnol. 32, 834-841, 2014) with EggNOG Mapper (v1.0.3) and BLAST (v2.6.0), respectively. The gene catalog was translated into protein sequences and clustered into gene families using usearch (uclust v8.1) at 50% identity and 50% coverage.

Identifying Metagenomic Features Associated with Blood Measurements.

The MSPs with low prevalence (<2%) and low average abundance (TPM<0.05) were removed and a linear model was used to identify MSPs (X; TPM+1) or KEGG pathways (aggregated TPM; pathways with <50% KOs detected on average were excluded) associated with each blood measurements (Y; U/L for ALT and AST, mg/dL for other measurements) adjusted by sex, age and BMI and related drug usage (Rx):

$$log ( Y ) ∼ log ( X ) + B M I + Age + Sex + RxLDL + RxCholesterol + RxGlucose .$$

To find accessory genes associated with blood measurements, a similar model was used with respect to the presence or absence of the gene (Z; 0 or 1 for absence or presence, respectively) adjusted by MSP abundance in addition:

$$log ( Y ) ∼ Z + log ( X ) + B M I + Age + Sex + RxLDL + RxCholesterol + RxGlucose .$$

The model was only applied to the samples where the corresponding MSP had enough coverage (>75% of the core gene set was covered). The R method Im was used to obtain the slope estimates and their confidence intervals. Significant values were corrected for multiple testing using Benjamini-Hochberg correction (FDR).

### Identification of IsmA Encoders.

Previously reported ismA homologs were searched against the gene family representatives (uclust cluster centroids) with usearch (ublast). The corresponding cluster for the best hit (100% identity and 99.6% coverage) was noted and the host MSPs for the genes in such clusters were annotated as IsmA encoders. Samples with at least one of the IsmA encoders were noted as ismA positive.

### Protein Language Models and Structural Protein Queries.

The unannotated representative sequences of the 11,846 protein family clusters (failed to map by eggNOG-mapper or annotated to domains of unknown function or DUF) were further annotated using a protein language model, ProtTucker (v1.1.0; Euclidean distance <0.5), trained on 66,052 protein structures from the CATH-S100 database (v4.3). To accelerate the annotation, a pre-built index of pre-generated ProtTucker embeddings was used spanning four databases (SwissProt, PDB, CATH-S100 v4.3, SCOPe v2.08) for finding the nearest neighbors (Euclidean distance) of the query protein on Google Cloud's Vertex AI Matching Engine.

Protein sequences for 26 Oscillibacter MSPs (41,934 sequences) and isolates RJX3347 (3,407), RJX3711 (3,314), J115 (3,529) were processed with the protein structural embedding model of PROSE (https://github.com/tbepler/prose) using the command:
python embed_sequences.py --pool avg $input_fasta $output

This resulted in 6,165-dimensional embeddings for each amino acid in each sequence. The amino acid embeddings were then averaged across each protein to obtain its respective embedding. Proteins were compared using Euclidean distance (dPROSE). A subset of MSP proteins (13,696) was linked to with eggNOG annotations, which included EC number predictions. A sample of 3,000 proteins was used to compare the classification value of dPROSE in separating pairs of proteins sharing an EC annotation at any of its four levels of hierarchy.

Structural models for reference proteins ECOP170 (WP_078769004.1), CgT (WP_000237258.1) and respective genes in Oscillibacter spp. RJX3347_02204, RJX3711_01178, J115_02655, and RJX3347_02251 were generated using ColabFold: AlphaFold2 using default parameters. Five models were generated for each protein and highest-ranked models were used for structural alignments. Visualization, superimposition and RMSD value calculation was executed using the matchmaker function in ChimeraX (version 1.0) using default parameters.

### Metabolomics Data Analysis

### Retrieving Candidate Peak Identifiers Based on Molecular Formula Match.

Peaks not matching the internal database (unknown) were matched via adduct subtraction and molecular formula match to compounds, downloaded from Human Metabolome Database (HMDB) on Sep. 16, 2020. The measured m/z values were adjusted for method-specific adducts and molecular formulas matching to within 5 ppm were selected as candidate identifiers. When multiple molecular formulas matched the adduct-adjusted mass (as a result of multiple potential adducts), one with minimal ppm difference was selected. The following adducts are assumed for different LC-MS methods: [M−H](−) for negative mode methods (C18-neg, HILIC-neg) and [M+H](+), [M+NH4](+), [M+Na](+), [M−H20+H](+) for positive mode methods (C8-pos, HILIC-pos). The same procedure was used for metabolomics of bacterial cultures, including labeled cholesterol experiments, where a difference of 3.0093 was subtracted in addition to the adducts to account for 3×¹³C labeling. In stool, this yielded molecular formula matches for 62,439 peaks, with a median of 3 candidate annotations per peak (5th percentile: 1, 95th percentile: 30).

A nominal majority class was selected from all possible compound annotations in HMDB associated with each mass after adduct adjustment. Using the 568 known metabolites in stool as quality control, this approximation yielded the correct class for 76% of the mass peaks.

### Determining Variable Metabolite Peaks and Scaling.

The original metabolite intensity matrix contained 992 samples and 130,877 peaks with 68.0% of measured intensities (non-missing values). The 32.0% of missing values were replaced using half-minimum imputation as described previously (Franzosa et al. (2019). Gut microbiome structure and metabolic activity in inflammatory bowel disease. Nat Microbiol 4, 293-305.). A total of 130,877 peaks in biological subjects (n=899) were compared to pooled aliquots (n=110) yielding 119,563 peaks with larger coefficients of variation (CV) for samples than controls.

The coefficient of variation for a vector of raw peak intensities (M) is calculated as

$$CV ( M ) = sd [ log ( M + 1 ) ] / mean [ log ( M + 1 ) ] .$$

### Unsupervised Co-Abundance Clustering of Metabolites.

Metabolites were clustered based on their co-abundance across 899 samples. Imputed and scaled intensity values (see above), 40 principal component analysis and Leiden clustering with nearest neighbors were used. The resolution parameter was varied between 0.1 and 1000 and the value where the clusters with annotated metabolites best recapitulated the HMDB super class was chosen (p-value of Chi-Square test was used as a score). This resulted in 2,368 co-abundance clusters, with median cluster size of minimum cluster size of 4, median 46 and maximum of 175.

### Linking LC-MS Peaks in a Mass Shift Network.

Pairs of LC-MS peaks were linked into a molecular network using mass gains or losses with a method similar to previously described methods (Quinn et al. (2020). Global chemical effects of the microbiome include new bile-acid conjugations. Nature 579, 123-129; Hartmann et al. (2017). Meta-mass shift chemical profiling of metabolomes from coral reefs. Proc. Natl. Acad. Sci. U.S.A. 114, 11685-11690.). Peaks with reference standards or molecular formula matches were represented as nodes in a graph and the corresponding adduct-adjusted m/z was assumed as a nominal mass. An edge between two peaks is assumed if 1) their retention time difference is within 0.5 min, and 2) the difference in their neutral masses equals mass shifts representing putative chemical transformations: (H2, 2.016 m/z, CH2-H20, 3.955 m/z, 2H2, 4.030 m/z, C, 12.000 m/z, CH2, 14.016 m/z, CH3, 14.020 m/z, O, 15.995 m/z, CH4, 16.040 m/z, OH, 17.010 m/z, NH3, 17.030 m/z, H2O, 18.010 m/z, C2, 24.020 m/z, C2H2, 26.016 m/z, CO, 27.996 m/z, C2H4, 28.032 m/z, C2H6, 30.050 m/z, CH2O, 30.910 m/z, C2H2O, 42.009 m/z, C2H3O, 42.050 m/z, CO2, 43.990 m/z, CH2O2, 46.010 m/z, C2H2O2, 56.006 m/z, C3H4O, 56.025 m/z, C4H8, 56.060 m/z, C2H8, 56.064 m/z, SO3, 79.960 m/z, C5H8O4, 132.040 m/z, C6H10O4, 146.060 m/z, C6H10O5, 162.050 m/z, C12H18O11, 338.090 m/z). In addition, the observed versus theoretical difference in mass shifts should not exceed 5 ppm.

### Prediction of Cholesterol Derivatives and Glycosylated Cholesterols for MS/MS Extraction.

The mass shift network of LC-MS peaks was used to extract a subgraph consisting of direct neighbors to the reference cholesterol peak (369.3512 m/z, 7.52 min, C8-pos) and six other peaks with predicted formula C27H46O within HILIC-pos of C8-pos. This resulted in 238 candidate peaks that were prioritized for MS/MS analysis. In addition, peaks with adduct-adjusted mass corresponding to formulas C33H56O6, C33H54O7 and C33H56O7 were prioritized for MS/MS, resulting in a combined total of 262 compounds.

128 out of 262 peaks were extracted (3 C18-negative, 65 C8-positive, 60 HILIC-positive), and MS/MS spectra were obtained for 92 (3 C18-negative, 65 C8-positive, 24 HILIC-positive). SIRIUS/CSI: FingerID predicted identifies for 85 peaks (2 C18-negative, 59 C8-positive, 24 HILIC-positive), yielding at least one HMDB identifier for 40 peaks (24 C8-positive, 16 HILIC-positive), where 29 peaks had a single match, 9 peaks had 2 matches and 2 peaks had 3 matches.

The putative peak for each metabolite was selected as the MS/MS annotation with highest CSI Finger ID score. Where HMDB prediction was not available, the most likely MS/MS-predicted molecular formula was used. Where MS/MS prediction was unsuccessful, the closest neutral mass molecular formula in HMDB was used subject to 5 ppm tolerance. The remains of the peaks were left unannotated. In total, the cholesterol and its derivatives spanned 6 peaks with annotated standards, 19 MS/MS annotations, 36 MS/MS formula matches, 170 m/z formula matches and 8 unannotated peaks.

### Alignment Between LC-MS Peaks in Cholesterol and +3× ¹³C-Labeled Cholesterol Feeding Experiments.

Comparison with experiments using unlabeled cholesterol is performed by matching metabolic peaks using retention time and mass (delta ppm <5) or mass shift of 3.0093 mass units (delta ppm <5). The comparison identified 210 shifted peak pairs in experiments with RJX3347, RJX3711, J115, and Bacteroides thetaiotaomicron VPI-5482 (“Shift”).

Identifying metabolite peak intensities associated with metagenomic species and blood measurements. The filtered metabolite peaks and their scaled intensities (see above) were used for association analyses. The Spearman correlation coefficient was used to rank associations between metabolite intensities and MSP abundances (MSPs with prevalence <2% or average TPM <0.05 were excluded). To prioritize a subset of Oscillibacter spp. associated with cholesterol, the following model was used with elastic net regularization coupled by 10-fold cross validation (Y: stool cholesterol, X1 . . . . Xn: abundances of Oscillibacter MSPs in TPM+1, ismA: total abundance of IsmA encoders in TPM+1):

$$log ( y ) ∼ log ( X 1 ) + … + log ( Xn ) + log ( ismA ) + B M I + Age + Sex + RxLDL + RxTriglycerides .$$

The association between metabolites (M) and blood measurements (Y: HbA1c, ALT, AST, albumin, creatinine, cholesterol, HDL, LDL, triglycerides, glucose, CRP) was estimated by:

$$log ( Y ) ∼ log ( M ) + B M I + Age + Sex + RxLDL + RxTriglycerides + RxGlucose + RxAntihypertensives .$$

The R method cv.glmnet (glmnet package) and Im was used to fit the two models, respectively. Significant values for the second model were corrected for multiple testing using Benjamini-Hochberg correction (FDR) at level 5% unless explicitly stated.

Oscillibacter sp. Growth Experiment

### Genome Sequencing and Assembly.

Oscillibacter spp. (RJX3347, RJX3711, J115) pellets were prepared by centrifuging the liquid culture above for 10 minutes at 5000×g and decanting the supernatant. The resultant pellet was submitted to the Microbial ‘Omics Core at the Broad Institute for DNA extraction and

Illumina whole-genome sequencing. Paired-end reads were trimmed to remove adaptors using Trim Galore! (--quality 15 --stringency 5 --length 50 --clip_R1 15 --clip_R2 15) and assembled into scaffolds using SPAdes (v3.13.0, --careful). Genes were predicted using Prokka (v1.14.3, --kingdom Bacteria--gcode 11) and annotated using EggNOG Mapper. Genome-wide average nucleotide identity (ANI) between assemblies was estimated using fastANI (v1.33, --fragLen 2000 --minFraction 0.1).

### Metabolomics Data Preparation.

Overnight cultures (three biological replicates) of Oscillibacter isolates (RJX3347, RJX3711, J115) and Bacteroides thetaiotaomicron VPI-5482 (control) were diluted to a starting O.D. 0.05 into fresh YCFAC media (Anaerobe Systems) supplemented with 150 μM cholesterol (Sigma-Aldrich, C8667-25G) or isotope-labeled cholesterol (chemical formula C2413C3H460, 13C labels at carbons 2, 3, and 4, Cambridge Isotope Laboratories, CLM-9139-PK). The cultures were grown at 37° C. in an anaerobic chamber (Coy Laboratory Products) with an atmosphere of 20% CO2, 2.5% H₂, and 75% N2 at 37° C. After 24 hours, 35 ml of each replicate and condition were collected and pelleted and 300 μl of the supernatant were removed and flash-frozen. After discarding the remaining supernatant, the pellets were washed once with cold PBS (pH 7.4) and pelleted again before storing at −80° C. for later submission for metabolomics analysis.

### Live-Cell Fluorescence Microscopy.

RJX3347, RJX3711, J115 and E. coli (RJX1193) were grown overnight in YCFAC media supplemented with 150 μM of the fluorescently-labeled TopFluor® Cholesterol (Avanti Polar lipids, 810255P-5 mg). One ml of each culture were centrifuged at 100 g to precipitate any insoluble TopFluor® Cholesterol particles, The supernatant was removed into fresh 1.5 ml microcentrifuges and centrifuged at 4000 g. Then, the supernatant was discarded, and the pellets were washed twice in pre-reduced PBS, before resuspending in PBS containing the membrane dye FM 4-64 (N-(3-Triethylammoniumpropyl)-4-(6-(4-(Diethylamino) Phenyl) Hexatrienyl) Pyridinium Dibromide) (Thermofisher, T13320) (10 μg/ml). 4 μl of the cell cultures were anaerobically spread on 2% (w/v) agarose pads prepared from PBS (pH 7.4) using a Frame-Seal (Bio-Rad, SLF0601). The pads were covered with a 22×22 mm #1 coverslip and sealed with VALAB (1:1:1 of vaseline, lanolin, and beeswax). Cells were imaged by an epifluorescence microscopy using a Nikon Ti2-E inverted microscope equipped with a CSU-W1 spinning disc confocal and Andor Zyla 4.2 sCMOS camera. A 100× 1.40 N.A. oil objective was used for imaging. Excitation/emission filters were FITC/TRITC for TopFluor® Cholesterol membrane imaging, respectively. Excitation light transmission was set to 40% for FITC with exposure time 300 ms and 50% for TRITC with exposure time 700 ms. Microscopy images were processed similarly by adjusting the brightness and contrast using the Fiji software.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A composition comprising or consisting of i) a CgT polypeptide or polynucleotide; ii) an Oscillibacter or Dysosmobacter IsmA polypeptide or polynucleotide; or iii) a combination of a CgT polypeptide or polynucleotide and an Oscillibacter or Dysosmobacter IsmA polypeptide or polynucleotide; and a pharmaceutically acceptable excipient.

2. The composition of claim 1, wherein the IsmA polypeptide has at least about 85%, 90%, or 95% amino acid sequence identity to a polypeptide encoded by Oscillibacter gene RJX3347_02204 or RJX3711_01778, or Dysosmobacter gene J115_02655 and the CgT polypeptide has at least 85%, 90%, or 95% amino acid sequence identity to an Oscillibacter or Dysosmobacter CgT polypeptide.

3. A composition comprising an effective amount of an IsmA polypeptide having at least 85% amino acid sequence identity to a polypeptide encoded by Oscillibacter gene RJX3347_02204 or RJX3711_01778, or Dysosmobacter gene J115_02655 and an effective amount of an CgT polypeptide having at least 85%, 90%, or 95% amino acid sequence identity to a polypeptide encoded by Oscillibacter gene RJX3347_02251 or Dysosmobacter gene J115 17675.

4. A composition comprising or consisting of an isolated Oscillibacter species and an excipient.

5. The composition of claim 4, wherein the Oscillibacter species expresses an IsmA polypeptide.

6. The composition of claim 4, wherein the Oscillibacter species expresses a CgT polypeptide.

7. The composition of claim 4, wherein the composition further comprises a Eubacterium species expressing an IsmA polypeptide.

8. The composition of claim 7, wherein the Eubacterium species is Eubacterium coprostanoligenes.

9. A composition comprising or consisting of an isolated Oscillibacter species and an isolated Eubacterium species each expressing IsmA.

10. The composition of claim 9, wherein the composition is formulated in a powder, bolus, gel, capsule, liquid, food stuff, or suppository.

11. A recombinant microbial cell wherein the cell comprises a heterologous polynucleotide encoding an IsmA polypeptide.

12. The recombinant microbial cell of claim 11, wherein the recombinant microbial cell is selected from the phyla Firmicutes, Bacteroidetes, Actinobacteria, Bacteroidetes, Proteobacteria, Fusobacteria, Verrucomicrobia, Euryarchaeota, and Ascomycota.

13. A composition comprising the recombinant microbial cell of claim 11, wherein the composition is formulated for delivery to the small intestine, the large intestine, the colon, or the rectum.

14. A therapeutic combination comprising the composition of claim 1, and a low density lipoprotein (LDL) cholesterol lowering agent.

15. The composition of claim 14, wherein the LDL cholesterol lowering agent is one or more of a statin, a cholesterol absorption inhibitor, a bile acid sequestrant, a PCSK9 inhibitor, an adenosine triphosphate-citrate lyase (ACL) inhibitor, or a microsomal triglyceride transfer protein (MTP) inhibitor.

16. A method of reducing plasma triglycerides, plasma cholesterol, and/or serum C-reactive protein in a subject, the method comprising administering to the subject the composition of claim 1.

17. A method of treating cardiovascular disease, cholesterol related disorders, or diseases associated with or characterized by increased levels of plasma triglycerides, plasma cholesterol, or serum C-reactive protein in a subject or reducing the propensity of the subject to develop cardiovascular disease, cholesterol related disorders, or diseases associated with or characterized by increased levels of plasma triglycerides, plasma cholesterol, or serum C-reactive protein, the method comprising administering to the subject the composition of claim 1.

18. A kit comprising the composition of claim 1, and instructions for their use in the treatment of cardiovascular disease, cholesterol related disorders, or diseases associated with or characterized by increased levels of plasma triglycerides, plasma cholesterol, or serum C-reactive protein.

19. An expression vector comprising a polynucleotide encoding an IsmA polypeptide or CgT polypeptide.

20. A method of treating hypercholesterolemia, cardiovascular disease, cholesterol related disorders, lowering plasma cholesterol, or diseases associated with or characterized by increased levels of plasma triglycerides, plasma cholesterol, or serum C-reactive protein in a subject, the method comprising administering to the subject the composition of claim 1, wherein the subject has previously been or is concurrently being administered an LDL cholesterol lowering agent.
